(19) United States
(12) Patent Application Publication
(10) Pub. No.: US 2018/0360940 A1
(43) Pub. Date: Dec. 20, 2018

(54) LISTERIA-BASED IMMUNOTHERAPY AND METHODS OF USE THEREOF

(71) Applicant: ADVAXIS, INC., Princeton, NJ (US)

(72) Inventors: Robert PETIT, Newtown, PA (US); Brandon CODER, Princeton, NJ (US)

(21) Appl. No.: 15/782,023

(22) PCT Filed: Dec. 16, 2016
(86) PCT No.: PCT/US2016/067331

§ 371 Date: Jun. 6, 2018

Related U.S. Application Data
(60) Provisional application No. 62/268,255, filed on Dec. 16, 2015.

(51) Int. Cl. A61K 39/00; A61P 35/04; A61K 39/12; C07K 14/025

(52) U.S. Cl. CPC A61K 39/001194; A61P 35/04; A61K 39/001106; A61K 39/12; A61K 2039/585; A61K 2039/523; A61K 2039/522; A61K 2039/55594; C07K 14/025

(57) ABSTRACT

The disclosure relates to the combined use of an immunotherapeutic composition comprising recombinant Listeria strains expressing a heterologous antigen fused to a truncated listeriolysin O (tLLO), a truncated ActA protein, or a PEST amino acid sequence and an antibiotic regimen, which may be sequentially administered in order to prevent the persistence, seeding of Listeria and/or formation of Listeria biofilms while allowing for an anti-tumor/anti-cancer or anti infectious disease immunotherapeutic response to take place. Disclosed are also methods of inducing an immune response, and treating, inhibiting, or suppressing cancer or tumors comprising administering the above composition.

### FIELD OF TECHNOLOGY

The disclosure relates to the combined use of an immunotherapeutic composition comprising recombinant Listeria strains expressing a heterologous antigen fused to a truncated listeriolysin O (tLLO), a truncated ActA protein, or a PEST amino acid sequence and an antibiotic regimen, which may be sequentially administered in order to prevent the persistence, seeding of Listeria and/or formation of Listeria biofilms while allowing for an anti-tumor/anti-cancer or anti infectious disease immunotherapeutic response to take place. Disclosed are also methods of inducing an immune response, and treating, inhibiting, or suppressing cancer or tumors comprising administering the above composition.

### BACKGROUND

A great deal of pre-clinical evidence and early clinical trial data suggests that the anti-tumor capabilities of the immune system can be harnessed to treat patients with established cancers. The vaccine strategy takes advantage of tumor antigens associated with various types of cancers Immunizing with live vaccines such as viral or bacterial vectors expressing a tumor-associated antigen is one strategy for eliciting strong CTL responses against tumors.

Listeria monocytogenes (Lm) is a gram positive, facultative intracellular bacterium that has direct access to the cytoplasm of antigen presenting cells, such as macrophages and dendritic cells, largely due to the pore-forming activity of listeriolysin-O (LLO). LLO is secreted by Lm following engulfment by the cells and perforates the phagolysosomal membrane, allowing the bacterium to escape the vacuole and enter the cytoplasm. LLO is very efficiently presented to the immune system via MHC class I molecules. Furthermore, Lm-derived peptides also have access to MHC class II presentation via the phagolysosome.

L. monocytogenes is able to attach to and colonize various surfaces, such as stainless steel, glass, and polystyrene, and to contaminate food products during processing. Biofilms of L. monocytogenes are associated with important ecological advantages, such as protection against biocide action. Several molecular determinants, such as flagella, biofilm-associated proteins (Bap), SecA2, and cell-cell communication systems, have been shown to be involved in biofilm construction within the species. While no exopolysaccharidic components have been evidenced in the L. monocytogenes biofilm matrix, extracellular DNA (eDNA) has been shown to participate in initial cellular adhesion and biofilm organization under specific growth conditions. Biofilm formation by the species is highly dependent on environmental conditions, such as variations in temperature, pH, and nutrients. L. monocytogenes is primarily an opportunistic pathogen that leads to 3 patterns of systemic infection: isolated bacteremia, central nervous system infection, and maternal-fetal infection. Although localized infections have seldom been reported, 36 bone and joint infections have been described in the literature, all as isolated case reports and some with literature reviews, and all of which may be biofilm associated. Further, the recent increase of sporadic and cluster-associated systemic listeriosis cases in Europe (since 2006 in France), particularly in the elderly who more frequently receive prosthetic joints, raised concern about the increase of bone and joint listeriosis cases.

Infectious diseases such as Malaria, Tuberculosis and HIV-1, or other chronic or latent viral infections, amongst others, remain tremendous disease burdens in much of the world's population. Despite decades of effort, there are no vaccines for malaria or HIV-1 and the same holds true for other chronic or latent viral infections. In addition, the majority of individuals in sub-Saharan countries, with prevalence exceeding 90% in many areas of Africa, are infected with one or more species of parasitic helminths that suppress immune responses, skew the host immune system of human and animals to T-helper type 2 (Th2), and suppress vaccine-specific responses. Hence, there exists a need to develop improved anti-infectious disease and anti-pararistic disease immunotherapies, including bacteria-based immunotherapies, with improved safety and efficacy.

Similarly, cancer and pre-malignant conditions leading to the same also remain a tremendous disease burden in the world's population. However, cancer is a very complex disease where each cancer requires a specific type of treatment and thus combined therapeutic approaches are more likely to succeed. Therefore, there is also a need to develop improved anti-tumor/anti-cancer bacteria-based immunotherapies with improved safety and efficacy. The present disclosure meets this need by providing a Listeria-based immunotherapy treatment modality that includes administration of an antibiotic regimen in order to eliminate Listeria persistence in the host and/or prevent the potential for biofilm formation in the same following treatment.

### SUMMARY

In one aspect, the disclosure relates to a method of preventing persistence of a Listeria strain on a tissue within a subject following administration of a Listeria-based immunotherapy regimen, the method comprising the step of administering an effective amount of a regimen of antibiotics following administration of the recombinant Listeria-based immunotherapy, thereby preventing the persistence of the Listeria strain within the subject.

In a related aspect, administering the antibiotic regimen prevents seeding or adherence of the Listeria strain. In another aspect, administering the antibiotic regimen prevents biofilm formation of the Listeria strain on a tissue within the subject. In another aspect, the antibiotic is poorly taken up within intact cells or is able to penetrate cells in order to clear intracellular bacteria.

In one aspect, a Listeria-based immunotherapy that is administered to a subject as part of the disclosed methods elicits an anti-disease immune response in the subject. In a related aspect, administration of the antibiotic regimen comprises administration after the anti-disease response has initiated. In another related aspect, administering of the antibiotic regimen does not interfere with the anti-disease immune response in the subject. In yet another related aspect, administering the antibiotic regimen clears the presence of the Listeria strain within the subject.

In one aspect, administering the antibiotic regimen comprises administration after a therapeutic goal resulting from the administration of the Listeria-based immunotherapy has been achieved. In a related aspect, the therapeutic goal comprises achieving an anti-disease immune response. In another related aspect, the therapeutic goal comprises achieving tumor or cancer regression.

Other features and advantages of the present disclosure will become apparent from the following detailed description examples and figures. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The subject matter regarded disclosed herein is particularly pointed out and distinctly claimed in the concluding portion of the specification. The disclosure, however, both as to organization and method of operation, together with objects, features, and advantages thereof, may best be understood by reference to the following detailed description when read with the accompanying drawings in which:

FIG. 1A-B. (FIG. 1A) Schematic representation of the chromosomal region of the Lmdd-143 and LmddA-143 after klk3 integration and actA deletion; (FIG. 1B) The klk3 gene is integrated into the Lmdd and LmddA chromosome. PCR from chromosomal DNA preparation from each construct using klk3 specific primers amplifies a band of 714 bp corresponding to the klk3 gene, lacking the secretion signal sequence of the wild type protein.

FIGS. 2A-D. (FIG. 2A) Map of the pADV134 plasmid. (FIG. 2B) Proteins from LmddA-134 culture supernatant were precipitated, separated in a SDS-PAGE, and the LLO-E7 protein detected by Western-blot using an anti-E7 monoclonal antibody. The antigen expression cassette consists of hly promoter, ORF for truncated LLO and human PSA gene (klk3). (FIG. 11C) Map of the pADV142 plasmid. (FIG. 2D) Western blot showed the expression of LLO-PSA fusion protein using anti-PSA and anti-LLO antibody.

FIGS. 3A-B. (FIG. 3A) Plasmid stability in vitro of LmddA-LLO-PSA if cultured with and without selection pressure (D-alanine). Strain and culture conditions are listed first and plates used for CFU determination are listed after. (FIG. 3B) Clearance of LmddA-LLO-PSA in vivo and assessment of potential plasmid loss during this time. Bacteria were injected i.v. and isolated from spleen at the time point indicated. CFUs were determined on BHI and BHI+D-alanine plates.

FIGS. 4A-B. (FIG. 4A) In vivo clearance of the strain LmddA-LLO-PSA after administration of 10⁸ CFU in C57BL/6 mice. The number of CFU were determined by plating on BHI/str plates. The limit of detection of this method was 100 CFU. (FIG. 4B) Cell infection assay of J774 cells with 10403S, LmddA-LLO-PSA and XFL7 strains.

FIGS. 5A-E. (FIG. 5A) PSA tetramer-specific cells in the splenocytes of naive and LmddA-LLO-PSA immunized mice on day 6 after the booster dose. (FIG. 5B) Intracellular cytokine staining for IFN-γ in the splenocytes of naive and LmddA-LLO-P SA immunized mice were stimulated with PSA peptide for 5 h. Specific lysis of EL4 cells pulsed with PSA peptide with in vitro stimulated effector T cells from LmddA-LLO-PSA immunized mice and naive mice at different effector/target ratio using a caspase based assay (FIG. 5C) and a europium based assay (FIG. 5D). Number of IFNγ spots in naive and immunized splenocytes obtained after stimulation for 24 h in the presence of PSA peptide or no peptide (FIG. 5E).

FIGS. 6A-C. Immunization with LmddA-142 induces regression of Tramp-C1-PSA (TPSA) tumors. Mice were left untreated (n=8) (FIG. 6A) or immunized i.p. with LmddA-142 (1×10⁸ CFU/mouse) (n=8) (FIG. 6B) or Lm-LLO-PSA (n=8), (FIG. 6C) on days 7, 14 and 21. Tumor sizes were measured for each individual tumor and the values expressed as the mean diameter in millimeters. Each line represents an individual mouse.

FIGS. 7A-B. (FIG. 7A) Analysis of PSA-tetramer⁺CD8⁺ T cells in the spleens and infiltrating T-PSA-23 tumors of untreated mice and mice immunized with either an Lm control strain or LmddA-LLO-PSA (LmddA-142). (FIG. 7B) Analysis of CD4⁺ regulatory T cells, which were defined as CD25⁺FoxP3⁺, in the spleens and infiltrating T-PSA-23 tumors of untreated mice and mice immunized with either an Lm control strain or LmddA-LLO-PSA.

FIGS. 8A-B. (FIG. 8A) Schematic representation of the chromosomal region of the Lmdd-143 and LmddA-143 after klk3 integration and actA deletion; (FIG. 8B) The klk3 gene is integrated into the Lmdd and LmddA chromosome. PCR from chromosomal DNA preparation from each construct using klk3 specific primers amplifies a band of 760 bp corresponding to the klk3 gene.

FIGS. 9A-C. (FIG. 9A) Lmdd-143 and LmddA-143 secretes the LLO-PSA protein. Proteins from bacterial culture supernatants were precipitated, separated in a SDS-PAGE and LLO and LLO-PSA proteins detected by Western-blot using an anti-LLO and anti-PSA antibodies; (FIG. 9B) LLO produced by Lmdd-143 and LmddA-143 retains hemolytic activity. Sheep red blood cells were incubated with serial dilutions of bacterial culture supernatants and hemolytic activity measured by absorbance at 590 nm; (FIG. 9C) Lmdd-143 and LmddA-143 grow inside the macrophage-like J774 cells. J774 cells were incubated with bacteria for 1 hour followed by gentamicin treatment to kill extracellular bacteria. Intracellular growth was measured by plating serial dilutions of J774 lysates obtained at the indicated timepoints. Lm 10403S was used as a control in these experiments.

FIG. 10. Immunization of mice with Lmdd-143 and LmddA-143 induces a PSA-specific immune response. C57BL/6 mice were immunized twice at 1-week interval with 1×10⁸ CFU of Lmdd-143, LmddA-143 or LmddA-142 and 7 days later spleens were harvested. Splenocytes were stimulated for 5 hours in the presence of monensin with 1 μM of the PSA₆₅₋₇₄ peptide. Cells were stained for CD8, CD3, CD62L and intracellular IFN-γ and analyzed in a FACS Calibur cytometer.

FIGS. 11A-B. Construction of ADXS31-164. (FIG. 11A) Plasmid map of pAdv164, which harbors bacillus subtilis dal gene under the control of constitutive Listeria p60 promoter for complementation of the chromosomal dal-dat deletion in LmddA strain. It also contains the fusion of truncated LLO₍₁₋₄₄₁₎ to the chimeric human Her2/neu gene, which was constructed by the direct fusion of 3 fragments the Her2/neu: EC1 (aa 40-170), EC2 (aa 359-518) and ICI (aa 679-808). (FIG. 11B) Expression and secretion of tLLO-ChHer2 was detected in Lm-LLO-ChHer2 (Lm-LLO-138) and LmddA-LLO-ChHer2 (ADXS31-164) by western blot analysis of the TCA precipitated cell culture supernatants blotted with anti-LLO antibody. A differential band of ˜104 KD corresponds to tLLO-ChHer2. The endogenous LLO is detected as a 58 KD band. Listeria control lacked ChHer2 expression.

FIGS. 12A-C. Immunogenic properties of ADXS31-164 (FIG. 12A) Cytotoxic T cell responses elicited by Her2/neu Listeria-based vaccines in splenocytes from immunized mice were tested using NT-2 cells as stimulators and 3T3/neu cells as targets. Lm-control was based on the LmddA background that was identical in all ways but expressed an irrelevant antigen (HPV16-E7). (FIG. 12B) IFN-γ secreted by the splenocytes from immunized FVB/N mice into the cell culture medium, measured by ELISA, after 24 hours of in vitro stimulation with mitomycin C treated NT-2 cells. (FIG. 12C) IFN-γ secretion by splenocytes from HLA-A2 transgenic mice immunized with the chimeric vaccine, in response to in vitro incubation with peptides from different regions of the protein. A recombinant ChHer2 protein was used as positive control and an irrelevant peptide or no peptide groups constituted the negative controls as listed in the figure legend. IFN-γ secretion was detected by an ELISA assay using cell culture supernatants harvested after 72 hours of co-incubation. Each data point was an average of triplicate data+/−standard error. *P value<0.001.

FIG. 13. Tumor Prevention Studies for Listeria-ChHer2/neu Vaccines Her2/neu transgenic mice were injected six times with each recombinant Listeria-ChHer2 or a control Listeria vaccine Immunizations started at 6 weeks of age and continued every three weeks until week 21. Appearance of tumors was monitored on a weekly basis and expressed as percentage of tumor free mice. *p<0.05, N=9 per group.

FIG. 14. Effect of immunization with ADXS31-164 on the % of Tregs in Spleens. FVB/N mice were inoculated s.c. with 1×10⁶ NT-2 cells and immunized three times with each vaccine at one week intervals. Spleens were harvested 7 days after the second immunization. After isolation of the immune cells, they were stained for detection of Tregs by anti CD3, CD4, CD25 and FoxP3 antibodies. Dot-plots of the Tregs from a representative experiment showing the frequency of CD25⁺/FoxP3⁺ T cells, expressed as percentages of the total CD3⁺ or CD3⁺CD4⁺ T cells across the different treatment groups.

FIGS. 15A-B. Effect of immunization with ADXS31-164 on the % of tumor infiltrating Tregs in NT-2 tumors. FVB/N mice were inoculated s.c. with 1×10⁶ NT-2 cells and immunized three times with each vaccine at one week intervals. Tumors were harvested 7 days after the second immunization. After isolation of the immune cells, they were stained for detection of Tregs by anti CD3, CD4, CD25 and FoxP3 antibodies. (FIG. 15A). Dot-plots of the Tregs from a representative experiment (FIG. 15B). Frequency of CD25⁺/FoxP3⁺ T cells, expressed as percentages of the total CD3⁺ or CD3⁺CD4⁺ T cells (left panel) and intratumoral CD8/Tregs ratio (right panel) across the different treatment groups. Data is shown as mean±SEM obtained from 2 independent experiments.

FIGS. 16A-C. Vaccination with ADXS31-164 can delay the growth of a breast cancer cell line in the brain. Balb/c mice were immunized thrice with ADXS31-164 or a control Listeria vaccine. EMT6-Luc cells (5,000) were injected intracranially in anesthetized mice. (FIG. 16A) Ex vivo imaging of the mice was performed on the indicated days using a Xenogen X-100 CCD camera. (FIG. 16B) Pixel intensity was graphed as number of photons per second per cm2 of surface area; this is shown as average radiance. (FIG. 16C) Expression of Her2/neu by EMT6-Luc cells, 4T1-Luc and NT-2 cell lines was detected by Western blots, using an anti-Her2/neu antibody. J774.A2 cells, a murine macrophage like cell line was used as a negative control.

FIGS. 17A-D Results from early time point administration of ampicillin at 2 hours, 4 hours, and 6 hours post-Lm vaccine. % PSA specific CD8 T cells (FIG. 17A), % SIINEFEKL specific CD8 T cells (FIG. 17B), # of PSA specific CD8 cells (FIG. 17C), and # of SIINEFEKL specific CD8 cells (FIG. 17D).

FIGS. 18A-B Splenocytes from early gentamicin treatment at 2 hours, 4 hours, or 6 hours +ampicillin 24 hour chase of LM-PSA-SVN treated mice. % PSA specific CD8 T cells (FIG. 18A) and % SIINEFEKL specific CD8 T cells (FIG. 18B).

FIG. 19 Lm treatment schedule for Example 17.

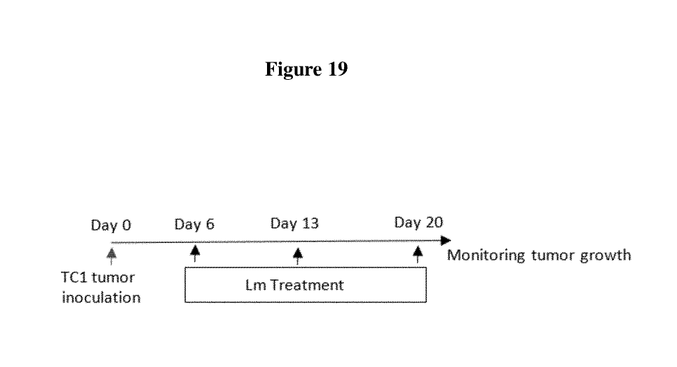

FIG. 20A-B Results for ADXS11-001 therapy with our without early ampicillin treatment. TC1 tumor regression (FIG. 20A) and % survival (FIG. 20B).

FIG. 21 Lm treatment schedule for Example 18.

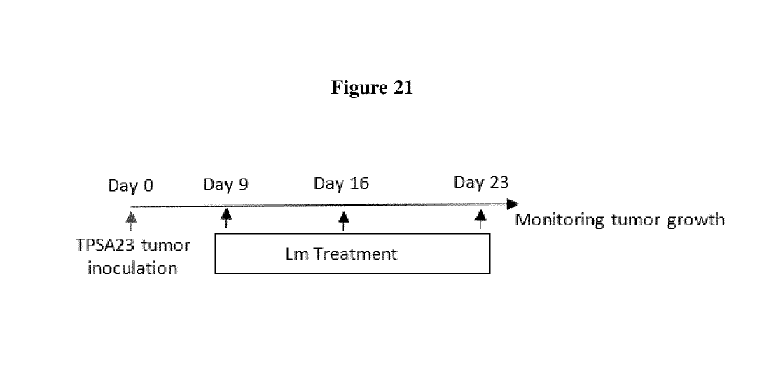

FIG. 22A-B Results for ADXS31-142 therapy with our without early ampicillin treatment. TC1 tumor regression (FIG. 22A) and % survival (FIG. 22B).

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

In the following detailed description, numerous specific details are set forth in order to provide a thorough understanding of the disclosure. However, it will be understood by those skilled in the art that the disclosure herein may be practiced without these specific details, as embodied herein. In other instances, well-known methods, procedures, and components have not been described in detail so as not to obscure the disclosure.

In one embodiment, disclosed herein is a method of preventing persistence of a Listeria strain on a tissue within a subject having a disease following administration of a Listeria-based immunotherapy regimen, the method comprising the step of administering an effective amount of a regimen of antibiotics following administration of the recombinant Listeria-based immunotherapy, thereby preventing the persistence of the Listeria strain within the subject.

In another embodiment, a Listeria strain disclosed herein comprises a nucleic acid molecule, the nucleic acid molecule comprises an open reading frame encoding a recombinant polypeptide, wherein the recombinant polypeptide comprises a heterologous antigen fused to an immunogenic protein or peptide.

In another embodiment, a Listeria strain disclosed herein comprises a nucleic acid molecule, the nucleic acid molecule comprises an open reading frame encoding a recombinant polypeptide, wherein the recombinant polypeptide comprises an immunogenic protein or peptide not fused to a heterologous antigen.

In another embodiment, a Listeria strain or Listeria-based immunotherapy regimen disclosed herein is described in PCT patent application numbers PCT/US2016/051748, PCT/US09/44538, PCT/US15/40911, PCT/US15/40855, PCT/US10/26257, PCT/US10/56534, PCT/US1²/₅1187, PCT/US2015/017559, PCT/US15/24048, PCT/US11/54613, PCT/US12/28757, PCT/US16/16452, PCT/US13/030521, PCT/US95/14741, PCT/US05/32682, PCT/US08/06048, PCT/US98/24357, PCT/US01/09736, PCT/US07/06292, PCT/US07/10635, PCT/US08/03067, PCT/US09/48085, PCT/US2004/000366, PCT/US2015/025690, PCT/US2015/016348, PCT/US15/18915, PCT/US15/55462, PCT/US15/40922, PCT/US15/66885, PCT/US15/40916, PCT/US15/55604, PCT/US2016/057220, PCT/US2015/066896, PCT/US2016/020571, PCT/US16/16455, PCT/US2016/032182, PCT/US2016/034301, PCT/US2016/052322, PCT/US05/28895, PCT/US08/04861, which are hereby incorporated by reference herein.

In another embodiment, an immunogenic protein or peptide disclosed herein comprises a truncated LLO protein, a truncated ActA protein or a PEST peptide.

In one embodiment, an antibiotic regimen disclosed herein prevents seeding or adherence of the Listeria strain to a tissue within a subject receiving an immunotherapy disclosed herein.

In one embodiment, administering an antibiotic regimen disclosed herein prevents persistence of a Listeria strain on a tissue within a subject. In one embodiment, administering an antibiotic regimen disclosed herein prevents seeding of a Listeria strain on a tissue within a subject. In another embodiment, administering an antibiotic regimen disclosed herein prevents biofilm formation of a Listeria strain on a tissue within a subject. In another embodiment, the Listeria strain is administered to the subject as part of a Listeria-based immunotherapy disclosed herein. In one embodiment, the subject has a disease. In another embodiment, the subject is a normal subject free from disease.

In another embodiment, a Listeria-based immunotherapy that is administered to a subject elicits an anti-disease immune response in said subject.

In one embodiment, an antibiotic regimen disclosed herein comprises administering at least one of the following: clindamycin, gentamicin, azithromycin, vancomycin, phosphomycin, linezolid, rifampicin, Meropenam Both, Bactrim, Moxifloxacin Both, minocycline, dapzone, trimethoprim/sulfa (Bactrim), telithromycin, pefloxacin, a beta-lactam, fusidic acid, a macrolide, a fluoroquinolone, ampicillin or any combination thereof. In one embodiment, an antibiotic regimen disclosed herein comprises administering at least ampicillin. In one embodiment, an antibiotic regimen disclosed herein comprises administering at least gentamicin. In one embodiment, an antibiotic regimen disclosed herein comprises administering at least ampicillin and gentamicin.

In one embodiment, administration of said antibiotic regimen to a subject comprises administration to the subject after an anti-disease immune response has initiated as a consequence of a Listeria-based immunotherapy that is administered to the subject. In another embodiment, administering of the antibiotic regimen does not interfere with an anti-disease immune response in the subject. In another embodiment, administration of the antibiotic regimen comprises administration after antigen presentation has taken place and following administration of a Listeria-based immunotherapy.

In one embodiment, an antibiotic disclosed herein is poorly taken up within intact cells in a subject. In one embodiment, an antibiotic that is poorly taken up within intact cells is referred to herein as an “extracellular antibiotic.” It will be appreciated by a skilled artisan that an extracellular antibiotic may encompass clindamycin, vancomycin, gentamycin, phosphomycin, azithromycin, linezolid or any others known in the art. It will be appreciated by a skilled artisan that the extracellular antibiotic is administered to a subject about 8 hours following administration of said recombinant Listeria-based immunotherapy and prior to seeding of a Listeria strain on a subject's tissue. In another embodiment, the extracellular antibiotic is administered within 1-2 hours following administration of said recombinant Listeria-based immunotherapy and prior to seeding of a Listeria strain on a subject's tissue. In another embodiment, the extracellular antibiotic is administered within 2-4 hours following administration of said recombinant Listeria-based immunotherapy and prior to seeding of a Listeria strain on a subject's tissue. In another embodiment, the extracellular antibiotic is administered within 4-6 hours following administration of said recombinant Listeria-based immunotherapy and prior to seeding of a Listeria strain on a subject's tissue. In another embodiment, the extracellular antibiotic is administered within 6-8 hours following administration of said recombinant Listeria-based immunotherapy and prior to seeding of a Listeria strain on a subject's tissue. In another embodiment, the extracellular antibiotic is administered 8-10 hours following administration of said recombinant Listeria-based immunotherapy and prior to seeding of a Listeria strain on a subject's tissue.

In one embodiment, an antibiotic administered to a subject following administration of a Listeria-based immunotherapy is able to penetrate cells in a subject in order to clear intracellular Listeria. In another embodiment, an antibiotic that is able to penetrate cells in a subject in order to clear intracellular bacteria such as Listeria is referred to herein as an “intracellular antibiotic.” It will be appreciated by a skilled artisan that an intracellular antibiotic may encompass rifampicin, beta-lactam, ampicillin, telithromycin, a macrolide, a fluoroquinolone or any others known in the art. It will also be appreciated by a skilled artisan that the intracellular antibiotic is administered to a subject about 8 hours following administration of a Listeria-based immunotherapy disclosed herein to clear all Listeria strains from the subject being treated. In another embodiment, the intracellular antibiotic is administered to a subject within 2-4 hours following administration of a Listeria-based immunotherapy disclosed herein to clear all Listeria strains from the subject being treated. In another embodiment, the intracellular antibiotic is administered to a subject within 4-6 hours following administration of a Listeria-based immunotherapy disclosed herein to clear all Listeria strains from the subject being treated. In another embodiment, the intracellular antibiotic is administered to a subject within 6-8 hours following administration of a Listeria-based immunotherapy disclosed herein to clear all Listeria strains from the subject being treated. In another embodiment, the intracellular antibiotic is administered to a subject within 8-10 hours following administration of a Listeria-based immunotherapy disclosed herein to clear all Listeria strains from the subject being treated. In another embodiment, the intracellular antibiotic is administered to a subject within 10-12 hours following administration of a Listeria-based immunotherapy disclosed herein to clear all Listeria strains from the subject being treated. In another embodiment, the intracellular antibiotic is administered to a subject within 12-14 hours following administration of a Listeria-based immunotherapy disclosed herein to clear all Listeria strains from the subject being treated. In another embodiment, the intracellular antibiotic is administered to a subject within 14-16 hours following administration of a Listeria-based immunotherapy disclosed herein to clear all Listeria strains from the subject being treated. In another embodiment, the intracellular antibiotic is administered to a subject within 16-18 hours following administration of a Listeria-based immunotherapy disclosed herein to clear all Listeria strains from the subject being treated. In another embodiment, the intracellular antibiotic is administered to a subject within 18-20 hours following administration of a Listeria-based immunotherapy disclosed herein to clear all Listeria strains from the subject being treated. In another embodiment, the intracellular antibiotic is administered to a subject within 20-22 hours following administration of a Listeria-based immunotherapy disclosed herein to clear all Listeria strains from the subject being treated. In another embodiment, the intracellular antibiotic is administered to a subject within 22-24 hours following administration of a Listeria-based immunotherapy disclosed herein to clear all Listeria strains from the subject being treated. In another embodiment, the intracellular antibiotic is administered to a subject within 24-48 hours following administration of a Listeria-based immunotherapy disclosed herein to clear all Listeria strains from the subject being treated. In another embodiment, the intracellular antibiotic is administered to a subject within 48-72 hours following administration of a Listeria-based immunotherapy disclosed herein to clear all Listeria strains from the subject being treated. In another embodiment, the intracellular antibiotic is administered to a subject until all Listeria strains are eradicated from the subject but after antigen has been presented by the Listeria strains in the subject.

In one embodiment, an extracellular antibiotic is administered on day 1 following administration of a Listeria-based immunotherapy and an extracellular antibiotic is administered thereafter on day 2, day 3, day 4, day 5, day 6 or day 7. It will be appreciated by a skilled artisan that repeat administration of an extracellular antibiotic after an initial administration and as needed, in order to clear Listeria strains from the subject, may be encompassed by and are contemplated by the methods disclosed herein. In one embodiment, administering an antibiotic that penetrate cells in a subject clears the presence of a Listeria strain within the subject. In one embodiment, administration of an antibiotic that penetrates cells in a subject is carried out after a therapeutic goal has been achieved using a Listeria-based immunotherapy disclosed herein. In another embodiment, a therapeutic goal comprises achieving an anti-disease immune response. In another embodiment, a therapeutic goal comprises achieving tumor or cancer regression.

In one embodiment, disclosed herein is a method of eliciting an anti-disease immune response in a subject, the method comprising the step of administering to said subject an effective amount of an immunogenic composition comprising a recombinant Listeria strain comprising a recombinant nucleic acid molecule, said nucleic acid molecule comprising a first open reading frame encoding a fusion polypeptide, wherein said fusion polypeptide comprises a truncated listeriolysin O (LLO) protein, a truncated ActA protein, or a PEST amino acid sequence fused to a heterologous antigen or an immunogenic fragment thereof, wherein each of said Listeria expresses a recombinant polypeptide, thereby eliciting an anti-disease immune response in said subject.

In one embodiment, disclosed herein is a method of eliciting an anti-disease immune response in a subject, the method comprising the step of administering to said subject an effective amount of an immunogenic composition comprising a recombinant Listeria strain comprising a recombinant nucleic acid molecule, said the nucleic acid molecule comprises an open reading frame encoding a recombinant polypeptide, wherein the recombinant polypeptide comprises a truncated listeriolysin O (LLO) protein, a truncated ActA protein, or a PEST amino acid sequence not fused to a heterologous antigen comprises, wherein each of said Listeria expresses a recombinant polypeptide, thereby eliciting an anti-disease immune response in said subject.

In another embodiment, disclosed herein is a method of eliciting an anti-disease immune response in a subject, the method comprising the step of administering to said subject an effective amount of an immunogenic composition comprising a recombinant Listeria strain, wherein said Listeria strain comprises a recombinant nucleic acid molecule, said nucleic acid molecule comprising a first open reading frame encoding a recombinant polypeptide, wherein said recombinant polypeptide comprises a truncated listeriolysin O (LLO) protein, a truncated ActA protein, or a PEST amino acid sequence fused to a heterologous antigen or an immunogenic fragment thereof, wherein said Listeria expresses said recombinant polypeptide, wherein said nucleic acid molecule comprises a second open reading frame encoding a metabolic enzyme, wherein said recombinant Listeria strain comprises mutations in endogenous genes encoding a D-alanine racemase (dal) and a D-amino acid transferase (dat) gene, and in a virulence gene encoding an ActA (actA) protein, thereby eliciting an anti-disease immune response in said subject.

In one embodiment, a heterologous antigen or fragment thereof comprises a neo-epitope, a PSA antigen, a chimeric HER2 antigen, an HPV strain 16 E7 or an HPV strain 18 E7, a mesothelin, an EGFRvIII, a NY-ESO-1 antigen or any combination thereof

In one embodiment, neoepitopes are generated and obtained as disclosed in any one of the following US applications (U.S. Ser. No. 62/166,591; U.S. Ser. No. 62/174,692; U.S. Ser. No. 62/218,936; U.S. Ser. No. 62/184,125.

In one embodiment, disclosed herein is a method of preventing persistence of a Listeria strain on a tissue within a subject following administration of a Listeria-based immunotherapy regimen, the method comprising the step of administering an effective amount of a regimen of antibiotics following administration of said recombinant Listeria-based immunotherapy, thereby preventing said persistence of said Listeria strain within said subject. In another embodiment, the antibiotic is administered to a subject within about 1 hour to about 8 hours following administration of a Listeria-based immunotherapy disclosed herein. In another embodiment, the antibiotic is administered to a subject within about 1 hour to about 6 hours following administration of a Listeria-based immunotherapy disclosed herein. In another embodiment, the antibiotic is administered to a subject within about 1 hour to about 4 hours following administration of a Listeria-based immunotherapy disclosed herein. In another embodiment, the antibiotic is administered to a subject within about 1 hour to about 12 hours following administration of a Listeria-based immunotherapy disclosed herein. In another embodiment, the antibiotic is administered to a subject within about 2 hours to about 8 hours following administration of a Listeria-based immunotherapy disclosed herein. In another embodiment, the antibiotic is administered to a subject within about 2 hours to about 6 hours following administration of a Listeria-based immunotherapy disclosed herein. In another embodiment, the antibiotic is administered to a subject within about 2 hours to about 4 hours following administration of a Listeria-based immunotherapy disclosed herein. In another embodiment, the antibiotic is administered to a subject within about 1 hour to about 24 hours following administration of a Listeria-based immunotherapy disclosed herein. In another embodiment, the antibiotic is administered to a subject within about 2 hours to about 24 hours following administration of a Listeria-based immunotherapy disclosed herein. In another embodiment, the antibiotic is administered to a subject within about 4 hours following administration of a Listeria-based immunotherapy disclosed herein. In another embodiment, the antibiotic is administered to a subject within about 8 hours following administration of a Listeria-based immunotherapy disclosed herein. In another embodiment, the antibiotic is administered to a subject within about 12 hours following administration of a Listeria-based immunotherapy disclosed herein.

In another embodiment, the Listeria strain comprises a nucleic acid molecule comprising an open reading frame encoding one or more peptides encoding one or more neoepitopes, wherein said one or more peptides are fused to an immunogenic protein or peptide. In another embodiment an immunogenic protein or peptide comprises a truncated LLO (tLLO), truncated ActA (tActA), or PEST amino acid sequence peptide.

In one embodiment, disclosed herein is a recombinant attenuated Listeria strain, wherein the Listeria strain comprises a nucleic acid sequence comprising one or more open reading frames encoding one or more peptides comprising one or more personalized neo-epitopes, wherein the neo-epitope(s) comprises immunogenic epitopes present in a disease or condition-bearing tissue or cell of a subject having the disease or condition. In another embodiment, one or more neoepitopes are present in a disease or condition-bearing tissue or cell of a subject having the disease or condition.

In another embodiment, administrating the Listeria strain to a subject having said disease or condition generates an immune response targeted to the subject's disease or condition.

In another embodiment, the strain is a personalized immunotherapy vector for said subject targeted to said subject's disease or condition.

In another embodiment, the peptides comprise at least two different neo-epitopes amino acid sequences.

In another embodiment, the peptides comprise one or more neo-epitopes repeats of the same amino acid sequence.

In another embodiment, the Listeria strain comprises one neo-epitope. In another embodiment, the Listeria strain comprises the neo-epitopes in the range of about 1-100. Alternativley, the Listeria strain comprises the neo-epitopes in the range of about 1-5, 5-10, 10-15, 15-20, 10-20, 20-30, 30-40,40-50, 50-60, 60-70, 70-80, 80-90, 90-100, 5-15, 5-20, 5-25, 15-20, 15-25, 15-30, 15-35, 20-25, 20-35, 20-45, 30-45, 30-55 ,40-55, 40-65, 50-65, 50-75, 60-75, 60-85, 70-85, 70-95, 80-95, 80-105 or 95-105. Alternativley, the Listeria strain comprises the neo-epitopes in the range of about 50-100. Alternativley, the Listeria strain comprises up to about 100 the neo-epitopes.

In another embodiment, the Listeria strain comprises above about 100 the neo-epitopes. In another embodiment, the Listeria strain comprises up to about 10 the neo-epitopes. In another embodiment, the Listeria strain comprises up to about 20 the neo-epitopes. In another embodiment, the Listeria strain comprises up to about 50 the neo-epitopes. Alternatively, the Listeria strain comprises about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 the neo-epitopes.

In one embodiment described herein, incorporation of amino acids in the range of about 5-30 amino acids flanking on each side of the detected mutation are generated. Additionally or alternatively, varying sizes of neo-epitope inserts are inserted in the range of about 8-27 amino acid sequence long. Additionally or alternatively, varying sizes of neo-epitope inserts are inserted in the range of about 5-50 amino acid sequence long.

In another embodiment, the neo-epitope sequences are tumor specific, metastases specific, bacterial infection specific, viral infection specific, and any combination thereof. Additionally or alternatively, the neo-epitope sequences are inflammation specific, immune regulation molecule epitope specific, T-cell specific, an autoimmune disease specific, Graft-versus-host disease (GvHD) specific, and any combination thereof.

In another embodiment, one or more neo-epitopes comprise linear neo-epitopes. Additionally or alternatively, one or more neo-epitopes comprise a solvent-exposed epitope.

In another embodiment, one or more neo-epitopes comprise a T-cell epitope.

In one embodiment, disclosed herein is a nucleic acid construct encoding a chimeric protein comprising the following elements: a N-terminal truncated LLO (tLLO) fused to a first neoepitope amino acid sequence, wherein said first neoepitope AA sequence is operatively linked to a second neoepitope AA sequence via a linker sequence, wherein said second neopitope AA sequence is operatively linked to at least one additional neoepitope amino acid sequence via a linker sequence, and wherein a last neoepitope is operatively linked to a histidine tag at the C-terminus via a linker sequence. In another embodiment, said elements are arranged or are operatively linked from N-terminus to C-terminus. In another embodiment, each nucleic acid construct comprises at least 1 stop codon following the sequence encoding said 6X histidine (HIS) tag. In another embodiment, each nucleic acid construct comprises 2 stop codonds following the sequence encoding said 6× histidine (HIS) tag. In another embodiment, said 6× histidine tag is operatively linked at the N-terminus to a SIINFEKL peptide. In another embodiment, said linker is a 4× glycine linker.

In another embodiment, the nucleic acid construct comprises at least one additional neoepitope amino acid sequence. In another embodiment, the nucleic acid construct comprises 2-10 additional neoepitopes, 10-15 additional neoepitopes, 10-25 additional neoepitopes, 25-40 additional neoepitopes, or 40-60 additional neoepitopes. In another embodiment, the nucleic acid construct comprises about 1-10, about 10-30, about 30-50, about 50-70, about 70-90, or up to about 100 neoepitopes. In another embodiment each neoepitope amino acid sequence is 1-10, 10-20, 20-30, or 30-40 amino acids long. In another embodiment, each neopitope amino acid sequence is 21 amino acids in length or is a “21 mer” neoepitope sequence.

In another embodiment, the nucleic acid construct encodes a recombinant polypeptide, chimeric protein or fusion polypeptide comprising an N-terminal truncated LLO fused to a 21 amino acid sequence of a neo-epitope flanked by a linker sequence and followed by at least one second neo epitope flanked by another linker and terminated by a SIINFEKL-6×His tag- and 2 stop codons closing the open reading frame: pHly-tLLO-21mer #1-4× glycine linker G1-21mer #2-4× glycine linker G2- . . . -SIINFEKL-6×His tag-2× stop codon. In another embodiment, expression of the above construct is driven by an hly promoter.

It will be appreciated by the skilled artisan that the term “nucleic acid” and grammatical equivalents thereof may refer to a molecule, which may include, but is not limited to, prokaryotic sequences, eukaryotic mRNA, cDNA from eukaryotic mRNA, genomic DNA sequences from eukaryotic (e.g., mammalian) DNA, and even synthetic DNA sequences. The term also refers to sequences that include any of the known base analogs of DNA and RNA.

In one embodiment, the compositions and methods of this disclosure are used for treating, preventing, inhibiting or suppressing a disease. In another embodiment, a disease disclosed herein comprises a tumor or cancer, an infectious disease, a premalignant condition, an autoimmune disease, or a metabolic disorder. It will be appreciated by the skilled artisan that the terms “cancer” and “tumor” may have all the same meanings and qualities.

In another embodiment, disclosed herein are compositions and methods for inducing an immune response against a tumor antigen. In another embodiment, the tumor antigen is a heterologous antigen. In another embodiment, the tumor antigen is a self-antigen. In another embodiment, provided herein are compositions and methods for inducing an immune response against an infectious disease antigen. In another embodiment, the infectious disease antigen is a heterologous antigen.

In another embodiment, an infectious disease comprises a parasitic infection, a bacteria infection, a chronic or latent viral infection.

In another embodiment, a disease disclosed herein comprises a neoplasia. In another embodiment, a disease disclosed herein comprises a dysplasia. In another embodiment, a disease disclosed herein comprises a non-malignant dysplastic conditions. In another embodiment, a disease disclosed herein comprises a cervical intraepithelial neoplasia (CIN), a vaginal intraepithelial neoplasia (VIN), or an anal intraepithelial neoplasia (AIN) or any other neoplasia known in the art.

In one embodiment, a premalignant condition comprises a dysplasia. In another embodiment, a premalignant condition comprises a neoplasia. In another embodiment,

In one embodiment, an immune response induced by the methods and compositions provided herein is a therapeutic one. In another embodiment it is a prophylactic immune response. In another embodiment, it is an enhanced immune response over methods available in the art for inducing an immune response in a subject afflicted with the diseases or conditions provided herein. In another embodiment, the immune response leads to clearance of the infectious disease afflicting the subject.

In one embodiment, the infectious disease is one caused by, but not limited to, any one of the following pathogens: leishmania, Entamoeba histolytica (which causes amebiasis), trichuris, BCG/Tuberculosis, Malaria, Plasmodium falciparum, plasmodium malariae, plasmodium vivax, Rotavirus, Cholera, Diptheria-Tetanus, Pertussis, Haemophilus influenzae, Hepatitis B, Human papilloma virus, Influenza seasonal), Influenza A (H1N1) Pandemic, Measles and Rubella, Mumps, Meningococcus A+C, Oral Polio Vaccines, mono, bi and trivalent, Pneumococcal, Rabies, Tetanus Toxoid, Yellow Fever, Bacillus anthracis (anthrax), Clostridium botulinum toxin (botulism), Yersinia pestis (plague), Variola major (smallpox) and other related pox viruses, Francisella tularensis (tularemia), Viral hemorrhagic fevers, Arenaviruses (LCM, Junin virus, Machupo virus, Guanarito virus, Lassa Fever), Bunyaviruses (Hantaviruses, Rift Valley Fever), Flaviruses (Dengue), Filoviruses (Ebola , Marburg), Burkholderia pseudomallei, Coxiella burnetii (Q fever), Brucella species (brucellosis), Burkholderia mallei (glanders), Chlamydia psittaci (Psittacosis), Ricin toxin (from Ricinus communis), Epsilon toxin of Clostridium perfringens, Staphylococcus enterotoxin B, Typhus fever (Rickettsia prowazekii), other Rickettsias, Food- and Waterborne Pathogens, Bacteria (Diarrheagenic E.coli, Pathogenic Vibrios, Shigella species, Salmonella BCG/, Campylobacter jejuni, Yersinia enterocolitica), Viruses (Caliciviruses, Hepatitis A, West Nile Virus, LaCrosse, California encephalitis, VEE, EEE, WEE, Japanese Encephalitis Virus, Kyasanur Forest Virus, Nipah virus, hantaviruses, Tickborne hemorrhagic fever viruses, Chikungunya virus, Crimean-Congo Hemorrhagic fever virus, Tickborne encephalitis viruses, Hepatitis B virus, Hepatitis C virus, Herpes Simplex virus (HSV), Human immunodeficiency virus (HIV), Human papillomavirus (HPV)), Protozoa (Cryptosporidium parvum, Cyclospora cayatanensis, Giardia lamblia, Entamoeba histolytica, Toxoplasma), Fungi (Microsporidia), Yellow fever, Tuberculosis, including drug-resistant TB, Rabies, Prions, Severe acute respiratory syndrome associated coronavirus (SARS-CoV), Coccidioides posadasii, Coccidioides immitis, Bacterial vaginosis, Chlamydia trachomatis, Cytomegalovirus, Granuloma inguinale, Hemophilus ducreyi, Neisseria gonorrhea, Treponema pallidum, Trichomonas vaginalis, or any other infectious disease known in the art that is not listed herein.

In one embodiment, the infectious disease is caused by a pathogenic protozoan or helminths. In another embodiment, pathogenic protozoans and helminths infections include: amebiasis; malaria; leishmaniasis; trypanosomiasis; toxoplasmosis; pneumocystis carinii; babesiosis; giardiasis; trichinosis; filariasis; schistosomiasis; nematodes; trematodes or flukes; and cestode (tapeworm) infections.

In another embodiment, the infectious disease is a livestock infectious disease. In another embodiment, livestock diseases can be transmitted to man and are called “zoonotic diseases.” In another embodiment, these diseases include, but are not limited to, Foot and mouth disease, West Nile Virus, rabies, canine parvovirus, feline leukemia virus, equine influenza virus, infectious bovine rhinotracheitis (IBR), pseudorabies, classical swine fever (CSF), IBR, caused by bovine herpesvirus type 1 (BHV-1) infection of cattle, and pseudorabies (Aujeszky's disease) in pigs, toxoplasmosis, anthrax, vesicular stomatitis virus, rhodococcus equi, Tularemia, Plague (Yersinia pestis), trichomonas.

It is to be understood that the methods disclosed herein may be used to treat any infectious disease, which in one embodiment, is bacterial, viral, parasitic, microbial, microorganism, pathogenic, or combination thereof, infection. In another embodiment, the methods of the present disclosure are for inhibiting or suppressing a bacterial, viral, parasitic, microbial, microorganism, pathogenic, or combination thereof, infection in a subject. In another embodiment, the present disclosure provides a method of eliciting a cytotoxic T-cell response against a bacterial, viral, parasitic, microbial, microorganism, pathogenic, or combination thereof, infection in a subject. In another embodiment, the present disclosure provides a method of inducing a Th1 immune response against a bacterial, viral, paratisic, microbial, microorganism, pathogenic, or combination thereof, infection in a Th1 unresponsive subject. In one embodiment, the infection is viral, which in one embodiment, is HIV. In one embodiment, the infection is bacterial, which in one embodiment, is mycobacteria, which in one embodiment, is tuberculosis. In one embodiment, the infection is eukaryotic, which in one embodiment, is plasmodium, which in one embodiment, is malaria. In one embodiment, the infectious disease or antigen used in the methods disclosed herein is any known in the art or any described in the following US applications (U.S. Ser. No. 13/876,810; U.S. Ser. No. 14/204,806; or U.S. Pat. No. 9,084,747, all of which are hereby incorporated by reference herein in their entirety.

A cancer that is the target of methods and compositions disclosed herein is, in another embodiment, a melanoma. In another embodiment, the cancer is a sarcoma. In another embodiment, the cancer is a carcinoma. In another embodiment, the cancer is a mesothelioma (e.g. malignant mesothelioma). In another embodiment, the cancer is a glioma. In another embodiment, the cancer is a germ cell tumor. In another embodiment, the cancer is a choriocarcinoma. In another embodiment, the cancer is pulmonary adenocarcinoma. In another embodiment, the cancer is colorectal adenocarcinoma. In another embodiment, the cancer is pulmonary squamous adenocarcinoma. In another embodiment, the cancer is gastric adenocarcinoma. In another embodiment, the cancer is an ovarian surface epithelial neoplasm (e.g. a benign, proliferative or malignant variety thereof). In another embodiment, the cancer is an oral squamous cell carcinoma. In another embodiment, the cancer is non small-cell lung carcinoma. In another embodiment, the cancer is an endometrial carcinoma. In another embodiment, the cancer is a prostate carcinoma. In another embodiment, the cancer is a non-small cell lung cancer (NSCLC). In another embodiment, the cancer is a hepatocellular carcinoma. In another embodiment, the cancer is a kaposis. In another embodiment, the cancer is a sarcoma. In another embodiment, the cancer is another carcinoma or sarcoma. In another embodiment, the cancer is a melanoma.

In another embodiment, a tumor or cancer disclosed herein is pancreatic tumor or cancer. In another embodiment, the tumor or cancer is ovarian tumor or cancer. In another embodiment, the tumor or cancer is gastric tumor or cancer. In another embodiment, the cancer is a carcinomatous lesion of the pancreas. In another embodiment, the tumor or cancer is a bladder tumor or cancer. In another embodiment, the tumor or cancer is a head and neck tumor or cancer. In another embodiment, the tumor or cancer is a colon tumor or cancer. In another embodiment, the tumor or cancer is a lung tumor or cancer. In another embodiment, the tumor or cancer is an ovarian tumor or cancer. In another embodiment, the tumor or cancer is an uterine tumor or cancer. In another embodiment, the tumor or cancer is a thyroid tumor or cancer. In another embodiment, the tumor or cancer is a thyroid tumor or cancer. In another embodiment, the tumor or cancer is a liver tumor or cancer. In another embodiment, the tumor or cancer is a renal tumor or cancer. In another embodiment, the cancer is a glioblastoma. In another embodiment, the tumor or cancer is an endometrial tumor or cancer. In another embodiment, the cancer is a metastasis. In one embodiment, the compositions and methods as disclosed herein can be used to treat solid tumors related to or resulting from any of the cancers as described hereinabove. In another embodiment, the tumor is a Wilms' tumor. In another embodiment, the tumor is a desmoplastic small round cell tumor. In another embodiment, the tumor or cancer is any other tumor or cancer known in the art.

In yet another embodiment, the compositions and methods of the present disclosure prevent the occurrence of escape mutations following treatment. In another embodiment, provided herein are compositions and methods for providing progression free survival to a subject suffering from a tumor or cancer. In another embodiment, disclosed herein are compositions and methods for immunizing a subject against a cancer or tumor. In another embodiment, disclosed herein are compositions and methods for immunizing a subject against a cancer or tumor. In another embodiment, the cancer is metastasis.

In another embodiment, the infectious disease is one caused by, but not limited to. any one of the following pathogens: BCG/Tuberculosis, Malaria, Plasmodium falciparum, plasmodium malariae, Plasmodium vivax, Rotavirus, Cholera, Diptheria-Tetanus, Pertussis, Haemophilus influenzae, Hepatitis B, Human papilloma virus, Influenza seasonal), influenza A (H1N1) Pandemic, Measles and Rubella, Mumps, Meningococcus A+C, Oral Polio Vaccines, mono, bi and trivalent, Pneumococcal, Rabies, Tetanus Toxoid, Yellow Fever, Bacillus anthracis (anthrax), Clostridium botulinum toxin (botulism), Yersinia pestis (plague), Variola major (smallpox) and other related pox viruses, Francisella tularensis (tularemia), Viral hemorrhagic fevers, Arenaviruses (LCM, Junin virus, Machupo virus, Guanarito virus, Lassa Fever), Bunyaviruses (Hantaviruses, Rift Valley Fever), Flaviruses (Dengue.), Filoviruses (Ebola Marburg), Burkholderia pseudomallei, Coxiella burnetii fever), Brucella species (brucellosis), Burkholderia mallei (glanders), Chlamydia psittaci (Psittacosis), Ricin toxin (from Ricinus communis), Epsilon toxin of Clostridium perfringens, Staphylococcus enterotoxin B, Typhus fever (Rickettsia prowazekii), other Rickettsias, Food- and Waterborne Pathogens, Bacteria (Diarrheagenic E.coli, Pathogenic Vibrios, Shigella species, Salmonella BCG/, Campylobacter jejuni, Yersinia enterocolitica), Viruses (Caliciviruses, Hepatitis A, West Nile Virus, LaCrosse, California encephalitis, VEE, EEE, WEE, Japanese Encephalitis Virus, Kyasanur Forest Virus, Nipah virus, hantaviruses, Tickbome hemorrhagic fever viruses, Chikungunya virus, Crimean-Congo Hemorrhagic fever virus, Tickborne encephalitis viruses, Hepatitis B virus, Hepatitis C virus, Herpes Simplex virus (HSV), Human immunodeficiency virus (HIV), Human papilloniavirus (HPV)), Protozoa (Cryptosporidium parvum, Cyclospora cayatanensis, Giardia. lamblia, Entamoeba histolytica, Toxoplasma), Fungi (Microsporidia), Yellow fever, Tuberculosis, including drug-resistant TB, Rabies, Prions, Severe acute respiratory syndrome associated coronavirus (SARS-CoV), Coccidioides posadasii, Coccidioides immitis, Bacterial is vaginosis, Chlamydia trachomatis, Cytomegalovirus, Granuloma inguinale, Hemophilus ducreyi, Neisseria gonorrhea, Treponema pallidum, Trichomonas vaginalis, or any other infectious disease known in the art that is not listed herein.

In another embodiment, a nucleic acid sequence encoding a recombinant polypeptide disclosed herein is cloned using DNA amplification methods such as polymerase chain reaction (PCR). In another embodiment, chemical synthesis is used to produce a single stranded oligonucleotide. This single stranded oligonucleotide is converted, in various embodiments, into double stranded DNA by hybridization with a complementary sequence, or by polymerization with a DNA polymerase using the single strand as a template. One of skill in the art would recognize that while chemical synthesis of DNA is limited to sequences of about 100 bases, longer sequences can be obtained by the ligation of shorter sequences. In another embodiment, subsequences are cloned and the appropriate subsequences cleaved using appropriate restriction enzymes. The fragments are then ligated to produce the desired DNA sequence.

In one embodiment, a nucleic acid sequence disclosed herein comprises a plasmid disclosed herein.

In one embodiment, nucleic acid sequences encoding recombinant polypeptides disclosed herein are transformed into a variety of host cells, including E. coli, other bacterial hosts, such as Listeria, yeast, and various higher eukaryotic cells such as the COS, CHO and HeLa cells lines and myeloma cell lines. In another embodiment, a nucleic acid sequence encoding a recombinant polypeptide disclosed herein is operably linked to appropriate expression control sequences for each host. Promoter/regulatory sequences are described in detail elsewhere herein. In another embodiment, a plasmid encoding a recombinant polypeptide disclosed herein further comprises additional promoter regulatory elements, as well as a ribosome binding site and a transcription termination signal. For eukaryotic cells, the control sequences will include a promoter and an enhancer derived from e g immunoglobulin genes, SV40, cytomegalovirus, etc., and a polyadenylation sequence. In another embodiment, the sequences include splice donor and acceptor sequences.

In one embodiment, the term “operably linked” means that the transcriptional and translational regulatory nucleic acid, is positioned relative to any coding sequences in such a manner that transcription is initiated. Generally, this will mean that the promoter and transcriptional initiation or start sequences are positioned 5′ to the coding region. In another embodiment, the term “operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences. In another embodiment, the term “operably linked” refers to the joining of several open reading frames in a transcription unit each encoding a protein or peptide so as to result in expression of a chimeric protein or polypeptide that functions as intended.

In one embodiment, the present disclosure provides a fusion polypeptide comprising a linker sequence. It will be understood by a skilled artisan that a “linker sequence” may encompass an amino acid sequence that joins two heterologous polypeptides, or fragments or domains thereof In general, a linker is an amino acid sequence that covalently links the polypeptides to form a fusion polypeptide. A linker typically includes the amino acids translated from the remaining recombination signal after removal of a reporter gene from a display vector to create a fusion protein comprising an amino acid sequence encoded by an open reading frame and the display protein. As appreciated by one of skill in the art, the linker can comprise additional amino acids, such as glycine and other small neutral amino acids.

In one embodiment, the term “recombinant polypeptide” and “fusion polypeptide” and grammatical variations thereof are used interchangeably herein. In another embodiment, recombinant or fusion polypeptides disclosed herein may be prepared by any suitable method, including, for example, cloning and restriction of appropriate sequences or direct chemical synthesis by methods discussed below. Alternatively, subsequences may be cloned and the appropriate subsequences cleaved using appropriate restriction enzymes. The fragments may then be ligated to produce the desired DNA sequence. In one embodiment, DNA encoding the antigen can be produced using DNA amplification methods, for example polymerase chain reaction (PCR). First, the segments of the native DNA on either side of the new terminus are amplified separately. The 5′ end of the one amplified sequence encodes the peptide linker, while the 3′ end of the other amplified sequence also encodes the peptide linker. Since the 5′ end of the first fragment is complementary to the 3′ end of the second fragment, the two fragments (after partial purification, e.g. on LMP agarose) can be used as an overlapping template in a third PCR reaction. The amplified sequence will contain codons, the segment on the carboxy side of the opening site (now forming the amino sequence), the linker, and the sequence on the amino side of the opening site (now forming the carboxyl sequence). The antigen is ligated into a plasmid.

It is to be understood by a skilled artisan that the terms “polypeptide” and “protein” have all the same meanings and qualifications for the intended purpose of their use herein. The terms “antigen,” “antigen peptide”, “antigenic polypeptide,” “antigen fragment,” or grammatical equivalents thereof are used interchangeably herein and, as will be appreciated by a skilled artisan, may encompass polypeptides, or peptides (including recombinant peptides) that are loaded onto and presented on MHC class I and/or class II molecules on a host's cell's surface and can be recognized or detected by an immune cell of the host, thereby leading to the mounting of an immune response against the polypeptide, peptide or cell presenting the same. Similarly, the immune response may also extend to other cells within the host, including diseased cells such as tumor or cancer cells that express the same polypeptides or peptides.

In one embodiment, an antigen may be foreign, that is, heterologous to the host and is referred to as a “heterologous antigen” herein. In another embodiment, a heterologous antigen is heterologous to a Listeria strain disclosed herein that recombinantly expresses said antigen. In another embodiment, a heterologous antigen is heterologous to the host and a Listeria strain disclosed herein that recombinantly expresses said antigen. In another embodiment, the antigen is a self-antigen, which is an antigen that is present in the host but the host does not elicit an immune response against it because of immunologic tolerance. It will be appreciated by a skilled artisan that a heterologous antigen as well as a self-antigen may encompass a tumor antigen, a tumor-associated antigen or an angiogenic antigen. In addition, a heterologous antigen may encompass an infectious disease antigen. In another embodiment, the terms “heterologous antigen,” “heterologous polypeptide,” and “antigenic polypeptide” are used interchangeably herein.

In one embodiment, the antigen from which the peptide disclosed herein is derived or which is comprised by a recombinant polypeptide disclosed herein is a tumor-associated antigen, which in one embodiment, is one of the following tumor antigens: a MAGE (Melanoma-Associated Antigen E) protein, e.g. MAGE 1, MAGE 2, MAGE 3, MAGE 4, a tyrosinase; a mutant ras protein; a mutant p53 protein; p97 melanoma antigen, a ras peptide or p53 peptide associated with advanced cancers; the HPV 16/18 antigens associated with cervical cancers, KLH antigen associated with breast carcinoma, CEA (carcinoembryonic antigen) associated with colorectal cancer, gp100, a MART1 antigen associated with melanoma, or the PSA antigen associated with prostate cancer. In another embodiment, the antigen for the compositions and methods as provided herein are melanoma-associated antigens, which in one embodiment are TRP-2, MAGE-1, MAGE-3, -100, tyrosinase, HSP-70, beta-HCG, or a combination thereof. Other tumor-associated antigens known in the art are also contemplated in the present disclosure.

In one embodiment, the peptide is derived from a chimeric Her2 antigen described in U.S. patent application Ser. No. 12/945,386, which is hereby incorporated by reference herein in its entirety. In one embodiment, the recombinant polypeptide comprises a chimeric Her2 antigen described in U.S. patent application Ser. No. 12/945,386, which is hereby incorporated by reference herein in its entirety.

In another embodiment, the peptide is derived from or the recombinant polypeptide comprises an antigen selected from a HPV-E7 (from either an HPV16 or HPV18 strain), a HPV-E6 (from either an HPV16 or HPV18 strain), Her-2/neu, NY-ESO-1, telomerase (TERT, SCCE, CEA, LMP-1, p53, carboxic anhydrase IX (CAIX), PSMA, a prostate stem cell antigen (PSCA), a HMW-MAA, WT-1, HIV-1 Gag, Proteinase 3, Tyrosinase related protein 2, PSA (prostate-specific antigen), EGFR-III, survivin, baculoviral inhibitor of apoptosis repeat-containing 5 (BIRCS), LMP-1, p53, PSMA, PSCA, Muc1, PSA (prostate-specific antigen), or a combination thereof.

In another embodiment, an HPV antigen disclosed hererin is one that is associated with papillomatous diseases (warts).

In one embodiment, the terms “recombinant Listeria” and “live-attenuated Listeria” are used interchangeably herein and refer to a Listeria comprising at least one attenuating mutation, deletion or inactivation that expresses one fusion protein of an antigen (PSA or cHER2) fused to a truncated LLO, truncated ActA or PEST amino acid sequence embodied herein. In another embodiment, a recombinant Listeria disclosed herein is a recombinant Listeria monocytogenes.

It will also be appreciated by a skilled artisan that the terms “antigenic portion thereof”, “a fragment thereof” and “immunogenic portion thereof” in regard to a protein, peptide or polypeptide are used interchangeably herein and may encompass a protein, polypeptide, peptide, including recombinant forms thereof comprising a domain or segment that leads to the mounting of an immune response when present in, or, in some embodiments, detected by, a host, either alone, or in the context of a fusion protein, as described herein.

The terms “nucleic acid,” “nucleotide,” “polynucleotide,” “nucleic acid sequence,” “nucleic acid molecule,” “oligonucleotide,” or “nucleotide molecule” are used interchangeably herein and may encompass a string of at least two base-sugar-phosphate combinations, as will be appreciated by a skilled artisan. The terms include, in one embodiment, DNA and RNA. It will be appreciated by the skilled artisan that the term “nucleic acid” and grammatical equivalents thereof may refer to a molecule, which may include, but is not limited to, prokaryotic sequences, eukaryotic mRNA, cDNA from eukaryotic mRNA, genomic DNA sequences from eukaryotic (e.g., mammalian) DNA, and even synthetic DNA sequences. The term also refers to sequences that include any of the known base analogs of DNA and RNA. It will also be appreciated by a skilled artisan that the terms may encompass the monomeric units of nucleic acid polymers. For example, RNA may be in the form of a tRNA (transfer RNA), snRNA (small nuclear RNA), rRNA (ribosomal RNA), mRNA (messenger RNA), anti-sense RNA, small inhibitory RNA (siRNA), micro RNA (miRNA) and ribozymes. The use of siRNA and miRNA has been described (Caudy A A et al, Genes & Devel 16: 2491-96 and references cited therein). DNA may be in form of plasmid DNA, viral DNA, linear DNA, or chromosomal DNA or derivatives of these groups. In addition, these forms of DNA and RNA may be single, double, triple, or quadruple stranded. The term may also encompass artificial nucleic acids that may contain other types of backbones but the same bases. The use of phosphothiorate nucleic acids and PNA are known to those skilled in the art, and are described in, for example, Neilsen P E, Curr Opin Struct Biol 9:353-57; and Raz N K et al Biochem Biophys Res Commun. 297:1075-84. The production and use of nucleic acids is known to those skilled in art and is described, for example, in Molecular Cloning, (2001), Sambrook and Russell, eds. and Methods in Enzymology: Methods for molecular cloning in eukaryotic cells (2003) Purchio and G. C. Fareed.

The terms “amino acid” or “amino acids” are understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” may include both D- and L-amino acids.

It will be appreciated by a skilled artisan that the term “open reading frame” or “ORF” may encompass a portion of an organism's genome which contains a sequence of bases that could potentially encode a protein. In another embodiment, the start and stop ends of the ORF are not equivalent to the ends of the mRNA, but they are usually contained within the mRNA. In one embodiment, ORFs are located between the start-code sequence (initiation codon) and the stop-codon sequence (termination codon) of a gene. Thus, in one embodiment, a nucleic acid molecule operably integrated into a genome as an open reading frame with an endogenous polypeptide is a nucleic acid molecule that has integrated into a genome in the same open reading frame as an endogenous polypeptide.

It will be appreciated by a skilled artisan that the term “endogenous” may encompass an item that has developed or originated within the reference organism or arisen from causes within the reference organism. For example, endogenous refers to native.

It will also be appreciated by a skilled artisan that the term “fragment” when in refernce to proteins/polypeptides may encompass a protein or polypeptide that is shorter or comprises fewer amino acids than the full length protein or polypeptide. In one embodiment, a fragment is an N-terminal fragment. In another embodiment, a fragment is a C-terminal fragment. In yet another embodiment, a fragment is an intrasequential section of the protein or peptide. It will be understood by a skilled artisan that a fragment disclosed herein is a functional fragment, which may encompass an immunogenic fragment. In one embodiment, a fragment has more than 5 amino acids. In another embodiment, a fragment has 10-20 amino acids, 20-50 amino acids, 50-100 amino acids, 100-200 amino acids, 200-350 amino acids, or 350-500 amino acids.

In an alternate embodiment, the term “fragment” refers to a nucleic acid sequence or amino acid sequence that is shorter or comprises fewer nucleotides or amino acids than the full length nucleic acid molecule or full length protein. In one embodiment, a fragment is a 5′-terminal fragment or N-terminal fragment (for proteins). In another embodiment, a fragment is a 3′-terminal fragment or C-terminal fragment (for proteins). In yet another embodiment, a fragment encodes an intrasequential section of the nucleic acid molecule or protein. In one embodiment, a fragment has more than 5 nucleotides or amino acid sequences. In another embodiment, a fragment has 10-20 nucleotides or amino acid sequences, 20-50 nucleotides or amino acid sequences, 50-100 nucleotides or amino acid sequences, 100-200 nucleotides or amino acid sequences, 200-350 nucleotides or amino acid sequences, 350-500 or 500-1000 nucleotides or amino acid sequences. In one embodiment, a fragment is an intrasequential section of the protein or peptide. It will be understood by a skilled artisan that a fragment disclosed herein is a functional fragment, which may encompass an immunogenic fragment. It will be appreciated by a skilled artisan that the term “functional” within the meaning of the disclosure, may encompass the innate ability of a protein, peptide, nucleic acid, fragment or a variant thereof to exhibit a biological activity. Such a biological activity may encompass having the potential to elicit an immune response when used as disclosed herein, an illustration of which may be to be used as part of a fusion protein). Such a biological function may encompass its binding property to an interaction partner, e.g., a membrane-associated receptor, or its trimerization property. In the case of functional fragments and the functional variants of the disclosure, these biological functions may in fact be changed, e.g., with respect to their specificity or selectivity, but with retention of the basic biological function.

It will be appreciated by a skilled artisan that the terms “fragment” or “functional fragment” may encompass an immunogenic fragment that is capable of eliciting an immune response when administered to a subject alone or as part of a pharmaceutical composition comprising a recombinant Listeria strain expressing said immunogenic fragment. In another embodiment, a functional fragment has biological activity as will be understood by a skilled artisan and as further disclosed herein.

In another embodiment, the recombinant nucleic acid backbone of a plasmid disclosed herein comprises SEQ ID NO: 1.

(SEQ ID NO: 1)
ggagtgtatactggcttactatgttggcactgatgagggtgtcagtgaag
tgcttcatgtggcaggagaaaaaaggctgcaccggtgcgtcagcagaata
tgtgatacaggatatattccgcttcctcgctcactgactcgctacgctcg
gtcgttcgactgcggcgagcggaaatggcttacgaacggggcggagattt
cctggaagatgccaggaagatacttaacagggaagtgagagggccgcggc
aaagccgtttttccataggctccgcccccctgacaagcatcacgaaatct
gacgctcaaatcagtggtggcgaaacccgacaggactataaagataccag
gcgtttccccctggcggctccctcgtgcgctctcctgttcctgcctttcg
gtttaccggtgtcattccgctgttatggccgcgtttgtctcattccacgc
ctgacactcagttccgggtaggcagttcgctccaagctggactgtatgca
cgaaccccccgttcagtccgaccgctgcgccttatccggtaactatcgtc
ttgagtccaacccggaaagacatgcaaaagcaccactggcagcagccact
ggtaattgatttagaggagttagtcttgaagtcatgcgccggttaaggct
aaactgaaaggacaagttttggtgactgcgctcctccaagccagttacct
cggttcaaagagttggtagctcagagaaccttcgaaaaaccgccctgcaa
ggcggttttttcgttttcagagcaagagattacgcgcagaccaaaacgat
ctcaagaagatcatcttattaatcagataaaatatttctagccctccttt
gattagtatattcctatcttaaagttacttttatgtggaggcattaacat
ttgttaatgacgtcaaaaggatagcaagactagaataaagctataaagca
agcatataatattgcgtttcatctttagaagcgaatttcgccaatattat
aattatcaaaagagaggggtggcaaacggtatttggcattattaggttaa
aaaatgtagaaggagagtgaaacccatgaaaaaaataatgctagttttta
ttacacttatattagttagtctaccaattgcgcaacaaactgaagcaaag
gatgcatctgcattcaataaagaaaattcaatttcatccatggcaccacc
agcatctccgcctgcaagtcctaagacgccaatcgaaaagaaacacgcgg
atgaaatcgataagtatatacaaggattggattacaataaaaacaatgta
ttagtataccacggagatgcagtgacaaatgtgccgccaagaaaaggtta
caaagatggaaatgaatatattgttgtggagaaaaagaagaaatccatca
atcaaaataatgcagacattcaagttgtgaatgcaatttcgagcctaacc
tatccaggtgctctcgtaaaagcgaattcggaattagtagaaaatcaacc
agatgttctccctgtaaaacgtgattcattaacactcagcattgatttgc
caggtatgactaatcaagacaataaaatagttgtaaaaaatgccactaaa
tcaaacgttaacaacgcagtaaatacattagtggaaagatggaatgaaaa
atatgctcaagcttatccaaatgtaagtgcaaaaattgattatgatgacg
aaatggcttacagtgaatcacaattaattgcgaaatttggtacagcattt
aaagctgtaaataatagcttgaatgtaaacttcggcgcaatcagtgaagg
gaaaatgcaagaagaagtcattagttttaaacaaatttactataacgtga
atgttaatgaacctacaagaccttccagatttttcggcaaagctgttact
aaagagcagttgcaagcgcttggagtgaatgcagaaaatcctcctgcata
tatctcaagtgtggcgtatggccgtcaagtttatttgaaattatcaacta
attcccatagtactaaagtaaaagctgcttttgatgctgccgtaagcgga
aaatctgtctcaggtgatgtagaactaacaaatatcatcaaaaattcttc
cttcaaagccgtaatttacggaggttccgcaaaagatgaagttcaaatca
tcgacggcaacctcggagacttacgcgatattttgaaaaaaggcgctact
tttaatcgagaaacaccaggagttcccattgcttatacaacaaacttcct
aaaagacaatgaattagctgttattaaaaacaactcagaatatattgaaa
caacttcaaaagcttatacagatggaaaaattaacatcgatcactctgga
ggatacgttgctcaattcaacatttcttgggatgaagtaaattatgatct
cgagactagttctagatttatcacgtacccatttccccgcatcttttatt
tttttaaatactttagggaaaaatggtttttgatttgcttttaaaggttg
tggtgtagactcgtctgctgactgcatgctagaatctaagtcactttcag
aagcatccacaactgactctttcgccacttttctcttatttgcttttgtt
ggtttatctggataagtaaggctttcaagctcactatccgacgacgctat
ggcttttcttctttttttaatttccgctgcgctatccgatgacagacctg
gatgacgacgctccacttgcagagttggtcggtcgactcctgaagcctct
tcatttatagccacatttcctgtttgctcaccgttgttattattgttatt
cggacctttctctgcttttgctttcaacattgctattaggtctgctttgt
tcgtatttttcactttattcgatttttctagttcctcaatatcacgtgaa
cttacttcacgtgcagtttcgtatcttggtcccgtatttacctcgcttgg
ctgctcttctgttttttcttcttcccattcatctgtgtttagactggaat
cttcgctatctgtcgctgcaaatattatgtcggggttaatcgtaatgcag
ttggcagtaatgaaaactaccatcatcgcacgcataaatctgtttaatcc
cacttatactccctcctcgtgatacgctaatacaacctttttagaacaag
gaaaattcggccttcattttcactaatttgttccgttaaaaattggatta
gcagttagttatcttcttaattagctaatataagaaaaaatattcatgaa
ttattttaagaatatcacttggagaattaatttttctctaacatttgtta
atcagttaaccccaactgcttcccaagcttcacccgggccactaactcaa
cgctagtagtggatttaatcccaaatgagccaacagaaccagaaccagaa
acagaacaagtaacattggagttagaaatggaagaagaaaaaagcaatga
tttcgtgtgaataatgcacgaaatcattgcttatttttttaaaaagcgat
atactagatataacgaaacaacgaactgaataaagaatacaaaaaaagag
ccacgaccagttaaagcctgagaaactttaactgcgagccttaattgatt
accaccaatcaattaaagaagtcgagacccaaaatttggtaaagtattta
attactttattaatcagatacttaaatatctgtaaacccattatatcggg
tttttgaggggatttcaagtctttaagaagataccaggcaatcaattaag
aaaaacttagttgattgccttttttgttgtgattcaactttgatcgtagc
ttctaactaattaattttcgtaagaaaggagaacagctgaatgaatatcc
cttttgttgtagaaactgtgcttcatgacggcttgttaaagtacaaattt
aaaaatagtaaaattcgctcaatcactaccaagccaggtaaaagtaaagg
ggctatttttgcgtatcgctcaaaaaaaagcatgattggcggacgtggcg
ttgttctgacttccgaagaagcgattcacgaaaatcaagatacatttacg
cattggacaccaaacgtttatcgttatggtacgtatgcagacgaaaaccg
ttcatacactaaaggacattctgaaaacaatttaagacaaatcaatacct
tctttattgattttgatattcacacggaaaaagaaactatttcagcaagc
gatattttaacaacagctattgatttaggttttatgcctacgttaattat
caaatctgataaaggttatcaagcatattttgttttagaaacgccagtct
atgtgacttcaaaatcagaatttaaatctgtcaaagcagccaaaataatc
tcgcaaaatatccgagaatattttggaaagtctttgccagttgatctaac
gtgcaatcattttgggattgctcgtataccaagaacggacaatgtagaat
tttttgatcccaattaccgttattctttcaaagaatggcaagattggtct
ttcaaacaaacagataataagggctttactcgttcaagtctaacggtttt
aagcggtacagaaggcaaaaaacaagtagatgaaccctggtttaatctct
tattgcacgaaacgaaattttcaggagaaaagggtttagtagggcgcaat
agcgttatgtttaccctctctttagcctactttagttcaggctattcaat
cgaaacgtgcgaatataatatgtttgagtttaataatcgattagatcaac
ccttagaagaaaaagaagtaatcaaaattgttagaagtgcctattcagaa
aactatcaaggggctaatagggaatacattaccattctttgcaaagcttg
ggtatcaagtgatttaaccagtaaagatttatttgtccgtcaagggtggt
ttaaattcaagaaaaaaagaagcgaacgtcaacgtgttcatttgtcagaa
tggaaagaagatttaatggcttatattagcgaaaaaagcgatgtatacaa
gccttatttagcgacgaccaaaaaagagattagagaagtgctaggcattc
ctgaacggacattagataaattgctgaaggtactgaaggcgaatcaggaa
attttctttaagattaaaccaggaagaaatggtggcattcaacttgctag
tgttaaatcattgttgctatcgatcattaaattaaaaaaagaagaacgag
aaagctatataaaggcgctgacagcttcgtttaatttagaacgtacattt
attcaagaaactctaaacaaattggcagaacgccccaaaacggacccaca
actcgatttgtttagctacgatacaggctgaaaataaaacccgcactatg
ccattacatttatatctatgatacgtgtttgtttttctttgctggctagc
ttaattgcttatatttacctgcaataaaggatttcttacttccattatac
tcccattttccaaaaacatacggggaacacgggaacttattgtacaggcc
acctcatagttaatggtttcgagccttcctgcaatctcatccatggaaat
atattcatccccctgccggcctattaatgtgacttttgtgcccggcggat
attcctgatccagctccaccataaattggtccatgcaaattcggccggca
attttcaggcgttttcccttcacaaggatgtcggtccctttcaattttcg
gagccagccgtccgcatagcctacaggcaccgtcccgatccatgtgtctt
tttccgctgtgtactcggctccgtagctgacgctctcgccttttctgatc
agtttgacatgtgacagtgtcgaatgcagggtaaatgccggacgcagctg
aaacggtatctcgtccgacatgtcagcagacgggcgaaggccatacatgc
cgatgccgaatctgactgcattaaaaaagccttttttcagccggagtcca
gcggcgctgttcgcgcagtggaccattagattctttaacggcagcggagc
aatcagctctttaaagcgctcaaactgcattaagaaatagcctctttctt
tttcatccgctgtcgcaaaatgggtaaatacccctttgcactttaaacga
gggttgcggtcaagaattgccatcacgttctgaacttcttcctctgtttt
tacaccaagtctgttcatccccgtatcgaccttcagatgaaaatgaagag
aaccttttttcgtgtggcgggctgcctcctgaagccattcaacagaataa
cctgttaaggtcacgtcatactcagcagcgattgccacatactccggggg
aaccgcgccaagcaccaatataggcgccttcaatccctttttgcgcagtg
aaatcgcttcatccaaaatggccacggccaagcatgaagcacctgcgtca
agagcagcctttgctgtttctgcatcaccatgcccgtaggcgtttgcttt
cacaactgccatcaagtggacatgttcaccgatatgttttttcatattgc
tgacattttcctttatcacggacaagtcaatttccgcccacgtatctctg
taaaaaggttttgtgctcatggaaaactcctctcttttttcagaaaatcc
cagtacgtaattaagtatttgagaattaattttatattgattaatactaa
gtttacccagttttcacctaaaaaacaaatgatgagataatagctccaaa
ggctaaagaggactataccaactatttgttaat.

In one embodiment, a recombinant Listeria strain disclosed herein comprises a full length LLO polypeptide, which in one embodiment, is hemolytic. In another embodiment, the recombinant Listeria strain comprises a non-hemolytic LLO polypeptide. In another embodiment, the polypeptide is an LLO fragment. In another embodiment, the polypeptide is a truncated LLO. In another embodiment, the oligopeptide is a complete LLO protein. In another embodiment, the polypeptide is any LLO protein or fragment thereof known in the art.

It will be appreciated by a skilled artisan that the terms “N-terminal LLO protein,” “LLO fragment” and “truncated LLO (tLLO)” have all the same meanings and qualifications for the intended purpose of their use herein and as such are used interchangeably herein.

In another embodiment, an LLO protein fragment is utilized in compositions and methods as disclosed herein. In one embodiment, a truncated LLO protein is encoded by the episomal expression vector as disclosed herein that expresses a polypeptide, that is, in one embodiment, an antigen, in another embodiment, an angiogenic factor, or, in another embodiment, both an antigen and angiogenic factor. In another embodiment, the LLO fragment is an N-terminal fragment.

In one embodiment, an amino acid sequence of a truncated LLO (tLLO) comprises SEQ ID NO: 2:

(SEQ ID NO: 2)
M K K I M L V F I T L I L V S L P I A Q Q T E A K
D A S A F N K E N S I S S M A P P A S P P A S P K
T P I E K K H A D E I D K Y I Q G L D Y N K N N V
L V Y H G D A V T N V P P R K G Y K D G N E Y I V
V E K K K K S I N Q N N A D I Q V V N A I S S L T
Y P G A L V K A N S E L V E N Q P D V L P V K R D
S L T L S I D L P G M T N Q D N K I V V K N A T K
S N V N N A V N T L V E R W N E K Y A Q A Y P N V
S A K I D Y D D E M A Y S E S Q L I A K F G T A F
K A V N N S L N V N F G A I S E G K M Q E E V I S
F K Q I Y Y N V N V N E P T R P S R F F G K A V T
K E Q L Q A L G V N A E N P P A Y I S S V A Y G R
Q V Y L K L S T N S H S T K V K A A F D A A V S G
K S V S G D V E L T N I I K N S S F K A V I Y G G
S A K D E V Q I I D G N L G D L R D I L K K G A T
F N R E T P G V P I A Y T T N F L K D N E L A V I
K N N S E Y I E T T S K A Y T D G K I N I D H S G
G Y V A Q F N I S W D E V N Y D.

In another embodiment, the LLO fragment comprises the sequence: MKKIMLVFITLILVSLPIAQQTEAKDASAFNKENSISSVAPPASPPASPKTPIEKKHADEI DKYIQGLDYNKNNVLVYHGDAVTNVPPRKGYKDGNEYIVVEKKKKSINQNNADIQV VNAISSLTYPGALVKANSELVENQPDVLPVKRDSLTLSIDLPGMTNQDNKIVVKNAT KSNVNNAVNTLVERWNEKYAQAYSNVSAKIDYDDEMAYSESQLIAKFGTAFKAVN NSLNVNFGAISEGKMQEEVISFKQIYYNVNVNEPTRPSRFFGKAVTKEQLQALGVNA ENPPAYISSVAYGRQVYLKLSTNSHSTKVKAAFDAAVSGKSVSGDVELTNIIKNSSFK AVIYGGSAKDEVQIIDGNLGDLRDILKKGATFNRETPGVPIAYTTNFLKDNELAVIKN NSEYIETTSKAYTD (SEQ ID NO: 3). In another embodiment, an LLO AA sequence of methods and compositions as disclosed herein comprises the sequence set forth in SEQ ID No: 3. In another embodiment, the LLO AA sequence is a homologue of SEQ ID No: 3. In another embodiment, the LLO AA sequence is a variant of SEQ ID No: 3. In another embodiment, the LLO AA sequence is a fragment of SEQ ID No: 3. In another embodiment, the LLO AA sequence is an isoform of SEQ ID No: 3.

In one embodiment, the LLO protein used in the compositions and methods as disclosed herein comprises the following sequence:

MKKIMLVFITLILVSLPIAQQTEAKDASAFNKENSISSMAPPASPPASPKTPIEK KHADEIDKYIQGLDYNKNNVLVYHGDAVTNVPPRKGYKDGNEYIVVEKKKKSINQ NNADIQVVNAISSLTYPGALVKANSELVENQPDVLPVKRDSLTLSIDLPGMTNQDNK IVVKNATKSNVNNAVNTLVERWNEKYAQAYPNVSAKIDYDDEMAYSESQLIAKFG TAFKAVNNSLNVNFGAISEGKMQEEVISFKQIYYNVNVNEPTRPSRFFGKAVTKEQL QALGVNAENPPAYISSVAYGRQVYLKLSTNSHSTKVKAAFDAAVSGKSVSGDVELT NIIKNSSFKAVIYGGSAKDEVQIIDGNLGDLRDILKKGATFNRETPGVPIAYTTNFLKD NELAVIKNNSEYIETTSKAYTDGKINIDHSGGYVAQFNISWDEVNYDPEGNEIVQHK NWSENNKSKLAHFTSSIYLPGNARNINVYAKECTGLAWEWWRTVIDDRNLPLVKNR NISIWGTTLYPKYSNKVDNPIE (GenBank Accession No. P13128; SEQ ID NO: 4; nucleic acid sequence is set forth in GenBank Accession No. X15127). The first 25 AA of the proprotein corresponding to this sequence are the signal sequence and are cleaved from LLO when it is secreted by the bacterium. Thus, in this embodiment, the full length active LLO protein is 504 residues long. In another embodiment, the above LLO fragment is used as the source of the LLO fragment incorporated in a vaccine as disclosed herein. In another embodiment, an LLO AA sequence of methods and compositions as disclosed herein comprises the sequence set forth in SEQ ID NO: 4. In another embodiment, the LLO AA sequence is a homologue of SEQ ID NO: 4. In another embodiment, the LLO AA sequence is a variant of SEQ ID NO: 4. In another embodiment, the LLO AA sequence is a fragment of SEQ ID NO: 4. In another embodiment, the LLO AA sequence is an isoform of SEQ ID NO: 4 disclosed herein.

The LLO protein used in the compositions and methods disclosed herein comprises, in another embodiment, the sequence: MKKIMLVFITLILVSLPIAQQTEAKDASAFNKENSISSVAPPASPPASPKTPIEKKHADEI DKYIQGLDYNKNNVLVYHGDAVTNVPPRKGYKDGNEYIVVEKKKKSINQNNADIQV VNAISSLTYPGALVKANSELVENQPDVLPVKRDSLTLSIDLPGMTNQDNKIVVKNAT KSNVNNAVNTLVERWNEKYAQAYSNVSAKIDYDDEMAYSESQLIAKFGTAFKAVN NSLNVNFGAISEGKMQEEVISFKQIYYNVNVNEPTRPSRFFGKAVTKEQLQALGVNA ENPPAYISSVAYGRQVYLKLSTNSHSTKVKAAFDAAVSGKSVSGDVELTNIIKNSSFK AVIYGGSAKDEVQIIDGNLGDLRDILKKGATFNRETPGVPIAYTTNFLKDNELAVIKN NSEYIETTSKAYTD (SEQ ID NO: 5). In another embodiment, an LLO AA sequence of methods and compositions as disclosed herein comprises the sequence set forth in SEQ ID NO: 5. In another embodiment, the LLO AA sequence is a homologue of SEQ ID NO: 5. In another embodiment, the LLO AA sequence is a variant of SEQ ID NO: 5. In another embodiment, the LLO AA sequence is a fragment of SEQ ID NO: 5. In another embodiment, the LLO AA sequence is an isoform of SEQ ID NO: 5.

In one embodiment, the amino acid sequence of endogenous LLO protein comprises SEQ ID NO: 6.

M K K I M L V F I T L I L V S L P I A Q Q T E A K D A S A F N K E N S I S S VA P P A S P P A S P K T P I E K K H A D E I D K Y I Q G L D Y N K N N V LV Y H G D A V T N V P P R K G Y K D G N E Y I V V E K K K K S I N Q N N A D I Q V V N A I S S L T Y P G A L V K A N S E L V E N Q P D V L PV K R D S L T L S I D L P G M T N Q D N K I V V K N A T K S N V N N A V N T L V E R W N E K Y A Q A Y S N V S A K I D Y D D E M A Y S E S Q L I A K F G T A F K A V N N S L N V N F G A I S E G K M Q E E V I S F K Q I Y Y N V N V N E P T R P S R F F G K A V T K E Q L Q A L G V N A E N P P A Y I S S V A Y G R Q V Y L K L S T N S H S T K V K A A F D A A V S G K S V S G D V E LT N I I K N S S F K A V I YG G S A K D E V Q I I D G N L G D L R D I L K K G A T F N R E T P G V P I A Y T T N F L K D N E L A V I K N N S E Y I E T T S K A Y T D G K I N I D H S G G Y V A Q F N I S W D E V N Y D P E G N E I V Q H K N W S E N N K S K L A H F T S S I Y L P G N A R N I N V Y A K E C T G L A W E W W R T V I D D R N L P L V K N R N I S I W G T TL Y P K Y S N K V D N P I E (SEQ ID NO: 6). In another embodiment, an LLO AA sequence of methods and compositions as disclosed herein comprises the sequence set forth in SEQ ID NO: 6. In another embodiment, the LLO AA sequence is a homologue of SEQ ID NO: 6. In another embodiment, the LLO AA sequence is a variant of SEQ ID NO: 6. In another embodiment, the LLO AA sequence is a fragment of SEQ ID NO: 6. In another embodiment, the LLO AA sequence is an isoform of SEQ ID NO: 6.

In one embodiment, the amino acid sequence of the LLO polypeptide of the compositions and methods as disclosed herein is from the Listeria monocytogenes 104035 strain, as set forth in Genbank Accession No.: ZP_01942330, EBA21833, or is encoded by the nucleic acid sequence as set forth in Genbank Accession No.: NZ_AARZ01000015 or AARZ01000015.1. In another embodiment, the LLO sequence for use in the compositions and methods as disclosed herein is from Listeria monocytogenes, which in one embodiment, is the 4b F2365 strain (in one embodiment, Genbank accession number: YP_012823), the EGD-e strain (in one embodiment, Genbank accession number: NP_463733), or any other strain of Listeria monocytogenes known in the art.

In another embodiment, the LLO sequence for use in the compositions and methods as disclosed herein is from Flavobacteriales bacterium HTCC2170 (in one embodiment, Genbank accession number: ZP_01106747 or EAR01433; in one embodiment, encoded by Genbank accession number: NZ_AAOC01000003). In one embodiment, proteins that are homologous to LLO in other species, such as alveolysin, which in one embodiment, is found in Paenibacillus alvei (in one embodiment, Genbank accession number: P23564 or AAA22224; in one embodiment, encoded by Genbank accession number: M62709) may be used in the compositions and methods as disclosed herein. Other such homologous proteins are known in the art.

In another embodiment, homologues of LLO from other species, including known lysins, or fragments thereof may be used to create a fusion protein of LLO with an antigen of the compositions and methods disclosed herein.

In another embodiment, the LLO fragment of methods and compositions disclosed herein comprisesa PEST domain. In another embodiment, the LLO fragment of methods and compositions disclosed herein comprises a putative PEST domain. In another embodiment, an LLO fragment that comprises a PEST sequence is utilized as part of a composition or in the methods as disclosed herein.

In another embodiment, the LLO fragment does not contain the activation domain at the carboxy terminus. In another embodiment, the LLO fragment does not include cysteine 484. In another embodiment, the LLO fragment does not contain the cholesterol binding domain (CBD). In another embodiment, the LLO fragment is a non-hemolytic fragment. In another embodiment, the LLO fragment is rendered non-hemolytic by deletion or mutation of the activation domain. In another embodiment, the LLO fragment is rendered non-hemolytic by deletion or mutation of cysteine 484. In another embodiment, an LLO sequence is rendered non-hemolytic by deletion or mutation at another location.

In another embodiment, the LLO fragment consists of about the first 441 AA of the LLO protein. In another embodiment, the LLO fragment comprises about the first 400-441 AA of the 529 AA full length LLO protein. In another embodiment, the LLO fragment corresponds to AA 1-441 of an LLO protein disclosed herein. In another embodiment, the LLO fragment consists of about the first 420 AA of LLO. In another embodiment, the LLO fragment corresponds to AA 1-420 of an LLO protein disclosed herein. In another embodiment, the LLO fragment consists of about AA 20-442 of LLO. In another embodiment, the LLO fragment corresponds to AA 20-442 of an LLO protein disclosed herein. In another embodiment, any ALLO without the activation domain comprising cysteine 484, and in particular without cysteine 484, are suitable for methods and compositions as disclosed herein.

In another embodiment, the LLO fragment corresponds to the first 400 AA of an LLO protein. In another embodiment, the LLO fragment corresponds to the first 300 AA of an LLO protein. In another embodiment, the LLO fragment corresponds to the first 200 AA of an LLO protein. In another embodiment, the LLO fragment corresponds to the first 100 AA of an LLO protein. In another embodiment, the LLO fragment corresponds to the first 50 AA of an LLO protein, which in one embodiment, comprises one or more PEST sequences.

In another embodiment, the LLO fragment is a non-hemolytic LLO. In another embodiment, the non-hemolytic LLO comprises one or more PEST sequences. In another embodiment, the non-hemolytic LLO comprises one or more putative PEST sequences.

In another embodiment, the LLO fragment contains residues of a homologous LLO protein that correspond to one of the above AA ranges. The residue numbers need not, in another embodiment, correspond exactly with the residue numbers enumerated above; e.g. if the homologous LLO protein has an insertion or deletion, relative to an LLO protein utilized herein.

In one embodiment, the recombinant Listeria strain as provided herein comprises a nucleic acid molecule encoding a tumor associated antigen. In one embodiment, a tumor associated antigen comprises a KLK3 polypeptide or a fragment thereof In one embodiment, the recombinant Listeria strain as provided herein comprises a nucleic acid molecule encoding KLK3 protein.

In another embodiment, a KLK3 protein comprises the sequence: MWVPVVFLTLSVTWIGAAPLILSRIVGGWECEKHSQPWQVLVASRGRAVCGGVLVH PQWVLTAAHCIRNKSVILLGRHSLFHPEDTGQVFQVSHSFPHPLYDMSLLKNRFLRPG DDSSHDLMLLRLSEPAELTDAVKVMDLPTQEPALGTTCYASGWGSIEPEEFLTPKKL QCVDLHVISNDVCAQVHPQKVTKFMLCAGRWTGGKSTCSGDSGGPLVCNGVLQGIT SWGSEPCALPERPSLYTKVVHYRKWIKDTIVANP (SEQ ID No: 7; GenBank Accession No. CAA3297). In another embodiment, the KLK3 protein is a homologue of SEQ ID No: 7. In another embodiment, the KLK3 protein is a variant of SEQ ID No: 7. In another embodiment, the KLK3 protein is an isomer of SEQ ID No: 7. In another embodiment, the KLK3 protein is a fragment of SEQ ID No: 7.

In another embodiment, a KLK3 protein comprises the sequence:

IVGGWECEKHSQPWQVLVASRGRAVCGGVLVHPQWVLTAAHCIRNKSVI LLGRHSLFHPEDTGQVFQVSHSFPHPLYDMSLLKNRFLRPGDDSSHDLMLLRLSEPAE LTDAVKVMDLPTQEPALGTTCYASGWGSIEPEEFLTPKKLQCVDLHVISNDVCAQVH PQKVTKFMLCAGRWTGGKSTCSGDSGGPLVCYGVLQGITSWGSEPCALPERPSLYTK VVHYRKWIKDTIVANP (SEQ ID No: 8). In another embodiment, the KLK3 protein is a homologue of SEQ ID No: 8. In another embodiment, the KLK3 protein is a variant of SEQ ID No: 8. In another embodiment, the KLK3 protein is an isomer of SEQ ID No: 8. In another embodiment, the KLK3 protein is a fragment of SEQ ID No: 8.

In another embodiment, a KLK3 protein comprises the sequence:

IVGGWECEKHSQPWQVLVASRGRAVCGGVLVHPQWVLTAAHCIRNKSVI LLGRHSLFHPEDTGQVFQVSHSFPHPLYDMSLLKNRFLRPGDDSSHDLMLLRLSEPAE LTDAVKVMDLPTQEPALGTTCYASGWGSIEPEEFLTPKKLQCVDLHVISNDVCAQVH PQKVTKFMLCAGRWTGGKSTCSGDSGGPLVCNGVLQGITSWGSEPCALPERPSLYTK VVHYRKWIKDTIVANP (SEQ ID No: 9; GenBank Accession No. AAA59995.1). In another embodiment, the KLK3 protein is a homologue of SEQ ID No: 9. In another embodiment, the KLK3 protein is a variant of SEQ ID No: 9. In another embodiment, the KLK3 protein is an isomer of SEQ ID No: 9. In another embodiment, the KLK3 protein is a fragment of SEQ ID No: 9.

In another embodiment, a KLK3 protein is encoded by a nucleotide molecule comprising the sequence:

ggtgtcttaggcacactggtcttggagtgcaaaggatctaggcacgtgaggctttgtatgaagaatcggggatcgtacc caccccctgtttctgtttcatcctgggcatgtctcctctgcctttgtcccctagatgaagtctccatgagctacaagggcctggtgcatccag ggtgatctagtaattgcagaacagcaagtgctagctctccctccccttccacagctctgggtgtgggagggggttgtccagcctccagc agcatggggagggccttggtcagcctctgggtgccagcagggcaggggcggagtcctggggaatgaaggttttatagggctcctgg gggaggctccccagccccaagcttaccacctgcacccggagagctgtgtcaccatgtgggtcccggttgtcttcctcaccctgtccgtg acgtggattggtgagaggggccatggttggggggatgcaggagagggagccagccctgactgtcaagctgaggctctttccccccc aacccagcaccccagcccagacagggagctgggctcttttctgtctctcccagccccacttcaagcccatacccccagtcccctccata ttgcaacagtcctcactcccacaccaggtccccgctccctcccacttaccccagaactttcttcccatttgcccagccagctccctgctcc cagctgctttactaaaggggaagttcctgggcatctccgtgtttctctttgtggggctcaaaacctccaaggacctctctcaatgccattgg ttccttggaccgtatcactggtccatctcctgagcccctcaatcctatcacagtctactgacttttcccattcagctgtgagtgtccaacccta tcccagagaccttgatgcttggcctcccaatcttgccctaggatacccagatgccaaccagacacctccttctttcctagccaggctatct ggcctgagacaacaaatgggtccctcagtctggcaatgggactctgagaactcctcattccctgactcttagccccagactcttcattca gtggcccacattttccttaggaaaaacatgagcatccccagccacaactgccagctctctgagtccccaaatctgcatccttttcaaaacc taaaaacaaaaagaaaaacaaataaaacaaaaccaactcagaccagaactgttttctcaacctgggacttcctaaactttccaaaacctt cctcttccagcaactgaacctcgccataaggcacttatccctggttcctagcaccccttatcccctcagaatccacaacttgtaccaagttt cccttctcccagtccaagaccccaaatcaccacaaaggacccaatccccagactcaagatatggtctgggcgctgtcttgtgtctcctac cctgatccctgggttcaactctgctcccagagcatgaagcctctccaccagcaccagccaccaacctgcaaacctagggaagattgac agaattcccagcctttcccagctccccctgcccatgtcccaggactcccagccttggttctctgcccccgtgtcttttcaaacccacatcct aaatccatctcctatccgagtcccccagttccccctgtcaaccctgattcccctgatctagcaccccctctgcaggcgctgcgcccctcat cctgtctcggattgtgggaggctgggagtgcgagaagcattcccaaccctggcaggtgcttgtggcctctcgtggcagggcagtctgc ggcggtgttctggtgcacccccagtgggtcctcacagctgcccactgcatcaggaagtgagtaggggcctggggtctggggagcag gtgtctgtgtcccagaggaataacagctgggcattttccccaggataacctctaaggccagccttgggactgggggagagagggaaa gttctggttcaggtcacatggggaggcagggttggggctggaccaccctccccatggctgcctgggtctccatctgtgtccctctatgtc tctttgtgtcgctttcattatgtctcttggtaactggcttcggttgtgtctctccgtgtgactattttgttctctctctccctctcttctctgtcttcagt ctccatatctccccctctctctgtccttctctggtccctctctagccagtgtgtctcaccctgtatctctctgccaggctctgtctctcggtctct gtctcacctgtgccttctccctactgaacacacgcacgggatgggcctgggggaccctgagaaaaggaagggctttggctgggcgcg gtggctcacacctgtaatcccagcactttgggaggccaaggcaggtagatcacctgaggtcaggagttcgagaccagcctggccaac tggtgaaaccccatctctactaaaaatacaaaaaattagccaggcgtggtggcgcatgcctgtagtcccagctactcaggagctgagg gaggagaattgcattgaacctggaggttgaggttgcagtgagccgagaccgtgccactgcactccagcctgggtgacagagtgagac tccgcctcaaaaaaaaaaaaaaaaaaaaaaaaaaaaaagaaaagaaaagaaaagaaaaggaagtgttttatccctgatgtgtgtgggt atgagggtatgagagggcccctctcactccattccttctccaggacatccctccactcttgggagacacagagaagggctggttccagc tggagctgggaggggcaattgagggaggaggaaggagaagggggaaggaaaacagggtatgggggaaaggaccctggggagc gaagtggaggatacaaccttgggcctgcaggcaggctacctacccacttggaaacccacgccaaagccgcatctacagctgagcca ctctgaggcctcccctccccggcggtccccactcagctccaaagtctctctcccttttctctcccacactttatcatcccccggattcctctc tacttggttctcattcttcctttgacttcctgcttccctttctcattcatctgttctcactttctgcctggttttgttcttctctctctctttctctggccc atgtctgtttctctatgtttctgtcttttctttctcatcctgtgtattttcggctcaccttgtttgtcactgttctcccctctgccctttcattctctctgc ccttttaccctcttccttttcccttggttctctcagttctgtatctgcccttcaccctctcacactgctgtttcccaactcgttgtctgtattttggcc tgaactgtgtcttcccaaccctgtgttttctcactgtttctttttctcttttggagcctcctccttgctcctctgtcccttctctctttccttatcatcct cgctcctcattcctgcgtctgcttcctccccagcaaaagcgtgatcttgctgggtcggcacagcctgtttcatcctgaagacacaggcca ggtatttcaggtcagccacagcttcccacacccgctctacgatatgagcctcctgaagaatcgattcctcaggccaggtgatgactccag ccacgacctcatgctgctccgcctgtcagagcctgccgagctcacggatgctgtgaaggtcatggacctgcccacccaggagccagc actggggaccacctgctacgcctcaggctggggcagcattgaaccagaggagtgtacgcctgggccagatggtgcagccgggagc ccagatgcctgggtctgagggaggaggggacaggactcctgggtctgagggaggagggccaaggaaccaggtggggtccagccc acaacagtgtttttgcctggcccgtagtcttgaccccaaagaaacttcagtgtgtggacctccatgttatttccaatgacgtgtgtgcgcaa gttcaccctcagaaggtgaccaagttcatgctgtgtgctggacgctggacagggggcaaaagcacctgctcggtgagtcatccctact cccaagatcttgagggaaaggtgagtgggaccttaattctgggctggggtctagaagccaacaaggcgtctgcctcccctgctcccca gctgtagccatgccacctccccgtgtctcatctcattccctccttccctcttctttgactccctcaaggcaataggttattcttacagcacaac tcatctgttcctgcgttcagcacacggttactaggcacctgctatgcacccagcactgccctagagcctgggacatagcagtgaacaga cagagagcagcccctcccttctgtagcccccaagccagtgaggggcacaggcaggaacagggaccacaacacagaaaagctgga gggtgtcaggaggtgatcaggctctcggggagggagaaggggtggggagtgtgactgggaggagacatcctgcagaaggtggga gtgagcaaacacctgcgcaggggaggggagggcctgcggcacctgggggagcagagggaacagcatctggccaggcctggga ggaggggcctagagggcgtcaggagcagagaggaggttgcctggctggagtgaaggatcggggcagggtgcgagagggaacaa aggacccctcctgcagggcctcacctgggccacaggaggacactgcttttcctctgaggagtcaggaactgtggatggtgctggaca gaagcaggacagggcctggctcaggtgtccagaggctgcgctggcctcctatgggatcagactgcagggagggagggcagcagg gatgtggagggagtgatgatggggctgacctgggggtggctccaggcattgtccccacctgggcccttacccagcctccctcacagg ctcctggccctcagtctctcccctccactccattctccacctacccacagtgggtcattctgatcaccgaactgaccatgccagccctgcc gatggtcctccatggctccctagtgccctggagaggaggtgtctagtcagagagtagtcctggaaggtggcctctgtgaggagccacg gggacagcatcctgcagatggtcctggcccttgtcccaccgacctgtctacaaggactgtcctcgtggaccctcccctctgcacagga gctggaccctgaagtcccttcctaccggccaggactggagcccctacccctctgttggaatccctgcccaccttcttctggaagtcggct ctggagacatttctctcttcttccaaagctgggaactgctatctgttatctgcctgtccaggtctgaaagataggattgcccaggcagaaac tgggactgacctatctcactctctccctgcttttacccttagggtgattctgggggcccacttgtctgtaatggtgtgcttcaaggtatcacgt catggggcagtgaaccatgtgccctgcccgaaaggccttccctgtacaccaaggtggtgcattaccggaagtggatcaaggacacca tcgtggccaacccctgagcacccctatcaagtccctattgtagtaaacttggaaccttggaaatgaccaggccaagactcaagcctccc cagttctactgacctttgtccttaggtgtgaggtccagggttgctaggaaaagaaatcagcagacacaggtgtagaccagagtgtttctta aatggtgtaattttgtcctctctgtgtcctggggaatactggccatgcctggagacatatcactcaatttctctgaggacacagttaggatg gggtgtctgtgttatttgtgggatacagagatgaaagaggggtgggatcc (SEQ ID No: 10; GenBank Accession No. X14810). In another embodiment, the KLK3 protein is encoded by residues 401 . . . 446, 888 . . . 1047, 3477 . . . 3763, 3907 . . . 4043, and 5413 . . . 5568 of SEQ ID No: 10. In another embodiment, the KLK3 protein is encoded by a homologue of SEQ ID No: 10. In another embodiment, the KLK3 protein is encoded by a variant of SEQ ID No: 10. In another embodiment, the KLK3 protein is encoded by an isomer of SEQ ID No: 10. In another embodiment, the KLK3 protein is encoded by a fragment of SEQ ID No: 10.

In another embodiment, a KLK3 protein comprises the sequence: MWVPVVFLTLSVTWIGAAPLILSRIVGGWECEKHSQPWQVLVASRGRAVCGGVLVH PQWVLTAAHCIRNKSVILLGRHSLFHPEDTGQVFQVSHSFPHPLYDMSLLKNRFLRPG DDSSHDLMLLRLSEPAELTDAVKVMDLPTQEPALGTTCYASGWGSIEPEEFLTPKKL QCVDLHVISNDVCAQVHPQKVTKFMLCAGRWTGGKSTC SWVILITELTMPALPMVL HGSLVPWRGGV (SEQ ID No: 11; GenBank Accession No. NP_001011218) In another embodiment, the KLK3 protein is a homologue of SEQ ID No: 11. In another embodiment, the KLK3 protein is a variant of SEQ ID No: 11. In another embodiment, the KLK3 protein is an isomer of SEQ ID No: 11. In another embodiment, the KLK3 protein is a fragment of SEQ ID No: 11.

In another embodiment, a KLK3 protein is encoded by a nucleotide molecule having the sequence:

agccccaagcttaccacctgcacccggagagctgtgtcaccatgtgggtcccggttgtcttcctcaccctgtccgtgac gtggattggtgctgcacccctcatcctgtctcggattgtgggaggctgggagtgcgagaagcattcccaaccctggcaggtgcttgtgg cctctcgtggcagggcagtctgcggcggtgttctggtgcacccccagtgggtcctcacagctgcccactgcatcaggaacaaaagcgt gatcttgctgggtcggcacagcctgtttcatcctgaagacacaggccaggtatttcaggtcagccacagcttcccacacccgctctacg atatgagcctcctgaagaatcgattcctcaggccaggtgatgactccagccacgacctcatgctgctccgcctgtcagagcctgccgag ctcacggatgctgtgaaggtcatggacctgcccacccaggagccagcactggggaccacctgctacgcctcaggctggggcagcat tgaaccagaggagttcttgaccccaaagaaacttcagtgtgtggacctccatgttatttccaatgacgtgtgtgcgcaagttcaccctcag aaggtgaccaagttcatgctgtgtgctggacgctggacagggggcaaaagcacctgctcgtgggtcattctgatcaccgaactgacca tgccagccctgccgatggtcctccatggctccctagtgccctggagaggaggtgtctagtcagagagtagtcctggaaggtggcctct gtgaggagccacggggacagcatcctgcagatggtcctggcccttgtcccaccgacctgtctacaaggactgtcctcgtggaccctcc cctctgcacaggagctggaccctgaagtcccttccccaccggccaggactggagcccctacccctctgttggaatccctgcccaccttc ttctggaagtcggctctggagacatttctctcttcttccaaagctgggaactgctatctgttatctgcctgtccaggtctgaaagataggatt gcccaggcagaaactgggactgacctatctcactctctccctgcttttacccttagggtgattctgggggcccacttgtctgtaatggtgtg cttcaaggtatcacgtcatggggcagtgaaccatgtgccctgcccgaaaggccttccctgtacaccaaggtggtgcattaccggaagtg gatcaaggacaccatcgtggccaacccctgagcacccctatcaaccccctattgtagtaaacttggaaccttggaaatgaccaggcca agactcaagcctccccagttctactgacctttgtccttaggtgtgaggtccagggttgctaggaaaagaaatcagcagacacaggtgta gaccagagtgtttcttaaatggtgtaattttgtcctctctgtgtcctggggaatactggccatgcctggagacatatcactcaatttctctgag gacacagataggatggggtgtctgtgttatttgtggggtacagagatgaaagaggggtgggatccacactgagagagtggagagtga catgtgctggacactgtccatgaagcactgagcagaagctggaggcacaacgcaccagacactcacagcaaggatggagctgaaaa cataacccactctgtcctggaggcactgggaagcctagagaaggctgtgagccaaggagggagggtcttcctttggcatgggatggg gatgaagtaaggagagggactggaccccctggaagctgattcactatggggggaggtgtattgaagtcctccagacaaccctcagatt tgatgatttcctagtagaactcacagaaataaagagctgttatactgtg (SEQ ID No: 12; GenBank Accession No. NM_001030047). In another embodiment, the KLK3 protein is encoded by residues 42-758 of SEQ ID No: 12. In another embodiment, the KLK3 protein is encoded by a homologue of

SEQ ID No: 12. In another embodiment, the KLK3 protein is encoded by a variant of SEQ ID No: 12. In another embodiment, the KLK3 protein is encoded by an isomer of SEQ ID No: 12. In another embodiment, the KLK3 protein is encoded by a fragment of SEQ ID No: 12.

In another embodiment, a KLK3 protein is encoded by a nucleotide molecule comprising the sequence: attgtgggaggctgggagtgcgagaagcattcccaaccctggcaggtgcttgtggcctctcgtggcagggcagtctgcggcggtgttc tggtgcacccccagtgggtcctcacagctgcccactgcatcaggaacaaaagcgtgatcttgctgggtcggcacagcctgtttcatcct gaagacacaggccaggtatttcaggtcagccacagcttcccacacccgctctacgatatgagcctcctgaagaatcgattcctcaggcc aggtgatgactccagccacgacctcatgctgctccgcctgtcagagcctgccgagctcacggatgctgtgaaggtcatggacctgccc acccaggagccagcactggggaccacctgctacgcctcaggctggggcagcattgaaccagaggagttcttgaccccaaagaaac ttcagtgtgtggacctccatgttatttccaatgacgtgtgtgcgcaagttcaccctcagaaggtgaccaagttcatgctgtgtgctggacgc tggacagggggcaaaagcacctgctcgggtgattctgggggcccacttgtctgttatggtgtgcttcaaggtatcacgtcatggggcag tgaaccatgtgccctgcccgaaaggccttccctgtacaccaaggtggtgcattaccggaagtggatcaaggacaccatcgtggccaac ccc (SEQ ID No: 13). In another embodiment, the KLK3 protein is encoded by a homologue of SEQ ID No: 13. In another embodiment, the KLK3 protein is encoded by a variant of SEQ

ID No: 13. In another embodiment, the KLK3 protein is encoded by an isomer of SEQ ID No: 13. In another embodiment, the KLK3 protein is encoded by a fragment of SEQ ID No: 13.

In another embodiment, the KLK3 protein is encoded by a sequence set forth in one of the following GenBank Accession Numbers: BC005307, AJ310938, AJ310937, AF335478, AF335477, M27274, and M26663. In another embodiment, the KLK3 protein is encoded by a sequence set forth in one of the above GenBank Accession Numbers.

In another embodiment, the KLK3 protein is encoded by a sequence set forth in one of the following GenBank Accession Numbers: NM_001030050, NM_001030049, NM_001030048, NM_001030047, NM_00848, AJ459782, AJ512346, or AJ459784. Each possibility represents a separate embodiment of the methods and compositions as provided herein. In one embodiment, the KLK3 protein is encoded by a variation of any of the sequences described herein wherein the sequence lacks MWVPVVFLTLSVTWIGAAPLILSR (SEQ ID NO: 53).

In another embodiment, the KLK3 protein has the sequence that comprises a sequence set forth in one of the following GenBank Accession Numbers: X13943, X13942, X13940, X13941, and X13944.

In another embodiment, the KLK3 protein is any other KLK3 protein known in the art. In another embodiment, the KLK3 peptide is any other KLK3 peptide known in the art. In another embodiment, the KLK3 peptide is a fragment of any other KLK3 peptide known in the art.

“KLK3 peptide” refers, in another embodiment, to a full-length KLK3 protein. In another embodiment, the term refers to a fragment of a KLK3 protein. In another embodiment, the term refers to a fragment of a KLK3 protein that is lacking the KLK3 signal peptide. In another embodiment, the term refers to a KLK3 protein that contains the entire KLK3 sequence except the KLK3 signal peptide. “KLK3 signal sequence” refers, in another embodiment, to any signal sequence found in nature on a KLK3 protein. In another embodiment, a KLK3 protein of methods and compositions as provided herein does not contain any signal sequence.

In another embodiment, the kallikrein-related peptidase 3 (KLK3 protein) that is the source of a KLK3 peptide for use in the methods and compositions disclosed herein is a PSA protein. In another embodiment, the KLK3 protein is a P-30 antigen protein. In another embodiment, the KLK3 protein is a gamma-seminoprotein protein. In another embodiment, the KLK3 protein is a kallikrein 3 protein. In another embodiment, the KLK3 protein is a semenogelase protein. In another embodiment, the KLK3 protein is a seminin protein. In another embodiment, the KLK3 protein is any other type of KLK3 protein that is known in the art.

In another embodiment, the KLK3 protein is a splice variant 1 KLK3 protein. In another embodiment, the KLK3 protein is a splice variant 2 KLK3 protein. In another embodiment, the KLK3 protein is a splice variant 3 KLK3 protein. In another embodiment, the KLK3 protein is a transcript variant 1 KLK3 protein. In another embodiment, the KLK3 protein is a transcript variant 2 KLK3 protein. In another embodiment, the KLK3 protein is a transcript variant 3 KLK3 protein. In another embodiment, the KLK3 protein is a transcript variant 4 KLK3 protein. In another embodiment, the KLK3 protein is a transcript variant 5 KLK3 protein. In another embodiment, the KLK3 protein is a transcript variant 6 KLK3 protein. In another embodiment, the KLK3 protein is a splice variant RP5 KLK3 protein. In another embodiment, the KLK3 protein is any other splice variant KLK3 protein known in the art. In another embodiment, the KLK3 protein is any other transcript variant KLK3 protein known in the art.

In another embodiment, the KLK3 protein is a mature KLK3 protein. In another embodiment, the KLK3 protein is a pro-KLK3 protein. In another embodiment, the leader sequence has been removed from a mature KLK3 protein of methods and compositions as provided herein.

In another embodiment, the KLK3 protein that is the source of a KLK3 peptide of methods and compositions as provided herein is a human KLK3 protein. In another embodiment, the KLK3 protein is a primate KLK3 protein. In another embodiment, the KLK3 protein is a KLK3 protein of any other species known in the art. In another embodiment, one of the above KLK3 proteins is referred to in the art as a “KLK3 protein.”

In one embodiment, a recombinant polypeptide disclosed herein comprising a truncated LLO fused to a PSA protein disclosed herein is encoded by a sequence comprising:

ATGAAAAAAATAATGCTAGTTTTTATTACACTTATATTAGTTAGTCTACCA ATTGCGCAACAAACTGAAGCAAAGGATGCATCTGCATTCAATAAAGAAAATTCAA TTTCATCCATGGCACCACCAGCATCTCCGCCTGCAAGTCCTAAGACGCCAATCGAA AAGAAACACGCGGATGAAATCGATAAGTATATACAAGGATTGGATTACAATAAAA ACAATGTATTAGTATACCACGGAGATGCAGTGACAAATGTGCCGCCAAGAAAAGG TTACAAAGATGGAAATGAATATATTGTTGTGGAGAAAAAGAAGAAATCCATCAAT CAAAATAATGCAGACATTCAAGTTGTGAATGCAATTTCGAGCCTAACCTATCCAGG TGCTCTCGTAAAAGCGAATTCGGAATTAGTAGAAAATCAACCAGATGTTCTCCCTG TAAAACGTGATTCATTAACACTCAGCATTGATTTGCCAGGTATGACTAATCAAGAC AATAAAATAGTTGTAAAAAATGCCACTAAATCAAACGTTAACAACGCAGTAAATA CATTAGTGGAAAGATGGAATGAAAAATATGCTCAAGCTTATCCAAATGTAAGTGC AAAAATTGATTATGATGACGAAATGGCTTACAGTGAATCACAATTAATTGCGAAAT TTGGTACAGCATTTAAAGCTGTAAATAATAGCTTGAATGTAAACTTCGGCGCAATC AGTGAAGGGAAAATGCAAGAAGAAGTCATTAGTTTTAAACAAATTTACTATAACG TGAATGTTAATGAACCTACAAGACCTTCCAGATTTTTCGGCAAAGCTGTTACTAAA GAGCAGTTGCAAGCGCTTGGAGTGAATGCAGAAAATCCTCCTGCATATATCTCAAG TGTGGCGTATGGCCGTCAAGTTTATTTGAAATTATCAACTAATTCCCATAGTACTAA AGTAAAAGCTGCTTTTGATGCTGCCGTAAGCGGAAAATCTGTCTCAGGTGATGTAG AACTAACAAATATCATCAAAAATTCTTCCTTCAAAGCCGTAATTTACGGAGGTTCC GCAAAAGATGAAGTTCAAATCATCGACGGCAACCTCGGAGACTTACGCGATATTTT GAAAAAAGGCGCTACTTTTAATCGAGAAACACCAGGAGTTCCCATTGCTTATACAA CAAACTTCCTAAAAGACAATGAATTAGCTGTTATTAAAAACAACTCAGAATATATT GAAACAACTTCAAAAGCTTATACAGATGGAAAAATTAACATCGATCACTCTGGAG GATACGTTGCTCAATTCAACATTTCTTGGGATGAAGTAAATTATGATCTCGAGattgtg ggaggctgggagtgcgagaagcattcccaaccctggcaggtgcttgtggcctctcgtggcagggcagtctgcggcggtoctggtgc acccccagtgggtcctcacagctgcccactgcatcaggaacaaaagcgtgatcttgctgggtcggcacagcctotcatcctgaagac acaggccaggtatttcaggtcagccacagatcccacacccgctctacgatatgagcctcctgaagaatcgattcctcaggccaggtga tgactccagccacgacctcatgctgctccgcctgtcagagcctgccgagctcacggatgctgtgaaggtcatggacctgcccacccag gagccagcactggggaccacctgctacgcctcaggctggggcagcattgaaccagaggagttcttgaccccaaagaaacttcagtgt gtggacctccatgttatttccaatgacgtgtgtgcgcaagttcaccctcagaaggtgaccaagttcatgctgtgtgctggacgctggaca gggggcaaaagcacctgctcgggtgattctgggggcccacttgtctgttatggtgtgcttcaaggtatcacgtcatggggcagtgaacc atgtgccctgcccgaaaggccttccctgtacaccaaggtggtgcattaccggaagtggatcaaggacaccatcgtggccaacccc (SEQ ID NO: 14). In another embodiment, the fusion protein is encoded by a homologue of SEQ ID No: 14. In another embodiment, the fusion protein is encoded by a variant of SEQ ID No: 14.

In another embodiment, the fusion protein is encoded by an isomer of SEQ ID No: 14. In one embodiment, the “ctcgag” sequence within the fusion protein represents a Xho I restriction site used to ligate the tumor antigen to truncated LLO in the plasmid.

In another embodiment, a recombinant polypeptide disclosed herein comprising a truncated LLO fused to a PSA protein disclosed herein comprises the following sequence:

(SEQ ID NO: 15)
MKKIMLVFITLILVSLPIAQQTEAKDASAFNKENSISSMAPPASPPASP
KTPIEKKHADEIDKYIQGLDYNKNNVLVYHGDAVTNVPPRKGYKDGNEY
IVVEKKKKSINQNNADIQWNAISSLTYPGALVKANSELVENQPDVLPVK
RDSLTLSIDLPGMTNQDNKIVVKNATKSNVNNAVNTLVERWNEKYAQAY
PNVSAKIDYDDEMAYSESQLIAKFGTAFKAVNNSLNVNFGAISEGKMQE
EVISFKQIYYNVNVNEPTRPSRFFGKAVTKEQLQALGVNAENPPAYISS
VAYGRQVYLKLSTNSHSTKVKAAFDAAVSGKSVSGDVELTNIIKNSSFK
AVIYGGSAKDEVQIIDGNLGDLRDILKKGATFNRETPGVPIAYTTNFLK
DNELAVIKNNSEYIETTSKAYTDGKINIDHSGGYVAQFNISWDEVNYDL
EIVGGWECEKHSQPWQVLVASRGRAVCGGVLVHPQWVLTAAHCIRNKSV
ILLGRHSLFHPEDTGQVFQVSIISFPHPLYDMSLLKNRFLRPGDDSSHD
LMLLRLSEPAELTDAVKVMDLPTQEPALGTTCYASGWGSIEPEEFLTPK
KLQCVDLHVISNDVCAQVHPQKVTKFMLCAGRWTGGKSTCSGDSGGPLV
CYGVLQGITSWGSEPCALPERPSLYTKVVHYRKWIKDTIVANP
(PSA sequence is underlined).

In another embodiment, the tLLO-PSA fusion protein is a homologue of SEQ ID NO: 15. In another embodiment, the tLLO-PSA fusion protein is a variant of SEQ ID NO: 15. In another embodiment, the tLLO-PSA fusion protein is an isomer of SEQ ID NO: 15. In another embodiment, the tLLO-PSA fusion protein is a fragment of SEQ ID NO: 15.

In another embodiment, a Her-2 protein is a protein referred to as “HER-2/neu,” “Erbb2,” “v-erb-b2,” “c-erb-b2,” “neu,” or “cNeu.”

In one embodiment, a heterologous antigen disclosed herein is a chimeric Her2/neu antigen or Her2-neu chimeric protein (cHER2). In another embodiment, a cHER2 harbors two of the extracellular and one intracellular fragments of Her2/neu antigen showing clusters of MHC-class I epitopes of the oncogene, where, in another embodiment, the chimeric protein, harbors 3 H2Dq and at least 17 of the mapped human MHC-class I epitopes of the Her2/neu antigen (fragments EC1, EC2, and IC1) as described in U.S. patent application Ser. No. 12/945,386, which is incorporated by reference herein in its entirety. In another embodiment, the Her2-neu chimeric protein is fused to the first 441 amino acids of the Listeria-monocytogenes listeriolysin O (LLO) protein and expressed and secreted by the Listeria monocytogenes attenuated auxotrophic strain LmddA. In another embodiment, the Her2-neu chimeric protein is fused to the first 441 amino acids of the Listeria-monocytogenes listeriolysin O (LLO) protein and is expressed from the chromosome of a recombinant Listeria disclosed herein, while an additional antigen is expressed from a plasmid present within the recombinant Listeria disclosed herein. In another embodiment, the Her2-neu chimeric protein is fused to the first 441 amino acids of the Listeria-monocytogenes listeriolysin O (LLO) protein and is expressed from a plasmid of a recombinant Listeria disclosed herein, while an additional antigen is expressed from the chromosome of the recombinant Listeria disclosed herein. In another embodiment, a recombinant Listeria disclosed herein is a Listeria monocytogenes attenuated auxotrophic strain LmddA.

In one embodiment, a chimeric HER2 protein is encoded by the following nucleic acid sequence set forth in SEQ ID NO:16

acccacctggacatgctccgccacctctaccagggctgccaggtggtgcagggaaacctggaactcacctacctgcccac caatgccagcctgtccttcctgcaggatatccaggaggtgcagggctacgtgctcatcgctcacaaccaagtgaggcaggtcccactg cagaggctgcggattgtgcgaggcacccagctctttgaggacaactatgccctggccgtgctagacaatggagacccgctgaacaat accacccctgtcacaggggcctccccaggaggcctgcgggagctgcagcttcgaagcctcacagagatcttgaaaggaggggtctt gatccagcggaacccccagctctgctaccaggacacgattttgtggaagaatatccaggagtttgctggctgcaagaagatctttggga gcctggcatttctgccggagagctttgatggggacccagcctccaacactgccccgctccagccagagcagctccaagtgtttgagac tctggaagagatcacaggttacctatacatctcagcatggccggacagcctgcctgacctcagcgtcttccagaacctgcaagtaatcc ggggacgaattctgcacaatggcgcctactcgctgaccctgcaagggctgggcatcagctggctggggctgcgctcactgagggaa ctgggcagtggactggccctcatccaccataacacccacctctgcttcgtgcacacggtgccctgggaccagctctttcggaacccgc accaagctctgctccacactgccaaccggccagaggacgagtgtgtgggcgagggcctggcctgccaccagctgtgcgcccgagg gcagcagaagatccggaagtacacgatgcggagactgctgcaggaaacggagctggtggagccgctgacacctagcggagcgat gcccaaccaggcgcagatgcggatcctgaaagagacggagctgaggaaggtgaaggtgatggatctggcgctffiggcacagtcta caagggcatctggatccctgatggggagaatgtgaaaattccagtggccatcaaagtgttgagggaaaacacatcccccaaagccaa caaagaaatcttagacgaagcatacgtgatggctggtgtgggctccccatatgtctcccgccttctgggcatctgcctgacatccacggt gcagctggtgacacagcttatgccctatggctgcctcttagac (SEQ ID NO: 16). In another embodiment, the cHER2 protein is encoded by a homologue of SEQ ID No: 16. In another embodiment, the cHER2 protein is encoded by a variant of SEQ ID No: 16. In another embodiment, the cHER2 protein is encoded by an isomer of SEQ ID No: 16. In another embodiment, the cHER2 protein is encoded by a fragment of SEQ ID No: 16.

In one embodiment, a chimeric HER2 protein comprises the sequence:

T H L D M L R H L Y Q G C Q V V Q G N L E L T Y L P T N A S L S F L Q D I Q E V Q G Y V L I A H N Q V R Q V P L Q R L R I V R G T Q L F E D N Y A L A V L D N G D P L N N T T P V T G A S P G G L R E L Q L R S L T E I L K G G V L I Q R N P Q L C Y Q D T I L W K N I Q E F A G C K K I F G S L A F L P E S F D G D P A S N T A P L Q P E Q L Q V F E T L E E I T G Y L Y I S A W P DS L P D L S V F Q N L Q V I R G R I L H N G A Y S L T L Q G L G I S W L G L R S L R E L G S G L A L I H H N T H L C F V H T V P W D Q L F R N P H Q A L L H T A N R P E D E C V G E G L A C H Q L C A R G Q Q K I R K Y T M R R L L Q E T E L V E P L T P S G A M P N Q A Q M R I L K E T E L R K V K V L G S G A F G T V Y K G I W I P D G E N V K I P V A I K V L R E N T S P K A N K E I L D E A Y V M A G V G S P Y V S R L L G I C L T S TV Q L V T Q L M P Y G C L L D (SEQ ID NO: 17). In another embodiment, the cHER2 protein is a homologue of SEQ ID No: 17. In another embodiment, the cHER2 protein is a variant of SEQ ID No: 17. In another embodiment, the cHER2 protein is an isomer of SEQ ID No: 17. In another embodiment, the cHER2 protein is a fragment of SEQ ID No: 17.

In one embodiment, the Her2 chimeric protein or fragment thereof of the methods and compositions provided herein does not include a signal sequence thereof. In another embodiment, omission of the signal sequence enables the Her2 fragment to be successfully expressed in Listeria, due the high hydrophobicity of the signal sequence.

In another embodiment, the fragment of a Her2 chimeric protein of methods and compositions of the present disclosure does not include a transmembrane domain (TM) thereof. In one embodiment, omission of the TM enables the Her-2 fragment to be successfully expressed in Listeria, due the high hydrophobicity of the TM.

In one embodiment, disclosed herein is a recombinant polypeptide comprising an N-terminal fragment of an LLO protein fused to a heterologous antigen disclosed herein or fused to a fragment thereof. In another embodiment, a Her-2 chimeric protein of the methods and compositions of the present disclosure is a human Her-2 chimeric protein. In another embodiment, the Her-2 protein is a mouse Her-2 chimeric protein. In another embodiment, the Her-2 protein is a rat Her-2 chimeric protein. In another embodiment, the Her-2 protein is a primate Her-2 chimeric protein. In another embodiment, the Her-2 protein is a Her-2 chimeric protein of any other animal species or combinations thereof known in the art.

In one embodiment, a Listeria strain LmddA244G disclosed herein comprises a nucleic acid sequence comprising an open reading frame encoding a cHER2 fused to an endogenous nucleic acid comprising an open reading frame encoding an LLO protein (see SEQ ID NO: 18).

atgaaaaaaataatgctagtttttattacacttatattagttagtctaccaattgcgcaacaaactgaagcaaaggatgcatetgc attcaataaagaaaattcaatttcatccgtggcaccaccagcatctccgcctgcaagtcctaagacgccaatcgaaaagaaacacgcgg atgaaatcgataagtatatacaaggattggattacaataaaaacaatgtattagtataccacggagatgcagtgacaaatgtgccgccaa gaaaaggttacaaagatggaaatgaatatattgttgtggagaaaaagaagaaatccatcaatcaaaataatgcagacattcaagttgtga atgcaatttcgagcctaacctatccaggtgctctcgtaaaagcgaattcggaattagtaguaautcaaccagatgttctccctgtaaaacg tgattcattaacactcagcattgatttgccaggtatgactaatcaagacaataaaatagttgtaaaaaatgccactaaatcaaacgttaaca acgcagtaaatacattagtggaaagatggaatgaaaaatatgctcaagcttattcaaatgtaagtgcaaaaattgattatgatgacgaaat ggcttacagtgaatcacaattaattgcgaaatttggtacagcatttaaagctgtaaataatagcttgaatgtaaacttcggcgcaatcagtg aagggaaaatgcaagaagaagtcattagttttaaacaaatttactataacgtgaatgttaatgaacctacaagaccttccagatttttcggc aaagctgttactaaagagcagtlgcaagcgcttggagtgaatgcagaaaatcctcctgcatatatctcaagtgtggcgtatggccgtcaa gttlatttgaaattatcaactaattcccatagtactaaagtaaaagctgcttttgatgctgccgtaagcggaaaatctgtctcaggtgatgtag aactaacaaatatcatcaaaaattcttccttcaaagccgtaatttacggaggttccgcaaaagatgaagttcaaatcatcgacggcaacct cggagacttacgcgatattttgaaaaaaggcgctacttttaategagaaacaccaggagttcecattgcttatacaacaaacttcctaaaa gacaatgaattagctgttattaaaaacaactcagaatatattgaaacaacttcaaaagcttatacagatggaaaaattaacatcgatcactct ggaggatacgttgctcaattcaacatttcttgggatgaagtaaaataagaacctgaaggtaacgaaattgttcaacataaaaacaggagcg aaaacaataaaagcaagctagctcatttcacatcgtccatctatttgcctggtaacgcgagaaatataaatgtttacgctaaagaatgcact ggtttagcttgggaatggtggagaacggtaattgatgaccggaacttaccacttgtgaaaaatagaaaiatctccatctggggcaccacg ctttaaccgaaataaagtaataaagtagataatccaatcgaagtcgacACCCCACCTGGACATGCTCCCGCCACCT CTACCAGGGCTGCCAGGTGGTGCAGGGAAACCTGGAACTCACCTACCTGCCCACC AATGCCAGCCTGTCCTTCCTGCAGGATATCCAGGAGGTGCAGGGCTACGTGCTCA TCGCTCACAACCAAGTGAGGCAGGTCCCACTGCAGAGGCTGCGGATTGTGCGAG GCACCCAGCTCTTTGAGGACAACTATGCCCTGGCCGTGCTAGACAATGGAGACCC GCTGAACAATACCACCCCTGTCACAGGGGCCTCCCCAGGAGGCCTGCGGGAGCT GCAGCTTCGAAGCCTCACAGAGATCTTGAAAGGAGGGGTCTTGATCCAGCGGAA CCCCCAGCTCTGCTACCAGGACACGATTTTGTGGAAGAATATCCAGGAGTTTGCT GGCTGCAAGAAGATCTTTGGGAGCCTGGCATTTCTGCCGGAGAGCTTTGATGGGG ACCCAGCTCCAACACATGCCCCGCTCCAGCCAGAGCAGCTCCAAGTGTTTGAGAC TCTGGAAGAGATCACAGGTTACCTATACATCTCAGCCATGGCCGGACAGCCTGCCT GACCTCAGCGTCTTCCAGAACCTGCAAGTAATCCGGGGACGAATTCTGCACAATG GCGCCTACTCGCTGACCCTGCAAGGGCTGGGCATCAGCTGGTGGGGCTGCGCTC ACTGAGGGAACTGGGCAGTGGACTGGCCCTCATCCACCATAACACCCACCTCTGC TTCGTGCACACGGTGCCCTGGGACCAGCTCTTTCGGAACCCGCACCAAGCTCTGC TCCACACTGCCAACCGGCCAGAGGACGAGTGTGTGGGCGAGGGCCTGGCCTGCC ACCAGCTGTGCGCCCGAGGGCAGCAGAAGATCCGGAAGTACACGATGCGGAGAC TGCTGCAGGAAACGGAGCTGGTGGAGCCGCTGACACCTAGCGGAGCGATGCCCA ACCAGGCGCAGATGCGGATCCTGAAAGAGACGGAGCTGAGGAAGGTGAAGGTGC TTGGATCTGGCGCTTTTGGCACAGTCTACAAGGGCATCTGGATCCCTGATGGGGA GAATGTGAAAATTCCAGTGGCCATCAAAGTGTTGAGGGAAAACACATCCCCCAA AGCCAACAAAGAAATCTTAGACGAAGCATACGTGATGGCTGGTGTGGGCTCCCC ATATGTCTCCCGCCTTCTGGGCATCTGCCTGACATCCACGGTGCAGCTGGTGACA CAGCTTATGCCCTATGGCTGCCTCTTAGAC (SEQ ID NO: 18),

where the UPPERCASE sequences represents the nucleic acid sequence encoding a cHER2, the lower case sequences represent the sequence encoding an endogenous LLO protein and the underlined “gtcgac” sequence represents the Sal I restriction site used to ligate the tumor antigen to the endogenous LLO. In one embodiment, the endogenous LLO-cHER18 fusion is a homolog of SEQ ID NO: 18. In another embodiment, the endogenous LLO-cHER18 fusion is a variant of SEQ ID NO: 18. In another embodiment, the endogenous LLO-cHER18 fusion is an isomer of SEQ ID NO: 18.

In one embodiment, the amino acid sequence of the fusion between a cHER2 and an endogenous LLO comprises SEQ ID NO: 19. MKKIMLVFITLILVSLPIAQQTEAKDASAFNKENSISSVAPPASPPASPKTPIEKKHADEI DKYIQGLDYNKNNVLVYHGDAVTNVPPRKGYKDGNEYIVVEKKKKSINQNNADIQV VNAISSLTYPGALVKANSELVENQPDVLPVKRDSLTLSIDLPGMTNQDNKIVVKNAT KSNVNNAVNTLVERWNEKYAQAYSNVSAKIDYDDEMAYSESQLIAKFGTAFKAVN NSLNVNFGAISEGKMQEEVISFKQIYYNVNVNEPTRPSRFFGKAVTKEQLQALGVNA ENPPAYISSVAYGRQVYLKLSTNSHSTKVKAAFDAAVSGKSVSGDVELTNIIKNSSFK AVIYGGSAKDEVQIIDGNLGDLRDILKKGATFNRETPGVPIAYTTNFLKDNELAVIKN NSEYIETTSKAYTDGKGNIDHSGGYVAQFNISWDEVNYDPEGNEIVQHKNWSENNKS KLAHFTSSIYLPGNARNINVYAKECTGLAWEWWRTVIDDRNLPLVKNRNISIWGTTL YPKYSNKVDNPIEVDTHLDMLRHLYQGCQVVQGNLELTYLPTNASLSFLQDIQEVQG YVLIAHNQVRQVPLQRLRIVRGTQLFEDNYALAVLDNGDPLNNTTPVTGASPGGLRE LQLRSLTEILKGGVLIQRNPQLCYQDTILWKNIQEFAGCKKIFGSLAFLPESFDGDPAS NTAPLQPEQLQVFETLEEITGYLYISAWPDSLPDLSVFQNLQVIRGRILHNGAYSLTLQ GLGISWLGLRSLRELGSGLALIMINTHLCFVHTVPWDQLFRNPHQALLHTANRPEDE CVGEGLACHQLCARGQQKIRKYTMRRLLQETELVEPLTP SGAMPNQAQMRILKETEL RKVKVLGSGAFGTVYKGIWIPDGENVKIPVAIKVLRENTSPKANKEILDEAYVMAGV GSPYVSRLLGICLTSTVQLVTQLMPYGCLLD (SEQ ID NO: 19). In one embodiment, the endogenous LLO-cHER2 fusion is a homolog of SEQ ID NO: 19. In another embodiment, the endogenous LLO-cHER2 fusion is a variant of SEQ ID NO: 19. In another embodiment, the endogenous LLO-cHER2 fusion is an isomer of SEQ ID NO: 19.

In one embodiment, LmddA164 comprises a nucleic acid sequence comprising an open reading frame encoding tLLO fused to cHER2, wherein said nucleic acid sequence comprises SEQ ID NO: 20:

(SEQ ID NO: 20)
atgaaaaaaataatgctagtttttattacacttatattagttagtctac
caattgcgcaacaaactgaagcaaaggatgcatctgcattcaataaaga
aaattcaatttcatccatggcaccaccagcatctccgcctgcaagtcct
aagacgccaatcgaaaagaaacacgcggatgaaatcgataagtatatac
aaggattggattacaataaaaacaatgtattagtataccacggagatgc
agtgacaaatgtgccgccaagaaaaggttacaaagatggaaatgaatat
attgttgtggagaaaaagaagaaatccatcaatcaaaataatgcagaca
ttcaagttgtgaatgcaatttcgagcctaacctatccaggtgctctcgt
aaaagcgaattcggaattagtagaaaatcaaccagatgttctccctgta
aaacgtgattcattaacactcagcattgatttgccaggtatgactaatc
aagacaataaaatagttgtaaaaaatgccactaaatcaaacgttaacaa
cgcagtaaatacattagtggaaagatggaatgaaaaatatgctcaagct
tatccaaatgtaagtgcaaaaattgattatgatgacgaaatggcttaca
gtgaatcacaattaattgcgaaatttggtacagcatttaaagctgtaaa
taatagcttgaatgtaaacttcggcgcaatcagtgaagggaaaatgcaa
gaagaagtcattagttttaaacaaatttactataacgtgaatgttaatg
aacctacaagaccttccagatttttcggcaaagctgttactaaagagca
gttgcaagcgcttggagtgaatgcagaaaatcctcctgcatatatctca
agtgtggcgtatggccgtcaagtttatttgaaattatcaactaattccc
atagtactaaagtaaaagctgcttttgatgctgccgtaagcggaaaatc
tgtctcaggtgatgtagaactaacaaatatcatcaaaaattcttccttc
aaagccgtaatttacggaggttccgcaaaagatgaagttcaaatcatcg
acggcaacctcggagacttacgcgatattttgaaaaaaggcgctacttt
taatcgagaaacaccaggagttcccattgcttatacaacaaacttccta
aaagacaatgaattagctgttattaaaaacaactcagaatatattgaaa
caacttcaaaagcttatacagatggaaaaattaacatcgatcactctgg
aggatacgttgctcaattcaacatttcttgggatgaagtaaattatgat
ctcgagACCCACCTGGACATGCTCCGCCACCTCTACCAGGGCTGCCAGG
TGGTGCAGGGAAACCTGGAACTCACCTACCTGCCCACCAATGCCAGCCT
GTCCTTCCTGCAGGATATCCAGGAGGTGCAGGGCTACGTGCTCATCGCT
CACAACCAAGTGAGGCAGGTCCCACTGCAGAGGCTGCGGATTGTGCGAG
GCACCCAGCTCTTTGAGGACAACTATGCCCTGGCCGTGCTAGACAATGG
AGACCCGCTGAACAATACCACCCCTGTCACAGGGGCCTCCCCAGGAGGC
CTGCGGGAGCTGCAGCTTCGAAGCCTCACAGAGATCTTGAAAGGAGGGG
TCTTGATCCAGCGGAACCCCCAGCTCTGCTACCAGGACACGATTTTGTG
GAAGAATATCCAGGAGTTTGCTGGCTGCAAGAAGATCTTTGGGAGCCTG
GCATTTCTGCCGGAGAGCTTTGATGGGGACCCAGCCTCCAACACTGCCC
CGCTCCAGCCAGAGCAGCTCCAAGTGTTTGAGACTCTGGAAGAGATCAC
AGGTTACCTATACATCTCAGCATGGCCGGACAGCCTGCCTGACCTCAGC
GTCTTCCAGAACCTGCAAGTAATCCGGGGACGAATTCTGCACAATGGCG
CCTACTCGCTGACCCTGCAAGGGCTGGGCATCAGCTGGCTGGGGCTGCG
CTCACTGAGGGAACTGGGCAGTGGACTGGCCCTCATCCACCATAACACC
CACCTCTGCTTCGTGCACACGGTGCCCTGGGACCAGCTCTTTCGGAACC
CGCACCAAGCTCTGCTCCACACTGCCAACCGGCCAGAGGACGAGTGTGT
GGGCGAGGGCCTGGCCTGCCACCAGCTGTGCGCCCGAGGGCAGCAGAAG
ATCCGGAAGTACACGATGCGGAGACTGCTGCAGGAAACGGAGCTGGTGG
AGCCGCTGACACCTAGCGGAGCGATGCCCAACCAGGCGCAGATGCGGAT
CCTGAAAGAGACGGAGCTGAGGAAGGTGAAGGTGCTTGGATCTGGCGCT
TTTGGCACAGTCTACAAGGGCATCTGGATCCCTGATGGGGAGAATGTGA
AAATTCCAGTGGCCATCAAAGTGTTGAGGGAAAACACATCCCCCAAAGC
CAACAAAGAAATCTTAGACGAAGCATACGTGATGGCTGGTGTGGGCTCC
CCATATGTCTCCCGCCTTCTGGGCATCTGCCTGACATCCACGGTGCAGC
TGGTGACACAGCTTATGCCCTATGGCTGCCTCTTAGAC,

wherein the UPPERCASE sequences encode cHER2, the lowercase sequences encode tLLO and the underlined “ctcgag” sequence represents the Xho I restriction site used to ligate the tumor antigen to truncated LLO in the plasmid. In another embodiment, plasmid pAdv168 comprises SEQ ID NO: 20. In one embodiment, the truncated LLO-cHER2 fusion is a homolog of SEQ ID NO: 20. In another embodiment, the truncated LLO-cHER2 fusion is a variant of SEQ ID NO: 20. In another embodiment, the truncated LLO-cHER2 fusion is an isomer of SEQ ID NO: 20.

In one embodiment, an amino acid sequence of a recombinant protein comprising tLLO fused to a cHER2 comprises SEQ ID NO: 21: MKKIMLVFITLILVSLPIAQQTEAKDASAFNKENSISSMAPPASPPASPKTPIEKKHADE IDKYGLDYNKNNVLVYHGDAVTNVPPRKGYKDGNEYIVVEKKKKSINQNNADIQ VVNAIISSLTYPGALVKANSELVENQPDVLPVKRDSLTLSIDLPGMTNQDNKIVVKNA TKSNVNNAVNLVERWNEKYAQAYPNVSAKIDYDDEMAYSESLIAKFGTAFKAV NNSLNVNFGAISEGKMQEEVISFKQTYYNVNVNEPTRPSRFFGKAVTKEQLQALGVN AENPPAYISSVAYGRQVYLKLSTNSHSTKVKAAFDAAVSGKSVSGDVELTNIIKNSSF KAVIYGGSAKDEVQIIDGNLGDLRDILKKGATFNRETPGVPIAYTTNFLKDNELAVIK NNSEYIETTS KAYTDGKINIDHSGGYVAQFNISWDEVNYDLETHLDMLRHLYQGCQV VQGNLELTYLPTNASLSFLQDIQEVQGYVLIAHNQVRQVPLQRLRIVRGTQLFEDNY ALAVLDNGDPLNNTTPVTGASPGGLRELQLRSLTEILKGGVLIQRNPQLCYQDTILWK NIQEFAGCKKIFGSLAFLPESFDGDPASNTAPLQPEQLQVFETLEEITGYLYISAWPDSL PDLSVFQNLQVIRGRILHNGAYSLTLQGLGISWLGLRSLRELGSGLALIHHNTHLCFV HTVPWDQLFRNPHQALLHTANRPEDECVGEGLACHQLCARGQQKIRKYTMRRLLQE TELVEPLTPSGAMPNQAQMRILKETELRKVKVLGSGAFGTVYKGIWIPDGENVKIPV AIKVLRENTSPKANKEILDEAYVMAGVGSPYVSRLLGICLTSTVQLVTQLMPYGCLL D (SEQ ID NO: 21). In one embodiment, the truncated LLO-cHER2 fusion is a homolog of SEQ ID NO: 21. In another embodiment, the truncated LLO-cHER2 fusion is a variant of SEQ ID NO: 21. In another embodiment, the truncated LLO-cHER2 fusion is an isomer of SEQ ID NO: 21.

In one embodiment, the antigens are heterologous antigens to the bacteria host carrying the plasmid. In another embodiment, the antigens are heterologous antigens to the Listeria host carrying the plasmid.

In one embodiment, disclosed herein is an immunotherapeutic or immunogenic composition comprising a recombinant Listeria strain and an adjuvant, cytokine, chemokine, or a combination thereof. In one embodiment, disclosed herein is a vaccine comprising a recombinant Listeria strain and an adjuvant, cytokine, chemokine, or a combination thereof. In another embodiment, disclosed herein is a pharmaceutical formulation comprising a recombinant Listeria strain and an adjuvant, cytokine, chemokine, or a combination thereof.

In one embodiment of the present disclosure, a recombinant Listeria disclosed herein is a recombinant Listeria strain comprising a nucleic acid molecule, said nucleic acid molecule encoding a heterologous antigenic polypeptide or fragment thereof, wherein the nucleic acid molecule is integrated into the Listeria genome in an open reading frame with an endogenous LLO gene. In another embodiment, nucleic acid molecule is operably integrated into the Listeria genome as an open reading frame with an endogenous nucleic acid sequence encoding an LLO protein, an ActA protein or a PEST sequence. In one embodiment, the nucleic acid molecule is operably integrated into the Listeria genome as an open reading frame with a nucleic acid sequence encoding LLO. In another embodiment, the nucleic acid molecule is operably integrated into the Listeria genome as an open reading frame with a nucleic acid sequence encoding ActA. In one embodiment, the integration does not eliminate the functionality of LLO. In another embodiment, the integration does not eliminate the functionality of ActA. In one embodiment, the functionality of LLO or ActA is its native functionality.

In another embodiment, a recombinant Listeria strain disclosed herein comprises a mutation, deletion or inactivation in the endogenous dal, dat and an actA genes. In another embodiment, the recombinant Listeria strain comprises a mutation in the actA and inlB genes. In one embodiment, the recombinant Listeria strain provided herein is attenuated. In another embodiment, the recombinant Listeria lacks the actA virulence gene. In another embodiment, the recombinant Listeria lacks the prfA virulence gene. In another embodiment, the recombinant Listeria lacks the inlB gene. In another embodiment, the recombinant Listeria lacks both, the actA and inlB genes. In another embodiment, the recombinant Listeria strain comprises an inactivating mutation of the endogenous actA gene. In another embodiment, the recombinant Listeria strain comprises an inactivating mutation of the endogenous inlB gene. In another embodiment, the recombinant Listeria strain comprises an inactivating mutation of the endogenous inlC gene. In another embodiment, the recombinant Listeria strain comprises an inactivating mutation of the endogenous actA and inlB genes. In another embodiment, the recombinant Listeria strain comprises an inactivating mutation of the endogenous actA and inlC genes. In another embodiment, the recombinant Listeria strain comprises an inactivating mutation of the endogenous actA, inlB, and inlC genes. In another embodiment, the recombinant Listeria strain comprises an inactivating mutation of the endogenous actA, inlB, and inlC genes. In another embodiment, the recombinant Listeria strain comprises an inactivating mutation of the endogenous actA, inlB, and inlC genes. In another embodiment, the recombinant Listeria strain comprises an inactivating mutation in any single gene or combination of the following genes: actA, dal, dat, inlB, inlC, prfA, plcA, plcB.

In one embodiment, the LLO functionality is allowing the organism to escape from the phagolysosome, while in another embodiment, the LLO functionality is enhancing the immunogenicity of a polypeptide to which it is fused. In one embodiment, a recombinant Listeria disclosed herein retains LLO function, which in one embodiment, is hemolytic function and in another embodiment, is antigenic function. Other functions of LLO are known in the art, as are methods and assays for evaluating LLO functionality.

In one embodiment, a recombinant Listeria disclosed herein has attenuated virulence. In another embodiment, a recombinant Listeria disclosed herein is avirulent. In one embodiment, a recombinant Listeria of disclosed herein is sufficiently virulent to escape the phagolysosome and enter the cytosol. In one embodiment, a recombinant Listeria disclosed herein expresses a fused antigen-LLO protein. Thus, in one embodiment, the integration of the nucleic acid molecule into the Listeria genome does not disrupt the structure nor, in another embodiment, the function of the endogenous LLO gene, or ActA gene. In one embodiment, the integration of a nucleic acid molecule into the Listeria genome does not disrupt the ability of said Listeria to escape the phagolysosome.

In one embodiment, the Listeria genome comprises a deletion of the endogenous actA gene, which in one embodiment is a virulence factor. In one embodiment, such a deletion provides a more attenuated and thus safer Listeria strain for human use. According to this embodiment, the antigenic polypeptide is integrated in frame with LLO in the Listeria chromosome. In another embodiment, the integrated nucleic acid molecule is integrated into the actA locus. In another embodiment, the chromosomal nucleic acid encoding ActA is replaced by a nucleic acid molecule encoding an antigen. In another embodiment, the Listeria strain comprises an inactivation of the endogenous actA gene. In another embodiment, the Listeria strain comprises an truncation of the endogenous actA gene. In another embodiment, the Listeria strain comprises a non-functional replacement of the endogenous actA gene. In another embodiment, the Listeria strain comprises a substitution of the endogenous actA gene. All of the above-mentioned modifications fall within the scope of what is considered to be a “mutation” of the endogenous actA gene.

In another embodiment, the Listeria strain disclosed herein comprises a mutation, deletion or an inactivation of the endogenous dal/dat and actA genes and such a Listeria strain is referred to herein as an “LmddA” strain.

In one embodiment, a nucleic acid molecule disclosed herein is plasmid vector that does not integrate in a Listeria chromosome. In another embodiment, the nucleic acid molecule is a vector designed for site-specific homologous recombination into the Listeria genome. In another embodiment, the construct or heterologous gene is integrated into the Listerial chromosome using homologous recombination.

Techniques for homologous recombination are well known in the art, and are described, for example, in Frankel, F R, Hegde, S, Lieberman, J, and Y Paterson. Induction of a cell-mediated immune response to HIV gag using Listeria monocytogenes as a live vaccine vector. J. Immunol. 155: 4766 - 4774. 1995; Mata, M, Yao, Z, Zubair, A, Syres, K and Y Paterson, Evaluation of a recombinant Listeria monocytogenes expressing an HIV protein that protects mice against viral challenge. Vaccine 19:1435-45, 2001; Boyer, J D, Robinson, T M, Maciag, P C, Peng, X, Johnson, R S, Pavlakis, G, Lewis, M G, Shen, A, Siliciano, R, Brown, C R, Weiner, D, and Y Paterson. DNA prime Listeria boost induces a cellular immune response to SIV antigens in the Rhesus Macaque model that is capable of limited suppression of SIV239 viral replication. Virology. 333: 88-101, 2005. In another embodiment, homologous recombination is performed as described in U.S. Pat. No. 6,855,320. In another embodiment, a temperature sensitive plasmid is used to select the recombinants.

In another embodiment, a nucleic acid molecule disclosed herein is integrated into the Listerial chromosome using transposon insertion. Techniques for transposon insertion are well known in the art, and are described, inter alia, by Sun et al. (Infection and Immunity 1990, 58: 3770-3778) in the construction of DP-L967. Transposon mutagenesis has the advantage, in one embodiment, that a stable genomic insertion mutant can be formed. In another embodiment, the position in the genome where the foreign gene has been inserted by transposon mutagenesis is unknown.

In another embodiment, a nucleic acid molecule disclosed herein is integrated into the Listerial chromosome using phage integration sites (Lauer P, Chow M Y et al, Construction, characterization, and use of two LM site-specific phage integration vectors. J Bacteriol 2002; 184(15): 4177-86). In another embodiment, an integrase gene and attachment site of a bacteriophage (e.g. U153 or PSA listeriophage) is used to insert the heterologous gene into the corresponding attachment site, which can be any appropriate site in the genome (e.g. comK or the 3′ end of the arg tRNA gene). In another embodiment, endogenous prophages are cured from the attachment site utilized prior to integration of the construct or heterologous gene. In another embodiment, this method results in single-copy integrants.

In another embodiment, a nucleic acid molecule of disclosed herein is operably linked to a promoter/regulatory sequence. In one embodiment, the promoter/regulatory sequence is present on an episomal plasmid cmprising said nucleic acid sequence. In one embodiment, an endogenous Listeria promoter/regulatory sequence controls the expression of a nucleic acid sequence of the methods and compositions of the present disclosure.

In one embodiment, a fusion polypeptide disclosed herein is expressed from an hly promoter, a prfA promoter, an actA promoter, or a p60 promoter or any other suitable promoter known in the art. In another embodiment, a nucleic acid sequence disclosed herein is operably linked to a promoter, regulatory sequence, or a combination thereof that drives expression of the encoded peptide in the Listeria strain. Promoter, regulatory sequences, and combinations thereof useful for driving constitutive expression of a gene are well known in the art and include, but are not limited to, for example, the P_{hlyA}, P_{actA}, hly, and p60 promoters of Listeria, the Streptococcus bac promoter, the Streptomyces griseus sgiA promoter, and the B. thuringiensis phaZ promoter. In another embodiment, inducible and tissue specific expression of the nucleic acid encoding a peptide as disclosed herein is accomplished by placing the nucleic acid encoding the peptide under the control of an inducible or tissue-specific promoter/regulatory sequence. Examples of tissue-specific or inducible regulatory sequences, promoters, and combinations thereof which are useful for his purpose include, but are not limited to the MMTV LTR inducible promoter, and the SV40 late enhancer/promoter. In another embodiment, a promoter that is induced in response to inducing agents such as metals, glucocorticoids, and the like, is utilized. Thus, it will be appreciated that the disclosure includes the use of any promoter or regulatory sequence, which is either known or unknown, and which is capable of driving expression of the desired protein operably linked thereto. In one embodiment, a regulatory sequence is a promoter, while in another embodiment, a regulatory sequence is an enhancer, while in another embodiment, a regulatory sequence is a suppressor, while in another embodiment, a regulatory sequence is a repressor, while in another embodiment, a regulatory sequence is a silencer.

In one embodiment, the nucleic acid construct used for integration to the Listeria genome contains an integration site. In one embodiment, the site is a PhSA (phage from Scott A) attPP′ integration site. PhSA is, in another embodiment, the prophage of L. monocytogenes strain ScottA (Loessner, M. J., I. B. Krause, T. Henle, and S. Scherer. 1994. Structural proteins and DNA characteristics of 14 Listeria typing bacteriophages. J. Gen. Virol. 75:701-710, incorporated herein by reference), a serotype 4b strain that was isolated during an epidemic of human listeriosis. In another embodiment, the site is any another integration site known in the art.

In another embodiment, the nucleic acid construct contains an integrase gene. In another embodiment, the integrase gene is a PhSA integrase gene. In another embodiment, the integrase gene is any other integrase gene known in the art.

In one embodiment, the nucleic acid construct is a plasmid. In another embodiment, the nucleic acid construct is a shuttle plasmid. In another embodiment, the nucleic acid construct is an integration vector. In another embodiment, the nucleic acid construct is a site-specific integration vector. In another embodiment, the nucleic acid construct is any other type of nucleic acid construct known in the art.

The integration vector of methods and compositions disclosed herein is, in another embodiment, a phage vector. In another embodiment, the integration vector is a site-specific integration vector. In another embodiment, the vector further comprises an attPP' site.

In another embodiment, the integration vector is a U153 vector. In another embodiment, the integration vector is an A118 vector. In another embodiment, the integration vector is a PhSA vector.

In another embodiment, the vector is an A511 vector (e.g. GenBank Accession No: X91069). In another embodiment, the vector is an A006 vector. In another embodiment, the vector is a B545 vector. In another embodiment, the vector is a B053 vector. In another embodiment, the vector is an A020 vector. In another embodiment, the vector is an A500 vector (e.g. GenBank Accession No: X85009). In another embodiment, the vector is a B051 vector. In another embodiment, the vector is a B052 vector. In another embodiment, the vector is a B054 vector. In another embodiment, the vector is a B055 vector. In another embodiment, the vector is a B056 vector. In another embodiment, the vector is a B101 vector. In another embodiment, the vector is a B110 vector. In another embodiment, the vector is a B111 vector. In another embodiment, the vector is an A153 vector. In another embodiment, the vector is a D441 vector. In another embodiment, the vector is an A538 vector. In another embodiment, the vector is a B653 vector. In another embodiment, the vector is an A513 vector. In another embodiment, the vector is an A507 vector. In another embodiment, the vector is an A502 vector. In another embodiment, the vector is an A505 vector. In another embodiment, the vector is an A519 vector. In another embodiment, the vector is a B604 vector. In another embodiment, the vector is a C703 vector. In another embodiment, the vector is a B025 vector. In another embodiment, the vector is an A528 vector. In another embodiment, the vector is a B024 vector. In another embodiment, the vector is a B012 vector. In another embodiment, the vector is a B035 vector. In another embodiment, the vector is a C707 vector.

In another embodiment, the vector is an A005 vector. In another embodiment, the vector is an A620 vector. In another embodiment, the vector is an A640 vector. In another embodiment, the vector is a B021 vector. In another embodiment, the vector is an HS047 vector. In another embodiment, the vector is an H10G vector. In another embodiment, the vector is an H8/73 vector. In another embodiment, the vector is an H19 vector. In another embodiment, the vector is an H21 vector. In another embodiment, the vector is an H43 vector. In another embodiment, the vector is an H46 vector. In another embodiment, the vector is an H107 vector. In another embodiment, the vector is an H108 vector. In another embodiment, the vector is an H110 vector. In another embodiment, the vector is an H83/84 vector. In another embodiment, the vector is an H312 vector. In another embodiment, the vector is an H340 vector. In another embodiment, the vector is an H387 vector. In another embodiment, the vector is an H39¹/₇3 vector. In another embodiment, the vector is an H684/74 vector. In another embodiment, the vector is an H924A vector. In another embodiment, the vector is an fMLUP5 vector. In another embodiment, the vector is a syn (=P35) vector. In another embodiment, the vector is a 00241 vector. In another embodiment, the vector is a 00611 vector. In another embodiment, the vector is a 02971A vector. In another embodiment, the vector is a 02971C vector. In another embodiment, the vector is a 5/476 vector. In another embodiment, the vector is a 5/911 vector. In another embodiment, the vector is a 5/939 vector. In another embodiment, the vector is a 5/11302 vector. In another embodiment, the vector is a 5/11605 vector. In another embodiment, the vector is a 5/11704 vector. In another embodiment, the vector is a 184 vector. In another embodiment, the vector is a 575 vector. In another embodiment, the vector is a 633 vector. In another embodiment, the vector is a 699/694 vector. In another embodiment, the vector is a 744 vector. In another embodiment, the vector is a 900 vector. In another embodiment, the vector is a 1090 vector. In another embodiment, the vector is a 1317 vector. In another embodiment, the vector is a 1444 vector. In another embodiment, the vector is a 1652 vector. In another embodiment, the vector is a 1806 vector. In another embodiment, the vector is a 1807 vector. In another embodiment, the vector is a 1921/959 vector. In another embodiment, the vector is a 1921/11367 vector. In another embodiment, the vector is a 1921/11500 vector. In another embodiment, the vector is a 1921/11566 vector. In another embodiment, the vector is a 1921/12460 vector. In another embodiment, the vector is a 1921/12582 vector. In another embodiment, the vector is a 1967vector. In another embodiment, the vector is a 2389 vector. In another embodiment, the vector is a 2425 vector. In another embodiment, the vector is a 2671 vector. In another embodiment, the vector is a 2685 vector. In another embodiment, the vector is a 3274 vector. In another embodiment, the vector is a 3550 vector. In another embodiment, the vector is a 3551 vector. In another embodiment, the vector is a 3552 vector. In another embodiment, the vector is a 4276 vector. In another embodiment, the vector is a 4277 vector. In another embodiment, the vector is a 4292 vector. In another embodiment, the vector is a 4477 vector. In another embodiment, the vector is a 5337 vector. In another embodiment, the vector is a 5348/11363 vector. In another embodiment, the vector is a 5348/1846 vector. In another embodiment, the vector is a 5348/12430 vector. In another embodiment, the vector is a 5348/12434 vector. In another embodiment, the vector is a 10072 vector. In another embodiment, the vector is a 11355C vector. In another embodiment, the vector is a 11711A vector. In another embodiment, the vector is a 12029 vector. In another embodiment, the vector is a 12981 vector. In another embodiment, the vector is a 13441 vector. In another embodiment, the vector is a 90666 vector. In another embodiment, the vector is a 9088 vector. In another embodiment, the vector is a 93253 vector. In another embodiment, the vector is a 907515 vector. In another embodiment, the vector is a 910716 vector. In another embodiment, the vector is a NN-Listeria vector. In another embodiment, the vector is a 01761 vector. In another embodiment, the vector is a 4211 vector. In another embodiment, the vector is a 4286 vector. In another embodiment, the integration vector is any other site-specific integration vector known in the art that is capable of infecting Listeria.

In another embodiment, a plasmid disclosed herein does not confer antibiotic resistance to the Listeria strain. In another embodiment, an integration vector or integrative plasmid does not contain an antibiotic resistance gene.

In another embodiment, disclosed herein is a recombinant nucleic acid encoding a recombinant polypeptide. In one embodiment, the nucleic acid comprises a sequence sharing at least 80% homology with a nucleic acid encoding a recombinant polypeptide disclosed herein. In another embodiment, the nucleic acid comprises a sequence sharing at least 85% homology with a nucleic acid encoding a recombinant polypeptide disclosed herein. In another embodiment, the nucleic acid comprises a sequence sharing at least 90% homology with a nucleic acid encoding a recombinant polypeptide disclosed herein. In another embodiment, the nucleic acid comprises a sequence sharing at least 95% homology with a nucleic acid encoding a recombinant polypeptide disclosed herein. In another embodiment, the nucleic acid comprises a sequence sharing at least 97% homology with a nucleic acid encoding a recombinant polypeptide disclosed herein. In another embodiment, the nucleic acid comprises a sequence sharing at least 99% homology with a nucleic acid encoding a recombinant polypeptide disclosed herein.

In one embodiment, a plasmid disclosed herein is an episomal plasmid that remains extrachromosomal. In another embodiment, the plasmid is an integrative plasmid.

In another embodiment, the method disclosed herein comprises expressing the antigens and fusion proteins disclosed herein under conditions conducive to protein expression.

It will be appreciated by a skilled artisan that the nucleic acids disclosed herein comprise DNA vectors, RNA vectors, plasmids (extrachromosomal and/or integrative), etc., that may be used in the methods disclosed herein for generating any of the compositions disclosed herein.

In one embodiment, a heterologous antigen disclosed herein is associated with the local tissue environment that is further associated with the development of or metastasis of cancer. In another embodiment, the heterologous antigen disclosed herein is associated with tumor immune evasion or resistance to cancer.

In one embodiment, a recombinant Listeria strain disclosed herein comprises an episomal expression vector comprising a nucleic acid molecule encoding a heterologous antigen. In another embodiment, the nucleic acid molecule is present in said episomal expression vector in an open reading frame with a truncated LLO, truncated ActA or a PEST amino acid sequence.

In another embodiment, an episomal expression vector disclosed herein comprises an antigen fused in frame to a nucleic acid sequence encoding a truncated LLO, truncated ActA or PEST amino acid sequence. In one embodiment, the antigen is a neoantigen, an HPV strain 17 E7, an HPV strain 18 E7, a PSA or a chimeric HER2 (cHER2). In another embodiment, fusion of an antigen to any LLO sequence that includes one of the PEST AA sequences enumerated herein can enhance cell mediated immunity against a heterologous antigen.

In another embodiment, either a whole E7 protein or a fragment thereof is fused to a LLO protein, ActA protein, or PEST amino acid sequence-containing peptide to generate a recombinant polypeptide disclosed herein. The E7 protein that is utilized (either whole or as the source of the fragments) comprises the amino acid sequence set forth in SEQ ID NO: 22: H G D T P T L H E Y M L D L Q P E T T D L Y C Y E Q L N D S S E E E D E I D G P A G Q A E P D R A H Y N I V T F C C K C D S T L R L C V Q S T H VD I R T L E D L L M G T L G I V C P I C S Q K P (SEQ ID NO: 22). In another embodiment, the E7 protein is a homologue of SEQ ID No: 22. In another embodiment, the E7 protein is a variant of SEQ ID No: 22. In another embodiment, the E7 protein is an isomer of SEQ ID No: 22. In another embodiment, the E7 protein is a fragment of SEQ ID No: 22. In another embodiment, the E7 protein is a fragment of a homologue of SEQ ID No: 22. In another embodiment, the E7 protein is a fragment of a variant of SEQ ID No: 22. In another embodiment, the E7 protein is a fragment of an isomer of SEQ ID No: 22.

In another embodiment, the amino acid sequence of a truncated LLO fused to an E7 protein comprises the following amino acid sequence:

(SEQ ID NO: 23)
MKKIMLVFITLILVSLPIAQQTEAKDASAFNKENSISSMAPPASPPASP
KTPIEKKHADEIDKYIQGLDYNKNNVLVYHGDAVTNVPPRKGYKDGNEY
IVVEKKKKSINQNNADIQVVNAISSLTYPGALVKANSELVENQPDVLPV
KRDSLTLSIDLPGMTNQDNKIVVKNATKSNVNNAVNTLVERWNEKYAQA
YPNVSAKIDYDDEMAYSESQLIAKFGTAFKAVNNSLNVNFGAISEGKMQ
EEVISFKQIYYNVNVNEPTRPSRFFGKAVTKEQLQALGVNAENPPAYIS
SVAYGRQVYLKLSTNSHSTKVKAAFDAAVSGKSVSGDVELTNIIKNSSF
KAVIYGGSAKDEVQIIDGNLGDLRDILKKGATFNRETPGVPIAYTTNFL
KDNELAVIKNNSEYIETTSKAYTDGKINIDHSGGYVAQFNISWDEVNYD
LEHGDTPTLHEYMLDLQPETTDLYCYEQLNDSSEEEDEIDGPAGQAEPD
RAHYNIVTFCCKCDSTLRLCVQSTHVDIRTLEDLLMGTLGIVCPICSQK
P.

In another embodiment, the fusion protien of tLLO-E7 is a homologue of SEQ ID No: 23. In another embodiment, the fusion protein is a variant of SEQ ID No: 23. In another embodiment, the tLLO-E7 fusion protein is an isomer of SEQ ID No: 23. In another embodiment, the tLLO-E7 fusion protein is a fragment of SEQ ID No: 23. In another embodiment, the tLLO-E7 fusion protein is a fragment of a homologue of SEQ ID No: 23. In another embodiment, the tLLO-E7 fusion protein is a fragment of a variant of SEQ ID No: 23. In another embodiment, the tLLO-E7 fusion protein is a fragment of an isomer of SEQ ID No: 23.

In another embodiment, a PEST AA sequence is a PEST sequence from a Listeria ActA protein. In another embodiment, a PEST sequence comprises KTEEQPSEVNTGPR (SEQ ID NO: 24), KASVTDTSEGDLDSSMQSADESTPQPLK (SEQ ID NO: 25), KNEEVNASDFPPPPTDEELR (SEQ ID NO: 26), or RGGIPTSEEFSSLNSGDFTDDENSETTEEEIDR (SEQ ID NO: 27). In another embodiment, the PEST sequence is from Listeria seeligeri cytolysin, encoded by the lso gene. In another embodiment, the PEST sequence comprises RSEVTISPAETPESPPATP (SEQ ID NO: 28). In another embodiment, the PEST sequence is from Streptolysin O protein of Streptococcus sp. In another embodiment, the PEST sequence is from Streptococcus pyogenes Streptolysin O, e.g. KQNTASTETTTTNEQPK (SEQ ID NO: 29) at AA 35-51. In another embodiment, the PEST sequence is from Streptococcus equisimilis Streptolysin O, e.g. KQNTANTETTTTNEQPK (SEQ ID NO: 30) at AA 38-54. In another embodiment, the PEST sequence has a sequence selected from SEQ ID NO: 24-30. In another embodiment, the PEST sequence has a sequence selected from SEQ ID NO: 24-30. In another embodiment, the PEST sequence is another PEST AA sequence derived from a prokaryotic organism. In another embodiment, the PEST sequence is any other PEST sequence known in the art, including, but not limited to, those disclosed in United States Patent Publication No. 2014/0186387, which is incorporated by reference herein in its entirety.

Identification of PEST sequences is well known in the art, and is described, for example in Rogers S et al (Amino acid sequences common to rapidly degraded proteins: the PEST hypothesis. Science 1986; 234(4774):364-8, incorporated herein by reference) and Rechsteiner M et al (PEST sequences and regulation by proteolysis. Trends Biochem Sci 1996; 21(7):267-71, incorporated herein by reference). “PEST sequence” refers, in another embodiment, to a region rich in proline (P), glutamic acid (E), serine (S), and threonine (T) residues. In another embodiment, the PEST sequence is flanked by one or more clusters containing several positively charged amino acids. In another embodiment, the PEST sequence mediates rapid intracellular degradation of proteins containing it. In another embodiment, the PEST sequence fits an algorithm disclosed in Rogers et al. In another embodiment, the PEST sequence fits an algorithm disclosed in Rechsteiner et al. In another embodiment, the PEST sequence contains one or more internal phosphorylation sites, and phosphorylation at these sites precedes protein degradation. In one embodiment, a sequence referred to herein as a PEST sequence is a PEST sequence.

In one embodiment, PEST sequences of prokaryotic organisms are identified in accordance with methods such as described by, for example Rechsteiner and Rogers (1996, Trends Biochem. Sci. 21:267-271) for Lm and in Rogers S et al (Science 1986; 234(4774):364-8). Alternatively, PEST AA sequences from other prokaryotic organisms can also be identified based on this method. Other prokaryotic organisms wherein PEST AA sequences would be expected to include, but are not limited to, other Listeria species. In one embodiment, the PEST sequence fits an algorithm disclosed in Rogers et al. In another embodiment, the PEST sequence fits an algorithm disclosed in Rechsteiner et al. In another embodiment, the PEST sequence is identified using the PEST-find program.

In another embodiment, identification of PEST motifs is achieved by an initial scan for positively charged amino acids R, H, and K within the specified protein sequence. All amino acids between the positively charged flanks are counted and only those motifs are considered further, which contain a number of amino acids equal to or higher than the window-size parameter. In another embodiment, a PEST sequence must contain at least 1 P, 1 D or E, and at least 1 S or T.

In another embodiment, the quality of a PEST motif is refined by means of a scoring parameter based on the local enrichment of critical amino acids as well as the motifs hydrophobicity. Enrichment of D, E, P, S and T is expressed in mass percent (w/w) and corrected for 1 equivalent of D or E, 1 of P and 1 of S or T. In another embodiment, calculation of hydrophobicity follows in principle the method of J. Kyte and R. F. Doolittle (Kyte, J and Dootlittle, R F. J. Mol. Biol. 157, 105 (1982), incorporated herein by reference. For simplified calculations, Kyte-Doolittle hydropathy indices, which originally ranged from −4.5 for arginine to +4.5 for isoleucine, are converted to positive integers, using the following linear transformation, which yielded values from 0 for arginine to 90 for isoleucine.

Hydropathy index=10*Kyte-Doolittle hydropathy index+45

In another embodiment, a potential PEST motif's hydrophobicity is calculated as the sum over the products of mole percent and hydrophobicity index for each amino acid species. The desired PEST score is obtained as combination of local enrichment term and hydrophobicity term as expressed by the following equation:

PEST score=0.55*DEPST−0.5*hydrophobicity index.

In another embodiment, “PEST sequence,” “PEST sequence,” “PEST amino acid sequence” or “PEST sequence peptide” are used interchangeably here and refer to a peptide having a score of at least +5, using the above algorithm. In another embodiment, the term refers to a peptide having a score of at least 6. In another embodiment, the peptide has a score of at least 7. In another embodiment, the score is at least 8. In another embodiment, the score is at least 9. In another embodiment, the score is at least 10. In another embodiment, the score is at least 11. In another embodiment, the score is at least 12. In another embodiment, the score is at least 13. In another embodiment, the score is at least 14. In another embodiment, the score is at least 15. In another embodiment, the score is at least 8. In another embodiment, the score is at least 17. In another embodiment, the score is at least 18. In another embodiment, the score is at least 19. In another embodiment, the score is at least 20. In another embodiment, the score is at least 21. In another embodiment, the score is at least 22. In another embodiment, the score is at least 22. In another embodiment, the score is at least 24. In another embodiment, the score is at least 24. In another embodiment, the score is at least 25. In another embodiment, the score is at least 26. In another embodiment, the score is at least 27. In another embodiment, the score is at least 28. In another embodiment, the score is at least 29. In another embodiment, the score is at least 30. In another embodiment, the score is at least 32. In another embodiment, the score is at least 35. In another embodiment, the score is at least 38. In another embodiment, the score is at least 40. In another embodiment, the score is at least 45.

In another embodiment, the PEST sequence is identified using any other method or algorithm known in the art, e.g the CaSPredictor (Garay-Malpartida H M, Occhiucci J M, Alves J, Belizario J E. Bioinformatics. 2005 June; 21 Suppl 1:i169-76). In another embodiment, the following method is used:

A PEST index is calculated for each stretch of appropriate length (e.g. a 30-35 amino acid stretch) by assigning a value of 1 to the amino acids Ser, Thr, Pro, Glu, Asp, Asn, or Gln. The coefficient value (CV) for each of the PEST residue is 1 and for each of the other amino acids (non-PEST) is 0.

In another embodiment, the PEST sequence is any other PEST sequence known in the art.

In one embodiment, the present disclosure provides fusion proteins, which in one embodiment, are expressed by Listeria. In one embodiment, such fusion proteins comprise fusions to a tLLO, a truncated ActA or a PEST sequence. It will be understood by a skilled artisan that the term “PEST sequence” may encompass cases wherein a protein fragment comprises a PEST sequence having surrounding sequences other than the PEST sequence. In another embodiment, the protein fragment consists of the PEST sequence. Thus, in another embodiment, “fusion” refers to two peptides or protein fragments either linked together at their respective ends or embedded one within the other. It will be appreciated by a skilled artisan that the term “fused” may also encompass an operable linkage by covalent bonding. In one embodiment, the term encompasses recombinant fusion (of nucleic acid sequences or open reading frames thereof). In another embodiment, the term encompasses chemical conjugation.

In another embodiment, a recombinant Listeria strain of the methods and compositions disclosed herein comprise a nucleic acid molecule operably integrated into the Listeria genome as an open reading frame with an endogenous ActA sequence. In another embodiment, a recombinant Listeria strain of the methods and compositions as disclosed herein comprise an episomal expression vector comprising a nucleic acid molecule encoding fusion protein comprising an antigen fused to an ActA or a truncated ActA. In one embodiment, the expression and secretion of the antigen is under the control of an actA promoter and ActA signal sequence and it is expressed as fusion to 1-233 amino acids of ActA (truncated ActA or tActA). In another embodiment, the truncated ActA consists of the first 390 amino acids of the wild type ActA protein as described in U.S. Pat. No. 7,655,238, which is incorporated by reference herein in its entirety. In another embodiment, the truncated ActA is an ActA-N100 or a modified version thereof (referred to as ActA-N100*) in which a PEST motif has been deleted and containing the nonconservative QDNKR substitution as described in US Patent Publication Serial No. 2014/0186387, which is incorporated by reference herein in its entirety.

In another embodiment, the LmddA strain disclosed herein comprises a mutation.

In one embodiment, an antigen of the methods and compositions disclosed herein is fused to an ActA protein, which in one embodiment, is an N-terminal fragment of an ActA protein, which in one embodiment, comprises or consists of the first 390 AA of ActA, in another embodiment, the first 418 AA of ActA, in another embodiment, the first 50 AA of ActA, in another embodiment, the first 100 AA of ActA, which in one embodiment, comprise a PEST sequence disclosed herein. In another embodiment, an N-terminal fragment of an ActA protein utilized in methods and compositions as disclosed herein comprises or consists of the first 150 AA of ActA, in another embodiment, the first approximately 200 AA of ActA, which in one embodiment comprises 2 PEST sequences as described herein. In another embodiment, an N-terminal fragment of an ActA protein utilized in methods and compositions as disclosed herein comprises or consists of the first 250 AA of ActA, in another embodiment, the first 300 AA of ActA. In another embodiment, the ActA fragment contains residues of a homologous ActA protein that correspond to one of the above AA ranges. The residue numbers need not, in another embodiment, correspond exactly with the residue numbers enumerated above; e.g. if the homologous ActA protein has an insertion or deletion, relative to an ActA protein utilized herein, then the residue numbers can be adjusted accordingly, as would be routine to a skilled artisan using sequence alignment tools such as NCBI BLAST that are well-known in the art.

In another embodiment, the N-terminal portion of the ActA protein comprises 1, 2, 3, or 4 PEST sequences, which in one embodiment are the PEST sequences specifically mentioned herein, or their homologs, disclosed herein or other PEST sequences as can be determined using the methods and algorithms described herein or by using alternative methods known in the art.

In one embodiment, the terms “N-terminal ActA” and “truncated ActA” are used interchangeably herein.

In one embodiment, an N-terminal fragment of an ActA protein utilized in methods and compositions as disclosed herein has, in another embodiment, the sequence set forth in SEQ ID NO: 31: MRAMMVVFITANCITINPDIIFAATDSEDS SLNTDEWEEEKTEEQPSEVNTGPRYETAR EVSSRDIKELEKSNKVRNTNKADLIAMLKEKAEKGPNINNNNSEQTENAAINEEASG ADRPAIQVERRHPGLPSDSAAEIKKRRKAIASSDSELESLTYPDKPTKVNKKKVAKES VADASESDLDSSMQSADESSPQPLKANQQPFFPKVFKKIKDAGKWVRDKIDENPEVK KAIVDKSAGLIDQLLTKKKSEEVNASDFPPPPTDEELRLALPETPMLLGFNAPATSEPS SFEFPPPPTDEELRLALPETPMLLGFNAPATSEPSSFEFPPPPTEDELEIIRETASSLDSSF TRGDLASLRNAINRHSQNFSDFPPIPTEEELNGRGGRP (SEQ ID NO: 31). In another embodiment, the ActA fragment comprises the sequence set forth in SEQ ID NO: 31. In another embodiment, the ActA fragment is any other ActA fragment known in the art. In another embodiment, the ActA protein is a homologue of SEQ ID NO: 31. In another embodiment, the ActA protein is a variant of SEQ ID NO: 31. In another embodiment, the ActA protein is an isoform of SEQ ID NO: 31. In another embodiment, the ActA protein is a fragment of SEQ ID NO: 31. In another embodiment, the ActA protein is a fragment of a homologue of SEQ ID NO: 31. In another embodiment, the ActA protein is a fragment of a variant of SEQ ID NO: 31. In another embodiment, the ActA protein is a fragment of an isoform of SEQ ID NO: 31.

In another embodiment, the recombinant nucleotide encoding a fragment of an ActA protein comprises the sequence set forth in SEQ ID NO: 32: atgcgtgcgatgatggtggttttcattactgccaattgcattacgattaaccccgacataatatttgcagcgacagatagcgaagattcta gtctaaacacagatgaatgggaagaagaaaaaacagaagagcaaccaagcgaggtaaatacgggaccaagatacgaaactgcac gtgaagtaagttcacgtgatattaaagaactagaaaaatcgaataaagtgagaaatacgaacaaagcagacctaatagcaatgttgaa agaaaaagcagaaaaaggtccaaatatcaataataacaacagtgaacaaactgagaatgcggctataaatgaagaggcttcaggag ccgaccgaccagctatacaagtggagcgtcgtcatccaggattgccatcggatagcgcagcggaaattaaaaaaagaaggaaagc catagcatcatcggatagtgagcttgaaagccttacttatccggataaaccaacaaaagtaaataagaaaaaagtggcgaaagagtca gttgcggatgcttctgaaagtgacttagattctagcatgcagtcagcagatgagtcttcaccacaacctttaaaagcaaaccaacaacca tttttccctaaagtatttaaaaaaataaaagatgcggggaaatgggtacgtgataaaatcgacgaaaatcctgaagtaaagaaagcgatt gttgataaaagtgcagggttaattgaccaattattaaccaaaaagaaaagtgaagaggtaaatgcttcggacttcccgccaccacctac ggatgaagagttaagacttgctttgccagagacaccaatgcttcttggttttaatgctcctgctacatcagaaccgagctcattcgaatttc caccaccacctacggatgaagagttaagacttgctttgccagagacgccaatgcttcttggttttaatgctcctgctacatcggaaccga gctcgttcgaatttccaccgcctccaacagaagatgaactagaaatcatccgggaaacagcatcctcgctagattctagttttacaagag gggatttagctagtttgagaaatgctattaatcgccatagtcaaaatttctctgatttcccaccaatcccaacagaagaagagttgaacgg gagaggcggtagacca (SEQ ID NO: 32). In another embodiment, the recombinant nucleotide has the sequence set forth in SEQ ID NO: 32. In another embodiment, the recombinant nucleotide comprises any other sequence that encodes a fragment of an ActA protein.

An N-terminal fragment of an ActA protein utilized in methods and compositions as disclosed herein has, in another embodiment, the sequence set forth in SEQ ID NO: 33: MRAMMVVFITANCITINPDIIFAATDSEDSSLNTDEWEEEKTEEQPSEVNTGPRYETA REVSSRDIEELEKSNKVKNTNKADLIAMLKAKAEKGPNNNNNNGEQTGNVAINEEA SGVDRPTLQVERRHPGLSSDSAAEIKKRRKAIASSDSELESLTYPDKPTKANKRKVA KESVVDASESDLDSSMQSADESTPQPLKANQKPFFPKVFKKIKDAGKWVRDKIDENP EVKKAIVDKSAGLIDQLLTKKKSEEVNASDFPPPPTDEELRLALPETPMLLGFNAPTP SEPS SFEFPPPPTDEELRLALPETPMLLGFNAPATSEPSSFEFPPPPTEDELEIMRETAPS LDSSFTSGDLASLRSAINRHSENFSDFPLIPTEEELNGRGGRP (SEQ ID NO: 33), which in one embodiment is the first 390 AA for ActA from Listeria monocytogenes, strain 104035. In another embodiment, the ActA fragment comprises the sequence set forth in SEQ ID NO: 32. In another embodiment, the ActA fragment is any other ActA fragment known in the art. In another embodiment, the ActA protein is a homologue of SEQ ID NO: 33. In another embodiment, the ActA protein is a variant of SEQ ID NO: 33. In another embodiment, the ActA protein is an isoform of SEQ ID NO: 33. In another embodiment, the ActA protein is a fragment of SEQ ID NO: 33. In another embodiment, the ActA protein is a fragment of a homologue of SEQ ID NO: 33. In another embodiment, the ActA protein is a fragment of a variant of SEQ ID NO: 33. In another embodiment, the ActA protein is a fragment of an isoform of SEQ ID NO: 33.

In another embodiment, a truncated ActA protein comprises the sequence set forth in SEQ ID NO: 34:

(SEQ ID NO: 34)
A T D S E D S S L N T D E W E E E K T E E Q P S E
V N T G P R Y E T A R E V S S R D I E E L E K S N
K V K N T N K A D L I A M L K A K A E K G P N N N
N N N G E Q T G N V A I N E E A S G V D R P T L Q
V E R R H P G L S S D S A A E I K K R R K A I A S
S D S E L E S L T Y P D K P T K A N K R K V A K E
S V V D A S E S D L D S S M Q S A D E S T P Q P L
K A N Q K P F F P K V F K K I K D A G K W V R D K.

In another embodiment, a truncated ActA sequence disclosed herein is further fused to an hly signal peptide at the N-terminus. In another embodiment, the truncated ActA fused to hly signal peptide comprises SEQ ID NO: 35: M K K I M L V F I T L I L V S L P I A Q Q T E A S R A T D S E D S S L N T D E W E E E K T E E Q P S E V N T G P R Y E T A R E V S S R D I E E L E K S N K V K N T N K A D L I A M L K A K A E K G P N N N N N N G E Q T G N V A I N E E A S G V D R P T L Q V E R R H P GL S S D S A A E I K KR R K A I AS S D S E L ES L T Y P D K P T K A N K R K V A K E S V V D A S E S DL D S S M Q S A D E S T P Q P L K A N Q K P F F P K V F K K I K D A G K W V R D K (SEQ ID NO: 35). In another embodiment, a truncated ActA as set forth in SEQ ID NO: 34 is referred to as LA229.

In another embodiment, the recombinant nucleotide encoding a fragment of an ActA protein comprises the sequence set forth in SEQ ID NO: 36: atgcgtgcgatgatggtagtfficattactgccaactgcattacgattaaccccgacataatatttgcagcgacagatagcgaagattcca gtctaaacacagatgaatgggaagaagaaaaaacagaagagcagccaagcgaggtaaatacgggaccaagatacgaaactgcacg tgaagtaagttcacgtgatattgaggaactagaaaaatcgaataaagtgaaaaatacgaacaaagcagacctaatagcaatgttgaaag caaaagcagagaaaggtccgaataacaataataacaacggtgagcaaacaggaaatgtggctataaatgaagaggcttcaggagtcg accgaccaactctgcaagtggagcgtcgtcatccaggtctgtcatcggatagcgcagcggaaattaaaaaaagaagaaaagccatag cgtcgtcggatagtgagcttgaaagccttacttatccagataaaccaacaaaagcaaataagagaaaagtggcgaaagagtcagttgtg gatgcttctgaaagtgacttagattctagcatgcagtcagcagacgagtctacaccacaacctttaaaagcaaatcaaaaaccattfficc ctaaagtatttaaaaaaataaaagatgcggggaaatgggtacgtgataaaatcgacgaaaatcctgaagtaaagaaagcgattgttgat aaaagtgcagggttaattgaccaattattaaccaaaaagaaaagtgaagaggtaaatgcttcggacttcccgccaccacctacggatga agagttaagacttgctttgccagagacaccgatgcttctcggffitaatgctcctactccatcggaaccgagctcattcgaatttccgccgc cacctacggatgaagagttaagacttgctttgccagagacgccaatgcttcttggttttaatgctcctgctacatcggaaccgagctcattc gaatttccaccgcctccaacagaagatgaactagaaattatgcgggaaacagcaccttcgctagattctagttttacaagcggggattta gctagtttgagaagtgctattaatcgccatagcgaaaatttctctgatttcccactaatcccaacagaagaagagttgaacgggagaggc ggtagacca (SEQ ID NO: 36), which in one embodiment, is the first 1170 nucleotides encoding ActA in Listeria monocytogenes 10403S strain. In another embodiment, the recombinant nucleotide has the sequence set forth in SEQ ID NO: 36. In another embodiment, the recombinant nucleotide comprises any other sequence that encodes a fragment of an ActA protein.

In another embodiment, the ActA fragment is another ActA fragment known in the art, which in one embodiment, is any fragment comprising a PEST sequence. Thus, in one embodiment, the ActA fragment is amino acids 1-100 of the ActA sequence. In another embodiment, the ActA fragment is amino acids 1-200 of the ActA sequence. In another embodiment, the ActA fragment is amino acids 200-300 of the ActA sequence. In another embodiment, the ActA fragment is amino acids 300-400 of the ActA sequence. In another embodiment, the ActA fragment is amino acids 1-300 of the ActA sequence. In another embodiment, a recombinant nucleotide as disclosed herein comprises any other sequence that encodes a fragment of an ActA protein. In another embodiment, the recombinant nucleotide comprises any other sequence that encodes an entire ActA protein.

In one embodiment, the ActA sequence for use in the compositions and methods as disclosed herein is from Listeria monocytogenes, which in one embodiment, is the EGD strain, the 10403S strain (Genbank accession number: DQ054585) the NICPBP 54002 strain (Genbank accession number: EU394959), the S3 strain (Genbank accession number: EU394960), the NCTC 5348 strain (Genbank accession number: EU394961), the NICPBP 54006 strain (Genbank accession number: EU394962), the M7 strain (Genbank accession number: EU394963), the S19 strain (Genbank accession number: EU394964), or any other strain of Listeria monocytogenes which is known in the art.

In one embodiment, the sequence of the deleted actA region in the strain, LmddAactA is as follows:

(SEQ ID NO: 37)
gcgccaaatcattggttgattggtgaggatgtctgtgtgcgtgggtcgcg
agatgggcgaataagaagcattaaagatcctgacaaatataatcaagcgg
ctcatatgaaagattacgaatcgcttccactcacagaggaaggcgactgg
ggcggagttcattataatagtggtatcccgaataaagcagcctataatac
tatcactaaacttggaaaagaaaaaacagaacagctttattttcgcgcct
taaagtactatttaacgaaaaaatcccagtttaccgatgcgaaaaaagcg
cttcaacaagcagcgaaagatttatatggtgaagatgcttctaaaaaagt
tgctgaagcttgggaagcagttggggttaactgattaacaaatgttagag
aaaaattaattctccaagtgatattcttaaaataattcatgaatattttt
tcttatattagctaattaagaagataactaactgctaatccaatttttaa
cggaacaaattagtgaaaatgaaggccgaattttccttgttctaaaaagg
ttgtattagcgtatcacgaggagggagtataagtgggattaaacagattt
atgcgtgcgatgatggtggttttcattactgccaattgcattacgattaa
ccccgacgtcgacccatacgacgttaattcttgcaatgttagctattggc
gtgttctctttaggggcgtttatcaaaattattcaattaagaaaaaataa
ttaaaaacacagaacgaaagaaaaagtgaggtgaatgatatgaaattcaa
aaaggtggttctaggtatgtgcttgatcgcaagtgttctagtctttccgg
taacgataaaagcaaatgcctgttgtgatgaatacttacaaacacccgca
gctccgcatgatattgacagcaaattaccacataaacttagttggtccgc
ggataacccgacaaatactgacgtaaatacgcactattggctttttaaac
aagcggaaaaaatactagctaaagatgtaaatcatatgcgagctaattta
atgaatgaacttaaaaaattcgataaacaaatagctcaaggaatatatga
tgcggatcataaaaatccatattatgatactagtacatttttatctcatt
tttataatcctgatagagataatacttatttgccgggttttgctaatgcg
aaaataacaggagcaaagtatttcaatcaatcggtgactgattaccgaga
agggaa.

In one embodiment, the underlined region contains actA sequence element that is present in the LmddΔactA strain. In one embodiment, the bold sequence gtcgac represent the site of junction of the N-T and C-T sequence.

The immune response induced by methods and compositions disclosed herein is, in another embodiment, a T cell response. In another embodiment, the immune response comprises a T cell response. In another embodiment, the response is a CD8+ T cell response. In another embodiment, the response comprises a CD8⁺ T cell response.

In one embodiment, a recombinant Listeria of the compositions and methods as disclosed herein comprise an angiogenic polypeptide. In another embodiment, anti-angiogenic approaches to cancer therapy are very promising, and in one embodiment, one type of such anti-angiogenic therapy targets pericytes. In another embodiment, molecular targets on vascular endothelial cells and pericytes are important targets for antitumor therapies. In another embodiment, the platelet-derived growth factor receptor (PDGF-B/PDGFR-β) signaling is important to recruit pericytes to newly formed blood vessels. Thus, in one embodiment, angiogenic polypeptides disclosed herein inhibit molecules involved in pericyte signaling, which in one embodiment, is PDGFR-β.

In one embodiment, a cancer immunotherapy disclosed herein generate effector T cells that are able to infiltrate the tumor, destroy tumor cells and eradicate the disease. In one embodiment, naturally occurring tumor infiltrating lymphocytes (TILs) are associated with better prognosis in several tumors, such as colon, ovarian and melanoma. In colon cancer, tumors without signs of micrometastasis have an increased infiltration of immune cells and a Th1 expression profile, which correlate with an improved survival of patients. Moreover, the infiltration of the tumor by T cells has been associated with success of immunotherapeutic approaches in both pre-clinical and human trials. In one embodiment, the infiltration of lymphocytes into the tumor site is dependent on the up-regulation of adhesion molecules in the endothelial cells of the tumor vasculature, generally by proinflammatory cytokines, such as IFN-γ, TNF-α and IL-1. Several adhesion molecules have been implicated in the process of lymphocyte infiltration into tumors, including intercellular adhesion molecule 1 (ICAM-1), vascular endothelial cell adhesion molecule 1 (V-CAM-1), vascular adhesion protein 1 (VAP-1) and E-selectin. However, these cell-adhesion molecules are commonly down-regulated in the tumor vasculature. Thus, in one embodiment, cancer vaccines as disclosed herein increase TILs, up-regulate adhesion molecules (in one embodiment, ICAM-1, V-CAM-1, VAP-1, E-selectin, or a combination thereof), up-regulate proinflammatory cytokines (in one embodiment, IFN-γ, TNF-α, IL-1, or a combination thereof), or a combination thereof.

In one embodiment, the compositions and methods as disclosed herein provide anti-angiogenesis therapy, which in one embodiment, may improve immunotherapy strategies. In one embodiment, the compositions and methods as disclosed herein circumvent endothelial cell anergy in vivo by up-regulating adhesion molecules in tumor vessels and enhancing leukocyte-vessel interactions, which increases the number of tumor infiltrating leukocytes, such as CD8⁺ T cells. Interestingly, enhanced anti-tumor protection correlates with an increased number of activated CD4⁺ and CD8⁺ tumor-infiltrating T cells and a pronounced decrease in the number of regulatory T cells in the tumor upon VEGF blockade.

In one embodiment, delivery of anti-angiogenic antigen simultaneously with a tumor-associated antigen to a host afflicted by a tumor as described herein, will have a synergistic effect in impacting tumor growth and a more potent therapeutic efficacy.

In another embodiment, targeting pericytes through vaccination will lead to cytotoxic T lymphocyte (CTL) infiltration, destruction of pericytes, blood vessel destabilization and vascular inflammation, which in another embodiment is associated with up-regulation of adhesion molecules in the endothelial cells that are important for lymphocyte adherence and transmigration, ultimately improving the ability of lymphocytes to infiltrate the tumor tissue. In another embodiment, concomitant delivery of a tumor-specific antigen generate lymphocytes able to invade the tumor site and kill tumor cells.

In one embodiment, the platelet-derived growth factor receptor (PDGF-B/PDGFR-β) signaling is important to recruit pericytes to newly formed blood vessels. In another embodiment, inhibition of VEGFR-2 and PDGFR-β concomitantly induces endothelial cell apoptosis and regression of tumor blood vessels, in one embodiment, approximately 40% of tumor blood vessels.

In another embodiment, a recombinant Listeria strain disclosed herein is an auxotrophic Listeria strain. In another embodiment, said auxotrophic Listeria strain is a dal/dat mutant. In another embodiment, the nucleic acid molecule is stably maintained in the recombinant bacterial strain in the absence of antibiotic selection.

In one embodiment, auxotrophic mutants useful as vaccine vectors may be generated in a number of ways. In another embodiment, D-alanine auxotrophic mutants can be generated, in one embodiment, via the disruption of both the dal gene and the dat gene to generate an attenuated auxotrophic strain of Listeria which requires exogenously added D-alanine for growth.

In one embodiment, the generation of AA strains of Listeria deficient in D-alanine, for example, may be accomplished in a number of ways that are well known to those of skill in the art, including deletion mutagenesis, insertion mutagenesis, and mutagenesis which results in the generation of frameshift mutations, mutations which cause premature termination of a protein, or mutation of regulatory sequences which affect gene expression. In another embodiment, mutagenesis can be accomplished using recombinant DNA techniques or using traditional mutagenesis technology using mutagenic chemicals or radiation and subsequent selection of mutants. In another embodiment, deletion mutants are preferred because of the accompanying low probability of reversion of the auxotrophic phenotype. In another embodiment, mutants of D-alanine which are generated according to the protocols disclosed herein may be tested for the ability to grow in the absence of D-alanine in a simple laboratory culture assay. In another embodiment, those mutants which are unable to grow in the absence of this compound are selected for further study.

In another embodiment, in addition to the aforementioned D-alanine associated genes, other genes involved in synthesis of a metabolic enzyme, as disclosed herein, may be used as targets for mutagenesis of Listeria.

In another embodiment, a recombinant nucleic acid molecule in a Listeria strain disclosed herein comprises a second open reading frame encoding a metabolic enzyme. In one embodiment, said recombinant Listeria strain comprises an episomal expression vector comprising a metabolic enzyme that complements a gene mutation, gene deletion or gene inactivation, or auxotrophy in said recombinant Listeria strain. In another embodiment, the construct is contained in the Listeria strain in an episomal fashion. In another embodiment, the foreign antigen is expressed from a vector harbored by the recombinant Listeria strain. In another embodiment, said episomal expression vector lacks an antibiotic resistance marker. In one embodiment, an antigen of the methods and compositions as disclosed herein is genetically fused to an oligopeptide comprising a PEST sequence. In another embodiment, said endogenous polypeptide comprising a PEST sequence is LLO. In another embodiment, said endogenous polypeptide comprising a PEST sequence is ActA.

In another embodiment, the metabolic enzyme complements an endogenous metabolic gene that is lacking in the remainder of the chromosome of the recombinant bacterial strain. In one embodiment, the endogenous metabolic gene is mutated in the chromosome. In another embodiment, the endogenous metabolic gene is deleted from the chromosome. In one embodiment, the endogenous metabolic gene comprises a mutation, deletion or inactivation in the chromosome. In another embodiment, said metabolic enzyme is an amino acid metabolism enzyme. In another embodiment, said metabolic enzyme catalyzes a formation of an amino acid used for a cell wall synthesis in said recombinant Listeria strain. In another embodiment, said metabolic enzyme is an alanine racemase enzyme. In another embodiment, said metabolic enzyme is a D-amino acid transferase enzyme.

In another embodiment, the metabolic enzyme catalyzes the formation of an amino acid (AA) used in cell wall synthesis. In another embodiment, the metabolic enzyme catalyzes synthesis of an AA used in cell wall synthesis. In another embodiment, the metabolic enzyme is involved in synthesis of an AA used in cell wall synthesis. In another embodiment, the AA is used in cell wall biogenesis.

In another embodiment, the metabolic enzyme is a synthetic enzyme for D-glutamic acid, a cell wall component.

In another embodiment, the metabolic enzyme is encoded by an alanine racemase gene (dal) gene. In another embodiment, the dal gene encodes alanine racemase, which catalyzes the reaction L-alanine H D-alanine.

The dal gene of methods and compositions of the methods and compositions as disclosed herein is encoded, in another embodiment, by the sequence:

atggtgacaggctggcatcgtccaacatggattgaaatagaccgcgcagcaattcgcgaaaatataaaaaatgaacaaaat aaactcccggaaagtgtcgacttatgggcagtagtcaaagctaatgcatatggtcacggaattatcgaagttgctaggacggcgaaaga agctggagcaaaaggffictgcgtagccattttagatgaggcactggctcttagagaagctggatttcaagatgactttattcttgtgcttgg tgcaaccagaaaagaagatgctaatctggcagccaaaaaccacatttcacttactgtttttagagaagattggctagagaatctaacgcta gaagcaacacttcgaattcatttaaaagtagatagcggtatggggcgtctcggtattcgtacgactgaagaagcacggcgaattgaagc aaccagtactaatgatcaccaattacaactggaaggtatttacacgcattttgcaacagccgaccagctagaaactagttattttgaacaa caattagctaagttccaaacgattttaacgagtttaaaaaaacgaccaacttatgttcatacagccaattcagctgatcattgttacagcca caaatcgggtttgatgcgattcgctttggtatttcgatgtatggattaactccctccacagaaatcaaaactagcttgccgtttgagcttaaa cctgcacttgcactctataccgagatggttcatgtgaaagaacttgcaccaggcgatagcgttagctacggagcaacttatacagcaaca gagcgagaatgggttgcgacattaccaattggctatgcggatggattgattcgtcattacagtggtttccatgttttagtagacggtgaacc agctccaatcattggtcgagtttgtatggatcaaaccatcataaaactaccacgtgaatttcaaactggttcaaaagtaacgataattggca aagatcatggtaacacggtaacagcagatgatgccgctcaatatttagatacaattaattatgaggtaacttgtttgttaaatgagcgc ata cctagaaaatacatccattag (SEQ ID NO: 38; GenBank Accession No: AF038438). In another embodiment, the nucleotide encoding dal is homologous to SEQ ID NO: 38. In another embodiment, the nucleotide encoding dal is a variant of SEQ ID NO: 38. In another embodiment, the nucleotide encoding dal is a fragment of SEQ ID NO: 38. In another embodiment, the dal protein is encoded by any other dal gene known in the art.

In another embodiment, the dal protein comprises the following amino acid sequence: MVTGWHRPTWIEIDRAAIRENIKNEQNKLPESVDLWAVVKANAYGHGIIEVARTAKE AGAKGFCVAILDEALALREAGFQDDFILVLGATRKEDANLAAKNHISLTVFREDWLE NLTLEATLRIHLKVDSGMGRLGIRTTEEARRIEATSTNDHQLQLEGIYTHFATADQLE TSYFEQQLAKFQTILTSLKKRPTYVHTANSAASLLQPQIGFDAIRFGISMYGLTPSTEIK TSLPFELKPALALYTEMVHVKELAPGDSVSYGATYTATEREWVATLPIGYADGLIRH YSGFHVLVDGEPAPIIGRVCMDQTIIKLPREFQTGSKVTIIGKDHGNTVTADDAAQYL DTINYEVTCLLNERIPRKYIH (SEQ ID NO: 39; GenBank Accession No: AF037428). In another embodiment, the dal protein is homologous to SEQ ID NO: 39. In another embodiment, the dal protein is a variant of SEQ ID NO: 39. In another embodiment, the dal protein is an isomer of SEQ ID NO: 39. In another embodiment, the dal protein is a fragment of SEQ ID NO: 39. In another embodiment, the dal protein is a fragment of a homologue of SEQ ID NO: 39. In another embodiment, the dal protein is a fragment of a variant of SEQ ID NO: 39. In another embodiment, the dal protein is a fragment of an isomer of SEQ ID NO: 39.

In another embodiment, the dal protein is any other Listeria dal protein known in the art. In another embodiment, the dal protein is any other gram-positive dal protein known in the art. In another embodiment, the dal protein is any other dal protein known in the art.

In another embodiment, the dal protein of methods and compositions as disclosed herein retains its enzymatic activity. In another embodiment, the dal protein retains 90% of wild-type activity. In another embodiment, the dal protein retains 80% of wild-type activity. In another embodiment, the dal protein retains 70% of wild-type activity. In another embodiment, the dal protein retains 60% of wild-type activity. In another embodiment, the dal protein retains 50% of wild-type activity. In another embodiment, the dal protein retains 40% of wild-type activity. In another embodiment, the dal protein retains 30% of wild-type activity. In another embodiment, the dal protein retains 20% of wild-type activity. In another embodiment, the dal protein retains 10% of wild-type activity. In another embodiment, the dal protein retains 5% of wild-type activity.

In another embodiment, the metabolic enzyme is encoded by a D-amino acid aminotransferase gene (dat). D-glutamic acid synthesis is controlled in part by the dat gene, which is involved in the conversion of D-glu+pyr to alpha-ketoglutarate+D-ala, and the reverse reaction.

In another embodiment, a dat gene utilized in the present disclosure has the sequence set forth in GenBank Accession Number AF038439. In another embodiment, the dat gene is any another dat gene known in the art.

The dat gene of methods and compositions of the methods and compositions disclosed herein is encoded, in another embodiment, by the sequence: atgaaagtattagtaaataaccatttagttgaaagagaagatgccacagttgacattgaagaccgcggatatcagtttggtgatggtgtat atgaagtagttcgtctatataatggaaaattctttacttataatgaacacattgatcgcttatatgctagtgcagcaaaaattgacttagttattc cttattccaaagaagagctacgtgaattacttgaaaaattagttgccgaaaataatatcaatacagggaatgtctatttacaagtgactcgtg gtgttcaaaacccacgtaatcatgtaatccctgatgatttccctctagaaggcgttttaacagcagcagctcgtgaagtacctagaaacga gcgtcaattcgttgaaggtggaacggcgattacagaagaagatgtgcgctggttacgctgtgatattaagagcttaaaccttttaggaaat attctagcaaaaaataaagcacatcaacaaaatgctttggaagctattttacatcgcggggaacaagtaacagaatgttctgcttcaaacg tttctattattaaagatggtgtattatggacgcatgcggcagataacttaatcttaaatggtatcactcgtcaagttatcattgatgttgcgaaa aagaatggcattcctgttaaagaagcggatttcactttaacagaccttcgtgaagcggatgaagtgttcatttcaagtacaactattgaaatt acacctattacgcatattgacggagttcaagtagctgacggaaaacgtggaccaattacagcgcaacttcatcaatattttgtagaagaaa tcactcgtgcatgtggcgaattagagtttgcaaaataa (SEQ ID NO: 40; GenBank Accession No: AF038438). In another embodiment, the nucleotide encoding dat is homologous to SEQ ID NO: 40. In another embodiment, the nucleotide encoding dat is a variant of SEQ ID NO: 40. In another embodiment, the nucleotide encoding dat is a fragment of SEQ ID NO: 40. In another embodiment, the dat protein is encoded by any other dat gene known in the art.

In another embodiment, the dat protein comprises the following amino acid sequence: MKVLVNNHLVEREDATVDIEDRGYQFGDGVYEVVRLYNGKFFTYNEHIDRLYASAA KIDLVIPYSKEELRELLEKLVAENNINTGNVYLQVTRGVQNPRNHVIPDDFPLEGVLT AAAREVPRNERQFVEGGTAITEEDVRWLRCDIKSLNLLGNILAKNKAHQQNALEAIL HRGEQVTECSASNVSIIKDGVLWTHAADNLILNGITRQVIIDVAKKNGIPVKEADFTLT DLREADEVFISSTTIEITPITHIDGVQVADGKRGPITAQLHQYFVEEITRACGELEFAK (SEQ ID NO: 41; GenBank Accession No: AF038439). In another embodiment, the dat protein is homologous to SEQ ID NO: 41. In another embodiment, the dat protein is a variant of SEQ ID NO: 41. In another embodiment, the dat protein is an isomer of SEQ ID NO: 41. In another embodiment, the dat protein is a fragment of SEQ ID NO: 41. In another embodiment, the dat protein is a fragment of a homologue of SEQ ID NO: 41. In another embodiment, the dat protein is a fragment of a variant of SEQ ID NO: 41. In another embodiment, the dat protein is a fragment of an isomer of SEQ ID NO: 41.

In another embodiment, the Dat protein is any other Listeria dat protein known in the art. In another embodiment, the Dat protein is any other gram-positive dat protein known in the art. In another embodiment, the Dat protein is any other dat protein known in the art.

In another embodiment, the Dat protein of methods and compositions of the methods and compositions as disclosed herein retains its enzymatic activity. In another embodiment, the Dat protein retains 90% of wild-type activity. In another embodiment, the Dat protein retains 80% of wild-type activity. In another embodiment, the Dat protein retains 70% of wild-type activity. In another embodiment, the Dat protein retains 60% of wild-type activity. In another embodiment, the Dat protein retains 50% of wild-type activity. In another embodiment, the Dat protein retains 40% of wild-type activity. In another embodiment, the Dat protein retains 30% of wild-type activity. In another embodiment, the dat protein retains 20% of wild-type activity. In another embodiment, the Dat protein retains 10% of wild-type activity. In another embodiment, the Dat protein retains 5% of wild-type activity.

In another embodiment, the metabolic enzyme is encoded by dga. D-glutamic acid synthesis is also controlled in part by the dga gene, and an auxotrophic mutant for D-glutamic acid synthesis will not grow in the absence of D-glutamic acid (Pucci et al, 1995, J Bacteriol. 177: 336-342). In another rembodiment, the recombinant Listeria is auxotrophic for D-glutamic acid. A further example includes a gene involved in the synthesis of diaminopimelic acid. Such synthesis genes encode beta-semialdehyde dehydrogenase, and when inactivated, renders a mutant auxotrophic for this synthesis pathway (Sizemore et al, 1995, Science 270: 299-302). In another embodiment, the dga protein is any other Listeria dga protein known in the art. In another embodiment, the dga protein is any other gram-positive dga protein known in the art.

In another embodiment, the metabolic enzyme is encoded by an alr (alanine racemase) gene. In another embodiment, the metabolic enzyme is any other enzyme known in the art that is involved in alanine synthesis. In another embodiment, the metabolic enzyme is any other enzyme known in the art that is involved in L-alanine synthesis. In another embodiment, the metabolic enzyme is any other enzyme known in the art that is involved in D-alanine synthesis. In another rembodiment, the recombinant Listeria is auxotrophic for D-alanine. Bacteria auxotrophic for alanine synthesis are well known in the art, and are described in, for example, E. coli (Strych et al, 2002, J. Bacteriol. 184:4321-4325), Corynebacterium glutamicum (Tauch et al, 2002, J. Biotechnol 99:79-91), and Listeria monocytogenes (Frankel et al, U.S. Pat. No. 6,099,848)), Lactococcus species, and Lactobacillus species, (Bron et al, 2002, Appl Environ Microbiol, 68: 5663-70). In another embodiment, any D-alanine synthesis gene known in the art is inactivated.

In another embodiment, the metabolic enzyme is an amino acid aminotransferase enzyme.

In another embodiment, the metabolic enzyme is encoded by serC, a phosphoserine aminotransferase. In another embodiment, the metabolic enzyme is encoded by asd (aspartate beta-semialdehyde dehydrogenase), involved in synthesis of the cell wall constituent diaminopimelic acid. In another embodiment, the metabolic enzyme is encoded by gsaB-glutamate-1-semialdehyde aminotransferase, which catalyzes the formation of 5-aminolevulinate from (S)-4-amino-5-oxopentanoate. In another embodiment, the metabolic enzyme is encoded by HemL, which catalyzes the formation of 5-aminolevulinate from (S)-4-amino-5-oxopentanoate. In another embodiment, the metabolic enzyme is encoded by aspB, an aspartate aminotransferase that catalyzes the formation of oxalozcetate and L-glutamate from L-aspartate and 2-oxoglutarate. In another embodiment, the metabolic enzyme is encoded by argF-1, involved in arginine biosynthesis. In another embodiment, the metabolic enzyme is encoded by aroE, involved in amino acid biosynthesis. In another embodiment, the metabolic enzyme is encoded by aroB, involved in 3-dehydroquinate biosynthesis. In another embodiment, the metabolic enzyme is encoded by aroD, involved in amino acid biosynthesis. In another embodiment, the metabolic enzyme is encoded by aroC, involved in amino acid biosynthesis. In another embodiment, the metabolic enzyme is encoded by hisB, involved in histidine biosynthesis. In another embodiment, the metabolic enzyme is encoded by hisD, involved in histidine biosynthesis. In another embodiment, the metabolic enzyme is encoded by hisG, involved in histidine biosynthesis. In another embodiment, the metabolic enzyme is encoded by metX, involved in methionine biosynthesis. In another embodiment, the metabolic enzyme is encoded by proB, involved in proline biosynthesis. In another embodiment, the metabolic enzyme is encoded by argR, involved in arginine biosynthesis. In another embodiment, the metabolic enzyme is encoded by argJ, involved in arginine biosynthesis. In another embodiment, the metabolic enzyme is encoded by thil, involved in thiamine biosynthesis. In another embodiment, the metabolic enzyme is encoded by LMOf2365_1652, involved in tryptophan biosynthesis. In another embodiment, the metabolic enzyme is encoded by aroA, involved in tryptophan biosynthesis. In another embodiment, the metabolic enzyme is encoded by ilvD, involved in valine and isoleucine biosynthesis. In another embodiment, the metabolic enzyme is encoded by ilvC, involved in valine and isoleucine biosynthesis. In another embodiment, the metabolic enzyme is encoded by leuA, involved in leucine biosynthesis. In another embodiment, the metabolic enzyme is encoded by dapF, involved in lysine biosynthesis. In another embodiment, the metabolic enzyme is encoded by thrB, involved in threonine biosynthesis (all GenBank Accession No. NC_002973).

In another embodiment, the metabolic enzyme is a tRNA synthetase. In another embodiment, the metabolic enzyme is encoded by the trpS gene, encoding tryptophanyltRNA synthetase. In another embodiment, the metabolic enzyme is any other tRNA synthetase known in the art.

In another embodiment, a recombinant Listeria strain disclosed herein has been passaged through an animal host. In another embodiment, the passaging maximizes efficacy of the strain as a vaccine vector. In another embodiment, the passaging stabilizes the immunogenicity of the Listeria strain. In another embodiment, the passaging stabilizes the virulence of the Listeria strain. In another embodiment, the passaging increases the immunogenicity of the Listeria strain. In another embodiment, the passaging increases the virulence of the Listeria strain. In another embodiment, the passaging removes unstable sub-strains of the Listeria strain. In another embodiment, the passaging reduces the prevalence of unstable sub-strains of the Listeria strain. In another embodiment, the passaging attenuates the strain, or in another embodiment, makes the strain less virulent. Methods for passaging a recombinant Listeria strain through an animal host are well known in the art, and are described, for example, in U.S. patent application Ser. No. 10/541,614.

The recombinant Listeria strain of the methods and compositions as disclosed herein is, in another embodiment, a recombinant Listeria monocytogenes strain. In another embodiment, the Listeria strain is a recombinant Listeria seeligeri strain. In another embodiment, the Listeria strain is a recombinant Listeria grayi strain. In another embodiment, the Listeria strain is a recombinant Listeria ivanovii strain. In another embodiment, the Listeria strain is a recombinant Listeria murrayi strain. In another embodiment, the Listeria strain is a recombinant Listeria welshimeri strain. In another embodiment, the Listeria strain is a recombinant strain of any other Listeria species known in the art. In another embodiment, the sequences of Listeria proteins for use in the methods and compositions disclosed herein are from any of the above-described strains.

In one embodiment, a Listeria monocytogenes strain as disclosed herein is the EGD strain, the 10403S strain, the NICPBP 54002 strain, the S3 strain, the NCTC 5348 strain, the NICPBP 54006 strain, the M7 strain, the S19 strain, or another strain of Listeria monocytogenes which is known in the art.

In another embodiment, the recombinant Listeria strain is a vaccine strain, which in one embodiment, is a bacterial vaccine strain.

In another embodiment, the recombinant Listeria strain utilized in methods of the present disclosure has been stored in a frozen cell bank. In another embodiment, the recombinant Listeria strain has been stored in a lyophilized cell bank.

In another embodiment, the cell bank of methods and compositions of the present disclosure is a master cell bank. In another embodiment, the cell bank is a working cell bank. In another embodiment, the cell bank is Good Manufacturing Practice (GMP) cell bank. In another embodiment, the cell bank is intended for production of clinical-grade material. In another embodiment, the cell bank conforms to regulatory practices for human use. In another embodiment, the cell bank is any other type of cell bank known in the art.

“Good Manufacturing Practices” are defined, in another embodiment, by (21 CFR 210-211) of the United States Code of Federal Regulations. In another embodiment, “Good Manufacturing Practices” are defined by other standards for production of clinical-grade material or for human consumption; e.g. standards of a country other than the United States..

In another embodiment, a recombinant Listeria strain utilized in methods of the present disclosure is from a batch of vaccine doses.

In another embodiment, a recombinant Listeria strain utilized in methods of the present disclosure is from a frozen stock produced by a method disclosed herein.

In another embodiment, a recombinant Listeria strain utilized in methods of the present disclosure is from a lyophilized stock produced by a method disclosed herein.

In another embodiment, a cell bank, frozen stock, or batch of vaccine doses of the present disclosure exhibits viability upon thawing of greater than 90%. In another embodiment, the thawing follows storage for cryopreservation or frozen storage for 24 hours. In another embodiment, the storage is for 2 days. In another embodiment, the storage is for 3 days. In another embodiment, the storage is for 4 days. In another embodiment, the storage is for 1 week. In another embodiment, the storage is for 2 weeks. In another embodiment, the storage is for 3 weeks. In another embodiment, the storage is for 1 month. In another embodiment, the storage is for 2 months. In another embodiment, the storage is for 3 months. In another embodiment, the storage is for 5 months. In another embodiment, the storage is for 6 months. In another embodiment, the storage is for 9 months. In another embodiment, the storage is for 1 year.

In another embodiment, a cell bank, frozen stock, or batch of vaccine doses of the present disclosure is cryopreserved by a method that comprises growing a culture of the Listeria strain in a nutrient media, freezing the culture in a solution comprising glycerol, and storing the Listeria strain at below −20 degrees Celsius. In another embodiment, the temperature is about −70 degrees Celsius. In another embodiment, the temperature is about ⁻70 -⁻80 degrees Celsius.

In another embodiment of methods and compositions of the present disclosure, the culture (e.g. the culture of a Listeria strain that is used to produce a batch of Listeria vaccine doses) is inoculated from a cell bank. In another embodiment, the culture is inoculated from a frozen stock. In another embodiment, the culture is inoculated from a starter culture. In another embodiment, the culture is inoculated from a colony. In another embodiment, the culture is inoculated at mid-log growth phase. In another embodiment, the culture is inoculated at approximately mid-log growth phase. In another embodiment, the culture is inoculated at another growth phase.

In another embodiment of methods and compositions of the present disclosure, the solution used for freezing contains glycerol in an amount of 2-20%. In another embodiment, the amount is 2%. In another embodiment, the amount is 20%. In another embodiment, the amount is 1%. In another embodiment, the amount is 1.5%. In another embodiment, the amount is 3%. In another embodiment, the amount is 4%. In another embodiment, the amount is 5%. In another embodiment, the amount is 2%. In another embodiment, the amount is 2%. In another embodiment, the amount is 7%. In another embodiment, the amount is 9%. In another embodiment, the amount is 10%. In another embodiment, the amount is 12%. In another embodiment, the amount is 14%. In another embodiment, the amount is 16%. In another embodiment, the amount is 18%. In another embodiment, the amount is 222%. In another embodiment, the amount is 25%. In another embodiment, the amount is 30%. In another embodiment, the amount is 35%. In another embodiment, the amount is 40%.

In another embodiment, the solution used for freezing contains another colligative additive or additive with anti-freeze properties, in place of glycerol. In another embodiment, the solution used for freezing contains another colligative additive or additive with anti-freeze properties, in addition to glycerol. In another embodiment, the additive is mannitol. In another embodiment, the additive is DMSO. In another embodiment, the additive is sucrose. In another embodiment, the additive is any other colligative additive or additive with anti-freeze properties that is known in the art.

In one embodiment, a vaccine is a composition which elicits an immune response to an antigen or polypeptide in the composition as a result of exposure to the composition. In another embodiment, the vaccine additionally comprises an adjuvant, cytokine, chemokine, or combination thereof In another embodiment, the vaccine or composition additionally comprises antigen presenting cells (APCs), which in one embodiment are autologous, while in another embodiment, they are allogeneic to the subject.

In one embodiment, a “vaccine” is a composition which elicits an immune response in a host to an antigen or polypeptide in the composition as a result of exposure to the composition. In one embodiment, the immune response is to a particular antigen or to a particular epitope on the antigen. In one embodiment, the vaccine may be a peptide vaccine, in another embodiment, a DNA vaccine. In another embodiment, the vaccine may be contained within and, in another embodiment, delivered by, a cell, which in one embodiment is a bacterial cell, which in one embodiment, is a Listeria. In one embodiment, a vaccine may prevent a subject from contracting or developing a disease or condition, wherein in another embodiment, a vaccine may be therapeutic to a subject having a disease or condition. In one embodiment, a vaccine of the present disclosure comprises a composition of the present disclosure and an adjuvant, cytokine, chemokine, or combination thereof.

In another embodiment, the present disclosure provides an immunogenic composition comprising a recombinant Listeria of the present disclosure. In another embodiment, the immunogenic composition of methods and compositions of the present disclosure comprises a recombinant vaccine vector of the present disclosure. In another embodiment, the immunogenic composition comprises a plasmid of the present disclosure. In another embodiment, the immunogenic composition comprises an adjuvant. In one embodiment, a vector of the present disclosure is administered as part of a vaccine composition.

In another embodiment, a vaccine of the present disclosure is delivered with an adjuvant. In one embodiment, the adjuvant favors a predominantly Th1-mediated immune response. In another embodiment, the adjuvant favors a Th1-type immune response. In another embodiment, the adjuvant favors a Th1-mediated immune response. In another embodiment, the adjuvant favors a cell-mediated immune response over an antibody-mediated response. In another embodiment, the adjuvant is any other type of adjuvant known in the art. In another embodiment, the immunogenic composition induces the formation of a T cell immune response against the target protein.

In another embodiment, the adjuvant is MPL. In another embodiment, the adjuvant is QS21. In another embodiment, the adjuvant comprises a granulocyte/macrophage colony-stimulating factor (GM-CSF) protein or a nucleotide molecule encoding a GM-CSF protein. In another embodiment, the adjuvant is a TLR agonist. In another embodiment, the adjuvant is a TLR4 agonist. In another embodiment, the adjuvant is monophosphoryl lipid A. In another embodiment, the adjuvant is a TLR9 agonist. In another embodiment, the adjuvant is Resiquimod®. In another embodiment, the adjuvant is imiquimod. In another embodiment, the adjuvant is a CpG oligonucleotide. In another embodiment, the adjuvant is a cytokine or a nucleic acid encoding same. In another embodiment, the adjuvant is a chemokine or a nucleic acid encoding same. In another embodiment, the adjuvant is IL-12 or a nucleic acid encoding same. In another embodiment, the adjuvant is IL-6 or a nucleic acid encoding same. In another embodiment, the adjuvant is a lipopolysaccharide. In another embodiment, the adjuvant is as described in Fundamental Immunology, 5th ed (August 2003): William E. Paul (Editor); Lippincott Williams & Wilkins Publishers; Chapter 43: Vaccines, G J V Nossal, which is hereby incorporated by reference. In another embodiment, the adjuvant is any other adjuvant known in the art.

In one embodiment, disclosed herein is a method of inducing an immune response to an antigen in a subject comprising administering a recombinant Listeria strain to said subject.

In one embodiment, disclosed herein is a method of inducing an anti-angiogenic immune response to an antigen in a subject comprising administering a recombinant Listeria strain to said subject. In another embodiment, said recombinant Listeria strain comprises a first and second nucleic acid molecule. In another embodiment, each said nucleic acid molecule encodes a heterologous antigen. In yet another embodiment, said first nucleic acid molecule is operably integrated into the Listeria genome as an open reading frame with an endogenous polypeptide comprising a PEST sequence.

In one embodiment, disclosed herein is a method of treating, suppressing, or inhibiting at least one cancer in a subject comprising administering a recombinant Listeria strain disclosed herein to said subject. In another embodiment, said recombinant Listeria strain comprises a nucleic acid molecule. In another embodiment, said nucleic acid molecule encodes a heterologous antigen disclosed herein. In yet another embodiment, said nucleic acid molecule is present in a plasmid in said Listeria. In another embodiment, said nucleic acid molecule is operably integrated into the Listeria genome as an open reading frame with a nucleic acid sequence encoding an endogenous polypeptide comprising a truncated LLO, a truncated ActA or a PEST amino acid sequence. In another embodiment, at least one of said antigens is expressed by at least one cell of a cancer cells. In another embodiment, a method of treating reduces or halts metastasis of a tumor or cancer. In another embodiment, a method of treating reduces or halts the growth of said tumor or said cancer.

In one embodiment, disclosed herein is a method of reducing or ameliorating an incidence of infectious disease in a subject comprising administering a recombinant Listeria strain disclosed herein to said subject.

In one embodiment, disclosed herein is a method of delaying the onset to a cancer in a subject comprising administering a recombinant Listeria strain to said subject. In another embodiment, disclosed herein is a method of delaying the progression to a cancer in a subject comprising administering a recombinant Listeria strain to said subject. In another embodiment, disclosed herein is a method of extending the remission to a cancer in a subject comprising administering a recombinant Listeria strain to said subject. In another embodiment, disclosed herein is a method of decreasing the size of an existing tumor in a subject comprising administering a recombinant Listeria strain to said subject. In another embodiment, disclosed herein is a method of preventing the growth of an existing tumor in a subject comprising administering a recombinant Listeria strain to said subject. In another embodiment, disclosed herein is a method of preventing the growth of new or additional tumors in a subject comprising administering a recombinant Listeria strain to said subject.

In one embodiment, cancer or tumors may be prevented in specific populations known to be susceptible to a particular cancer or tumor. In one embodiment, such susceptibilty may be due to environmental factors, such as smoking, which in one embodiment, may cause a population to be subject to lung cancer, while in another embodiment, such susceptbility may be due to genetic factors, for example a population with BRCA1/2 mutations may be susceptible, in one embodiment, to breast cancer, and in another embodiment, to ovarian cancer. In another embodiment, one or more mutations on chromosome 8q24, chromosome 17q12, and chromosome 17q24.3 may increase susceptibility to prostate cancer, as is known in the art. Other genetic and environmental factors contributing to cancer susceptibility are known in the art.

In one embodiment, the recombinant Listeria strain is administered to the subject at a dose of 1×10⁶-1×10⁷ CFU. In another embodiment, the recombinant Listeria strain is administered to the subject at a dose of 1×10⁷-1×10⁸ CFU. In another embodiment, the recombinant Listeria strain is administered to the subject at a dose of 1×10⁸-3.31×10¹⁰ CFU. In another embodiment, the recombinant Listeria strain is administered to the subject at a dose of 1×10⁹-3.31×10¹⁰ CFU. In another embodiment, the dose is 5-500×10⁸ CFU. In another embodiment, the dose is 7-500×10⁸ CFU. In another embodiment, the dose is 10-500×10⁸ CFU. In another embodiment, the dose is 20-500×10⁸ CFU. In another embodiment, the dose is 30-500×10⁸ CFU. In another embodiment, the dose is 50-500×10⁸ CFU. In another embodiment, the dose is 70-500×10⁸ CFU. In another embodiment, the dose is 100-500×10⁸ CFU. In another embodiment, the dose is 150-500×10⁸ CFU. In another embodiment, the dose is 5-300×10⁸ CFU. In another embodiment, the dose is 5-200×10⁸ CFU. In another embodiment, the dose is 5-15×10⁸ CFU. In another embodiment, the dose is 5-100×10⁸ CFU. In another embodiment, the dose is 5-70×10⁸ CFU. In another embodiment, the dose is 5-50×10⁸ CFU. In another embodiment, the dose is 5-30×10⁸ CFU. In another embodiment, the dose is 5-20×10⁸ CFU. In another embodiment, the dose is 1-30×10⁹ CFU. In another embodiment, the dose is 1-20×10⁹CFU. In another embodiment, the dose is 2-30×10⁹ CFU. In another embodiment, the dose is 1-10×10⁹ CFU. In another embodiment, the dose is 2-10×10⁹ CFU. In another embodiment, the dose is 3-10×10⁹ CFU. In another embodiment, the dose is 2-7×10⁹ CFU. In another embodiment, the dose is 2-5×10⁹ CFU. In another embodiment, the dose is 3-5×10⁹ CFU.

In another embodiment, the dose is 1×10⁷ organisms. In another embodiment, the dose is 1.5×10⁷ organisms. In another embodiment, the dose is 2×10⁸ organisms. In another embodiment, the dose is 3×10⁷ organisms. In another embodiment, the dose is 4×10⁷ organisms. In another embodiment, the dose is 5×10⁷ organisms. In another embodiment, the dose is 6×10⁷ organisms. In another embodiment, the dose is 7×10⁷ organisms. In another embodiment, the dose is 8×10⁷ organisms. In another embodiment, the dose is 10×10⁷ organisms. In another embodiment, the dose is 1.5×10⁸ organisms. In another embodiment, the dose is 2×10⁸ organisms. In another embodiment, the dose is 2.5×10⁸ organisms. In another embodiment, the dose is 3×10⁸ organisms. In another embodiment, the dose is 3.3×10⁸ organisms. In another embodiment, the dose is 4×10⁸ organisms. In another embodiment, the dose is 5×10⁸ organisms.

In another embodiment, the dose is 1×10⁹ organisms. In another embodiment, the dose is 1.5×10⁹ organisms. In another embodiment, the dose is 2×10⁹ organisms. In another embodiment, the dose is 3×10⁹ organisms. In another embodiment, the dose is 4×10⁹ organisms. In another embodiment, the dose is 5×10⁹ organisms. In another embodiment, the dose is 6×10⁹ organisms. In another embodiment, the dose is 7×10⁹ organisms. In another embodiment, the dose is 8×10⁹ organisms. In another embodiment, the dose is 10×10⁹ organisms. In another embodiment, the dose is 1.5×10¹⁰ organisms. In another embodiment, the dose is 2×10¹⁰ organisms. In another embodiment, the dose is 2.5×10¹⁰ organisms. In another embodiment, the dose is 3×10¹⁰ organisms. In another embodiment, the dose is 3.3×10¹⁰ organisms. In another embodiment, the dose is 4×10¹⁰ organisms. In another embodiment, the dose is 5×10¹⁰ organisms.

In one embodiment, the methods disclosed herein comprise boosting a subject with a Listeria-based immunotherapy disclosed herein. It will be appreciated by the skilled artisan that the term “Boosting” may encompass administering an additional Liseria-based immunotherapy, immunogenic composition, or recombinant Listeria strain dose to a subject. In another embodiment of methods of the present disclosure, 2 boosts (or a total of 3 inoculations) are administered. In another embodiment, 3 boosts are administered. In another embodiment, 4 boosts are administered. In another embodiment, 5 boosts are administered. In another embodiment, 6 boosts are administered. In another embodiment, more than 6 boosts are administered.

In one embodiment, an antibiotic regimen is administered following each boost with a Listeria-based immunotherapy or immunogenic composition disclosed herein.

In another embodiment, a method of present disclosure further comprises the step of boosting the subject with a recombinant Listeria strain, an oncolytic virus, CAR T cells, a therapeutic or immunomodulatory monoclonal antibody, TKI, an immune checkpoint inhibitor or Receptor engineered T cells. In another embodiment, the recombinant Listeria strain used in the booster inoculation is the same as the strain used in the initial “priming” inoculation. In another embodiment, the booster strain is different from the priming strain. In another embodiment, the recombinant immune checkpoint inhibitor used in the booster inoculation is the same as the inhibitor used in the initial “priming” inoculation. In another embodiment, the booster inhibitor is different from the priming inhibitor. In another embodiment, the same doses are used in the priming and boosting inoculations. In another embodiment, a larger dose is used in the booster. In another embodiment, a smaller dose is used in the booster. In another embodiment, the methods of the present disclosure further comprise the step of administering to the subject a booster dose. In one embodiment, the booster dose follows a single priming dose. In another embodiment, a single booster dose is administered after the priming doses. In another embodiment, two booster doses are administered after the priming doses. In another embodiment, three booster doses are administered after the priming doses. In one embodiment, the period between a prime and a boost strain is experimentally determined by the skilled artisan. In another embodiment, the period between a prime and a boost strain is 1 week, in another embodiment it is 2 weeks, in another embodiment, it is 3 weeks, in another embodiment, it is 4 weeks, in another embodiment, it is 5 weeks, in another embodiment it is 6-8 weeks, in yet another embodiment, the boost strain is administered 8-10 weeks after the prime strain.

In another embodiment, a method of the present disclosure further comprises boosting the subject with a immunogenic composition comprising an attenuated Listeria strain provided herein. In another embodiment, a method of the present disclosure comprises the step of administering a booster dose of the immunogenic composition comprising the attenuated Listeria strain provided herein. In another embodiment, the booster dose is an alternate form of said immunogenic composition. In another embodiment, the methods of the present disclosure further comprise the step of administering to the subject a booster immunogenic composition. In one embodiment, the booster dose follows a single priming dose of said immunogenic composition. In another embodiment, a single booster dose is administered after the priming dose. In another embodiment, two booster doses are administered after the priming dose. In another embodiment, three booster doses are administered after the priming dose. In one embodiment, the period between a prime and a boost dose of an immunogenic composition comprising the attenuated Listeria provided herein is experimentally determined by the skilled artisan. In another embodiment, the dose is experimentally determined by a skilled artisan. In another embodiment, the period between a prime and a boost dose is 1 week, in another embodiment it is 2 weeks, in another embodiment, it is 3 weeks, in another embodiment, it is 4 weeks, in another embodiment, it is 5 weeks, in another embodiment it is 6-8 weeks, in yet another embodiment, the boost dose is administered 8-10 weeks after the prime dose of the immunogenic composition.

Heterologous “prime boost” strategies have been effective for enhancing immune responses and protection against numerous pathogens. Schneider et al., Immunol. Rev. 170:29-38 (1999); Robinson, H. L., Nat. Rev. Immunol. 2:239-50 (2002); Gonzalo, R. M. et al., Vaccine 20:1226-31 (2002); Tanghe, A., Infect. Immun 69:3041-7 (2001). Providing antigen in different forms in the prime and the boost injections appears to maximize the immune response to the antigen. DNA vaccine priming followed by boosting with protein in adjuvant or by viral vector delivery of DNA encoding antigen appears to be the most effective way of improving antigen specific antibody and CD4+ T-cell responses or CD8+T-cell responses respectively. Shiver J. W. et al., Nature 415: 331-5 (2002); Gilbert, S. C. et al., Vaccine 20:1039-45 (2002); Billaut-Mulot, O. et al., Vaccine 19:95-102 (2000); Sin, J. I. et al., DNA Cell Biol. 18:771-9 (1999). Recent data from monkey vaccination studies suggests that adding CRL1005 poloxamer (12 kDa, 5% POE), to DNA encoding the HIV gag antigen enhances T-cell responses when monkeys are vaccinated with an HIV gag DNA prime followed by a boost with an adenoviral vector expressing HIV gag (Ads-gag). The cellular immune responses for a DNA/poloxamer prime followed by an Ad5-gag boost were greater than the responses induced with a DNA (without poloxamer) prime followed by Ad5-gag boost or for Ad5-gag only. Shiver, J. W. et al. Nature 415:331-5 (2002). U.S. Patent Appl. Publication No. US 2002/0165172 A1 describes simultaneous administration of a vector construct encoding an immunogenic portion of an antigen and a protein comprising the immunogenic portion of an antigen such that an immune response is generated. The document is limited to hepatitis B antigens and HIV antigens. Moreover, U.S. Pat. No. 6,500,432 is directed to methods of enhancing an immune response of nucleic acid vaccination by simultaneous administration of a polynucleotide and polypeptide of interest. According to the patent, simultaneous administration means administration of the polynucleotide and the polypeptide during the same immune response, preferably within 0-10 or 3-7 days of each other. The antigens contemplated by the patent include, among others, those of Hepatitis (all forms), HSV, HIV, CMV, EBV, RSV, VZV, HPV, polio, influenza, parasites (e.g., from the genus Plasmodium), and pathogenic bacteria (including but not limited to M. tuberculosis, M. leprae, Chlamydia, Shigella, B. burgdorferi, enterotoxigenic E. coli, S. typhosa, H. pylori, V. cholerae, B. pertussis, etc.). All of the above references are herein incorporated by reference in their entireties.

In one embodiment, a treatment protocol of the present disclosure is therapeutic. In another embodiment, the protocol is prophylactic. In another embodiment, the compositions of the present disclosure are used to protect people at risk for cancer such as breast cancer or other types of tumors because of familial genetics or other circumstances that predispose them to these types of ailments as will be understood by a skilled artisan. In another embodiment, the vaccines are used as a cancer immunotherapy after debulking of tumor growth by surgery, conventional chemotherapy or radiation treatment. Following such treatments, the immunotherapy disclosed herein is administered so that the CTL response to the tumor antigen of the immunotherapy destroys remaining metastases and prolongs remission from the cancer. In another embodiment, an immunotherapy disclosed herein is used to effect the growth of previously established tumors and to kill existing tumor cells.

In one embodiment, a nucleic acid molecule disclsoed herein encodes a heterologous antigen and the method is for treating, inhibiting or suppressing prostate cancer. In another embodiment, the a nucleic acid molecule encodes a heterologous antigen and the method is for treating, inhibiting or suppressing ovarian cancer. In another embodiment, the nucleic acid molecule encodes a heterologous antigen and the method is treating, inhibiting, or suppressing metastasis of prostate cancer, which in one embodiment, comprises metastasis to bone, and in another embodiment, comprises metastasis to other organs. In another embodiment, the nucleic acid molecule encodes a heterologous antigen and the method is for treating, inhibiting or suppressing metastasis of prostate cancer to bones. In yet another embodiment the method is for treating, inhibiting, or suppressing metastatis of prostate cancer to other organs. In another embodiment, the nucleic acid molecule encodes a heterologous antigen and the method is for treating, inhibiting or suppressing breast cancer. In another embodiment, the nucleic acid molecule encodes a heterologous antigen and the method is for treating, inhibiting or suppressing both prostate or breast cancer. In another embodiment, the nucleic acid molecule encodes a heterologous antigen or functional fragment thereof is expressed by or derived from an infectious pathogen and the method is for reducing or ameliorating an infectious disease.

In one embodiment, an an immunogenic composition or a therapeutic method disclosed herein is for treating, inhibiting or suppressing prostate cancer. In another embodiment, an immunogenic composition or a therapeutic method disclosed herein is for treating, inhibiting or suppressing ovarian cancer. In another embodiment, an immunogenic composition or a therapeutic method disclosed herein is for treating, inhibiting or suppressing breast cancer. In another embodiment, an immunogenic composition or a therapeutic method disclosed herein is for treating, inhibiting, or suppressing metastasis of prostate cancer, which in one embodiment, comprises metastasis to bone, and in another embodiment, comprises metastasis to other organs. In another embodiment, an immunogenic composition or a therapeutic method disclosed herein is for treating, inhibiting or suppressing metastasis of prostate cancer to bones. In yet another embodiment, an immunogenic composition or a therapeutic method disclosed herein is for treating, inhibiting, or suppressing metastatis of prostate cancer to other organs. In another embodiment, an immunogenic composition or a therapeutic method is for treating, inhibiting or suppressing breast cancer. In another embodiment, an immunogenic composition or a therapeutic method is for treating, inhibiting or suppressing both prostate and breast cancer.

In one embodiment, a method disclosed herein comprises treating a subject having a disease disclosed herein. In another embodiment, a method disclosed herein comprises treating a subject having a tumor or cancer. In another embodiment, the treating reduces or halts the growth of said tumor or said cancer. In another embodiment, the treating reduces or halts metastasis of said tumor or said cancer. In another embodiment, the treating elicits and maintains an anti-tumor or anti-cancer immune response in said subject.

In one embodiment, a method of treatment disclosed herein extends the survival time of a subject receiving the treatment.

Methods for assessing efficacy of prostate cancer vaccines are well known in the art, and are described, for example, in Dzojic H et al (Adenovirus-mediated CD40 ligand therapy induces tumor cell apoptosis and systemic immunity in the TRAMP-C2 mouse prostate cancer model. Prostate. 2006 Jun. 1; 66(8):831-8), Naruishi K et al (Adenoviral vector-mediated RTVP-1 gene-modified tumor cell-based vaccine suppresses the development of experimental prostate cancer. Cancer Gene Ther. 2006 July; 13(7):658-63), Sehgal I et al (Cancer Cell Int. 2006 Aug. 23; 6:21), and Heinrich JE et al (Vaccination against prostate cancer using a live tissue factor deficient cell line in Lobund-Wistar rats. Cancer Immunol Immunother 2007; 56(5):725-30).

In another embodiment, the prostate cancer model used to test methods and compositions as disclosed herein is the TPSA23 (derived from TRAMP-C1 cell line stably expressing PSA) mouse model. In another embodiment, the prostate cancer model is a 178-2 BMA cell model. In another embodiment, the prostate cancer model is a PAIII adenocarcinoma cells model. In another embodiment, the prostate cancer model is a PC-3M model. In another embodiment, the prostate cancer model is any other prostate cancer model known in the art.

In another embodiment, the immunotherapy disclosed herein is tested in human subjects, and efficacy is monitored using methods well known in the art, e.g. directly measuring CD4⁺ and CD8⁺ T cell responses, or measuring disease progression, e.g. by determining the number or size of tumor metastases, or monitoring disease symptoms (cough, chest pain, weight loss, etc). Methods for assessing the efficacy of a prostate cancer vaccine in human subjects are well known in the art, and are described, for example, in Uenaka A et al (T cell immunomonitoring and tumor responses in patients immunized with a complex of cholesterol-bearing hydrophobized pullulan (CHP) and NY-ESO-1 protein. Cancer Immun. 2007 Apr. 19; 7:9) and Thomas-Kaskel AK et al (Vaccination of advanced prostate cancer patients with PSCA and PSA peptide-loaded dendritic cells induces DTH responses that correlate with superior overall survival. Int J Cancer. 2006 Nov. 15; 119(10):2428-34).

In another embodiment, the present disclosure provides a method of treating benign prostate hyperplasia (BPH) in a subject. In another embodiment, the present disclosure provides a method of treating Prostatic Intraepithelial Neoplasia (PIN) in a subject

In one embodiment, disclosed herein is a recombinant Listeria strain comprising a nucleic acid molecule operably integrated into the Listeria genome. In another embodiment said nucleic acid molecule encodes (a) an endogenous polypeptide comprising a PEST sequence and (b) a polypeptide comprising an antigen in an open reading frame.

In one embodiment, disclosed herein is a method of treating, suppressing, or inhibiting at least one tumor in a subject, comprising administering a recombinant Listeria strain to said subject.

In one embodiment, the term “antigen” refers to a substance that when placed in contact with an organism, results in a detectable immune response from the organism. An antigen may be a lipid, peptide, protein, carbohydrate, nucleic acid, or combinations and variations thereof.

In one embodiment, “variant” refers to an amino acid or nucleic acid sequence (or in other embodiments, an organism or tissue) that is different from the majority of the population but is still sufficiently similar to the common mode to be considered to be one of them, for example splice variants.

In one embodiment, “isoform” refers to a version of a molecule, for example, a protein, with only slight differences compared to another isoform, or version, of the same protein. In one embodiment, isoforms may be produced from different but related genes, or in another embodiment, may arise from the same gene by alternative splicing. In another embodiment, isoforms are caused by single nucleotide polymorphisms.

In one embodiment, “immunogenicity” or “immunogenic” is used herein to refer to the innate ability of a protein, peptide, nucleic acid, antigen or organism to elicit an immune response in an animal when the protein, peptide, nucleic acid, antigen or organism is administered to the animal. Thus, “enhancing the immunogenicity” in one embodiment, refers to increasing the ability of a protein, peptide, nucleic acid, antigen or organism to elicit an immune response in an animal when the protein, peptide, nucleic acid, antigen or organism is administered to an animal. The increased ability of a protein, peptide, nucleic acid, antigen or organism to elicit an immune response can be measured by, in one embodiment, a greater number of antibodies to a protein, peptide, nucleic acid, antigen or organism, a greater diversity of antibodies to an antigen or organism, a greater number of T-cells specific for a protein, peptide, nucleic acid, antigen or organism, a greater cytotoxic or helper T-cell response to a protein, peptide, nucleic acid, antigen or organism, and the like.

In one embodiment, a “homologue” refers to a nucleic acid or amino acid sequence which shares a certain percentage of sequence identity with a particular nucleic acid or amino acid sequence. In one embodiment, a sequence useful in the composition and methods as disclosed herein may be a homologue of a particular LLO sequence or N-terminal fragment thereof, ActA sequence or N-terminal fragment thereof, or PEST sequence described herein or known in the art. In one embodiment, such a homolog maintains In another embodiment, a sequence useful in the composition and methods as disclosed herein may be a homologue of an antigenic polypeptide disclosed herein, which in one embodiment, is PSA, or cHER2 functional fragments thereof In one embodiment, a homolog of a polypeptide and, in one embodiment, the nucleic acid encoding such a homolog, of the present disclosure maintains the functional characteristics of the parent polypeptide. For example, in one embodiment, a homolog of an antigenic polypeptide of the present disclosure maintains the antigenic characteristic of the parent polypeptide. In another embodiment, a sequence useful in the composition and methods as disclosed herein may be a homologue of any sequence described herein. In one embodiment, a homologue shares at least 70% identity with a particular sequence. In another embodiment, a homologue shares at least 72% identity with a particular sequence. In another embodiment, a homologue shares at least 75% identity with a particular sequence. In another embodiment, a homologue shares at least 78% identity with a particular sequence. In another embodiment, a homologue shares at least 80% identity with a particular sequence. In another embodiment, a homologue shares at least 82% identity with a particular sequence. In another embodiment, a homologue shares at least 83% identity with a particular sequence. In another embodiment, a homologue shares at least 85% identity with a particular sequence. In another embodiment, a homologue shares at least 87% identity with a particular sequence. In another embodiment, a homologue shares at least 88% identity with a particular sequence. In another embodiment, a homologue shares at least 90% identity with a particular sequence. In another embodiment, a homologue shares at least 92% identity with a particular sequence. In another embodiment, a homologue shares at least 93% identity with a particular sequence. In another embodiment, a homologue shares at least 95% identity with a particular sequence. In another embodiment, a homologue shares at least 96% identity with a particular sequence. In another embodiment, a homologue shares at least 97% identity with a particular sequence. In another embodiment, a homologue shares at least 98% identity with a particular sequence. In another embodiment, a homologue shares at least 99% identity with a particular sequence. In another embodiment, a homologue shares 100% identity with a particular sequence.

In one embodiment, it is to be understood that a homolog of any of the sequences as disclosed herein and/or as described herein is considered to be a part of the disclosure.

In one embodiment, “treating” refers to both therapeutic treatment and prophylactic or preventative measures, wherein the object is to prevent or lessen the targeted pathologic condition or disorder as described herein. Thus, in one embodiment, treating may include directly affecting or curing, suppressing, inhibiting, preventing, reducing the severity of, delaying the onset of, reducing symptoms associated with the disease, disorder or condition, or a combination thereof. Thus, in one embodiment, “treating” refers inter alia to delaying progression, expediting remission, inducing remission, augmenting remission, speeding recovery, increasing efficacy of or decreasing resistance to alternative therapeutics, or a combination thereof. In one embodiment, “preventing” or “impeding” refers, inter alia, to delaying the onset of symptoms, preventing relapse to a disease, decreasing the number or frequency of relapse episodes, increasing latency between symptomatic episodes, or a combination thereof In one embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof. All embodiments disclosed herein also include methods of reducing the persistence of a Listeria strain. In one embodiment, the term “reducing the persistence of” refers to decreasing Listeria CFU count, decreasing Listeria seeding, decreasing Listeria adherence, or decreasing Listeria biofilm formation as compared to a Listeria-based immunotherapy regimen that does not include administering a regimen of antibiotics, and wherein the regimen of antibiotics does not alter the immunogenicity of the Listeria strain.

In one embodiment, symptoms are primary, while in another embodiment, symptoms are secondary. In one embodiment, “primary” refers to a symptom that is a direct result of a particular disease or disorder, while in one embodiment, “secondary” refers to a symptom that is derived from or consequent to a primary cause. In one embodiment, the compounds for use in the present disclosure treat primary or secondary symptoms or secondary complications. In another embodiment, “symptoms” may be any manifestation of a disease or pathological condition.

In some embodiments, the term “comprising” refers to the inclusion of other recombinant polypeptides, amino acid sequences, or nucleic acid sequences, as well as inclusion of other polypeptides, amino acid sequences, or nucleic acid sequences, that may be known in the art, which in one embodiment may comprise antigens or Listeria polypeptides, amino acid sequences, or nucleic acid sequences. In some embodiments, the term “consisting essentially of” refers to a composition for use in the methods as disclosed herein, which has the specific recombinant polypeptide, amino acid sequence, or nucleic acid sequence, or fragment thereof. However, other polypeptides, amino acid sequences, or nucleic acid sequences may be included that are not involved directly in the utility of the recombinant polypeptide(s). In some embodiments, the term “consisting” refers to a composition for use in the methods as disclosed herein having a particular recombinant polypeptide, amino acid sequence, or nucleic acid sequence, or fragment or combination of recombinant polypeptides, amino acid sequences, or nucleic acid sequences or fragments as disclosed herein, in any form or embodiment disclosed herein.

In one embodiment, the immunogenic compositions for use in the methods as disclosed herein are administered intravenously. In another embodiment, the immunotherapy disclosed herein is administered orally, whereas in another embodiment, the vaccine is administered parenterally (e.g., subcutaneously, intramuscularly, and the like).

Further, in another embodiment, the compositions or vaccines are administered as a suppository, for example a rectal suppository or a urethral suppository. Further, in another embodiment, the pharmaceutical compositions are administered by subcutaneous implantation of a pellet. In a further embodiment, the pellet provides for controlled release of an agent over a period of time. In yet another embodiment, the pharmaceutical compositions are administered in the form of a capsule.

In one embodiment, the route of administration may be parenteral. In another embodiment, the route may be intra-ocular, conjunctival, topical, transdermal, intradermal, subcutaneous, intraperitoneal, intravenous, intra-arterial, vaginal, rectal, intratumoral, parcanceral, transmucosal, intramuscular, intravascular, intraventricular, intracranial, inhalation (aerosol), nasal aspiration (spray), intranasal (drops), sublingual, oral, aerosol or suppository or a combination thereof. For intranasal administration or application by inhalation, solutions or suspensions of the compounds mixed and aerosolized or nebulized in the presence of the appropriate carrier suitable. Such an aerosol may comprise any agent described herein. In one embodiment, the compositions as set forth herein may be in a form suitable for intracranial administration, which in one embodiment, is intrathecal and intracerebroventricular administration. In one embodiment, the regimen of administration will be determined by skilled clinicians, based on factors such as exact nature of the condition being treated, the severity of the condition, the age and general physical condition of the patient, body weight, and response of the individual patient, etc.

In one embodiment, parenteral application, particularly suitable are injectable, sterile solutions, preferably oily or aqueous solutions, as well as suspensions, emulsions, or implants, including suppositories and enemas. Ampoules are convenient unit dosages. Such a suppository may comprise any agent described herein.

In one embodiment, sustained or directed release compositions can be formulated, e.g., liposomes or those wherein the active compound is protected with differentially degradable coatings, e.g., by microencapsulation, multiple coatings, etc. Such compositions may be formulated for immediate or slow release. It is also possible to freeze-dry the new compounds and use the lyophilisates obtained, for example, for the preparation of products for injection.

In one embodiment, for liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil.

In one embodiment, compositions of this disclosure are pharmaceutically acceptable. In one embodiment, the term “pharmaceutically acceptable” refers to any formulation which is safe, and provides the appropriate delivery for the desired route of administration of an effective amount of at least one compound for use in the present disclosure. This term refers to the use of buffered formulations as well, wherein the pH is maintained at a particular desired value, ranging from pH 4.0 to pH 9.0, in accordance with the stability of the compounds and route of administration.

In one embodiment, a composition of or used in the methods of this disclosure may be administered alone or within a composition. In another embodiment, compositions of this disclosure admixture with conventional excipients, i.e., pharmaceutically acceptable organic or inorganic carrier substances suitable for parenteral, enteral (e.g., oral) or topical application which do not deleteriously react with the active compounds may be used. In one embodiment, suitable pharmaceutically acceptable carriers include but are not limited to water, salt solutions, alcohols, gum arabic, vegetable oils, benzyl alcohols, polyethylene glycols, gelatine, carbohydrates such as lactose, amylose or starch, magnesium stearate, talc, silicic acid, viscous paraffin, white paraffin, glycerol, alginates, hyaluronic acid, collagen, perfume oil, fatty acid monoglycerides and diglycerides, pentaerythritol fatty acid esters, hydroxy methylcellulose, polyvinyl pyrrolidone, etc. In another embodiment, the pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. In another embodiment, they can also be combined where desired with other active agents, e.g., vitamins.

In one embodiment, the compositions for use of the methods and compositions disclosed herein may be administered with a carrier/diluent. Solid carriers/diluents include, but are not limited to, a gum, a starch (e.g., corn starch, pregeletanized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof.

In one embodiment, an immunogenic compositions of the methods and compositions disclosed herein may comprise an attenuated Listeria strain disclosed herein and one or more additional compounds effective in preventing or treating cancer. In some embodiments, the additional compound may comprise a compound useful in chemotherapy, which in one embodiment, is Cisplatin. In another embodiment, Ifosfamide, Fluorouracilor5-FU, Irinotecan, Paclitaxel (Taxol), Docetaxel, Gemcitabine, Topotecan or a combination thereof, may be administered with a composition as disclosed herein for use in the methods as disclosed herein. In another embodiment, Amsacrine, Bleomycin, Busulfan, Capecitabine, Carboplatin, Carmustine, Chlorambucil, Cisplatin, Cladribine, Clofarabine, Crisantaspase, Cyclophosphamide, Cytarabine, Dacarbazine, Dactinomycin, Daunorubicin, Docetaxel, Doxorubicin, Epirubicin, Etoposide, Fludarabine, Fluorouracil, Gemcitabine, Gliadelimplants, Hydroxycarbamide, Idarubicin, Ifosfamide, Irinotecan, Leucovorin, Liposomaldoxorubicin, Liposomaldaunorubicin, Lomustine, Melphalan, Mercaptopurine, Mesna, Methotrexate, Mitomycin, Mitoxantrone, Oxaliplatin, Paclitaxel, Pemetrexed, Pentostatin, Procarbazine, Raltitrexed, Satraplatin, Streptozocin, Tegafur-uracil, Temozolomide, Teniposide, Thiotepa, Tioguanine, Topotecan, Treosulfan, Vinblastine, Vincristine, Vindesine, Vinorelbine, or a combination thereof, may be administered with a composition as disclosed herein for use in the methods as disclosed herein.

In another embodiment, the additional compound is an immune checkpoint inhibitor selected from the list comprising, a PD1 inhibitor, a PD-L1 inhibitor, a CTLA-4 inhibitor. In another embodiment, the additional compound is an immune stimulator selected from the list comprising an anti-41BB agonist antibody or an anti-CD40 agonist antibody.

In another embodiment, fusion proteins disclosed herein are prepared by a process comprising subcloning of appropriate sequences, followed by expression of the resulting nucleotide. In another embodiment, subsequences are cloned and the appropriate subsequences cleaved using appropriate restriction enzymes. The fragments are then ligated, in another embodiment, to produce the desired DNA sequence. In another embodiment, DNA encoding the fusion protein is produced using DNA amplification methods, for example polymerase chain reaction (PCR). First, the segments of the native DNA on either side of the new terminus are amplified separately. The 5′ end of the one amplified sequence encodes the peptide linker, while the 3′ end of the other amplified sequence also encodes the peptide linker. Since the 5′ end of the first fragment is complementary to the 3′ end of the second fragment, the two fragments (after partial purification, e.g. on LMP agarose) can be used as an overlapping template in a third PCR reaction. The amplified sequence will contain codons, the segment on the carboxy side of the opening site (now forming the amino sequence), the linker, and the sequence on the amino side of the opening site (now forming the carboxyl sequence). The insert is then ligated into a plasmid. In another embodiment, a similar strategy is used to produce a protein wherein an HMW-MAA fragment is embedded within a heterologous peptide.

In another embodiment, gene or protein expression is determined by methods that are well known in the art which in another embodiment comprise real-time PCR, northern blotting, immunoblotting, etc. In another embodiment, expression of an antigen disclosed herein is controlled by an inducible system, while in another embodiment, expression is controlled by a constitutive promoter. In another embodiment, inducible expression systems are well known in the art.

Methods for transforming bacteria are well known in the art, and include calcium-chloride competent cell-based methods, electroporation methods, bacteriophage-mediated transduction, chemical, and physical transformation techniques (de Boer et al, 1989, Cell 56:641-649; Miller et al, 1995, FASEB J., 9:190-199; Sambrook et al. 1989, Molecular Cloning: A Laboratory Manual, Cold Spring Harbor Laboratory, New York; Ausubel et al., 1997, Current Protocols in Molecular Biology, John Wiley & Sons, New York; Gerhardt et al., eds., 1994, Methods for General and Molecular Bacteriology, American Society for Microbiology, Washington, DC; Miller, 1992, A Short Course in Bacterial Genetics, Cold Spring Harbor Laboratory Press, Cold Spring Harbor, N.Y.) In another embodiment, the Listeria strain disclosed herein is transformed by electroporation.

In one embodiment, disclosed herein is a method of inducing an immune response to an antigen in a subject comprising administering a recombinant Listeria strain to said subject, wherein said recombinant Listeria strain comprises a nucleic acid molecule encoding a heterologous antigenic polypeptide or fragment thereof, wherein said first nucleic acid molecule is operably integrated into the Listeria genome as an open reading frame with a nucleic acid encoding an endogenous polypeptide comprising an LLO protein, ActA protein or a PEST sequence. In another embodiment, disclosed herein is a method of inducing an immune response to an antigen in a subject comprising administering a recombinant Listeria strain to said subject, wherein said recombinant Listeria strain comprises a nucleic acid molecule encoding recombinant polypeptide comprising a heterologous antigenic polypeptide or fragment thereof, wherein said recombinant polypeptide further ccomprises an LLO protein, ActA protein or a PEST sequence.

In another embodiment, disclosed herein is a method of inhibiting the onset of cancer, said method comprising the step of administering a recombinant Listeria composition that expresses a recombinant polypeptide comprising a heterologous antigen disclosed herein.

In another embodiment, disclosed herein is a method of inhibiting the onset of cancer, said method comprising the step of administering a recombinant Listeria composition that expresses a recombinant polypeptide comprising a heterologous antigen specifically expressed in said cancer.

In one embodiment, disclosed herein is a method of treating a subject having a tumor or cancer, said method comprising the step of administering a pharmaceutical composition or formulation comprising a recombinant Listeria disclosed herein that expresses a recombinant polypeptide comprising a heterologous antigen disclosed herein.

In one embodiment, administration of an immunogenic composition or treatment modality disclosed herein induces epitope spreading to additional tumor associated antigens.

In another embodiment, disclosed herein is a method of ameliorating symptoms that are associated with a cancer in a subject, said method comprising the step of administering an immunogenic composition or treatment modality disclosed herein.

In one embodiment, disclosed herein is a method of protecting a subject from cancer, said method comprising the step of administering an immunogenic composition or treatment modality disclosed herein.

In another embodiment, disclosed herein is a method of delaying onset of cancer, said method comprising the step of administering an immunogenic composition or treatment modality disclosed herein. In another embodiment, disclosed herein is a method of treating metastatic cancer, said method comprising the step of administering an immunogenic composition or treatment modality disclosed herein.. In another embodiment, disclosed herein is a method of preventing metastatic cancer or micrometastatis, said method comprising the step of administering an immunogenic composition or treatment modality disclosed herein. In another embodiment, the recombinant Listeria composition is administered intravenously, orally or parenterally.

In another embodiment, a pharmaceutical composition comprising the recombinant Listeria disclosed herein is administered intravenously, subcutaneuosly, intranasally, intramuscularly, or injected into a tumor site or into a tumor.

In one embodiment, “antigenic polypeptide” refers to a polypeptide, peptide or recombinant peptide as described hereinabove that is foreign to a host and leads to the mounting of an immune response when present in, or, in another embodiment, detected by, the host.

“Stably maintained” refers, in another embodiment, to maintenance of a nucleic acid molecule or plasmid in the absence of selection (e.g. antibiotic selection) for 10 generations, without detectable loss. In another embodiment, the period is 15 generations. In another embodiment, the period is 20 generations. In another embodiment, the period is 25 generations. In another embodiment, the period is 30 generations. In another embodiment, the period is 40 generations. In another embodiment, the period is 50 generations. In another embodiment, the period is 60 generations. In another embodiment, the period is 80generations. In another embodiment, the period is 100 generations. In another embodiment, the period is 150 generations. In another embodiment, the period is 200 generations. In another embodiment, the period is 300 generations. In another embodiment, the period is 500 generations. In another embodiment, the period is more than 500 generations. In another embodiment, the nucleic acid molecule or plasmid is maintained stably in vitro (e.g. in culture). In another embodiment, the nucleic acid molecule or plasmid is maintained stably in vivo. In another embodiment, the nucleic acid molecule or plasmid is maintained stably both in vitro and in vitro.

In one embodiment, the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” may include both D- and L-amino acids.

In one embodiment of the methods and compositions disclosed herein, the term “recombination site” or “site-specific recombination site” refers to a sequence of bases in a nucleic acid molecule that is recognized by a recombinase (along with associated proteins, in some cases) that mediates exchange or excision of the nucleic acid segments flanking the recombination sites. The recombinases and associated proteins are collectively referred to as “recombination proteins” see, e.g., Landy, A., (Current Opinion in Genetics & Development) 3:699-707; 1993).

A “phage expression vector” or “phagemid” refers to any phage-based recombinant expression system for the purpose of expressing a nucleic acid sequence of the methods and compositions as disclosed herein in vitro or in vivo, constitutively or inducibly, in any cell, including prokaryotic, yeast, fungal, plant, insect or mammalian cell. A phage expression vector typically can both reproduce in a bacterial cell and, under proper conditions, produce phage particles. The term includes linear or circular expression systems and encompasses both phage-based expression vectors that remain episomal or integrate into the host cell genome.

It will be appreciated by a skilled artisan that the term “operably linked” may mean that the transcriptional and translational regulatory nucleic acid, is positioned relative to any coding sequences in such a manner that transcription is initiated. Generally, this will mean that the promoter and transcriptional initiation or start sequences are positioned 5′ to the coding region.

“Transforming,” in one embodiment, refers to engineering a bacterial cell to take up a plasmid or other heterologous DNA molecule. In another embodiment, “transforming” refers to engineering a bacterial cell to express a gene of a plasmid or other heterologous DNA molecule.

In another embodiment, conjugation is used to introduce genetic material and/or plasmids into bacteria. Methods for conjugation are well known in the art, and are described, for example, in Nikodinovic J et al (A second generation snp-derived Escherichia coli-Streptomyces shuttle expression vector that is generally transferable by conjugation. Plasmid. 2006 November; 56(3):223-7) and Auchtung J M et al (Regulation of a Bacillus subtilis mobile genetic element by intercellular signaling and the global DNA damage response. Proc Natl Acad Sci USA. 2005 Aug. 30; 102(35):12554-9). disclosed herein

“Metabolic enzyme” refers, in another embodiment, to an enzyme involved in synthesis of a nutrient required by the host bacteria. In another embodiment, the term refers to an enzyme required for synthesis of a nutrient required by the host bacteria. In another embodiment, the term refers to an enzyme involved in synthesis of a nutrient utilized by the host bacteria. In another embodiment, the term refers to an enzyme involved in synthesis of a nutrient required for sustained growth of the host bacteria. In another embodiment, the enzyme is required for synthesis of the nutrient. disclosed herein

It will be appreciated by a skilled artisan that the term “attenuation,” may encompass a diminution in the ability of the bacterium to cause disease in an animal. In other words, the pathogenic characteristics of the attenuated Listeria strain have been lessened compared with wild-type Listeria, although the attenuated Listeria is capable of growth and maintenance in culture. Using as an example the intravenous inoculation of Balb/c mice with an attenuated Listeria, the lethal dose at which 50% of inoculated animals survive (LD.sub.50) is preferably increased above the LD.sub.50 of wild-type Listeria by at least about 10-fold, more preferably by at least about 100-fold, more preferably at least about 1,000 fold, even more preferably at least about 10,000 fold, and most preferably at least about 100,000-fold. An attenuated strain of Listeria is thus one which does not kill an animal to which it is administered, or is one which kills the animal only when the number of bacteria administered is vastly greater than the number of wild type non-attenuated bacteria which would be required to kill the same animal. An attenuated bacterium should also be construed to mean one which is incapable of replication in the general environment because the nutrient required for its growth is not present therein. Thus, the bacterium is limited to replication in a controlled environment wherein the required nutrient is provided. The attenuated strains of the present disclosure are therefore environmentally safe in that they are incapable of uncontrolled replication.

In one embodiment, the Listeria disclosed herein expresses a heterologous polypeptide, as described herein, in another embodiment, the recombinant Listeria disclosed herein secretes a heterologous polypeptide. In another embodiment, the Listeria as disclosed herein expresses and secretes a heterologous polypeptide. In another embodiment, the Listeria as disclosed herein comprises a heterologous polypeptide, and in another embodiment, comprises a nucleic acid that encodes a recombinant polypeptide comprising a heterologous polypeptide.

In one embodiment, Listeria strains disclosed herein may be used in the preparation of vaccines or immunotherapies described herein.

In one embodiment, the vaccines of the methods and compositions disclosed herein may be administered to a host vertebrate animal, preferably a mammal, and more preferably a human, either alone or in combination with a pharmaceutically acceptable carrier. In another embodiment, the vaccine is administered in an amount effective to induce an immune response to the Listeria strain itself or to a heterologous antigen which the Listeria species has been modified to express. In another embodiment, the amount of vaccine to be administered may be routinely determined by one of skill in the art when in possession of the present disclosure. In another embodiment, a pharmaceutically acceptable carrier may include, but is not limited to, sterile distilled water, saline, phosphate buffered solutions or bicarbonate buffered solutions. In another embodiment, the pharmaceutically acceptable carrier selected and the amount of carrier to be used will depend upon several factors including the mode of administration, the strain of Listeria and the age and disease state of the vaccinee. In another embodiment, administration of the vaccine may be by an oral route, or it may be parenteral, intranasal, intramuscular, intravascular, intrarectal, intraperitoneal, or any one of a variety of well-known routes of administration. In another embodiment, the route of administration may be selected in accordance with the type of infectious agent or tumor to be treated.

As used herein, the singular form “a,” “an” and “the” include plural references unless the context clearly dictates otherwise. For example, the term “a compound” or “at least one compound” may include a plurality of compounds, including mixtures thereof.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. In one embodiment, the term “subject” does not exclude an individual that is healthy in all respects and does not have or show signs of disease or disorder.

In one embodiment, disclosed herein are kits comprising the pharmaceutical compositions or formulations comprising the recombinant Listeria disclosed herein.

Specific embodiments described herein include:
1. A method of preventing persistence of a Listeria strain on a tissue within a subject having a disease following administration of a Listeria-based immunotherapy regimen, the method comprising the step of administering an effective amount of a regimen of antibiotics following administration of said recombinant Listeria-based immunotherapy, thereby preventing said persistence of said Listeria strain within said subject.
2. The method of embodiment 1, wherein said Listeria strain comprises a nucleic acid molecule, said nucleic acid molecule comprising an open reading frame encoding a recombinant polypeptide, said recombinant polypeptide comprising a heterologous antigen or fragment thereof fused to an immunogenic protein or peptide.
3. The method of any one of embodiments 1-2, wherein said immunogenic protein or peptide comprises a truncated LLO protein, a truncated ActA protein or a PEST peptide.
4. The method of any one of embodiments 1-3, wherein administering said antibiotic regimen prevents seeding or adherence of said Listeria strain.
5. The method of any one of embodiments 1-4, wherein administering said antibiotic regimen prevents biofilm formation of said Listeria strain.
6. The method of any one of embodiments 1-5, wherein said antibiotic regimen comprises at least one of the following: clindamycin, gentamicin, azithromycin, vancomycin, phosphomycin, linezolid, rifampicin, minocycline, telithromycin, pefloxacin, a beta-lactam, fusidic acid, a macrolide, a fluoroquinolone, Meropenam Both, Moxifloxacin Both, ampicillin, dapzone, trimethoprim/sulfa (Bactrim) or any combination thereof
7. The method of embodiment 6, wherein the antibiotic is poorly taken up within intact cells.
8. The method of embodiment 6, wherein the antibiotic is able to penetrate cells in order to clear intracellular bacteria.
9. The method of any one of embodiments 1-7, wherein said administering of said antibiotic regimen comprises doing so within 1-8 hours following administration of said recombinant Listeria strain immunotherapy.
10. The method of any one of embodiments 1-6 or 8, wherein said administering of said antibiotic regimen comprises doing so within 2-24 hours following administration of said recombinant Listeria strain immunotherapy or until said Listeria strain is eradicated from said subject but after antigen has been presented in said subject.
11. The method of any one of embodiments 1-6 or 8, wherein administration of said antibiotic regimen comprises administration after a therapeutic goal resulting from said administration of said Listeria strain immunotherapy has been achieved.
12. The method of embodiment 11, wherein said therapeutic goal comprises achieving an anti-disease immune response.
13. The method of embodiment 11, wherein said therapeutic goal comprises achieving tumor or cancer regression.
14. The method of any one of embodiments 1-13, wherein said Listeria strain immunotherapy that is administered to a subject elicits an anti-disease immune response in said subject.
15. The method of any one of embodiments 1-14, wherein administration of said antibiotic regimen comprises administration after said anti-disease response has initiated.
16. The method of any one of embodiments 1-14, wherein said administering of said antibiotic regimen does not interfere with said anti-disease immune response in said subject.
17. The method of embodiment 10, wherein said administering of said antibiotic regimen clears the presence of said Listeria strain within said subject.
18. The method of any one of embodiments 1-17, wherein said heterologous antigen comprises a PSA antigen, a chimeric HER2 antigen, an HPV strain 16 E7 or an HPV strain 18 E7.
19. The method of embodiment 18, wherein said PSA comprises SEQ ID NO: 8.
20. The method of embodiment 18, wherein said cHER2 comprises SEQ ID NO: 17.
21. The method of embodiment 18, wherein said HPV-E7 antigen comprises SEQ ID NO:
22. The method of any one of embodiments 2-21, wherein said recombinant polypeptide comprises a truncated LLO fused to a PSA antigen comprising the amino acid sequence set forth in SEQ ID NO: 15.
23. The method of embodiment 18, wherein said recombinant polypeptide comprises a truncated LLO fused to a cHER2 antigen comprising the amino acid sequence set forth in SEQ ID NO: 21.
24. The method of embodiment 18, wherein said recombinant polypeptide comprises a truncated LLO fused to an HPV-E7 antigen comprising the amino acid sequence set forth in SEQ ID NO: 23.
25. The method of any one of embodiments 1-24, wherein said nucleic acid molecule is in a plasmid in said recombinant Listeria strain.
26. The method of embodiment 25, wherein said plasmid is an integrative plasmid.
27. The method of embodiment 25, wherein said plasmid is an episomal plasmid.
28. The method of embodiment 25, wherein said plasmid is stably maintained in said recombinant Listeria strain in the absence of antibiotic selection.
29. The method of any one of embodiments 25-28, wherein said plasmid does not confer antibiotic resistance upon said recombinant Listeria.
30. The method of any one of embodiments 1-29, wherein said recombinant Listeria strain is attenuated.
31. The method of embodiment 30, wherein said attenuated Listeria comprises a mutation, deletion, replacement, disruption or inactivation in an endogenous gene or genes.
32. The method of embodiment 31, wherein said endogenous gene comprises an actA virulence gene.
33. The method of any one of embodiments 31-32, wherein said endogenous gene comprises a D-alanine racemase (Dal) gene or a D-amino acid transferase (Dat) gene.
34. The method of any one of embodiments 31-33, wherein said endogenous genes comprise the actA, dal, and dat genes.
35. The method of any one of embodiments 2-34, wherein said recombinant nucleic acid molecule in said Listeria strain comprises a second open reading frame.
36. The method of embodiment 35, wherein said second open reading frame encodes a metabolic enzyme.
37. The method of embodiment 36, wherein said metabolic is an alanine racemase enzyme or a D-amino acid transferase enzyme.
38. The method of any one of embodiments 2-37, wherein said recombinant polypeptide is expressed from an hly promoter, a prfA promoter, an actA promoter, or a p60 promoter.
39. The method of any one of embodiments 1-38, wherein said recombinant Listeria strain is a recombinant Listeria monocytogenes strain.
40. The method of any one of embodiments 1-39, wherein said recombinant Listeria strain has been passaged through an animal host.
41. The method of any one of embodiments 1-40, wherein said administration induces epitope spreading to additional tumor antigens.
42. The method of any one of embodiments 1-41, wherein said disease comprises a tumor or cancer, a premalignant condition, an infectious disease or a parasitic disease.
43. The method of embodiment 42, wherein said tumor or cancer comprises a breast tumor or cancer, a gastric tumor or cancer, an prostate tumor or cancer, a brain tumor or cancer, a cervical tumor or cancer, an endometrial tumor or cancer, a glioblastoma, a lung cancer, a bladder tumor or cancer, a pancreatic tumor or cancer, melanoma, a colorectal tumor or cancer, or any combination thereof.
44. The method of embodiment 43, wherein said tumor or said cancer is a metastasis.
45. The method of any one of embodiments 43-44, wherein said method comprises treating a subject having said tumor or cancer.
46. The method of embodiment 45, wherein said treating reduces or halts the growth of said tumor or said cancer.
47. The method of any one of embodiments 45-46, wherein said treating reduces or halts metastasis of said tumor or said cancer.
48. The method of any one of embodiments 45-47, wherein said treating elicits and maintains an anti-tumor or anti-cancer immune response in said subject.
49. The method of any one of embodiments 45-48, wherein said treating extends the survival time of said subject.
50. A method of preventing persistence of a Listeria strain on a tissue within a subject having a disease following administration of a Listeria-based immunotherapy regimen, the method comprising the step of administering an effective amount of a regimen of antibiotics following administration of said recombinant Listeria-based immunotherapy, thereby preventing said persistence of said Listeria strain within said subject.
51. The method of embodiment 50, wherein said Listeria strain comprises a nucleic acid molecule, said nucleic acid molecule comprising an open reading frame encoding one or more peptides encoding one or more neoepitopes, wherein said one or more peptides are fused to an immunogenic protein or peptide.
52. The method of any one of embodiments 50-51, wherein said immunogenic protein or peptide comprises a truncated LLO protein, a truncated ActA protein or a PEST peptide.
53. The method of any one of embodiments 50-52, wherein administering said antibiotic regimen prevents seeding or adherence of said Listeria strain.
54. The method of any one of embodiments 50-53, wherein administering said antibiotic regimen prevents biofilm formation of said Listeria strain.
55. The method of any one of embodiments 50-54, wherein said antibiotic regimen comprises at least one of the following: clindamycin, gentamicin, azithromycin, vancomycin, phosphomycin, linezolid, rifampicin, minocycline, telithromycin, pefloxacin, a beta-lactam, fusidic acid, a macrolide, a fluoroquinolone, ampicillin, Meropenam Both, Moxifloxacin Both, dapzone, trimethoprim/sulfa (Bactrim) or any combination thereof.
56. The method of embodiment 55, wherein the antibiotic is poorly taken up within intact cells.
57. The method of embodiment 56, wherein the antibiotic is able to penetrate cells in order to clear intracellular bacteria.
58. The method of any one of embodiments 50-57, wherein said administering of said antibiotic regimen comprises doing so within 1-8 hours following administration of said recombinant Listeria strain immunotherapy.
59. The method of any one of embodiments 50-57 or 58, wherein said administering of said antibiotic regimen comprises doing so within 2-24 hours following administration of said recombinant Listeria strain immunotherapy or until said Listeria strain is eradicated from said subject but after antigen has been presented in said subject.
60. The method of any one of embodiments 50-57 or 58, wherein administration of said antibiotic regimen comprises administration after a therapeutic goal resulting from said administration of said Listeria strain immunotherapy has been achieved.
61. The method of embodiment 60, wherein said therapeutic goal comprises achieving an anti-disease immune response.
62. The method of embodiment 60, wherein said therapeutic goal comprises achieving tumor or cancer regression.
63. The method of any one of embodiments 50-62, wherein said Listeria strain immunotherapy that is administered to a subject elicits an anti-disease immune response in said subject.
64. The method of any one of embodiments 50-64, wherein administration of said antibiotic regimen comprises administration after said anti-disease response has initiated.
65. The method of any one of embodiments 50-64, wherein said administering of said antibiotic regimen does not interfere with said anti-disease immune response in said subject.
66. The method of embodiment 65, wherein said administering of said antibiotic regimen clears the presence of said Listeria strain within said subject.
67. The method of any one of embodiments 50-66, wherein said one or more neoepitopes are present in a disease or condition-bearing tissue or cell of a subject having said disease or condition.
68. The method of any one of embodiments 50-67, wherein said nucleic acid molecule is in a plasmid in said recombinant Listeria strain.
69. The method of embodiment 68, wherein said plasmid is an integrative plasmid.
70. The method of embodiment 68, wherein said plasmid is an episomal plasmid.
71. The method of embodiment 68, wherein said plasmid is stably maintained in said recombinant Listeria strain in the absence of antibiotic selection.
72. The method of any one of embodiments 68-71, wherein said plasmid does not confer antibiotic resistance upon said recombinant Listeria.
73. The method of any one of embodiments 50-72, wherein said recombinant Listeria strain is attenuated.
74. The method of embodiment 73, wherein said attenuated Listeria comprises a mutation, deletion, replacement, disruption or inactivation in an endogenous gene or genes.
75. The method of embodiment 74, wherein said endogenous gene comprises an actA virulence gene.
76. The method of any one of embodiments 74-75, wherein said endogenous gene comprises a D-alanine racemase (Dal) gene or a D-amino acid transferase (Dat) gene.
77. The method of any one of embodiments 74-76, wherein said endogenous genes comprise the actA, dal, and dat genes.
78. The method of any one of embodiments 51-77, wherein said recombinant nucleic acid molecule in said Listeria strain comprises a second open reading frame.
79. The method of embodiment 78, wherein said second open reading frame encodes a metabolic enzyme.
80. The method of embodiment 79, wherein said metabolic is an alanine racemase enzyme or a D-amino acid transferase enzyme.
81. The method of any one of embodiments 51-80, wherein said one or more peptides are expressed from an hly promoter, a prfA promoter, an actA promoter, or a p60 promoter.
82. The method of any one of embodiments 50-81, wherein said recombinant Listeria strain is a recombinant Listeria monocytogenes strain.
83. The method of any one of embodiments 50-82, wherein said recombinant Listeria strain has been passaged through an animal host.
84. The method of any one of embodiments 50-83, wherein said administration induces epitope spreading to additional tumor antigens.
85. The method of any one of embodiments 50-84, wherein said disease comprises a tumor or cancer, a premalignant condition, an infectious disease or a parasitic disease.
86. The method of embodiment 85, wherein said tumor or cancer comprises a breast tumor or cancer, a gastric tumor or cancer, an prostate tumor or cancer, a brain tumor or cancer, a cervical tumor or cancer, an endometrial tumor or cancer, a glioblastoma, a lung cancer, a bladder tumor or cancer, a pancreatic tumor or cancer, melanoma, a colorectal tumor or cancer, or any combination thereof.
87. The method of embodiment 86, wherein said tumor or said cancer is a metastasis.
88. The method of any one of embodiments 85-88, wherein said method comprises treating a subject having said tumor or cancer.
89. The method of any one of embodiments 85-88, wherein said treating reduces or halts the growth of said tumor or said cancer.
90. The method of any one of embodiments 88-89, wherein said treating reduces or halts metastasis of said tumor or said cancer.
91. The method of any one of embodiments 88-90, wherein said treating elicits and maintains an anti-tumor or anti-cancer immune response in said subject.
92. The method of any one of embodiments 88-91, wherein said treating extends the survival time of said subject.

The following examples are presented in order to more fully illustrate the preferred embodiments of the disclosure. They should in no way be construed, however, as limiting the broad scope of the disclosure.

### EXAMPLES

### Example 1

### Construction of Attenuated Listeria strain-LmddAactA and Insertion of the Human klk3 Gene in Frame to the hly Gene in the Lmdd and Lmdda Strains Materials and Methods

A recombinant Lm was developed that secretes PSA fused to tLLO (Lm-LLO-PSA), which elicits a potent PSA-specific immune response associated with regression of tumors in a mouse model for prostate cancer, wherein the expression of tLLO-PSA is derived from a plasmid based on pGG55 (Table 1), which confers antibiotic resistance to the vector. We recently developed a new strain for the PSA vaccine based on the pADV142 plasmid, which has no antibiotic resistance markers, and referred as LmddA-142 (Table 1). This new strain is 10 times more attenuated than Lm-LLO-PSA. In addition, LmddA-142 was slightly more immunogenic and significantly more efficacious in regressing PSA expressing tumors than the Lm-LLO-PSA.

TABLE 1
Plasmids and strains

| Plasmids | Features |
|---|---|
| pGG55 | pAM401/pGB354 shuttle plasmid with gram(−) and |
| | gram(+) cm resistance, LLO-E7 expression cassette and a |
| | copy of Lm prf4 gene |
| pTV3 | Derived from pGG55 by deleting cm genes and inserting |
| | the Lm dal gene |
| pADV119 | Derived from pTV3 by deleting the prf4 gene |
| pADV134 | Derived from pADV119 by replacing the Lm dal gene by |
| | the Bacillus dal gene |
| pADV142 | Derived from pADV134 by replacing HPV8 e7 with klk3 |
| pADV88 | Derived from pADV134 by replacing HPV8 e7 with |
| | hmw-maa₂₈₀₋₂₂₅₈ |
| Strains | Genotype |
| 10403S | Wild-type Listeria monocytogenes:: str |
| XFL-7 | 10403S prfA⁽⁻⁾ |
| Lmdd | 10403S dal⁽⁻⁾ dat⁽⁻⁾ |
| LmddA | 10403S dal⁽⁻⁾ dat⁽⁻⁾ actA⁽⁻⁾ |
| LmddA- | 10403S dal⁽⁻⁾ dat⁽⁻⁾ actA⁽⁻⁾ pADV134 |
| 134 | |
| LmddA- | 10403S dal⁽⁻⁾ dat⁽⁻⁾ actA⁽⁻⁾ pADV142 |
| 142 | |
| Lmdd-143 | 10403S dal⁽⁻⁾ dat⁽⁻⁾ with klk3 fused to the hly gene in the |
| | chromosome |
| LmddA- | 10403S dal⁽⁻⁾ dat⁽⁻⁾ actA⁽⁻⁾ with klk3 fused to the hly gene |
| 143 | in the chromosome |
| LmddA-88 | 10403S dal⁽⁻⁾ dat⁽⁻⁾ actA⁽⁻⁾ pADV88 |
| Lmdd- | Lmdd-143 pADV134 |
| 143/134 | |
| LmddA- | LmddA-143 pADV134 |
| 143/134 | |
| Lmdd- | Lmdd-143 pADV88 |
| 143/88 | |
| LmddA- | LmddA-143 pADV88 |
| 143/88 | |

The sequence of the plasmid pAdv142 (6523 bp) was as follows:

(SEQ ID NO: 42)
cggagtgtatactggcttactatgttggcactgatgagggtgtcagtgaa
gtgcttcatgtggcaggagaaaaaaggctgcaccggtgcgtcagcagaat
atgtgatacaggatatattccgcttcctcgctcactgactcgctacgctc
ggtcgttcgactgcggcgagcggaaatggcttacgaacggggcggagatt
tcctggaagatgccaggaagatacttaacagggaagtgagagggccgcgg
caaagccgtttttccataggctccgcccccctgacaagcatcacgaaatc
tgacgctcaaatcagtggtggcgaaacccgacaggactataaagatacca
ggcgtttccccctggcggctccctcgtgcgctctcctgttcctgcctttc
ggtttaccggtgtcattccgctgttatggccgcgtttgtctcattccacg
cctgacactcagttccgggtaggcagttcgctccaagctggactgtatgc
acgaaccccccgttcagtccgaccgctgcgccttatccggtaactatcgt
cttgagtccaacccggaaagacatgcaaaagcaccactggcagcagccac
tggtaattgatttagaggagttagtcttgaagtcatgcgccggttaaggc
taaactgaaaggacaagttttggtgactgcgctcctccaagccagttacc
tcggttcaaagagttggtagctcagagaaccttcgaaaaaccgccctgca
aggcggttttttcgttttcagagcaagagattacgcgcagaccaaaacga
tctcaagaagatcatcttattaatcagataaaatatttctagccctcctt
tgattagtatattcctatcttaaagttacttttatgtggaggcattaaca
tttgttaatgacgtcaaaaggatagcaagactagaataaagctataaagc
aagcatataatattgcgtttcatctttagaagcgaatttcgccaatatta
taattatcaaaagagaggggtggcaaacggtatttggcattattaggtta
aaaaatgtagaaggagagtgaaacccatgaaaaaaataatgctagttttt
attacacttatattagttagtctaccaattgcgcaacaaactgaagcaaa
ggatgcatctgcattcaataaagaaaattcaatttcatccatggcaccac
cagcatctccgcctgcaagtcctaagacgccaatcgaaaagaaacacgcg
gatgaaatcgataagtatatacaaggattggattacaataaaaacaatgt
attagtataccacggagatgcagtgacaaatgtgccgccaagaaaaggtt
acaaagatggaaatgaatatattgttgtggagaaaaagaagaaatccatc
aatcaaaataatgcagacattcaagttgtgaatgcaatttcgagcctaac
ctatccaggtgctctcgtaaaagcgaattcggaattagtagaaaatcaac
cagatgttctccctgtaaaacgtgattcattaacactcagcattgatttg
ccaggtatgactaatcaagacaataaaatagttgtaaaaaatgccactaa
atcaaacgttaacaacgcagtaaatacattagtggaaagatggaatgaaa
aatatgctcaagcttatccaaatgtaagtgcaaaaattgattatgatgac
gaaatggcttacagtgaatcacaattaattgcgaaatttggtacagcatt
taaagctgtaaataatagcttgaatgtaaacttcggcgcaatcagtgaag
ggaaaatgcaagaagaagtcattagttttaaacaaatttactataacgtg
aatgttaatgaacctacaagaccttccagatttttcggcaaagctgttac
taaagagcagttgcaagcgcttggagtgaatgcagaaaatcctcctgcat
atatctcaagtgtggcgtatggccgtcaagtttatttgaaattatcaact
aattcccatagtactaaagtaaaagctgcttttgatgctgccgtaagcgg
aaaatctgtctcaggtgatgtagaactaacaaatatcatcaaaaattctt
ccttcaaagccgtaatttacggaggttccgcaaaagatgaagttcaaatc
atcgacggcaacctcggagacttacgcgatattttgaaaaaaggcgctac
ttttaatcgagaaacaccaggagttcccattgcttatacaacaaacttcc
taaaagacaatgaattagctgttattaaaaacaactcagaatatattgaa
acaacttcaaaagcttatacagatggaaaaattaacatcgatcactctgg
aggatacgttgctcaattcaacatttcttgggatgaagtaaattatgatc
tcgagattgtgggaggctgggagtgcgagaagcattcccaaccctggcag
gtgcttgtggcctctcgtggcagggcagtctgcggcggtgttctggtgca
cccccagtgggtcctcacagctgcccactgcatcaggaacaaaagcgtga
tcttgctgggtcggcacagcctgtttcatcctgaagacacaggccaggta
tttcaggtcagccacagcttcccacacccgctctacgatatgagcctcct
gaagaatcgattcctcaggccaggtgatgactccagccacgacctcatgc
tgctccgcctgtcagagcctgccgagctcacggatgctgtgaaggtcatg
gacctgcccacccaggagccagcactggggaccacctgctacgcctcagg
ctggggcagcattgaaccagaggagttcttgaccccaaagaaacttcagt
gtgtggacctccatgttatttccaatgacgtgtgtgcgcaagttcaccct
cagaaggtgaccaagttcatgctgtgtgctggacgetggacagggggcaa
aagcacctgctcgggtgattctgggggcccacttgtctgttatggtgtgc
ttcaaggtatcacgtcatggggcagtgaaccatgtgccctgcccgaaagg
ccttccctgtacaccaaggtggtgcattaccggaagtggatcaaggacac
catcgtggccaaccccTAAcccgggccactaactcaacgctagtagtgga
tttaatcccaaatgagccaacagaaccagaaccagaaacagaacaagtaa
cattggagttagaaatggaagaagaaaaaagcaatgatttcgtgtgaata
atgcacgaaatcattgcttatttttttaaaaagcgatatactagatataa
cgaaacaacgaactgaataaagaatacaaaaaaagagccacgaccagtta
aagcctgagaaactttaactgcgagccttaattgattaccaccaatcaat
taaagaagtcgagacccaaaatttggtaaagtatttaattactttattaa
tcagatacttaaatatctgtaaacccattatatcgggtttttgaggggat
ttcaagtctttaagaagataccaggcaatcaattaagaaaaacttagttg
attgccttttttgttgtgattcaactttgatcgtagcttctaactaatta
attttcgtaagaaaggagaacagctgaatgaatatcccttttgttgtaga
aactgtgcttcatgacggcttgttaaagtacaaatttaaaaatagtaaaa
ttcgctcaatcactaccaagccaggtaaaagtaaaggggctatttttgcg
tatcgctcaaaaaaaagcatgattggcggacgtggcgttgttctgacttc
cgaagaagcgattcacgaaaatcaagatacatttacgcattggacaccaa
acgtttatcgttatggtacgtatgcagacgaaaaccgttcatacactaaa
ggacattctgaaaacaatttaagacaaatcaataccttctttattgattt
tgatattcacacggaaaaagaaactatttcagcaagcgatattttaacaa
cagctattgatttaggttttatgcctacgttaattatcaaatctgataaa
ggttatcaagcatattttgttttagaaacgccagtctatgtgacttcaaa
atcagaatttaaatctgtcaaagcagccaaaataatctcgcaaaatatcc
gagaatattttggaaagtctttgccagttgatctaacgtgcaatcatttt
gggattgctcgtataccaagaacggacaatgtagaattttttgatcccaa
ttaccgttattctttcaaagaatggcaagattggtctttcaaacaaacag
ataataagggctttactcgttcaagtctaacggttttaagcggtacagaa
ggcaaaaaacaagtagatgaaccctggtttaatctcttattgcacgaaac
gaaattttcaggagaaaagggtttagtagggcgcaatagcgttatgttta
ccctctctttagcctactttagttcaggctattcaatcgaaacgtgcgaa
tataatatgtttgagtttaataatcgattagatcaacccttagaagaaaa
agaagtaatcaaaattgttagaagtgcctattcagaaaactatcaagggg
ctaatagggaatacattaccattctttgcaaagcttgggtatcaagtgat
ttaaccagtaaagatttatttgtccgtcaagggtggtttaaattcaagaa
aaaaagaagcgaacgtcaacgtgttcatttgtcagaatggaaagaagatt
taatggcttatattagcgaaaaaagcgatgtatacaagccttatttagcg
acgaccaaaaaagagattagagaagtgctaggcattcctgaacggacatt
agataaattgctgaaggtactgaaggcgaatcaggaaattttctttaaga
ttaaaccaggaagaaatggtggcattcaacttgctagtgttaaatcattg
ttgctatcgatcattaaattaaaaaaagaagaacgagaaagctatataaa
ggcgctgacagcttcgtttaatttagaacgtacatttattcaagaaactc
taaacaaattggcagaacgccccaaaacggacccacaactcgatttgttt
agctacgatacaggctgaaaataaaacccgcactatgccattacatttat
atctatgatacgtgtttgtttttctttgctggctagcttaattgcttata
tttacctgcaataaaggatttcttacttccattatactcccattttccaa
aaacatacggggaacacgggaacttattgtacaggccacctcatagttaa
tggtttcgagccttcctgcaatctcatccatggaaatatattcatccccc
tgccggcctattaatgtgacttttgtgcccggcggatattcctgatccag
ctccaccataaattggtccatgcaaattcggccggcaattttcaggcgtt
ttcccttcacaaggatgtcggtccctttcaattttcggagccagccgtcc
gcatagcctacaggcaccgtcccgatccatgtgtctttttccgctgtgta
ctcggctccgtagctgacgctctcgccttttctgatcagtttgacatgtg
acagtgtcgaatgcagggtaaatgccggacgcagctgaaacggtatctcg
tccgacatgtcagcagacgggcgaaggccatacatgccgatgccgaatct
gactgcattaaaaaagccttttttcagccggagtccagcggcgctgttcg
cgcagtggaccattagattctttaacggcagcggagcaatcagctcttta
aagcgctcaaactgcattaagaaatagcctctttctttttcatccgctgt
cgcaaaatgggtaaatacccctttgcactttaaacgagggttgcggtcaa
gaattgccatcacgttctgaacttcttcctctgtttttacaccaagtctg
ttcatccccgtatcgaccttcagatgaaaatgaagagaaccttttttcgt
gtggcgggctgcctcctgaagccattcaacagaataacctgttaaggtca
cgtcatactcagcagcgattgccacatactccgggggaaccgcgccaagc
accaatataggcgccttcaatccctttttgcgcagtgaaatcgcttcatc
caaaatggccacggccaagcatgaagcacctgcgtcaagagcagcctttg
ctgtttctgcatcaccatgcccgtaggcgtttgctttcacaactgccatc
aagtggacatgttcaccgatatgttttttcatattgctgacattttcctt
tatcgcggacaagtcaatttccgcccacgtatctctgtaaaaaggttttg
tgctcatggaaaactcctctcttttttcagaaaatcccagtacgtaatta
agtatttgagaattaattttatattgattaatactaagtttacccagttt
tcacctaaaaaacaaatgatgagataatagctccaaaggctaaagaggac
tataccaactatttgttaattaa.

This plasmid was sequenced at Genewiz facility from the E. coli strain on 2-20-08.

The strain Lm dal dat (Lmdd) was attenuated by the irreversible deletion of the virulence factor, ActA. An in-frame deletion of actA in the Lmdaldat (Lmdd) background was constructed to avoid any polar effects on the expression of downstream genes. The Lm dal dat AactA contains the first 19 amino acids at the N-terminal and 28 amino acid residues of the C-terminal with a deletion of 591 amino acids of ActA.

The actA deletion mutant was produced by amplifying the chromosomal region corresponding to the upstream (657 bp-oligo's Adv 271/272) and downstream (625 bp-oligo's Adv 273/274) portions of actA and joining by PCR. The sequence of the primers used for this amplification is given in the Table 2. The upstream and downstream DNA regions of actA were cloned in the pNEB193 at the EcoRI/PstI restriction site and from this plasmid, the EcoRI/PstI was further cloned in the temperature sensitive plasmid pKSV7, resulting in ΔactA/pKSV7 (pAdv120).

TABLE 2
Sequence of primers that was used
for the amplification of DNA sequences
upstream and downstream of actA

| Primer | Sequence | SEQ ID NO: |
|---|---|---|
| Adv271-actAF1 | cgGAATTCGGATCCgcgcca | 43 |
| | aatcattggttgattg | |
| Adv272-actAR1 | gcgaGTCGACgtcggggtta | 44 |
| | atcgtaatgcaattggc | |
| Adv273-actAF2 | gcgaGTCGACccatacgacg | 45 |
| | ttaattcttgcaatg | |
| Adv274-actAR2 | gataCTGCAGGGATCCttcc | 46 |
| | cttctcggtaatcagtcac | |

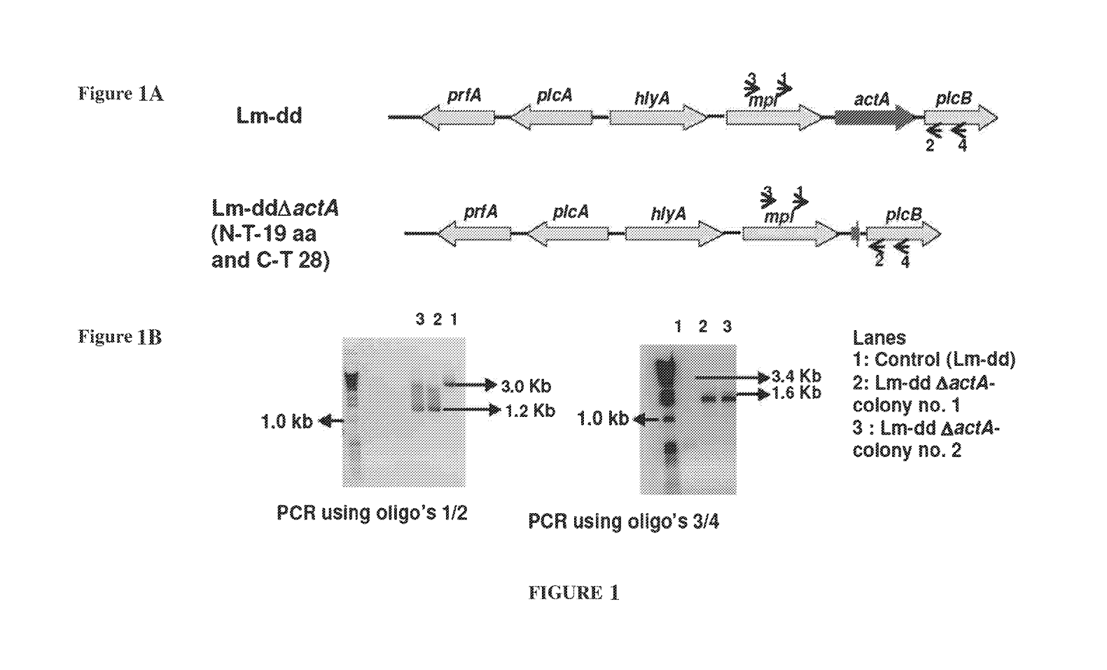

The deletion of the gene from its chromosomal location was verified using primers that bind externally to the actA deletion region, which are shown in FIG. 1 (A and B) as primer 3 (Adv 305-tgggatggccaagaaattc, SEQ ID NO: 47 and primer 4 (Adv304-ctaccatgtcttccgttgcttg; SEQ ID NO: 48) . The PCR analysis was performed on the chromosomal DNA isolated from Lmdd and LmddΔactA. The sizes of the DNA fragments after amplification with two different sets of primer pairs 1/2 and 3/4 in Lmdd chromosomal DNA was expected to be 3.0 Kb and 3.4 Kb. On the other hand, the expected sizes of PCR using the primer pairs 1/2 and 3/4 for the LmddΔactA was 1.2 Kb and 1.6 Kb. Thus, PCR analysis in FIG. 1 (A and B) confirms that the 1.8 kb region of actA was deleted in the LmddΔactA strain. DNA sequencing was also performed on PCR products to confirm the deletion of actA containing region in the strain, LmddΔactA.

### Example 2

### Construction of the Antibiotic-Independent Episomal Expression System for Antigen delivery by Lm Vectors

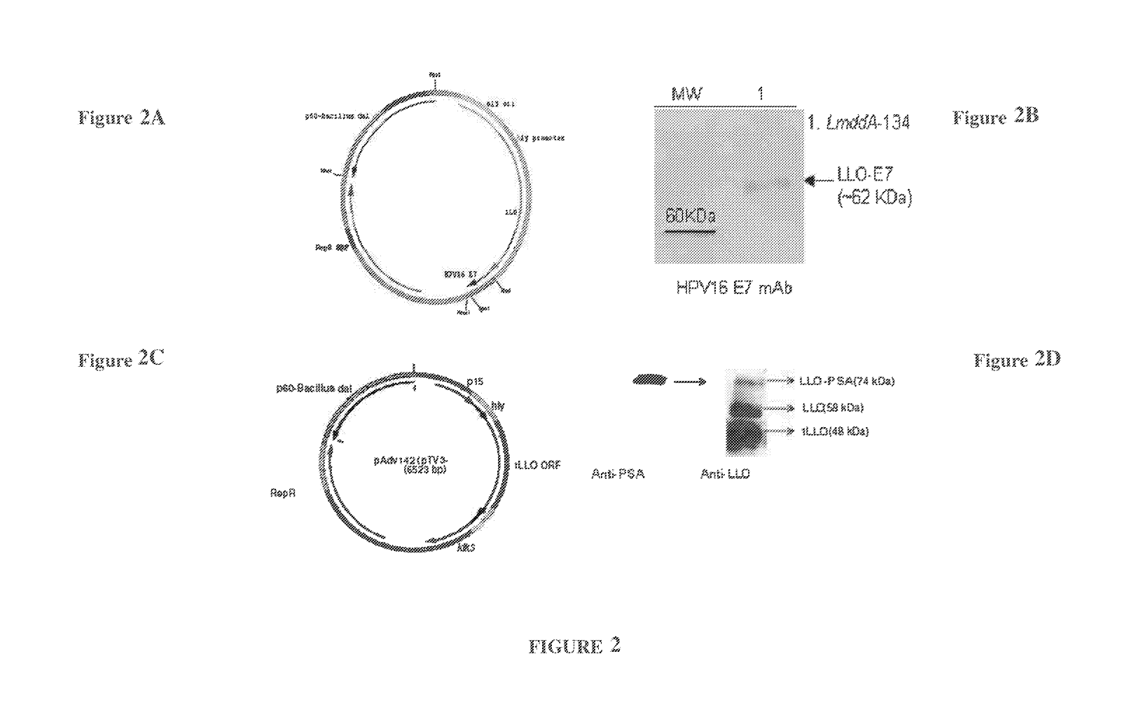

The antibiotic-independent episomal expression system for antigen delivery by Lm vectors (pAdv142) is the next generation of the antibiotic-free plasmid pTV3 (Verch et al., Infect Immun, 2004. 72(11):6418-25, incorporated herein by reference). The gene for virulence gene transcription activator, prfA was deleted from pTV3 since Listeria strain Lmdd contains a copy of prfA gene in the chromosome. Additionally, the cassette for p60-Listeria dal at the NheI/PacI restriction site was replaced by p60-Bacillus subtilis dal resulting in plasmid pAdv134 (FIG. 2A). The similarity of the Listeria and Bacillus dal genes is ˜30%, virtually eliminating the chance of recombination between the plasmid and the remaining fragment of the dal gene in the Lmdd chromosome. The plasmid pAdv134 contained the antigen expression cassette tLLO-E7. The LmddA strain was transformed with the pADV134 plasmid and expression of the LLO-E7 protein from selected clones confirmed by Western blot (FIG. 2B). The Lmdd system derived from the 10403S wild-type strain lacks antibiotic resistance markers, except for the Lmdd streptomycin resistance.

Further, pAdv134 was restricted with XhoI/XmaI to clone human PSA, klk3 resulting in the plasmid, pAdv142. The new plasmid, pAdv142 (FIG. 2C, Table 1) contains Bacillus dal (B-Dal) under the control of Listeria p60 promoter. The shuttle plasmid, pAdv142 complemented the growth of both E. coli ala drx MB2159 as well as Listeria monocytogenes strain Lmdd in the absence of exogenous D-alanine. The antigen expression cassette in the plasmid pAdv142 consists of hly promoter and LLO-PSA fusion protein (FIG. 2C).

The plasmid pAdv142 was transformed to the Listeria background strains, LmddactA strain resulting in Lm-ddA-LLO-PSA. The expression and secretion of LLO-PSA fusion protein by the strain, Lm-ddA-LLO-PSA was confirmed by Western Blot using anti-LLO and anti-PSA antibody (FIG. 2D). There was stable expression and secretion of LLO-PSA fusion protein by the strain, Lm-ddA-LLO-PSA after two in vivo passages.

### Example 3

### In Vitro and In Vivo Stability of the Strain LmddA-LLO-PSA

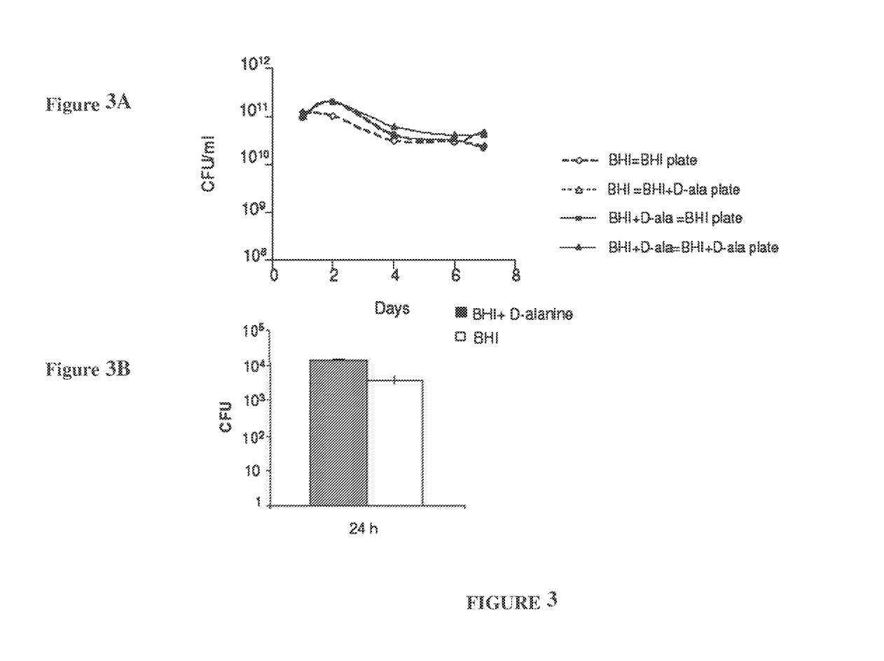

The in vitro stability of the plasmid was examined by culturing the LmddA-LLO-PSA Listeria strain in the presence or absence of selective pressure for eight days. The selective pressure for the strain LmddA-LLO-PSA is D-alanine. Therefore, the strain LmddA-LLO-PSA was passaged in Brain-Heart Infusion (BHI) and BHI+100 μg/ml D-alanine. CFUs were determined for each day after plating on selective (BHI) and non-selective (BHI+D-alanine) medium. It was expected that a loss of plasmid will result in higher CFU after plating on non-selective medium (BHI+D-alanine). As depicted in FIG. 3A, there was no difference between the number of CFU in selective and non-selective medium. This suggests that the plasmid pAdv142 was stable for at least 50 generations, when the experiment was terminated.

Plasmid maintenance in vivo was determined by intravenous injection of 5×10⁷ CFU LmddA-LLO-PSA, in C57BL/6 mice. Viable bacteria were isolated from spleens homogenized in PBS at 24 h and 48 h. CFUs for each sample were determined at each time point on BHI plates and BHI+100 mg/ml D-alanine. After plating the splenocytes on selective and non-selective medium, the colonies were recovered after 24 h. Since this strain is highly attenuated, the bacterial load is cleared in vivo in 24 h. No significant differences of CFUs were detected on selective and non-selective plates, indicating the stable presence of the recombinant plasmid in all isolated bacteria (FIG. 3B).

### Example 4

### In Vivo Passaging, Virulence and Clearance of the Strain LmddA-142 (LmddA-LLO-PSA)

LmddA-142 is a recombinant Listeria strain that secretes the episomally expressed tLLO-PSA fusion protein. To determine a safe dose, mice were immunized with LmddA-LLO-PSA at various doses and toxic effects were determined. LmddA-LLO-PSA caused minimum toxic effects (data not shown). The results suggested that a dose of 10⁸ CFU of LmddA-LLO-PSA was well tolerated by mice. Virulence studies indicate that the strain LmddA-LLO-PSA was highly attenuated.

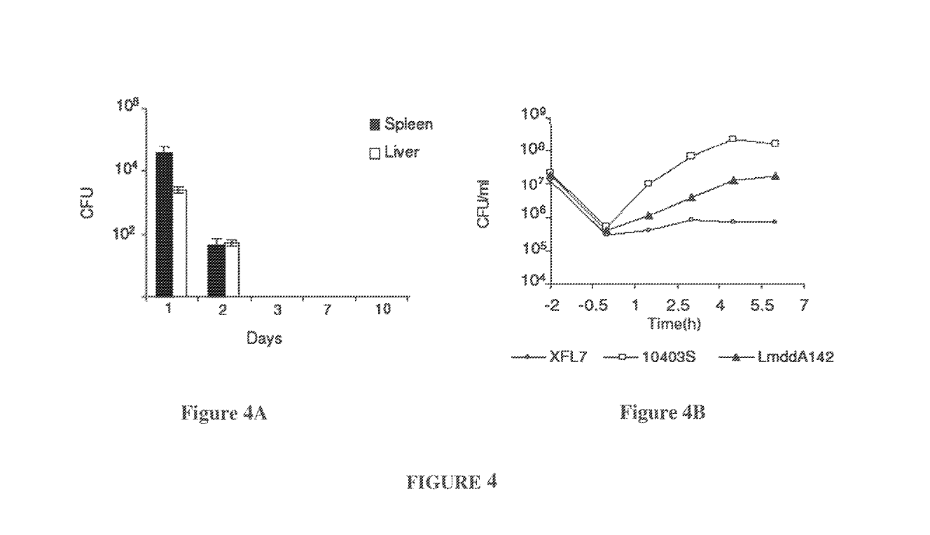

The in vivo clearance of LmddA-LLO-PSA after administration of the safe dose, 10⁸ CFU intraperitoneally in C57BL/6 mice, was determined. There were no detectable colonies in the liver and spleen of mice immunized with LmddA-LLO-PSA after day 2. Since this strain is highly attenuated, it was completely cleared in vivo at 48 h (FIG. 4A).

To determine if the attenuation of LmddA-LLO-PSA attenuated the ability of the strain LmddA-LLO-PSA to infect macrophages and grow intracellularly, a cell infection assay was performed. Mouse macrophage-like cell line such as J774A.1, were infected in vitro with Listeria constructs and intracellular growth was quantified. The positive control strain, wild type Listeria strain 10403S grows intracellularly, and the negative control XFL7, a prfA mutant, cannot escape the phagolysosome and thus does not grow in J774 cells. The intracytoplasmic growth of LmddA-LLO-PSA was slower than 10403S due to the loss of the ability of this strain to spread from cell to cell (FIG. 4B). The results indicate that LmddA-LLO-PSA has the ability to infect macrophages and grow intracytoplasmically.

### Example 5

### Immunogenicity of the Strain-LmddA-LLO-PSA in C57BL/6 Mice

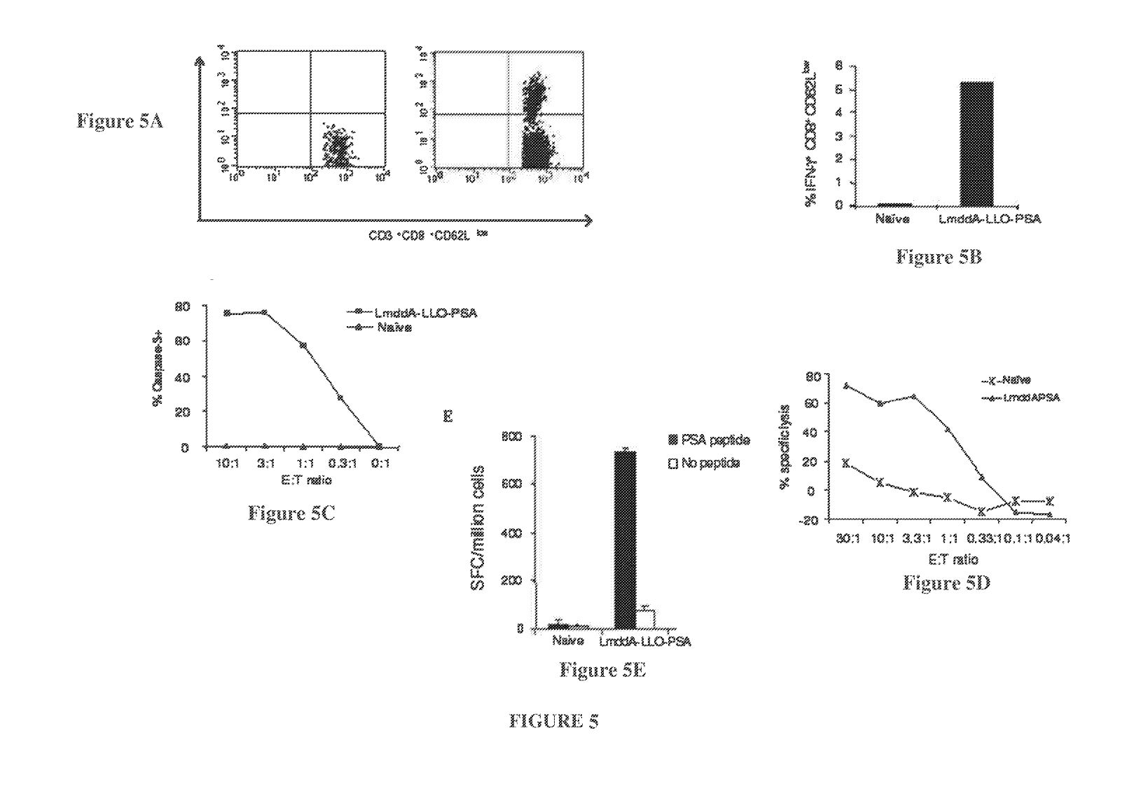

The PSA-specific immune responses elicited by the construct LmddA-LLO-PSA in C57BL/6 mice were determined using PSA tetramer staining. Mice were immunized twice with LmddA-LLO-PSA at one week intervals and the splenocytes were stained for PSA tetramer on day 6 after the boost. Staining of splenocytes with the PSA-specific tetramer showed that LmddA-LLO-PSA elicited 23% of PSA tetramer⁺CD8⁺CD62L^{low} cells (FIG. 5A). The functional ability of the PSA-specific T cells to secrete IFN-y after stimulation with PSA peptide for 5 h was examined using intracellular cytokine staining. There was a 200-fold increase in the percentage of CD8⁺CD62L^{low}IFN-γ secreting cells stimulated with PSA peptide in the LmddA-LLO-PSA group compared to the naïve mice (FIG. 5B), indicating that the LmddA-LLO-PSA strain is very immunogenic and primes high levels of functionally active PSA CD8⁺ T cell responses against PSA in the spleen.

To determine the functional activity of cytotoxic T cells generated against PSA after immunizing mice with LmddA-LLO-PSA, we tested the ability of PSA-specific CTLs to lyse cells EL4 cells pulsed with H-2D^{h} peptide in an in vitro assay. A FACS-based caspase assay (FIG. 5C) and Europium release (FIG. 5D) were used to measure cell lysis. Splenocytes of mice immunized with LmddA-LLO-PSA contained CTLs with high cytolytic activity for the cells that display PSA peptide as a target antigen.

Elispot was performed to determine the functional ability of effector T cells to secrete IFN-γ after 24 h stimulation with antigen. Using ELISpot, a 20-fold increase in the number of spots for IFN-γ in splenocytes from mice immunized with LmddA-LLO-PSA stimulated with specific peptide when compared to the splenocytes of the naïve mice was observed (FIG. 5E).

### Example 6

### Immunization with the LmddA -142 Strains Induces regression of a Tumor Expressing PSA and Infiltration of the Tumor by PSA-Specific CTLs

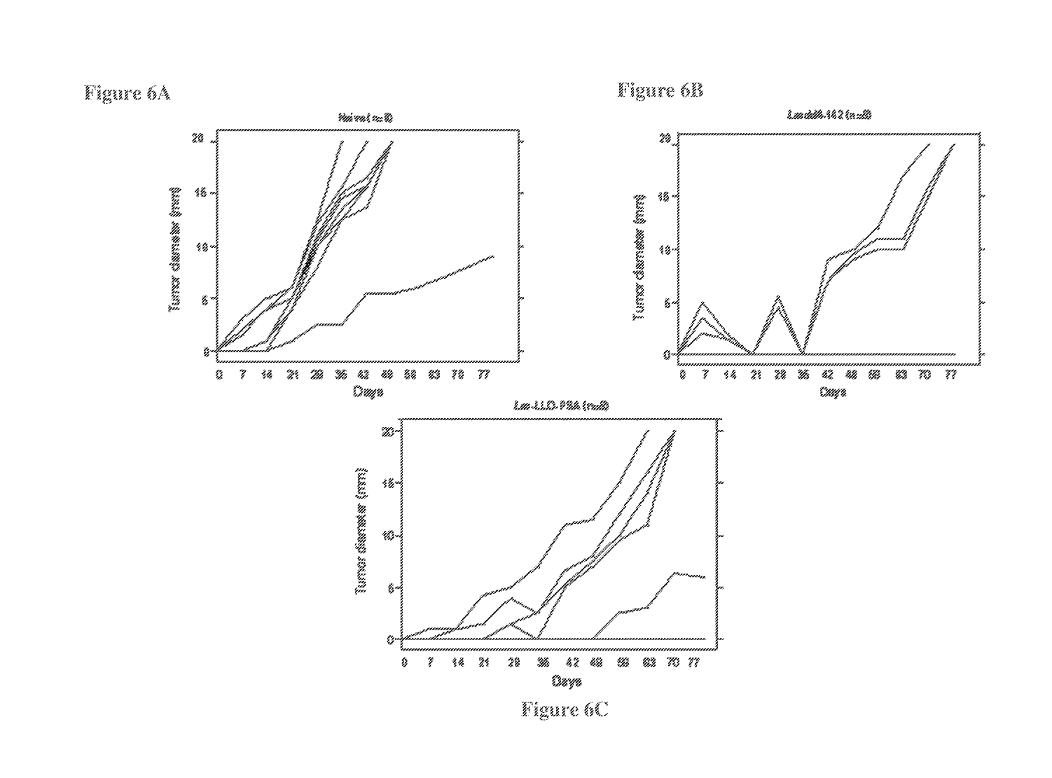

The therapeutic efficacy of the construct LmddA-142 (LmddA-LLO-PSA) was determined using a prostrate adenocarcinoma cell line engineered to express PSA (Tramp-C1-PSA (TPSA); Shahabi et al., 2008). Mice were subcutaneously implanted with 2×10⁶ TPSA cells. When tumors reached the palpable size of 4-6 mm, on day 6 after tumor inoculation, mice were immunized three times at one week intervals with 10⁸ CFU LmddA-142, 10⁷ CFU Lm-LLO-PSA (positive control) or left untreated. The naïve mice developed tumors gradually (FIG. 6A). The mice immunized with LmddA-142 were all tumor-free until day 35 and gradually 3 out of 8 mice developed tumors, which grew at a much slower rate as compared to the naive mice (FIG. 6B). Five out of eight mice remained tumor free through day 70. As expected, Lm-LLO-PSA-vaccinated mice had fewer tumors than naive controls and tumors developed more slowly than in controls (FIG. 6C). Thus, the construct LmddA-LLO-PSA could regress 60% of the tumors established by TPSA cell line and slow the growth of tumors in other mice. Cured mice that remained tumor free were rechallenged with TPSA tumors on day 68.

Immunization of mice with the LmddA-142 can control the growth and induce regression of 7-day established Tramp-C1 tumors that were engineered to express PSA in more than 60% of the experimental animals (FIG. 6B), compared to none in the untreated group (FIG. 6A). The LmddA-142 was constructed using a highly attenuated vector (LmddA) and the plasmid pADV142 (Table 1).

Further, the ability of PSA-specific CD8 lymphocytes generated by the LmddA-LLO-PSA construct to infiltrate tumors was investigated. Mice were subcutaneously implanted with a mixture of tumors and matrigel followed by two immunizations at seven day intervals with naive or control (Lm-LLO-E7) Listeria, or with LmddA-LLO-PSA. Tumors were excised on day 21 and were analyzed for the population of CD8⁺CD62L^{low} PSA^{tetramer+} and CD4⁺CD25⁺FoxP3⁺ regulatory T cells infiltrating in the tumors.

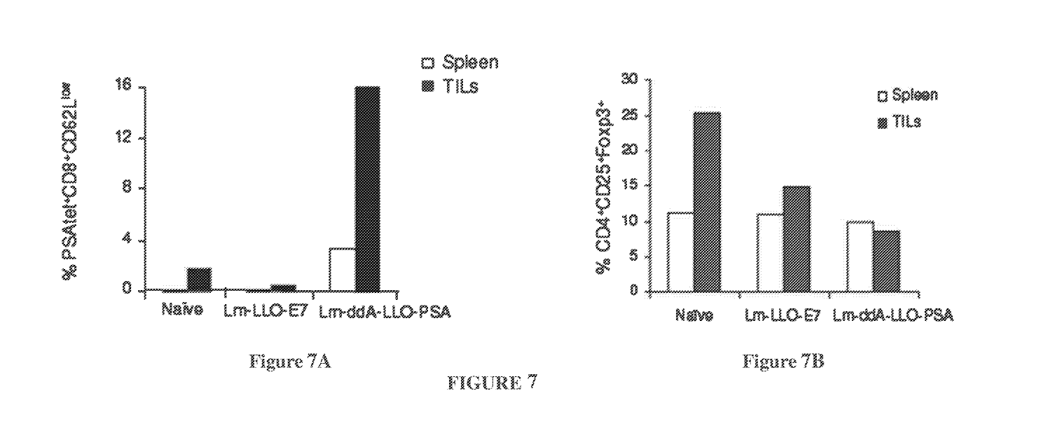

A very low number of CD8⁺CD62L^{low} PSA^{tetramer+} tumor infiltrating lymphocytes (TILs) specific for PSA that were present in the both naive and Lm-LLO-E7 control immunized mice was observed. However, there was a 10-30-fold increase in the percentage of PSA-specific CD8⁺CD62L^{low} PSA^{tetramer+} TILs in the mice immunized with LmddA-LLO-PSA (FIG. 7A). Interestingly, the population of CD8+CD62L^{low} PSA^{tetramer+} cells in spleen was 7.5 fold less than in tumor (FIG. 7A).

In addition, the presence of CD4⁺/CD25⁺/Foxp3⁺ T regulatory cells (Tregs) in the tumors of untreated mice and Listeria immunized mice was determined. Interestingly, immunization with Listeria resulted in a considerable decrease in the number of CD4⁺ CD25⁺FoxP3⁺ T-regs in tumor but not in spleen (FIG. 7B). However, the construct LmddA-LLO-PSA had a stronger impact in decreasing the frequency of CD4⁺CD25⁺FoxP3⁺ T-regs in tumors when compared to the naive and Lm-LLO-E7 immunized group (FIG. 7B).

Thus, the LmddA-142 vaccine can induce PSA-specific CD8⁺ T cells that are able to infiltrate the tumor site (FIG. 7A). Interestingly, immunization with LmddA-142 was associated with a decreased number of regulatory T cells in the tumor (FIG. 7B), probably creating a more favorable environment for an efficient anti-tumor CTL activity.

### Example 7

### Lmdd-143 and LmddA-143 Secretes a Functional LLO Despite the PSA Fusion

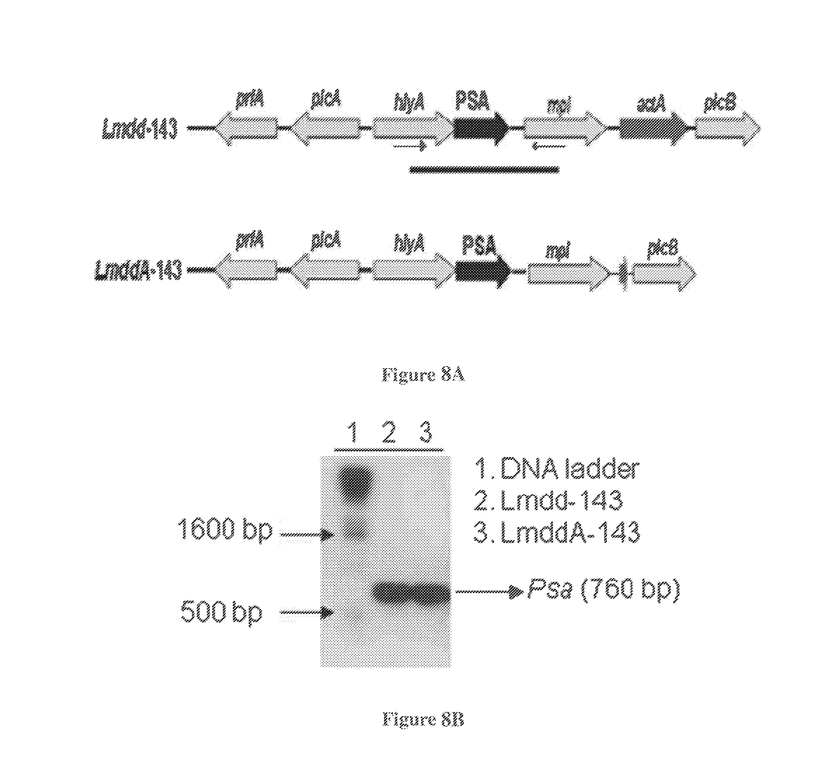

The Lmdd-143 and LmddA-143 contain the full-length human klk3 gene, which encodes the PSA protein, inserted by homologous recombination downstream and in frame with the hly gene in the chromosome. These constructs were made by homologous recombination using the pKSV7 plasmid (Smith and Youngman, Biochimie 1992; 74 (7-8) p705-711), which has a temperature-sensitive replicon, carrying the hly-klk3-mpl recombination cassette. Because of the plasmid excision after the second recombination event, the antibiotic resistance marker used for integration selection is lost. Additionally, the actA gene is deleted in the LmddA-143 strain (FIG. 8A). The insertion of klk3 in frame with hly into the chromosome was verified by PCR (FIG. 8B) and sequencing (data not shown) in both constructs.

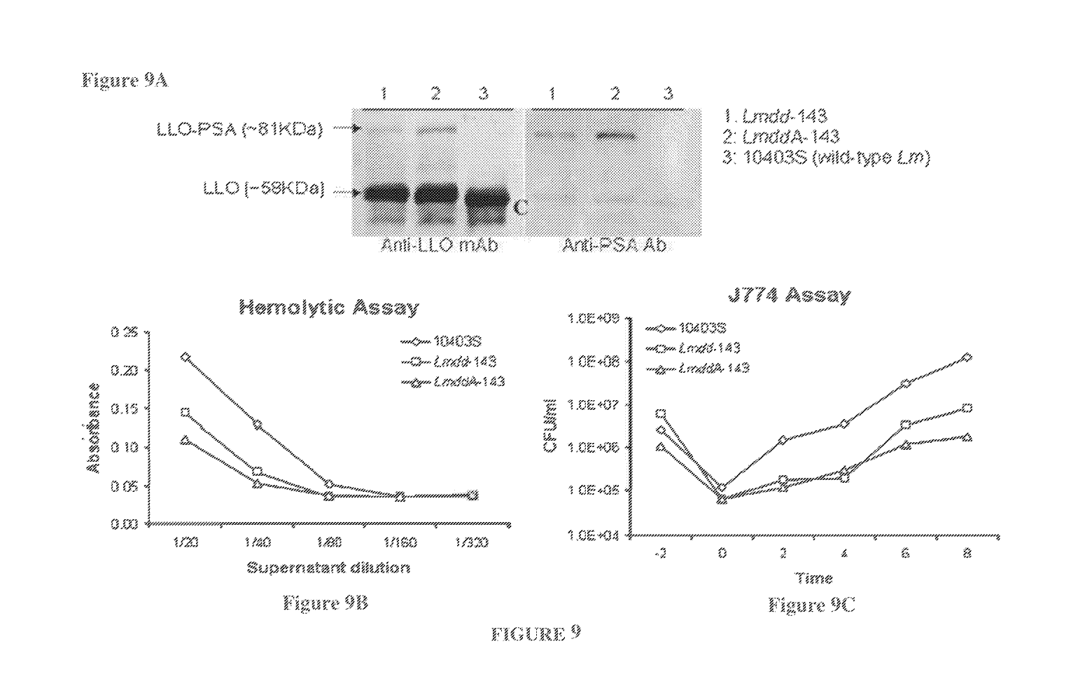

One important aspect of these chromosomal constructs is that the production of LLO-PSA would not completely abolish the function of LLO, which is required for escape of Listeria from the phagosome, cytosol invasion and efficient immunity generated by L. monocytogenes. Western-blot analysis of secreted proteins from Lmdd-143 and LmddA-143 culture supernatants revealed an ˜81 kDa band corresponding to the LLO-PSA fusion protein and an ˜60 kDa band, which is the expected size of LLO (FIG. 9A), indicating that LLO is either cleaved from the LLO-PSA fusion or still produced as a single protein by L. monocytogenes, despite the fusion gene in the chromosome. The LLO secreted by Lmdd-143 and LmddA-143 retained 50% of the hemolytic activity, as compared to the wild-type L. monocytogenes 10403S (FIG. 9B). In agreement with these results, both Lmdd-143 and LmddA-143 were able to replicate intracellularly in the macrophage-like J774 cell line (FIG. 9C).

### Example 8

### Both Lmdd-143 and LmddA-143 Elicit Cell-Mediated Immune Responses Against the PSA Antigen

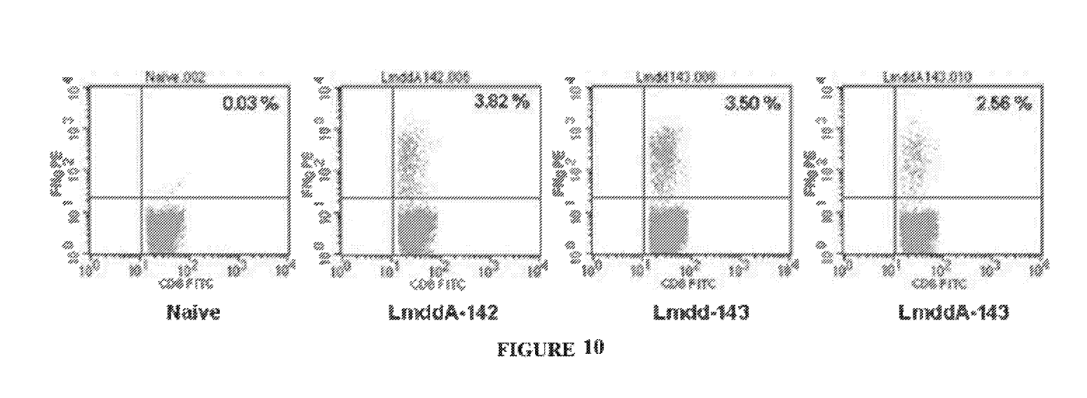

After showing that both Lmdd-143 and LmddA-143 were able to secrete PSA fused to LLO, the question of if these strains could elicit PSA-specific immune responses in vivo was investigated. C57B1/6 mice were either left untreated or immunized twice with the Lmdd-143, LmddA-143 or LmddA-142. PSA-specific CD8⁺ T cell responses were measured by stimulating splenocytes with the PSA65-74 peptide and intracellular staining for IFN-γ. As shown in FIG. 10, the immune response induced by the chromosomal and the plasmid-based vectors is similar.

### Example 9

### Generation of L. Monocytogenes Strains that Secrete LLO Fragments Fused to Her-2 Fragments: Construction of ADXS31-164

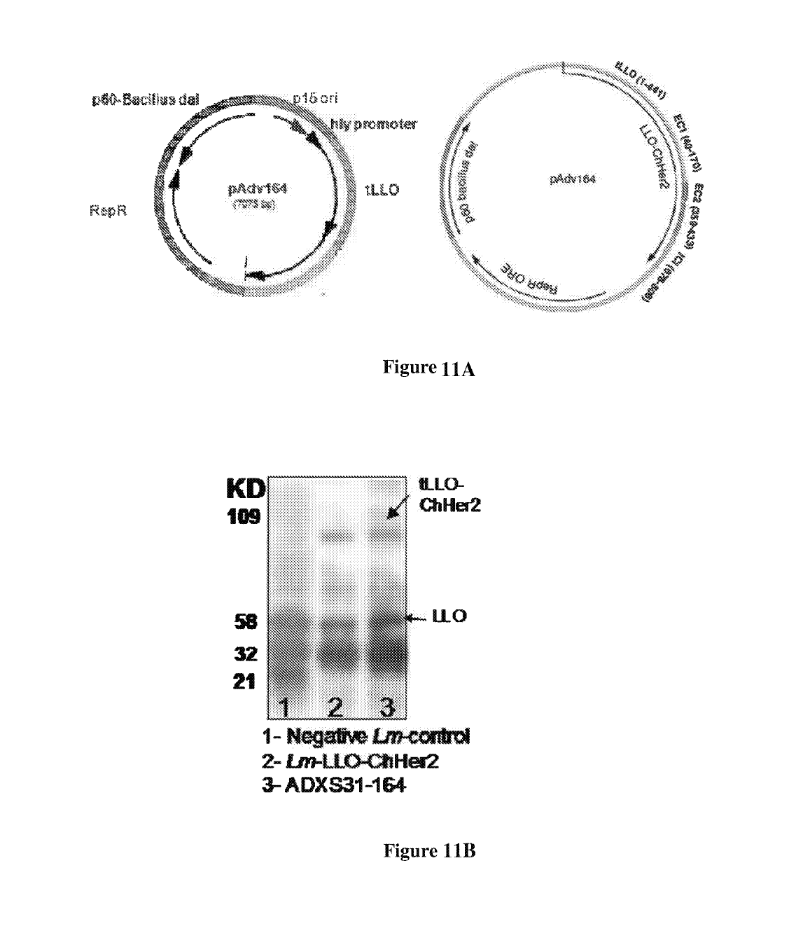

Construction of the chimeric Her2/neu gene (ChHer2) was as follows. Briefly, ChHer2 gene was generated by direct fusion of two extracellular (aa 40-170 and aa 359-433) and one intracellular fragment (aa 678-808) of the Her2/neu protein by SOEing PCR method. The chimeric protein harbors most of the known human MHC class I epitopes of the protein. ChHer2 gene was excised from the plasmid, pAdv138 (which was used to construct Lm-LLO-ChHer2) and cloned into LmddA shuttle plasmid, resulting in the plasmid pAdv84 (FIG. 11A). There are two major differences between these two plasmid backbones. 1) Whereas pAdv138 uses the chloramphenicol resistance marker (cat) for in vitro selection of recombinant bacteria, pAdv84 harbors the D-alanine racemase gene (dal) from bacillus subtilis, which uses a metabolic complementation pathway for in vitro selection and in vivo plasmid retention in LmddA strain which lacks the dal-dat genes. This vaccine platform was designed and developed to address FDA concerns about the antibiotic resistance of the engineered Listeria strains. 2) Unlike pAdv138, pAdv84 does not harbor a copy of the prfA gene in the plasmid (see sequence below and FIG. 11A), as this is not necessary for in vivo complementation of the Lmdd strain. The LmddA vaccine strain also lacks the actA gene (responsible for the intracellular movement and cell-to-cell spread of Listeria) so the recombinant vaccine strains derived from this backbone are 100 times less virulent than those derived from the Lmdd, its parent strain. LmddA-based vaccines are also cleared much faster (in less than 48 hours) than the Lmdd-based vaccines from the spleens of the immunized mice. The expression and secretion of the fusion protein tLLO-ChHer2 from this strain was comparable to that of the Lm-LLO-ChHer2 in TCA precipitated cell culture supernatants after 8 hours of in vitro growth (FIG. 11B) as a band of ˜104 KD was detected by an anti-LLO antibody using Western Blot analysis. The Listeria backbone strain expressing only tLLO was used as negative control.

pAdv84 sequence (7075 base pairs) (see FIGS. 11A and 11B):

(SEQ ID NO: 49)
cggagtgtatactggcttactatgttggcactgatgagggtgtcagtgaa
gtgcttcatgtggcaggagaaaaaaggctgcaccggtgcgtcagcagaat
atgtgatacaggatatattccgcttcctcgctcactgactcgctacgctc
ggtcgttcgactgcggcgagcggaaatggcttacgaacggggcggagatt
tcctggaagatgccaggaagatacttaacagggaagtgagagggccgcgg
caaagccgtttttccataggctccgcccccctgacaagcatcacgaaatc
tgacgctcaaatcagtggtggcgaaacccgacaggactataaagatacca
ggcgtttccccctggcggctccctcgtgcgctctcctgttcctgcctttc
ggtttaccggtgtcattccgctgttatggccgcgtttgtctcattccacg
cctgacactcagttccgggtaggcagttcgctccaagctggactgtatgc
acgaaccccccgttcagtccgaccgctgcgccttatccggtaactatcgt
cttgagtccaacccggaaagacatgcaaaagcaccactggcagcagccac
tggtaattgatttagaggagttagtcttgaagtcatgcgccggttaaggc
taaactgaaaggacaagttttggtgactgcgctcctccaagccagttacc
tcggttcaaagagttggtagctcagagaaccttcgaaaaaccgccctgca
aggcggttttttcgttttcagagcaagagattacgcgcagaccaaaacga
tctcaagaagatcatcttattaatcagataaaatatttctagccctcctt
tgattagtatattcctatcttaaagttacttttatgtggaggcattaaca
tttgttaatgacgtcaaaaggatagcaagactagaataaagctataaagc
aagcatataatattgcgtttcatctttagaagcgaatttcgccaatatta
taattatcaaaagagaggggtggcaaacggtatttggcattattaggtta
aaaaatgtagaaggagagtgaaacccatgaaaaaaataatgctagttttt
attacacttatattagttagtctaccaattgcgcaacaaactgaagcaaa
ggatgcatctgcattcaataaagaaaattcaatttcatccatggcaccac
cagcatctccgcctgcaagtcctaagacgccaatcgaaaagaaacacgcg
gatgaaatcgataagtatatacaaggattggattacaataaaaacaatgt
attagtataccacggagatgcagtgacaaatgtgccgccaagaaaaggtt
acaaagatggaaatgaatatattgttgtggagaaaaagaagaaatccatc
aatcaaaataatgcagacattcaagttgtgaatgcaatttcgagcctaac
ctatccaggtgctctcgtaaaagcgaattcggaattagtagaaaatcaac
cagatgttctccctgtaaaacgtgattcattaacactcagcattgatttg
ccaggtatgactaatcaagacaataaaatagttgtaaaaaatgccactaa
atcaaacgttaacaacgcagtaaatacattagtggaaagatggaatgaaa
aatatgctcaagcttatccaaatgtaagtgcaaaaattgattatgatgac
gaaatggcttacagtgaatcacaattaattgcgaaatttggtacagcatt
taaagctgtaaataatagcttgaatgtaaacttcggcgcaatcagtgaag
ggaaaatgcaagaagaagtcattagttttaaacaaatttactataacgtg
aatgttaatgaacctacaagaccttccagatttttcggcaaagctgttac
taaagagcagttgcaagcgcttggagtgaatgcagaaaatcctcctgcat
atatctcaagtgtggcgtatggccgtcaagtttatttgaaattatcaact
aattcccatagtactaaagtaaaagctgcttttgatgctgccgtaagcgg
aaaatctgtctcaggtgatgtagaactaacaaatatcatcaaaaattctt
ccttcaaagccgtaatttacggaggttccgcaaaagatgaagttcaaatc
atcgacggcaacctcggagacttacgcgatattttgaaaaaaggcgctac
ttttaatcgagaaacaccaggagttcccattgcttatacaacaaacttcc
taaaagacaatgaattagctgttattaaaaacaactcagaatatattgaa
acaacttcaaaagcttatacagatggaaaaattaacatcgatcactctgg
aggatacgttgctcaattcaacatttcttgggatgaagtaaattatgatc
tcgagacccacctggacatgctccgccacctctaccagggctgccaggtg
gtgcagggaaacctggaactcacctacctgcccaccaatgccagcctgtc
cttcctgcaggatatccaggaggtgcagggctacgtgctcatcgctcaca
accaagtgaggcaggtcccactgcagaggctgcggattgtgcgaggcacc
cagctctttgaggacaactatgccctggccgtgctagacaatggagaccc
gctgaacaataccacccctgtcacaggggcctccccaggaggcctgcggg
agctgcagcttcgaagcctcacagagatcttgaaaggaggggtcttgatc
cagcggaacccccagctctgctaccaggacacgattttgtggaagaatat
ccaggagtttgctggctgcaagaagatctttgggagcctggcatttctgc
cggagagctttgatggggacccagcctccaacactgccccgctccagcca
gagcagctccaagtgtttgagactctggaagagatcacaggttacctata
catctcagcatggccggacagcctgcctgacctcagcgtcttccagaacc
tgcaagtaatccggggacgaattctgcacaatggcgcctactcgctgacc
ctgcaagggctgggcatcagctggctggggctgcgctcactgagggaact
gggcagtggactggccctcatccaccataacacccacctctgcttcgtgc
acacggtgccctgggaccagctctttcggaacccgcaccaagctctgctc
cacactgccaaccggccagaggacgagtgtgtgggcgagggcctggcctg
ccaccagctgtgcgcccgagggcagcagaagatccggaagtacacgatgc
ggagactgctgcaggaaacggagctggtggagccgctgacacctagcgga
gcgatgcccaaccaggcgcagatgcggatcctgaaagagacggagctgag
gaaggtgaaggtgcttggatctggcgcttttggcacagtctacaagggca
tctggatccctgatggggagaatgtgaaaattccagtggccatcaaagtg
ttgagggaaaacacatcccccaaagccaacaaagaaatcttagacgaagc
atacgtgatggctggtgtgggctccccatatgtctcccgccttctgggca
tctgcctgacatccacggtgcagctggtgacacagcttatgccctatggc
tgcctcttagactaatctagacccgggccactaactcaacgctagtagtg
gatttaatcccaaatgagccaacagaaccagaaccagaaacagaacaagt
aacattggagttagaaatggaagaagaaaaaagcaatgatttcgtgtgaa
taatgcacgaaatcattgcttatttttttaaaaagcgatatactagatat
aacgaaacaacgaactgaataaagaatacaaaaaaagagccacgaccagt
taaagcctgagaaactttaactgcgagccttaattgattaccaccaatca
attaaagaagtcgagacccaaaatttggtaaagtatttaattactttatt
aatcagatacttaaatatctgtaaacccattatatcgggtttttgagggg
atttcaagtctttaagaagataccaggcaatcaattaagaaaaacttagt
tgattgccttttttgttgtgattcaactttgatcgtagcttctaactaat
taattttcgtaagaaaggagaacagctgaatgaatatcccttttgttgta
gaaactgtgcttcatgacggcttgttaaagtacaaatttaaaaatagtaa
aattcgctcaatcactaccaagccaggtaaaagtaaaggggctatttttg
cgtatcgctcaaaaaaaagcatgattggcggacgtggcgttgttctgact
tccgaagaagcgattcacgaaaatcaagatacatttacgcattggacacc
aaacgtttatcgttatggtacgtatgcagacgaaaaccgttcatacacta
aaggacattctgaaaacaatttaagacaaatcaataccttctttattgat
tttgatattcacacggaaaaagaaactatttcagcaagcgatattttaac
aacagctattgatttaggttttatgcctacgttaattatcaaatctgata
aaggttatcaagcatattttgttttagaaacgccagtctatgtgacttca
aaatcagaatttaaatctgtcaaagcagccaaaataatctcgcaaaatat
ccgagaatattttggaaagtctttgccagttgatctaacgtgcaatcatt
ttgggattgctcgtataccaagaacggacaatgtagaattttttgatccc
aattaccgttattctttcaaagaatggcaagattggtctttcaaacaaac
agataataagggctttactcgttcaagtctaacggttttaagcggtacag
aaggcaaaaaacaagtagatgaaccctggtttaatctcttattgcacgaa
acgaaattttcaggagaaaagggtttagtagggcgcaatagcgttatgtt
taccctctctttagcctactttagttcaggctattcaatcgaaacgtgcg
aatataatatgtttgagtttaataatcgattagatcaacccttagaagaa
aaagaagtaatcaaaattgttagaagtgcctattcagaaaactatcaagg
ggctaatagggaatacattaccattctttgcaaagcttgggtatcaagtg
atttaaccagtaaagatttatttgtccgtcaagggtggtttaaattcaag
aaaaaaagaagcgaacgtcaacgtgttcatttgtcagaatggaaagaaga
tttaatggcttatattagcgaaaaaagcgatgtatacaagccttatttag
cgacgaccaaaaaagagattagagaagtgctaggcattcctgaacggaca
ttagataaattgctgaaggtactgaaggcgaatcaggaaattttctttaa
gattaaaccaggaagaaatggtggcattcaacttgctagtgttaaatcat
tgttgctatcgatcattaaattaaaaaaagaagaacgagaaagctatata
aaggcgctgacagcttcgtttaatttagaacgtacatttattcaagaaac
tctaaacaaattggcagaacgccccaaaacggacccacaactcgatttgt
ttagctacgatacaggctgaaaataaaacccgcactatgccattacattt
atatctatgatacgtgtttgtttttctttgctggctagcttaattgctta
tatttacctgcaataaaggatttcttacttccattatactcccattttcc
aaaaacatacggggaacacgggaacttattgtacaggccacctcatagtt
aatggtttcgagccttcctgcaatctcatccatggaaatatattcatccc
cctgccggcctattaatgtgacttttgtgcccggcggatattcctgatcc
agctccaccataaattggtccatgcaaattcggccggcaattttcaggcg
ttttcccttcacaaggatgtcggtccctttcaattttcggagccagccgt
ccgcatagcctacaggcaccgtcccgatccatgtgtctttttccgctgtg
tactcggctccgtagctgacgctctcgccttttctgatcagtttgacatg
tgacagtgtcgaatgcagggtaaatgccggacgcagctgaaacggtatct
cgtccgacatgtcagcagacgggcgaaggccatacatgccgatgccgaat
ctgactgcattaaaaaagccttttttcagccggagtccagcggcgctgtt
cgcgcagtggaccattagattctttaacggcagcggagcaatcagctctt
taaagcgctcaaactgcattaagaaatagcctctttctttttcatccgct
gtcgcaaaatgggtaaatacccctttgcactttaaacgagggttgcggtc
aagaattgccatcacgttctgaacttcttcctctgtttttacaccaagtc
tgttcatccccgtatcgaccttcagatgaaaatgaagagaaccttttttc
gtgtggcgggctgcctcctgaagccattcaacagaataacctgttaaggt
cacgtcatactcagcagcgattgccacatactccgggggaaccgcgccaa
gcaccaatataggcgccttcaatccctttttgcgcagtgaaatcgcttca
tccaaaatggccacggccaagcatgaagcacctgcgtcaagagcagcctt
tgctgtttctgcatcaccatgcccgtaggcgtttgctttcacaactgcca
tcaagtggacatgttcaccgatatgttttttcatattgctgacattttcc
tttatcgcggacaagtcaatttccgcccacgtatctctgtaaaaaggttt
tgtgctcatggaaaactcctctcttttttcagaaaatcccagtacgtaat
taagtatttgagaattaattttatattgattaatactaagtttacccagt
tttcacctaaaaaacaaatgatgagataatagctccaaaggctaaagagg
actataccaactatttgttaattaa

### Example 10

### ADXS31-164 Is as Immunogenic As Lm-LLO-ChHER2

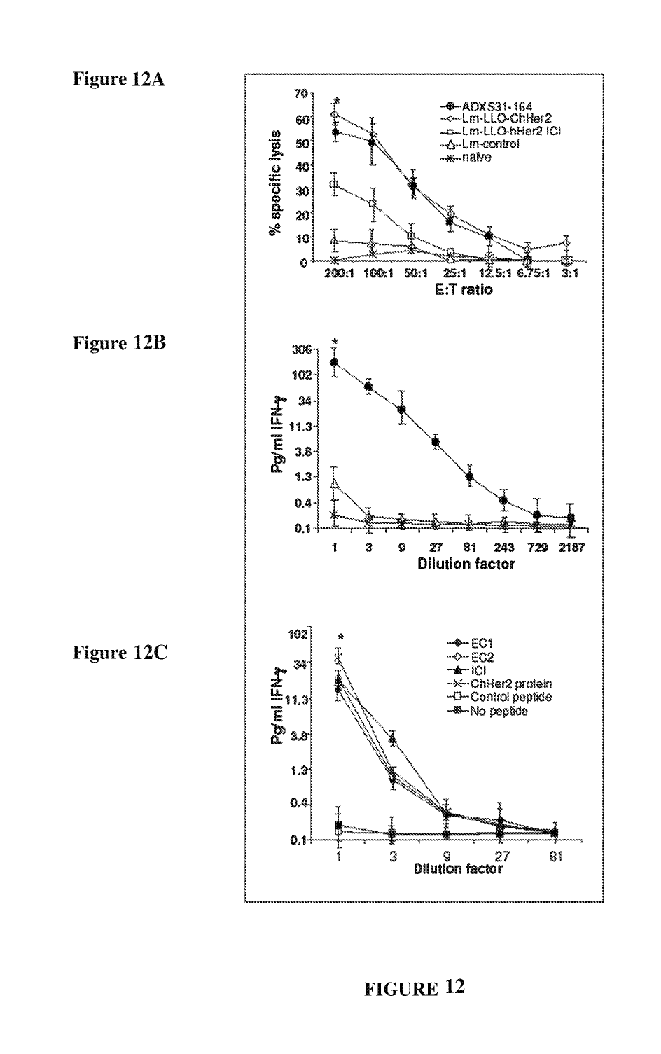

Immunogenic properties of ADXS31-164 in generating anti-Her2/neu specific cytotoxic T cells were compared to those of the Lm-LLO-ChHer2 vaccine in a standard CTL assay. Both vaccines elicited strong but comparable cytotoxic T cell responses toward Her2/neu antigen expressed by 3T3/neu target cells. Accordingly, mice immunized with a Listeria expressing only an intracellular fragment of Her2-fused to LLO showed lower lytic activity than the chimeras which contain more MHC class I epitopes. No CTL activity was detected in naive animals or mice injected with the irrelevant Listeria vaccine (FIG. 12A). ADXS31-164 was also able to stimulate the secretion of IFN-γ by the splenocytes from wild type FVB/N mice (FIG. 12B). This was detected in the culture supernatants of these cells that were co-cultured with mitomycin C treated NT-2 cells, which express high levels of Her2/neu antigen (FIG. 12C).

Proper processing and presentation of the human MHC class I epitopes after immunizations with ADXS31-164 was tested in HLA-A2 mice. Splenocytes from immunized HLA-A2 transgenics were co-incubated for 72 hours with peptides corresponding to mapped HLA-A2 restricted epitopes located at the extracellular (HLYQGCQVV SEQ ID NO: 50 or KIFGSLAFL SEQ ID NO: 51) or intracellular (RLLQETELV SEQ ID NO: 52) domains of the Her2/neu molecule (FIG. 12C). A recombinant ChHer2 protein was used as positive control and an irrelevant peptide or no peptide as negative controls. The data from this experiment show that ADXS31-164 is able to elicit anti-Her2/neu specific immune responses to human epitopes that are located at different domains of the targeted antigen.

### Example 11

### ADXS31-164 was More Efficacious than Lm-LLO-ChHER2 in Preventing the Onset of Spontaneous Mammary Tumors

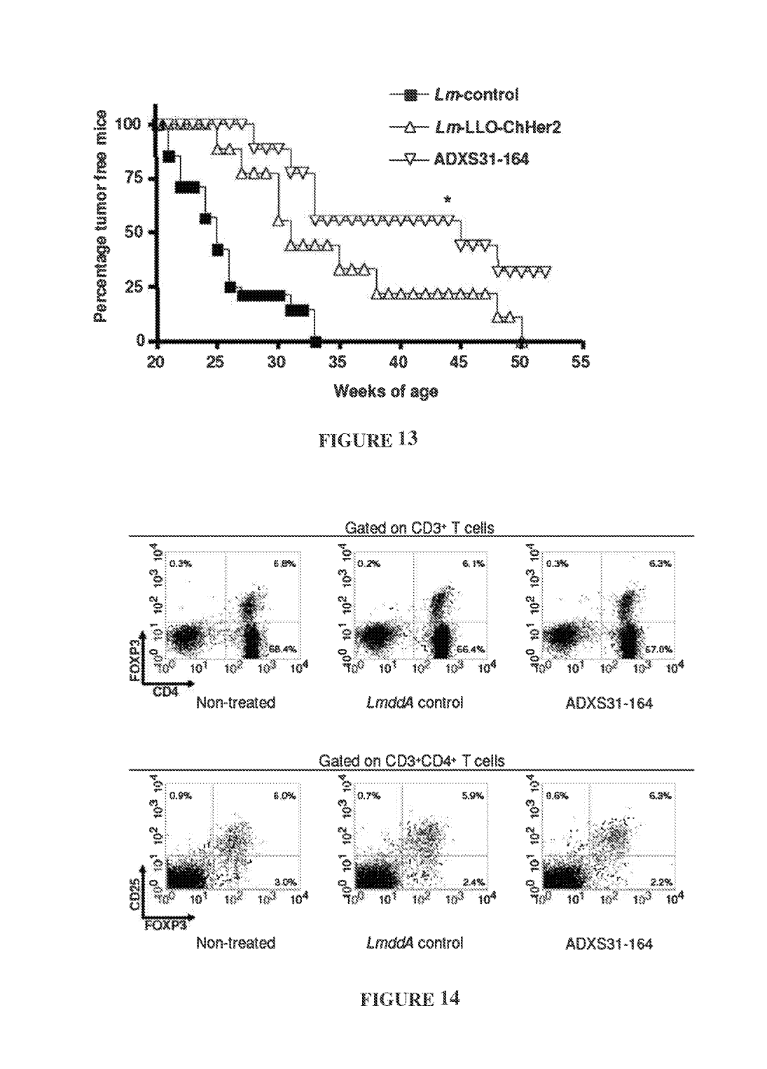

Anti-tumor effects of ADXS31-164 were compared to those of Lm-LLO-ChHer2 in Her2/neu transgenic animals which develop slow growing, spontaneous mammary tumors at 20-25 weeks of age. All animals immunized with the irrelevant Listeria-control vaccine developed breast tumors within weeks 21-25 and were sacrificed before week 33. In contrast, Liseria-Her2/neu recombinant vaccines caused a significant delay in the formation of the mammary tumors. On week 45, more than 50% of ADXS31-164 vaccinated mice (5 out of 9) were still tumor free, as compared to 25% of mice immunized with Lm-LLO-ChHer2. At week 52, 2 out of 8 mice immunized with ADXS31-164 still remained tumor free, whereas all mice from other experimental groups had already succumbed to their disease (FIG. 13). These results indicate that despite being more attenuated, ADXS31-164 is more efficacious than Lm-LLO-ChHer2 in preventing the onset of spontaneous mammary tumors in Her2/neu transgenic animals.

### Example 12

### Mutations in HER2/Neu Gene Upon Immunization with ADXS31-164

Mutations in the MHC class I epitopes of Her2/neu have been considered responsible for tumor escape upon immunization with small fragment vaccines or trastuzumab (Herceptin), a monoclonal antibody that targets an epitope in the extracellular domain of Her2/neu. To assess this, genomic material was extracted from the escaped tumors in the transgenic animals and sequenced the corresponding fragments of the neu gene in tumors immunized with the chimeric or control vaccines. Mutations were not observed within the Her-2/neu gene of any vaccinated tumor samples suggesting alternative escape mechanisms (data not shown).

### Example 13

### ADXS31-164 Causes A Significant Decrease in Intra-Tumoral T Regulatory Cells

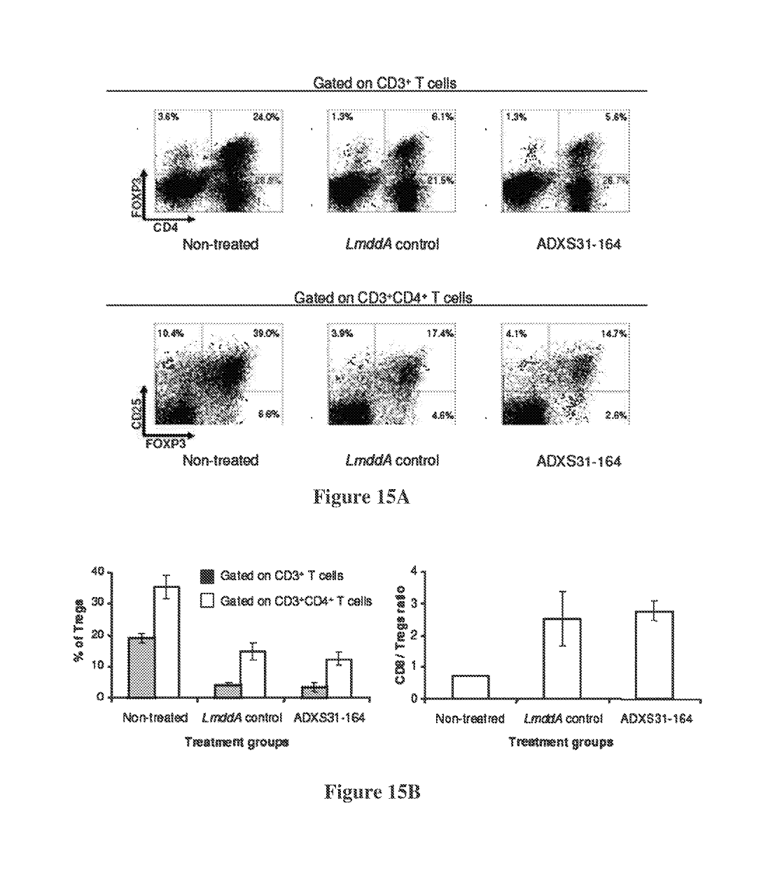

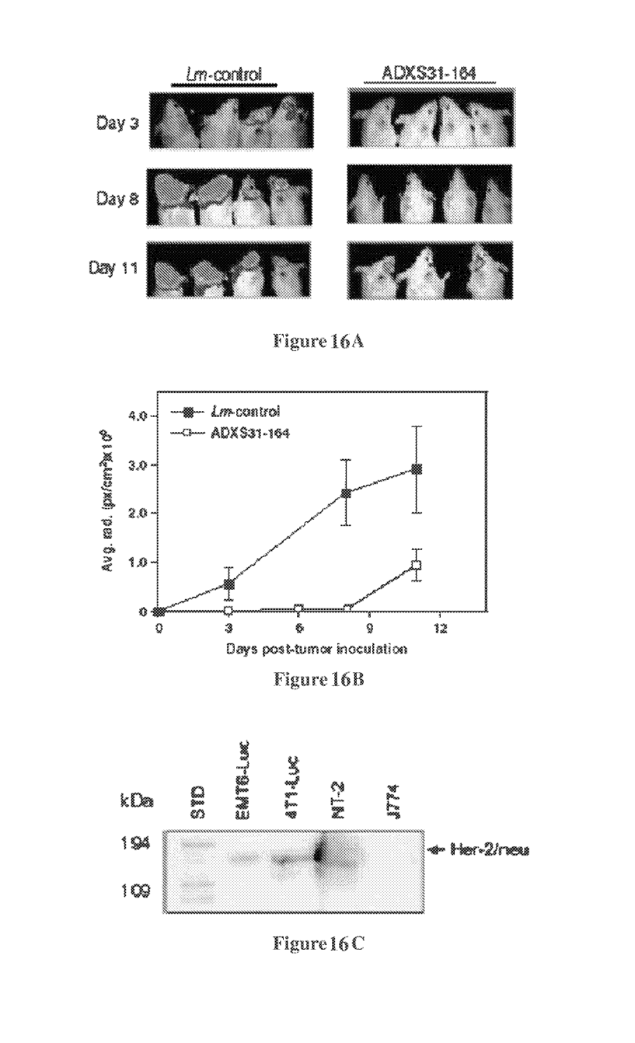

To elucidate the effect of ADXS31-164 on the frequency of regulatory T cells in spleens and tumors, mice were implanted with NT-2 tumor cells. Splenocytes and intra-tumoral lymphocytes were isolated after three immunizations and stained for Tregs, which were defined as CD3⁺/CD4⁺/CD25⁺/FoxP3⁺ cells, although comparable results were obtained with either FoxP3 or CD25 markers when analyzed separately. The results indicated that immunization with ADXS31-164 had no effect on the frequency of Tregs in the spleens, as compared to an irrelevant Listeria vaccine or the naive animals (FIG. 14). In contrast, immunization with the Listeria vaccines caused a considerable impact on the presence of Tregs in the tumors (FIG. 15A). Whereas in average 19.0% of all CD3⁺ T cells in untreated tumors were Tregs, this frequency was reduced to 4.2% for the irrelevant vaccine and 3.4% for ADXS31-164, a 5-fold reduction in the frequency of intra-tumoral Tregs (FIG. 15B). The decrease in the frequency of intra-tumoral Tregs in mice treated with either of the LmddA vaccines could not be attributed to differences in the sizes of the tumors. In a representative experiment, the tumors from mice immunized with ADXS31-164 were significantly smaller [mean diameter (mm)±SD, 6.71±0.43, n=51 than the tumors from untreated mice (8.69±0.98, n=5, p<0.01) or treated with the irrelevant vaccine (8.41±1.47, n=5, p=0.04), whereas comparison of these last two groups showed no statistically significant difference in tumor size (p=0.73). The lower frequency of Tregs in tumors treated with LmddA vaccines resulted in an increased intratumoral CD8/Tregs ratio, suggesting that a more favorable tumor microenvironment can be obtained after immunization with LmddA vaccines. However, only the vaccine expressing the target antigen HER2/neu (ADXS31-164) was able to reduce tumor growth, indicating that the decrease in Tregs has an effect only in the presence on antigen-specific responses in the tumor.

### Example 14

### Peripheral Immunization with ADXS31-164 Can Delay the Growth of a Metastatic Breast Cancer Cell Line in the Brain

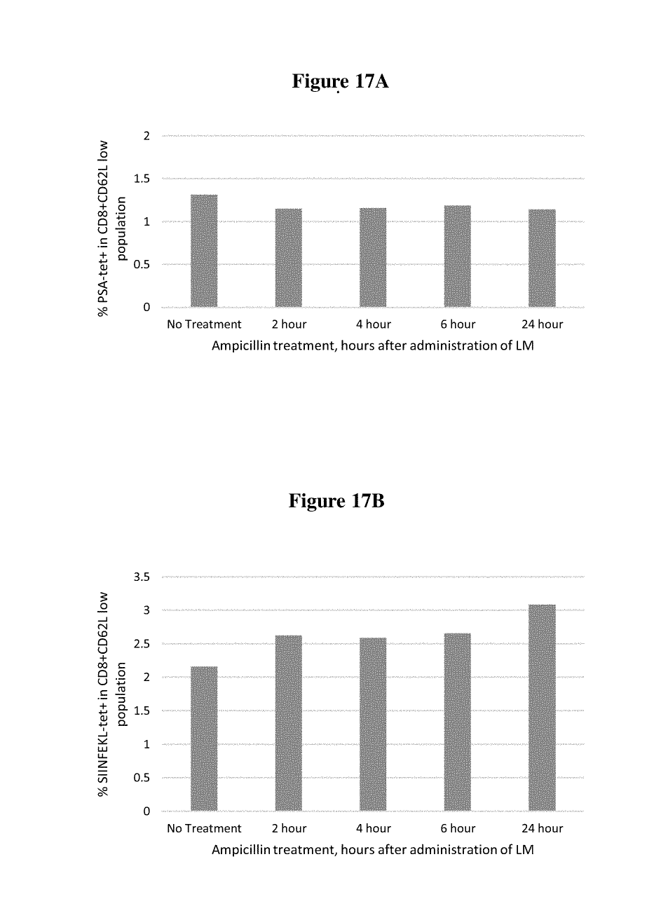

Mice were immunized IP with ADXS31-164 or irrelevant Lm-control vaccines and then implanted intra-cranially with 5,000 EMT6-Luc tumor cells, expressing luciferase and low levels of Her2/neu (FIG. 16A). Tumors were monitored at different times post-inoculation by ex vivo imaging of anesthetized mice. On day 8 post-tumor inoculation tumors were detected in all control animals, but none of the mice in ADXS31-164 group showed any detectable tumors (FIGS. 16A and 16B). ADXS31-164 could clearly delay the onset of these tumors, as on day 11 post-tumor inoculation all mice in negative control group had already succumbed to their tumors, but all mice in ADXS31-164 group were still alive and only showed small signs of tumor growth. These results strongly suggest that the immune responses obtained with the peripheral administration of ADXS31-164 could possibly reach the central nervous system and that LmddA-based vaccines might have a potential use for treatment of CNS tumors.

### Example 15

### Prevention of Biofilm Formation Following Administration of Listeria-Based Immunotherapies

### Materials and Methods

Mice are implanted with bone grafts prior to being administered with an inoculum of a Listeria immunotherapy (e.g. ADXS31-142 or ADXS31-164) in order to measure seeding of the Listeria on the bone grafts and subsequent biofilm formation.

On day 1, an experimental group of mice are inoculated with a Listeria immunotherapy dose and are then administered with an antibiotic that does not penetrate the cells such as clindamycin or gentamycin immediately after administration of the Listeria immunotherapy or up to 1 hr thereafter. 10, 12, 14, and 16 hours after administration of the Listeria immunotherapy mice are administered a second dose of antibiotics using antibiotics that penerate the host cells and eliminate intracellular bacteria, such as Ampicillin. On day 2, bone grafts are collected from and tissue is analyzed for the presence of biofilms. On day 1, a first group of control mice are inoculated with a Listeria immunotherapy dose and are left untreated without antibiotics until bone graft collection. On day 1, a second group of control mice are inoculated with a Listeria immunotherapy dose and also receive a dose of antibiotics 10, 12, 14, and 16 hours after administration of the Listeria immunotherapy using antibiotics that penerate the host cells and eliminate intracellular bacteria, such as Ampicillin in order to determine the efficacy of complete Listeria clearance. On day 2, bone grafts are collected from both control groups and tissue is analyzed for the presence of biofilms.

Spleens and liver are also collected from mice of all groups to determine the presence of Listeria.

Results

It is observed that in the Experimental group the Listeria immunotherapy is capable of presenting antigen to the immune cells and elicit an anti-tumor/anti-cancer immune response and following antibiotic treatment the Listeria is completely cleared from the mice.

### Example 16

### Early Time Point Administration of Antibiotics Does Not Alter the Immunogenicity of PSA-SVN Tag (P2 g6.1)

An assay was performed to examine the generation of SIINFEKL-specific and PSA-specific immunity in mice treated with ampicillin or gentamicin/ampicillin and immunized with PSA-SVN Tag. The SIINFEKL-specific immune response was detected by pentamer staining using the known T cell epitopes for C57BL/6 mice, H-2 Db PSA65-73 (HCIRNKSVI) and H-2 Kb OVA257-264 (SIINFEKL).

Prime-Harvest with Ampicillin: On Day 0, all groups were infected with PSA-SVN Tag LM IP, and then ampicillin was administered at 100 mg/kg IP at various time points from hour 2, 4, 6, 24. All Animals (except negative control) were given 1 dose of ampicillin at 24 hours. 7 days after Day 0 infection, mice were sacrificed and spleens were harvested and analyzed for specific T-cell epitopes to H-2 Db PSA₆₅₋₇₃ (HCIRNKSVI) and H-2 Kb OVA₂₅₇₋₂₆₄ (SIINFEKL).

TABLE 3
Groups and type of treatment

| Groups | | Doses of Amp | Dose of | Harvest |
|---|---|---|---|---|
| (5 mice/ | Vaccines | 100 mg/kg | Amp | Spleen and |
| group) | on Day 0 | on Day 0 | 100 mg/kg | Femur |
| 1 | PSA-SVN Tag | NONE | NONE | Day 7 |
| | (P2 g6.1) | | | |
| | (positive control) | | | |
| 2 | PSA-SVN Tag | 2 hour | 24 hour | Day 7 |
| | (P2 g6.1) + | | | |
| | ampicillin Hour 2 | | | |
| 3 | PSA-SVN Tag | 4 hour | 24 hour | Day 7 |
| | (P2 g6.1) + | | | |
| | ampicillin Hour 4 | | | |
| 4 | PSA-SVN Tag | 6 hour | 24 hour | Day 7 |
| | (P2 g6.1) + | | | |
| | ampicillin Hour 6 | | | |
| 5 | PSA-SVN Tag | NONE | 24 hour | Day 7 |
| | (P2 g6.1) + | | | |
| | (positive control) | | | |

Prime-Harvest with Gentamicin+Ampicillin chase: On Day 0, all groups were infected with PSA-SVN Tag LM IP, and then gentamicin was administered at 5 mg/kg IP at various time points from hour 2, 4, 6, 24. All Animals (except negative control) were given 1 dose of ampicillin (100 mg/kg IP) at 24 hours post vaccination. 7 days after Day 0 infection, mice were sacrificed and spleens were harvested and analyzed for specific T-cell epitopes to H-2 Db PSA₆₅₋₇₃ (HCIRNKSVI) and H-2 Kb OVA₂₅₇₋₂₆₄ (SIINFEKL)

TABLE 4
Groups and type of treatment

| Groups | | Doses of | | |
|---|---|---|---|---|
| (5 mice/ | Vaccines | Gentamicin | Doses of | Harvest |
| group) | on Day 0 | On Day 0 | Ampicillin | Spleen |
| 1 | PSA-SVN | NONE | NONE | Day 7 |
| | Tag (P2 g6.1) | | | |
| | (positive control) | | | |
| 2 | PSA-SVN | 2 hour | 24 hour | Day 7 |
| | Tag (P2 g6.1) + | | | |
| | gentamicin Hour 2 | | | |
| 3 | PSA-SVN | 4 hour | 24 hour | Day 7 |
| | Tag (P2 g6.1) + | | | |
| | gent Hour 4 | | | |
| 4 | PSA-SVN | 6 hour | 24 hour | Day 7 |
| | Tag (P2 g6.1) + | | | |
| | gent Hour 6 | | | |
| 5 | PSA-SVN | NONE | 24 hour | Day 7 |
| | Tag (P2 g6.1) + | | | |
| | ampicillin Hour | | | |
| | 24 hours (control) | | | |

### Treatment Preparation

PSA-SVN-Tag (P2 g6.1) -Titer: 1.7×10⁹ CFU/mL was prepared by: Thawing 1 vial from −80 C in 37 C water bath; Spinning at 14, 000 rpm for 2 min and discarding supernatant; Washing 2 times with 1 mL PBS and discard PBS; Re-suspending in PBS to a final concentration of 5×10⁸ CFU/mL.

Ampicillin A8351 Sigma, concentration: 10 mg/mL; solvent: sterile H2O; solution stability: 3 days. Working Concentration: (˜100 mg/kg) 10 mg/mL to (2 mg/200 uL/mouse IP).

Gentamicin G1272 Sigma (5 mg/kg) 0.1 mg/200 uL/mouse IP, concentration: 10 mg/mL; solvent: sterile H2O, solution stability: 5 days at 37 C; months 2-8 C. Working Concentration: 5 mg/kg (10 ul gent in 190 ul ddH20 per mouse IP).

### Harvesting: Preparing Isolated Splenocytes

The spleen from each mouse was collected in an individual tube containing 5 ml of c-RPMI medium. Detailed steps are described as follows: Harvest spleens using sterile forceps and scissors. Transport in c-RPMI to the lab; Pour each spleen into a sterile Petri dish; Mash each spleen in wash medium (RPMI only) using two glass slides or the back of plunger from a 3 mL syringe; Transfer cells in the medium to a 15 ml tube, for 1 or 2 spleens or 50 ml tubes if you have more than two spleens; Pellet cells at 1,000 RPM for 5 min at RT; Discard sup, re-suspend cells in the remaining wash buffer gently and add 2 ml RBC lysis buffer per spleen to the cell pellet. Mix cells gently with lysis buffer by tapping the tube and wait for 1 min; Immediately add 10 ml of c-RPMI medium to the cell suspension to deactivate lysis buffer; Spin cells at 1,000 for 5 min at RT; Pass the cells through a cell strainer and wash them one more time with 10 ml c-RPMI; Count cells using hemocytometer/moxi flow and check the viability by Trypan blue staining. Each spleen should yield ˜1-2×10⁸ cells; Divide the cells for staining. Immudex dextramer staining protocol (http://www.immudex.com/media/12135/tf1003.03_general_staining_procedure_mhc_dextra mer.pdf) was used with the one exception of adding the cell surface antibodies (CD8, CD62L) in 2.4G2 instead of staining buffer.

### Results

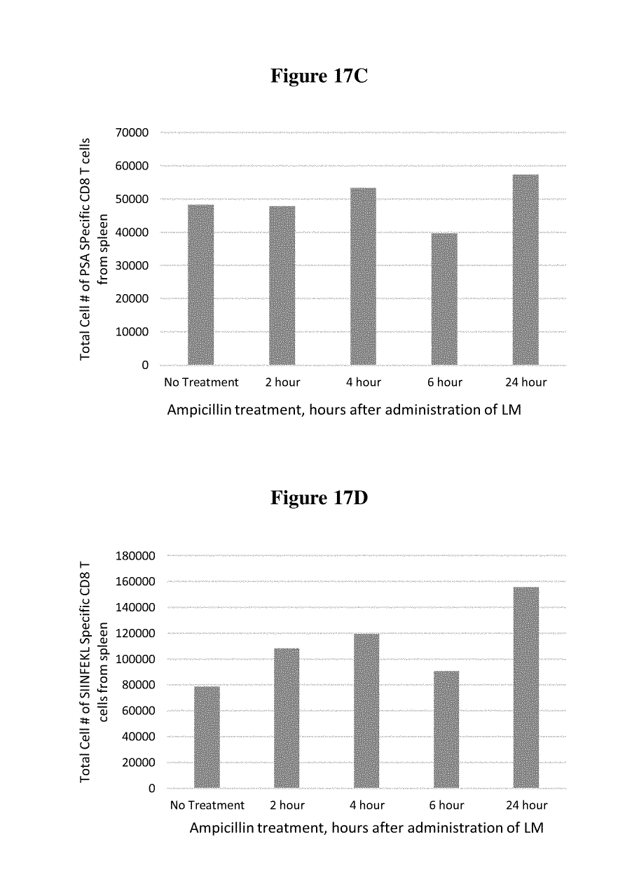

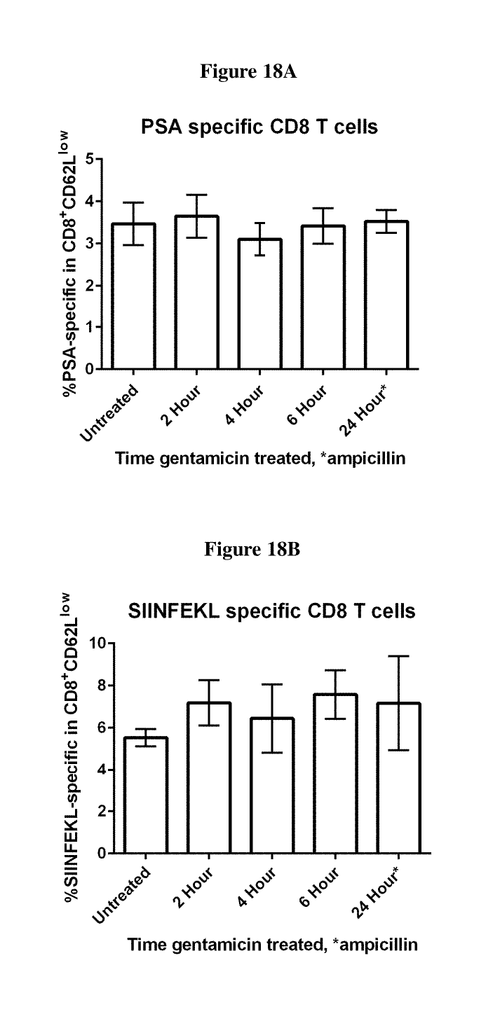

No significant difference between groups were found for both the early time point administration of ampicillin only (FIGS. 17A-D) or gentamicin plus 24 hour ampicillin chase (FIGS. 18A-B).

Effects of Early Time Point Administration of Antibiotics on Listeria monocytogenes Seeding in the Bone Marrow

The ability of Listeria monocytogenes to seed the bone marrow at various ampicillin or gentamicin dosing schedules will be ascertained via CFU counts. Bone marrow will be harvested from femurs and plated on Pen-Strep BHI plates in order to detect the presence of Listeria monocytogenes colonies. The femurs will be harvested from all mice; n=25 mice−50 femurs total. Bone marrow will be isolated from the femurs as follows: Harvest femurs using sterile forceps and scissors; transport in RPMI-1640 w/strep to the lab and place in sterile 12-well tissue culture plates (one femur per well); Cut the hind leg below the knee-joint through ligaments to remove off the tibia, ensuring that the epiphysis remains intact; Dissect the femur from surrounding muscles and remove excess tissue, keeping the ends of the bone intact; Remove any leftover muscle/tissue on the femur; Transfer the bones to culture medium RPMI-1640 w/strep in sterile 12-well tissue culture plates (one femur per well); Trim both ends of femurs carefully to expose the interior marrow shaft; Flush the contents of marrow with 2-3 mL of RPMI-1640 w/strep using 1 mL insulin syringes with 29G×½ needles; Collect the contents from each femur, separately, into a sterile 15 mL centrifuge tube w/5 mLs of BHI/Strep media. (The bones should appear white once all the marrow has been expelled out completely); Collect flushed femurs as well in separate 5 mL tubes w/BHI/strep media; Place samples @ 37 C O/N; Next day, if turbid, perform serial dilutions in duplicate on BHI/strep bacterial plates; and If any bacterial growth appears the following day, perform colony PCR.

### Example 17

### Effects of Administration of Ampicillin at Different Time Points After Lm Treatment of Drug Product ADXS11-001 (HPV 1.0)

A tumor regression study was initiated in C57BL/6 female mice using TC-1 lung epithelial cells to assess the therapeutic efficacy of different Lm treatment with or without ampicillin treatment and whether it alters the tumor microenvironment.

Tumor Inoculation: The tumor model used in this study is the TC-1 tumor model. TC-1 cells are cultured in complete medium. Complete medium for TC-1 cells: 450 ml RPMI 1640, 50 ml 10%FBS, 5 ml NEAA (100 uM), 5 ml L-Glutamine (2 uM), and 5 ml Pen/strep (100 U/mL) penicillin+100 ug/mL streptomycin) G418 (400 ug/mL) (added to cells when splitting). Two days prior to implanting tumor cells in mice, TC-1 cells were sub-cultured in complete media. On the day of the experiment (Day 0), cells were trypsinized and washed twice with media. Cells were counted and re-suspended at a concentration of 1×10⁵ cells/200 ul in PBS/mouse for injection. Tumor cells were injected subcutaneously in the right flank of each mouse. Tumors were measured twice a week until they reached a size of 12 mm in diameter. Once tumors met sacrifice criteria, mice were euthanized and tumors were excised and measured.

Vaccine/Ampicillin Treatment: On Day 6, when tumors are ˜5mm in size; vaccines and treatments began. All groups were infected with DP ADXS11-001 (HPV 1.0) Lm, intraperitoneally (IP), followed by ampicillin administered at 100mg/kg (IP) at various time points from 0-hr, 4-hr, 6-hr and 24-hr, respectively. Groups 4 and 5 received a 24-hr ampicillin treatment IP.

TABLE 5
Treatment Schedule

| | | Dose 1: | | |
|---|---|---|---|---|
| | TC-1 Tumor | Lm + Ampicillin | | |
| | Inoculation | Treatments at | Dose 2: | Dose 3: |
| Groups | 1 × 10⁵ cells/ | 1 week intervals | Lm + Ampicillin | Lm + Ampicillin |
| (10 mice/group) | 200 uL/mouse | (IP) | (100 mg/kg IP) | (100 mg/kg IP) |
| 1-LmddA-274 | Day 0 | Day 6 | Day 13 | Day 20 |
| ONLY NO Ab | | No Ab | No Ab | No Ab |
| (positive control) | | | | |
| 2-PBS ONLY | Day 0 | Day 6 | Day 13 | Day 20 |
| NO Ab | | NO Ab | NO Ab | NO Ab |
| 3-DP ADXS11-001 | Day 0 | Day 6 | Day 13 | Day 20 |
| ONLY (HPV 1.0) | | No Ab | No Ab | No Ab |
| NO Ab | | | | |
| 4-DP ADXS11-001 | Day 0 | Day 6 + Day 7 | Day 13 + 14 | Day 20 + Day 21 |
| (HPV 1.0) + | | (24-hr AMP) | (24-hr AMP) | (24-hr AMP) |
| ampicillin 4-hr | | | | |
| 5-DP ADXS11-001 | Day 0 | Day 6 + Day 7 | Day 13 + 14 | Day 20 + Day 21 |
| (HPV 1.0) + | | (24-hr AMP) | 20 Jan. 2016 | (24-hr AMP) |
| ampicillin 6-hr | | | (24-hr AMP) | |
| 6-DP ADXS11-001 | Day 0 | Day 6-no Ab + | Day 6 | Day 21 |
| (HPV 1.0) + | | Day 7 | 24-hr AMP only | 24-hr AMP only |
| ampicillin 24-hr | | (24-hr AMP only) | | |
| Groups 3-6 to | | | | |
| receive add'l doses | | Dose 4: Lm + | | |
| One week off in | | Ampicillin | Dose 5: | |
| between Dose 3 & | | Treatments at | Lm + | Dose 6/7: |
| Dose 4, and weekly | | 1 week | Ampicillin | Lm ONLY |
| for doses 5-7. | | intervals (IP) | (100 mg/kg IP) | NO AMP |
| | | Day 34 | Day 41 | Day 48 |

### Vaccine/Treatment Preparation

LmddA-274 was prepared as follows: Thaw 1 vial from −80 C in 37 C water bath; Spin at 14, 000 rpm for 2 min and discard supernatant; Wash 2 times with 1 mL PBS and discard PBS; and Re-suspend in PBS to a final concentration of 5×10⁸ CFU/mL.

DP ADXS11-001 (HPV 1.0) (Titer: 2.2×10⁹ CFU/mL) was prepared as follows: Thaw 1 vial from −80 C in 37 C water bath; Spin at 14, 000 rpm for 2 min and discard supernatant; Wash 2 times with 1 mL PBS and discard PBS; and Re-suspend in PBS to a final concentration of 5×10⁸ CFU/mL.

Ampicillin A8351 Sigma, concentration: 10 mg/mL, solvent: sterile H2O, solution stability: 3 days. Working Concentration: (˜100 mg/kg) 10 mg/mLto (2 mg/200 uL/mouse IP).

### Results

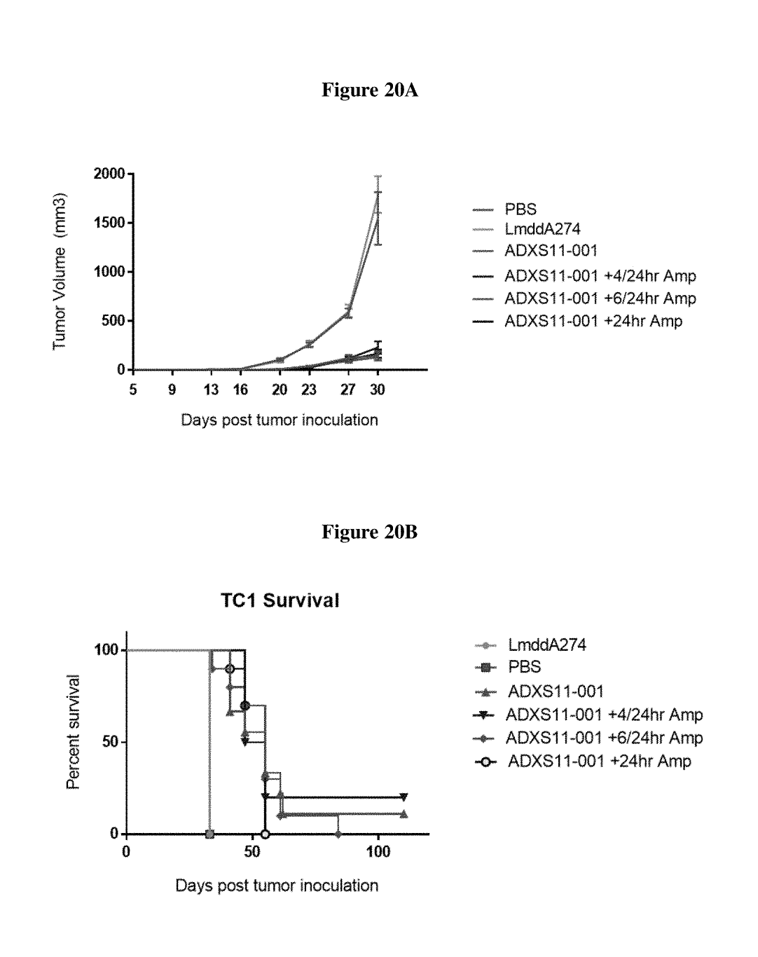

No significant difference between Lm treatment groups with or without ampicillin treatment for tumor regression (FIG. 20A) and survival (FIG. 20B).

### Example 18

### Effects of Administration of Ampicillin at Different Time Points After Lm Treatment of Drug Product ADXS31-142 (PSA 1.0)

A tumor regression study was initiated in C57BL/6 male mice using TPSA23 prostate cancer cells to assess the therapeutic efficacy of different Lm treatment with or without ampicillin treatment.

Tumor Inoculation: The tumor model used in this study is the TPSA23 tumor model. TPSA23 cells are cultured in complete medium. Complete medium for TPSA23 cells: 430 ml DMEM with Glucose, 45 ml FBS, 25 ml Nu-Serum IV, 5 ml L-Glutamine, 5 ml Na-bicarbonate, 0.005 mg/ml Bovine Insulin—Insulin stock (2.5 mg/ml) is prepared in acidified water (10 ml water+100 ul glacial acetic acid) (Add to the flask while splitting cells—6.4 uL/8 mLs media, 12.8 uL/16 mLs media, 19.2 uL/24 mLs media, etc.), and 10 nM Dehydroisoandrosterone (DHA)—DHA stock (10 mM) is prepared in ethanol. (Add to the flask while splitting cells—140 uL/70 mLs media, 70 uL/35 mLs media, 35 uL/17.5 mLs media, etc.). Two days prior to implanting tumor cells in mice, TPSA23 cells are sub-cultured in complete media. On the day of the experiment (Day 0), cells were be trypsinized and washed twice with media. Cells were counted and re-suspended at a concentration of 2×10⁶ cells/200 ul in PBS/mouse for injection. Tumor cells were injected subcutaneously in the right flank of each mouse. Tumors are measured twice a week until they reach a size of 12 mm in diameter. Once tumors met sacrifice criteria, mice were euthanized and tumors were excised and measured.

Vaccine/Ampicillin Treatment: On Day 9, when tumors are ˜9 mm in size; vaccines and treatments began. All groups were infected with DP ADXS31-142 (PSA 1.0) Lm, Intraperitoneally (IP), followed by ampicillin administered at 100 mg/kg (IP) at various time points from 0-hr, 4-hr, 6-hr and 24-hr, respectively. Groups 4 and 5 will received a 24-hr ampicillin treatment IP.

TABLE 6
Treatment Schedule

| | | Dose 1: | | |
|---|---|---|---|---|
| | | Vaccine/ | | |
| | TPSA23 | Ampicillin | | |
| | Tumor | Treatments at | Dose 2: | Dose 3: |
| Groups | Inoculation | 1 week | Lm + Ampicillin | Lm + Ampicillin |
| (10 mice/group) | 2 × 10⁶ | intervals (IP) | (100 mg/kg) IP | (100 mg/kg) IP |
| 1-LmddA-274 ONLY | Day 0 | Day 9 No Ab | Day 16 No Ab | Day 23 No Ab |
| NO Ab | | | | |
| (positive control) | | | | |
| 2-PBS ONLY | Day 0 | Day 9 | Day 16 | Day 23 |
| 3-DP ADXS31-142 | Day 0 | Day 9 No Ab | Day 16 No Ab | Day 23 No Ab |
| ONLY | | | | |
| (PSA 1.0) NO Ab | | | | |
| 4-DP ADXS31-142 | Day 0 | Day 9 + Day 10 | Day 16 + Day 17 | Day 23 + Day 24 |
| (PSA 1.0) + ampicillin | | (24-hr-AMP) | (24-hr-AMP) | (24-hr-AMP) |
| 4-hr and 24-hr | | | | |
| 5-DP ADXS31-142 | Day 0 | Day 9 + Day 10 | Day 16 + Day 17 | Day 23 + Day 24 |
| (PSA 1.0) + ampicillin | | (24-hr-AMP) | (24-hr-AMP) | (24-hr-AMP) |
| 6-hr and 24-hr | | | | |
| 6-DP ADXS31-142 | Day 0 | Day 9 | Day 16 | Day 23 |
| (PSA 1.0) + | | | | |
| ampicillin 24-hr | | | | |
| | | Dose 4: | | |
| Groups 3-6 will | | Vaccine/ | | |
| receive add'l doses | | Ampicillin | | |
| with one week off in | | Treatments at | Dose 5: | Dose 6: |
| between Dose 3 & | | 1 week | Lm + Ampicillin | Lm + Ampicillin |
| Dose 4 and 4-6 | | intervals (IP) | (100 mg/kg) IP | (100 mg/kg) IP |
| | | Day 37 | Day 51 | Day 65 |

### Vaccine/Treatment Preparation

LmddA-274 was prepared as follows: Thaw 1 vial from −80 C in 37 C water bath; Spin at 14, 000 rpm for 2min and discard supernatant; Wash 2 times with 1 mL PBS and discard PBS; and Re-suspend in PBS to a final concentration of 5×10⁸ CFU/mL.

DP ADXS31-142 (PSA 1.0) (Titer: 2×10⁹) was prepared as follows: Thaw 1 vial from −80 C in 37 C water bath; Spin at 14, 000 rpm for 2 min and discard supernatant; Wash 2 times with 1 mL PBS and discard PBS; and Re-suspend in PBS to a final concentration of 5×10⁸ CFU/mL.

Ampicillin A8351 Sigma, concentration: 10 mg/mL, solvent: sterile H2O, solution stability: 3 days. Working Concentration: (˜100 mg/kg) 10 mg/mL to (2 mg/200 uL/mouse IP).

### Results

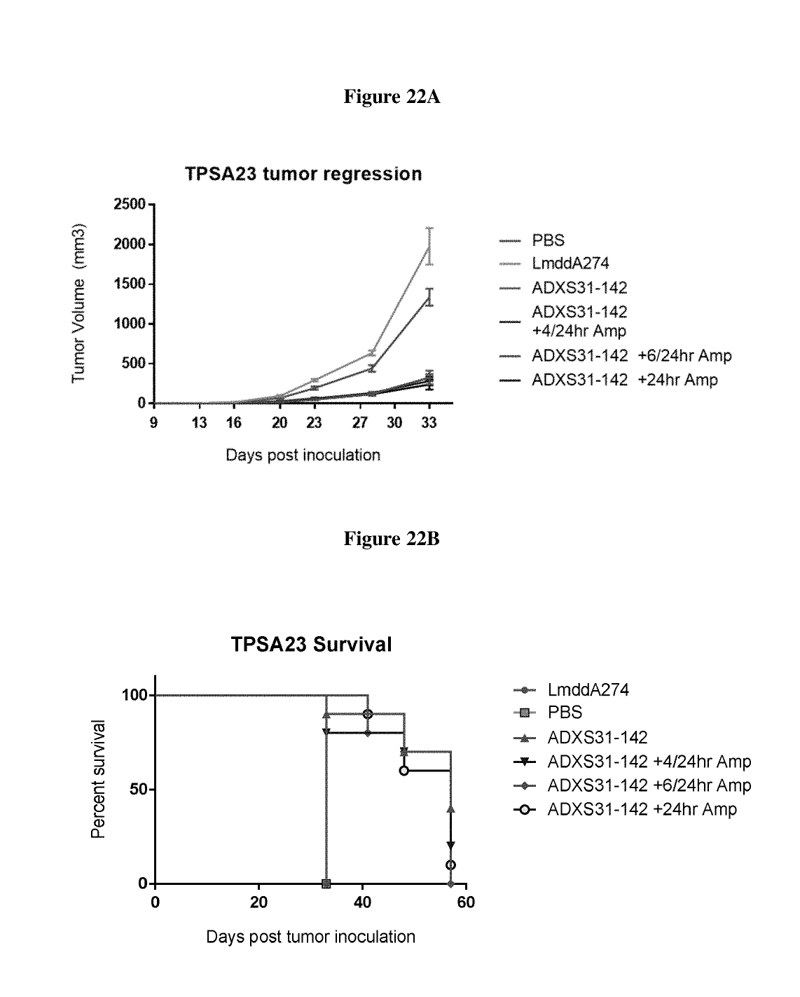

No significant difference between Lm treatment groups with or without ampicillin treatment for tumor regression (FIG. 22A) and survival (FIG. 22B).

Having described the embodiments of the disclosure with reference to the accompanying drawings, it is to be understood that the disclosure is not limited to the precise embodiments, and that various changes and modifications may be effected therein by those skilled in the art without departing from the scope or spirit of the disclosure as defined in the appended claims.

What is claimed is:

1. A method of preventing persistence of a Listeria strain on a tissue within a subject having a disease following administration of a Listeria-based immunotherapy regimen, the method comprising the step of administering an effective amount of a regimen of antibiotics following administration of said recombinant Listeria-based immunotherapy, wherein said Listeria strain comprises a nucleic acid molecule, said nucleic acid molecule comprising an open reading frame encoding a recombinant polypeptide, said recombinant polypeptide comprising a heterologous antigen or fragment thereof fused to an immunogenic protein or peptide, thereby preventing said persistence of said Listeria strain within said subject.

2. A method of preventing persistence of a Listeria strain on a tissue within a subject having a disease following administration of a Listeria-based immunotherapy regimen, the method comprising the step of administering an effective amount of a regimen of antibiotics following administration of said recombinant Listeria-based immunotherapy, wherein said Listeria strain comprises a nucleic acid molecule, said nucleic acid molecule comprising an open reading frame encoding one or more peptides encoding one or more neoepitopes, wherein said one or more peptides are fused to an immunogenic protein or peptide, thereby preventing said persistence of said Listeria strain within said subject.

3. A method of preventing persistence of a Listeria strain on a tissue within a subject having a disease following administration of a Listeria-based immunotherapy regimen, the method comprising the step of administering an effective amount of a regimen of antibiotics following administration of said recombinant Listeria-based immunotherapy, wherein said Listeria strain comprises a nucleic acid molecule, said nucleic acid molecule comprising an open reading frame encoding a recombinant polypeptide, said recombinant polypeptide comprising an immunogenic protein or peptide not fused to a heterologous antigen, thereby preventing said persistence of said Listeria strain within said subject.

4. The method of any one of claims 1-3, wherein said immunogenic protein or peptide comprises a truncated LLO protein, a truncated ActA protein or a PEST peptide.

5. The method of any one of claims 1-4, wherein administering said antibiotic regimen prevents seeding or adherence of said Listeria strain.

6. The method of any one of claims 1-4, wherein administering said antibiotic regimen prevents biofilm formation of said Listeria strain.

7. The method of any one of claims 1-6, wherein said antibiotic regimen comprises at least one of the following: clindamycin, gentamicin, azithromycin, vancomycin, phosphomycin, linezolid, rifampicin, minocycline, telithromycin, pefloxacin, a beta-lactam, fusidic acid, a macrolide, a fluoroquinolone, Meropenam Both, Moxifloxacin Both, ampicillin, dapzone, trimethoprim/sulfa (Bactrim) or any combination thereof.

8. The method of claim 7, wherein the antibiotic is poorly taken up within intact cells.

9. The method of claim 7, wherein the antibiotic is able to penetrate cells in order to clear intracellular bacteria.

10. The method of any one of claims 1-9, wherein said administering of said antibiotic regimen comprises doing so within about 1 hour to about 8 hours following administration of said recombinant Listeria strain immunotherapy.

11. The method of any one of claims 1-9, wherein said administering of said antibiotic regimen comprises doing so within about 1 hour to about 6 hours following administration of said recombinant Listeria strain immunotherapy.

12. The method of any one of claims 1-9, wherein said administering of said antibiotic regimen comprises doing so within about 1 hour to about 4 hours following administration of said recombinant Listeria strain immunotherapy.

13. The method of any one of claims 1-9, wherein said administering of said antibiotic regimen comprises doing so within about 1 hour to about 12 hours following administration of said recombinant Listeria strain immunotherapy.

14. The method of any one of claims 1-9, wherein said administering of said antibiotic regimen comprises doing so within about 2 hour to about 8 hours following administration of said recombinant Listeria strain immunotherapy.

15. The method of any one of claims 1-9, wherein said administering of said antibiotic regimen comprises doing so within about 2 hour to about 6 hours following administration of said recombinant Listeria strain immunotherapy.

16. The method of any one of claims 1-9, wherein said administering of said antibiotic regimen comprises doing so within about 2 hour to about 4 hours following administration of said recombinant Listeria strain immunotherapy.

17. The method of any one of claims 1-9, wherein said administering of said antibiotic regimen comprises doing so within about 1 hour to about 24 hours following administration of said recombinant Listeria strain immunotherapy.

18. The method of any one of claims 1-9, wherein said administering of said antibiotic regimen comprises doing so within 2-24 hours following administration of said recombinant Listeria strain immunotherapy or until said Listeria strain is eradicated from said subject but after antigen has been presented in said subject.

19. The method of any one of claims 1-9, wherein administration of said antibiotic regimen comprises administration after a therapeutic goal resulting from said administration of said Listeria strain immunotherapy has been achieved.

20. The method of claim 19, wherein said therapeutic goal comprises achieving an anti-disease immune response.

21. The method of claim 20, wherein said therapeutic goal comprises achieving tumor or cancer regression.

22. The method of any one of claims 1-21, wherein said Listeria strain immunotherapy that is administered to a subject elicits an anti-disease immune response in said subject.

23. The method of any one of claims 1-22, wherein administration of said antibiotic regimen comprises administration after said anti-disease response has initiated.

24. The method of any one of claims 1-22, wherein said administering of said antibiotic regimen does not interfere with said anti-disease immune response in said subject.

25. The method of claim 18, wherein said administering of said antibiotic regimen clears the presence of said Listeria strain within said subject.

26. The method of claim 1, wherein said heterologous antigen comprises a PSA antigen, a chimeric HER2 antigen, an HPV strain 16 E7 or an HPV strain 18 E7.

27. The method of claim 26, wherein said PSA comprises SEQ ID NO: 8.

28. The method of claim 26, wherein said chimeric HER2 comprises SEQ ID NO: 17.

29. The method of claim 26 wherein said HPV-E7 antigen comprises SEQ ID NO: 22.

30. The method of claim 26, wherein said recombinant polypeptide comprises a truncated LLO fused to a PSA antigen comprising the amino acid sequence set forth in SEQ ID NO:

15.

31. The method of claim 26, wherein said recombinant polypeptide comprises a truncated LLO fused to a cHER2 antigen comprising the amino acid sequence set forth in SEQ ID NO:

21.

32. The method of claim 26, wherein said recombinant polypeptide comprises a truncated LLO fused to an HPV-E7 antigen comprising the amino acid sequence set forth in SEQ ID NO: 23.

33. The method of claim 2, wherein said one or more neoepitopes are present in a disease or condition-bearing tissue or cell of a subject having said disease or condition.

34. The method of any one of claims 1-33, wherein said nucleic acid molecule is in a plasmid in said recombinant Listeria strain.

35. The method of claim 34, wherein said plasmid is an integrative plasmid.

36. The method of claim 34, wherein said plasmid is an episomal plasmid.

37. The method of claim 34, wherein said plasmid is stably maintained in said recombinant Listeria strain in the absence of antibiotic selection.

38. The method of any one of claims 34-37, wherein said plasmid does not confer antibiotic resistance upon said recombinant Listeria.

39. The method of any one of claims 1-38, wherein said recombinant Listeria strain is attenuated.

40. The method of claim 39, wherein said attenuated Listeria comprises a mutation, deletion, replacement, disruption or inactivation in an endogenous gene or genes.

41. The method of claim 40, wherein said endogenous gene comprises an actA virulence gene.

42. The method of any one of claims 40-41, wherein said endogenous gene comprises a D-alanine racemase (Dal) gene or a D-amino acid transferase (Dat) gene.

43. The method of any one of claims 40-42, wherein said endogenous genes comprise the actA, dal, and dat genes.

44. The method of any one of claims 1-43, wherein said recombinant nucleic acid molecule in said Listeria strain comprises a second open reading frame.

45. The method of claim 44, wherein said second open reading frame encodes a metabolic enzyme.

46. The method of claim 45, wherein said metabolic is an alanine racemase enzyme or a D-amino acid transferase enzyme.

47. The method of any one of claim 1 or 3, wherein said recombinant polypeptide is expressed from an hly promoter, a prf4 promoter, an actA promoter, or a p60 promoter.

48. The method of claim 2, wherein said one or more peptides are expressed from an hly promoter, a prf4 promoter, an actA promoter, or a p60 promoter.

49. The method of any one of claims 1-48, wherein said recombinant Listeria strain is a recombinant Listeria monocytogenes strain.

50. The method of any one of claims 1-49, wherein said recombinant Listeria strain has been passaged through an animal host.

51. The method of any one of claims 1-2, wherein said administration induces epitope spreading to additional tumor antigens.

52. The method of any one of claims 1-51, wherein said disease comprises a tumor or cancer, a premalignant condition, an infectious disease or a parasitic disease.

53. The method of claim 52, wherein said tumor or cancer comprises a breast tumor or cancer, a gastric tumor or cancer, an prostate tumor or cancer, a brain tumor or cancer, a cervical tumor or cancer, an endometrial tumor or cancer, a glioblastoma, a lung cancer, a bladder tumor or cancer, a pancreatic tumor or cancer, melanoma, a colorectal tumor or cancer, or any combination thereof.

54. The method of claim 53, wherein said tumor or said cancer is a metastasis.

55. The method of any one of claims 53-54 wherein said method comprises treating a subject having said tumor or cancer.

56. The method of claim 55, wherein said treating reduces or halts the growth of said tumor or said cancer.

57. The method of any one of claims 55-56, wherein said treating reduces or halts metastasis of said tumor or said cancer.

58. The method of any one of claims 55-57, wherein said treating elicits and maintains an anti-tumor or anti-cancer immune response in said subject.

59. The method of any one of claims 55-58, wherein said treating extends the survival time of said subject.


SEQUENCE LISTING (53 sequences)

SEQ ID NO: 1 - DNA, 6733 bp, Artificial Sequence
  recombinant nucleic acid backbone of plasmid
ggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagactagttctagatttatcacgtacccatttccccgcatcttttatttttttaaatactttagggaaaaatggtttttgatttgcttttaaaggttgtggtgtagactcgtctgctgactgcatgctagaatctaagtcactttcagaagcatccacaactgactctttcgccacttttctcttatttgcttttgttggtttatctggataagtaaggctttcaagctcactatccgacgacgctatggcttttcttctttttttaatttccgctgcgctatccgatgacagacctggatgacgacgctccacttgcagagttggtcggtcgactcctgaagcctcttcatttatagccacatttcctgtttgctcaccgttgttattattgttattcggacctttctctgcttttgctttcaacattgctattaggtctgctttgttcgtatttttcactttattcgatttttctagttcctcaatatcacgtgaacttacttcacgtgcagtttcgtatcttggtcccgtatttacctcgcttggctgctcttctgttttttcttcttcccattcatctgtgtttagactggaatcttcgctatctgtcgctgcaaatattatgtcggggttaatcgtaatgcagttggcagtaatgaaaactaccatcatcgcacgcataaatctgtttaatcccacttatactccctcctcgtgatacgctaatacaacctttttagaacaaggaaaattcggccttcattttcactaatttgttccgttaaaaattggattagcagttagttatcttcttaattagctaatataagaaaaaatattcatgaattattttaagaatatcacttggagaattaatttttctctaacatttgttaatcagttaaccccaactgcttcccaagcttcacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcacggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaat

SEQ ID NO: 2 - protein, 441 aa, Listeria monocytogenes
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 3 - protein, 416 aa, Listeria monocytogenes
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTD

SEQ ID NO: 4 - protein, 529 aa, Listeria monocytogenes
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 5 - protein, 416 aa, Listeria monocytogenes; identical to SEQ ID NO: 3

SEQ ID NO: 6 - protein, 529 aa, Listeria monocytogenes
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 7 - protein, 261 aa, Artificial Sequence
  KLK3 protein
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 8 - protein, 237 aa, Artificial Sequence
  KLK3 protein
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANP

SEQ ID NO: 9 - protein, 237 aa, Homo sapiens
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 10 - DNA, 5873 bp, Homo sapiens
ggtgtcttaggcacactggtcttggagtgcaaaggatctaggcacgtgaggctttgtatgaagaatcggggatcgtacccaccccctgtttctgtttcatcctgggcatgtctcctctgcctttgtcccctagatgaagtctccatgagctacaagggcctggtgcatccagggtgatctagtaattgcagaacagcaagtgctagctctccctccccttccacagctctgggtgtgggagggggttgtccagcctccagcagcatggggagggccttggtcagcctctgggtgccagcagggcaggggcggagtcctggggaatgaaggttttatagggctcctgggggaggctccccagccccaagcttaccacctgcacccggagagctgtgtcaccatgtgggtcccggttgtcttcctcaccctgtccgtgacgtggattggtgagaggggccatggttggggggatgcaggagagggagccagccctgactgtcaagctgaggctctttcccccccaacccagcaccccagcccagacagggagctgggctcttttctgtctctcccagccccacttcaagcccatacccccagtcccctccatattgcaacagtcctcactcccacaccaggtccccgctccctcccacttaccccagaactttcttcccatttgcccagccagctccctgctcccagctgctttactaaaggggaagttcctgggcatctccgtgtttctctttgtggggctcaaaacctccaaggacctctctcaatgccattggttccttggaccgtatcactggtccatctcctgagcccctcaatcctatcacagtctactgacttttcccattcagctgtgagtgtccaaccctatcccagagaccttgatgcttggcctcccaatcttgccctaggatacccagatgccaaccagacacctccttctttcctagccaggctatctggcctgagacaacaaatgggtccctcagtctggcaatgggactctgagaactcctcattccctgactcttagccccagactcttcattcagtggcccacattttccttaggaaaaacatgagcatccccagccacaactgccagctctctgagtccccaaatctgcatccttttcaaaacctaaaaacaaaaagaaaaacaaataaaacaaaaccaactcagaccagaactgttttctcaacctgggacttcctaaactttccaaaaccttcctcttccagcaactgaacctcgccataaggcacttatccctggttcctagcaccccttatcccctcagaatccacaacttgtaccaagtttcccttctcccagtccaagaccccaaatcaccacaaaggacccaatccccagactcaagatatggtctgggcgctgtcttgtgtctcctaccctgatccctgggttcaactctgctcccagagcatgaagcctctccaccagcaccagccaccaacctgcaaacctagggaagattgacagaattcccagcctttcccagctccccctgcccatgtcccaggactcccagccttggttctctgcccccgtgtcttttcaaacccacatcctaaatccatctcctatccgagtcccccagttccccctgtcaaccctgattcccctgatctagcaccccctctgcaggcgctgcgcccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaagtgagtaggggcctggggtctggggagcaggtgtctgtgtcccagaggaataacagctgggcattttccccaggataacctctaaggccagccttgggactgggggagagagggaaagttctggttcaggtcacatggggaggcagggttggggctggaccaccctccccatggctgcctgggtctccatctgtgtccctctatgtctctttgtgtcgctttcattatgtctcttggtaactggcttcggttgtgtctctccgtgtgactattttgttctctctctccctctcttctctgtcttcagtctccatatctccccctctctctgtccttctctggtccctctctagccagtgtgtctcaccctgtatctctctgccaggctctgtctctcggtctctgtctcacctgtgccttctccctactgaacacacgcacgggatgggcctgggggaccctgagaaaaggaagggctttggctgggcgcggtggctcacacctgtaatcccagcactttgggaggccaaggcaggtagatcacctgaggtcaggagttcgagaccagcctggccaactggtgaaaccccatctctactaaaaatacaaaaaattagccaggcgtggtggcgcatgcctgtagtcccagctactcaggagctgagggaggagaattgcattgaacctggaggttgaggttgcagtgagccgagaccgtgccactgcactccagcctgggtgacagagtgagactccgcctcaaaaaaaaaaaaaaaaaaaaaaaaaaaaaagaaaagaaaagaaaagaaaaggaagtgttttatccctgatgtgtgtgggtatgagggtatgagagggcccctctcactccattccttctccaggacatccctccactcttgggagacacagagaagggctggttccagctggagctgggaggggcaattgagggaggaggaaggagaagggggaaggaaaacagggtatgggggaaaggaccctggggagcgaagtggaggatacaaccttgggcctgcaggcaggctacctacccacttggaaacccacgccaaagccgcatctacagctgagccactctgaggcctcccctccccggcggtccccactcagctccaaagtctctctcccttttctctcccacactttatcatcccccggattcctctctacttggttctcattcttcctttgacttcctgcttccctttctcattcatctgtttctcactttctgcctggttttgttcttctctctctctttctctggcccatgtctgtttctctatgtttctgtcttttctttctcatcctgtgtattttcggctcaccttgtttgtcactgttctcccctctgccctttcattctctctgcccttttaccctcttccttttcccttggttctctcagttctgtatctgcccttcaccctctcacactgctgtttcccaactcgttgtctgtattttggcctgaactgtgtcttcccaaccctgtgttttctcactgtttctttttctcttttggagcctcctccttgctcctctgtcccttctctctttccttatcatcctcgctcctcattcctgcgtctgcttcctccccagcaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagtgtacgcctgggccagatggtgcagccgggagcccagatgcctgggtctgagggaggaggggacaggactcctgggtctgagggaggagggccaaggaaccaggtggggtccagcccacaacagtgtttttgcctggcccgtagtcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcggtgagtcatccctactcccaagatcttgagggaaaggtgagtgggaccttaattctgggctggggtctagaagccaacaaggcgtctgcctcccctgctccccagctgtagccatgccacctccccgtgtctcatctcattccctccttccctcttctttgactccctcaaggcaataggttattcttacagcacaactcatctgttcctgcgttcagcacacggttactaggcacctgctatgcacccagcactgccctagagcctgggacatagcagtgaacagacagagagcagcccctcccttctgtagcccccaagccagtgaggggcacaggcaggaacagggaccacaacacagaaaagctggagggtgtcaggaggtgatcaggctctcggggagggagaaggggtggggagtgtgactgggaggagacatcctgcagaaggtgggagtgagcaaacacctgcgcaggggaggggagggcctgcggcacctgggggagcagagggaacagcatctggccaggcctgggaggaggggcctagagggcgtcaggagcagagaggaggttgcctggctggagtgaaggatcggggcagggtgcgagagggaacaaaggacccctcctgcagggcctcacctgggccacaggaggacactgcttttcctctgaggagtcaggaactgtggatggtgctggacagaagcaggacagggcctggctcaggtgtccagaggctgcgctggcctcctatgggatcagactgcagggagggagggcagcagggatgtggagggagtgatgatggggctgacctgggggtggctccaggcattgtccccacctgggcccttacccagcctccctcacaggctcctggccctcagtctctcccctccactccattctccacctacccacagtgggtcattctgatcaccgaactgaccatgccagccctgccgatggtcctccatggctccctagtgccctggagaggaggtgtctagtcagagagtagtcctggaaggtggcctctgtgaggagccacggggacagcatcctgcagatggtcctggcccttgtcccaccgacctgtctacaaggactgtcctcgtggaccctcccctctgcacaggagctggaccctgaagtcccttcctaccggccaggactggagcccctacccctctgttggaatccctgcccaccttcttctggaagtcggctctggagacatttctctcttcttccaaagctgggaactgctatctgttatctgcctgtccaggtctgaaagataggattgcccaggcagaaactgggactgacctatctcactctctccctgcttttacccttagggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaagtccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagttaggatggggtgtctgtgttatttgtgggatacagagatgaaagaggggtgggatcc

SEQ ID NO: 11 - protein, 238 aa, Artificial Sequence
  KLK3 protein
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSWVILITELTMPALPMVLHGSLVPWRGGV

SEQ ID NO: 12 - DNA, 1906 bp, Homo sapiens
agccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgtgggtcattctgatcaccgaactgaccatgccagccctgccgatggtcctccatggctccctagtgccctggagaggaggtgtctagtcagagagtagtcctggaaggtggcctctgtgaggagccacggggacagcatcctgcagatggtcctggcccttgtcccaccgacctgtctacaaggactgtcctcgtggaccctcccctctgcacaggagctggaccctgaagtcccttccccaccggccaggactggagcccctacccctctgttggaatccctgcccaccttcttctggaagtcggctctggagacatttctctcttcttccaaagctgggaactgctatctgttatctgcctgtccaggtctgaaagataggattgcccaggcagaaactgggactgacctatctcactctctccctgcttttacccttagggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtg

SEQ ID NO: 13 - DNA, 711 bp, Artificial Sequence
  KLK3 protein
attgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaacccc

SEQ ID NO: 14 - DNA, 2040 bp, Artificial Sequence
  sequence encoding a truncated LLO fused to a PSA protein
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaacccc

SEQ ID NO: 15 - protein, 680 aa, Artificial Sequence [sequence omitted]

SEQ ID NO: 16 - DNA, 1257 bp, Artificial Sequence
  sequence encoding a chimeric HER2 protein
acccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaagaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagac

SEQ ID NO: 17 - protein, 419 aa, Artificial Sequence
  chimeric HER2 protein
THLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKNIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLD

SEQ ID NO: 18 - DNA, 2850 bp, Artificial Sequence
  LmddA244G, nucleic acid sequence comprising an open reading frame encoding a cHER2 fused to an endogenous nucleic acid comprising an open reading frame encoding an LLO protein
atgaaaaaaataatgctagtttttattacacttatattagttagtctaccaattgcgcaacaaactgaagcaaaggatgcatctgcattcaataaagaaaattcaatttcatccgtggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttattcaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatcctgaaggtaacgaaattgttcaacataaaaactggagcgaaaacaataaaagcaagctagctcatttcacatcgtccatctatttgcctggtaacgcgagaaatattaatgtttacgctaaagaatgcactggtttagcttgggaatggtggagaacggtaattgatgaccggaacttaccacttgtgaaaaatagaaatatctccatctggggcaccacgctttatccgaaatatagtaataaagtagataatccaatcgaagtcgacacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaagaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagac

SEQ ID NO: 19 - protein, 950 aa, Artificial Sequence
  cHER2 and endogenous LLO fusion
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIEVDTHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKNIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLD

SEQ ID NO: 20 - DNA, 2586 bp, Artificial Sequence
  LmddA164, nucleic acid sequence comprising an open reading frame encoding tLLO fused to cHER2
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaagaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagac

SEQ ID NO: 21 - protein, 862 aa, Artificial Sequence
  recombinant protein comprising tLLO fused to a cHER2
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDLETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKNIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLD

SEQ ID NO: 22 - protein, 97 aa, Human papillomavirus type 16
HGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 23 - protein, 540 aa, Artificial Sequence
  truncated LLO fused to an E7 protein
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDLEHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 24 - protein, 14 aa, Artificial Sequence
  PEST sequence
KTEEQPSEVNTGPR

SEQ ID NO: 25 - protein, 28 aa, Artificial Sequence
  PEST sequence
KASVTDTSEGDLDSSMQSADESTPQPLK

SEQ ID NO: 26 - protein, 20 aa, Artificial Sequence
  PEST sequence
KNEEVNASDFPPPPTDEELR

SEQ ID NO: 27 - protein, 33 aa, Artificial Sequence
  PEST sequence
RGGIPTSEEFSSLNSGDFTDDENSETTEEEIDR

SEQ ID NO: 28 - protein, 19 aa, Artificial Sequence
  PEST sequence
RSEVTISPAETPESPPATP

SEQ ID NO: 29 - protein, 17 aa, Streptococcus pyogenes
KQNTASTETTTTNEQPK

SEQ ID NO: 30 - protein, 17 aa, Streptococcus equisimilis
KQNTANTETTTTNEQPK

SEQ ID NO: 31 - protein, 390 aa, Listeria monocytogenes
MRAMMVVFITANCITINPDIIFAATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRP

SEQ ID NO: 32 - DNA, 1170 bp, Artificial Sequence
  recombinant sequence encoding a fragment of an ActA protein
atgcgtgcgatgatggtggttttcattactgccaattgcattacgattaaccccgacataatatttgcagcgacagatagcgaagattctagtctaaacacagatgaatgggaagaagaaaaaacagaagagcaaccaagcgaggtaaatacgggaccaagatacgaaactgcacgtgaagtaagttcacgtgatattaaagaactagaaaaatcgaataaagtgagaaatacgaacaaagcagacctaatagcaatgttgaaagaaaaagcagaaaaaggtccaaatatcaataataacaacagtgaacaaactgagaatgcggctataaatgaagaggcttcaggagccgaccgaccagctatacaagtggagcgtcgtcatccaggattgccatcggatagcgcagcggaaattaaaaaaagaaggaaagccatagcatcatcggatagtgagcttgaaagccttacttatccggataaaccaacaaaagtaaataagaaaaaagtggcgaaagagtcagttgcggatgcttctgaaagtgacttagattctagcatgcagtcagcagatgagtcttcaccacaacctttaaaagcaaaccaacaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccaatgcttcttggttttaatgctcctgctacatcagaaccgagctcattcgaatttccaccaccacctacggatgaagagttaagacttgctttgccagagacgccaatgcttcttggttttaatgctcctgctacatcggaaccgagctcgttcgaatttccaccgcctccaacagaagatgaactagaaatcatccgggaaacagcatcctcgctagattctagttttacaagaggggatttagctagtttgagaaatgctattaatcgccatagtcaaaatttctctgatttcccaccaatcccaacagaagaagagttgaacgggagaggcggtagacca

SEQ ID NO: 33 - protein, 390 aa, Listeria monocytogenes
MRAMMVVFITANCITINPDIIFAATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRP

SEQ ID NO: 34 - protein, 200 aa, Listeria monocytogenes
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDK

SEQ ID NO: 35 - protein, 226 aa, Artificial Sequence
  truncated ActA fused to hly signal peptide
MKKIMLVFITLILVSLPIAQQTEASRATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDK

SEQ ID NO: 36 - DNA, 1170 bp, Listeria monocytogenes
atgcgtgcgatgatggtagttttcattactgccaactgcattacgattaaccccgacataatatttgcagcgacagatagcgaagattccagtctaaacacagatgaatgggaagaagaaaaaacagaagagcagccaagcgaggtaaatacgggaccaagatacgaaactgcacgtgaagtaagttcacgtgatattgaggaactagaaaaatcgaataaagtgaaaaatacgaacaaagcagacctaatagcaatgttgaaagcaaaagcagagaaaggtccgaataacaataataacaacggtgagcaaacaggaaatgtggctataaatgaagaggcttcaggagtcgaccgaccaactctgcaagtggagcgtcgtcatccaggtctgtcatcggatagcgcagcggaaattaaaaaaagaagaaaagccatagcgtcgtcggatagtgagcttgaaagccttacttatccagataaaccaacaaaagcaaataagagaaaagtggcgaaagagtcagttgtggatgcttctgaaagtgacttagattctagcatgcagtcagcagacgagtctacaccacaacctttaaaagcaaatcaaaaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccgatgcttctcggttttaatgctcctactccatcggaaccgagctcattcgaatttccgccgccacctacggatgaagagttaagacttgctttgccagagacgccaatgcttcttggttttaatgctcctgctacatcggaaccgagctcattcgaatttccaccgcctccaacagaagatgaactagaaattatgcgggaaacagcaccttcgctagattctagttttacaagcggggatttagctagtttgagaagtgctattaatcgccatagcgaaaatttctctgatttcccactaatcccaacagaagaagagttgaacgggagaggcggtagacca

SEQ ID NO: 37 - DNA, 1256 bp, Artificial Sequence
  deleted actA region in the strain, Lmdd[delta]actA
gcgccaaatcattggttgattggtgaggatgtctgtgtgcgtgggtcgcgagatgggcgaataagaagcattaaagatcctgacaaatataatcaagcggctcatatgaaagattacgaatcgcttccactcacagaggaaggcgactggggcggagttcattataatagtggtatcccgaataaagcagcctataatactatcactaaacttggaaaagaaaaaacagaacagctttattttcgcgccttaaagtactatttaacgaaaaaatcccagtttaccgatgcgaaaaaagcgcttcaacaagcagcgaaagatttatatggtgaagatgcttctaaaaaagttgctgaagcttgggaagcagttggggttaactgattaacaaatgttagagaaaaattaattctccaagtgatattcttaaaataattcatgaatattttttcttatattagctaattaagaagataactaactgctaatccaatttttaacggaacaaattagtgaaaatgaaggccgaattttccttgttctaaaaaggttgtattagcgtatcacgaggagggagtataagtgggattaaacagatttatgcgtgcgatgatggtggttttcattactgccaattgcattacgattaaccccgacgtcgacccatacgacgttaattcttgcaatgttagctattggcgtgttctctttaggggcgtttatcaaaattattcaattaagaaaaaataattaaaaacacagaacgaaagaaaaagtgaggtgaatgatatgaaattcaaaaaggtggttctaggtatgtgcttgatcgcaagtgttctagtctttccggtaacgataaaagcaaatgcctgttgtgatgaatacttacaaacacccgcagctccgcatgatattgacagcaaattaccacataaacttagttggtccgcggataacccgacaaatactgacgtaaatacgcactattggctttttaaacaagcggaaaaaatactagctaaagatgtaaatcatatgcgagctaatttaatgaatgaacttaaaaaattcgataaacaaatagctcaaggaatatatgatgcggatcataaaaatccatattatgatactagtacatttttatctcatttttataatcctgatagagataatacttatttgccgggttttgctaatgcgaaaataacaggagcaaagtatttcaatcaatcggtgactgattaccgagaagggaa

SEQ ID NO: 38 - DNA, 1107 bp, Listeria monocytogenes
atggtgacaggctggcatcgtccaacatggattgaaatagaccgcgcagcaattcgcgaaaatataaaaaatgaacaaaataaactcccggaaagtgtcgacttatgggcagtagtcaaagctaatgcatatggtcacggaattatcgaagttgctaggacggcgaaagaagctggagcaaaaggtttctgcgtagccattttagatgaggcactggctcttagagaagctggatttcaagatgactttattcttgtgcttggtgcaaccagaaaagaagatgctaatctggcagccaaaaaccacatttcacttactgtttttagagaagattggctagagaatctaacgctagaagcaacacttcgaattcatttaaaagtagatagcggtatggggcgtctcggtattcgtacgactgaagaagcacggcgaattgaagcaaccagtactaatgatcaccaattacaactggaaggtatttacacgcattttgcaacagccgaccagctagaaactagttattttgaacaacaattagctaagttccaaacgattttaacgagtttaaaaaaacgaccaacttatgttcatacagccaattcagctgcttcattgttacagccacaaatcgggtttgatgcgattcgctttggtatttcgatgtatggattaactccctccacagaaatcaaaactagcttgccgtttgagcttaaacctgcacttgcactctataccgagatggttcatgtgaaagaacttgcaccaggcgatagcgttagctacggagcaacttatacagcaacagagcgagaatgggttgcgacattaccaattggctatgcggatggattgattcgtcattacagtggtttccatgttttagtagacggtgaaccagctccaatcattggtcgagtttgtatggatcaaaccatcataaaactaccacgtgaatttcaaactggttcaaaagtaacgataattggcaaagatcatggtaacacggtaacagcagatgatgccgctcaatatttagatacaattaattatgaggtaacttgtttgttaaatgagcgcatacctagaaaatacatccattag

SEQ ID NO: 39 - protein, 368 aa, Listeria monocytogenes
MVTGWHRPTWIEIDRAAIRENIKNEQNKLPESVDLWAVVKANAYGHGIIEVARTAKEAGAKGFCVAILDEALALREAGFQDDFILVLGATRKEDANLAAKNHISLTVFREDWLENLTLEATLRIHLKVDSGMGRLGIRTTEEARRIEATSTNDHQLQLEGIYTHFATADQLETSYFEQQLAKFQTILTSLKKRPTYVHTANSAASLLQPQIGFDAIRFGISMYGLTPSTEIKTSLPFELKPALALYTEMVHVKELAPGDSVSYGATYTATEREWVATLPIGYADGLIRHYSGFHVLVDGEPAPIIGRVCMDQTIIKLPREFQTGSKVTIIGKDHGNTVTADDAAQYLDTINYEVTCLLNERIPRKYIH

SEQ ID NO: 40 - DNA, 870 bp, Listeria monocytogenes
atgaaagtattagtaaataaccatttagttgaaagagaagatgccacagttgacattgaagaccgcggatatcagtttggtgatggtgtatatgaagtagttcgtctatataatggaaaattctttacttataatgaacacattgatcgcttatatgctagtgcagcaaaaattgacttagttattccttattccaaagaagagctacgtgaattacttgaaaaattagttgccgaaaataatatcaatacagggaatgtctatttacaagtgactcgtggtgttcaaaacccacgtaatcatgtaatccctgatgatttccctctagaaggcgttttaacagcagcagctcgtgaagtacctagaaacgagcgtcaattcgttgaaggtggaacggcgattacagaagaagatgtgcgctggttacgctgtgatattaagagcttaaaccttttaggaaatattctagcaaaaaataaagcacatcaacaaaatgctttggaagctattttacatcgcggggaacaagtaacagaatgttctgcttcaaacgtttctattattaaagatggtgtattatggacgcatgcggcagataacttaatcttaaatggtatcactcgtcaagttatcattgatgttgcgaaaaagaatggcattcctgttaaagaagcggatttcactttaacagaccttcgtgaagcggatgaagtgttcatttcaagtacaactattgaaattacacctattacgcatattgacggagttcaagtagctgacggaaaacgtggaccaattacagcgcaacttcatcaatattttgtagaagaaatcactcgtgcatgtggcgaattagagtttgcaaaataa

SEQ ID NO: 41 - protein, 289 aa, Listeria monocytogenes
MKVLVNNHLVEREDATVDIEDRGYQFGDGVYEVVRLYNGKFFTYNEHIDRLYASAAKIDLVIPYSKEELRELLEKLVAENNINTGNVYLQVTRGVQNPRNHVIPDDFPLEGVLTAAAREVPRNERQFVEGGTAITEEDVRWLRCDIKSLNLLGNILAKNKAHQQNALEAILHRGEQVTECSASNVSIIKDGVLWTHAADNLILNGITRQVIIDVAKKNGIPVKEADFTLTDLREADEVFISSTTIEITPITHIDGVQVADGKRGPITAQLHQYFVEEITRACGELEFAK

SEQ ID NO: 42 - DNA, 6523 bp, Artificial Sequence
  plasmid pAdv142
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaacccctaacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcgcggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaattaa

SEQ ID NO: 43 - DNA, 36 bp, Artificial Sequence
  Primer
cggaattcggatccgcgccaaatcattggttgattg

SEQ ID NO: 44 - DNA, 37 bp, Artificial Sequence
  Primer
gcgagtcgacgtcggggttaatcgtaatgcaattggc

SEQ ID NO: 45 - DNA, 35 bp, Artificial Sequence
  Primer
gcgagtcgacccatacgacgttaattcttgcaatg

SEQ ID NO: 46 - DNA, 39 bp, Artificial Sequence
  Primer
gatactgcagggatccttcccttctcggtaatcagtcac

SEQ ID NO: 47 - DNA, 19 bp, Artificial Sequence
  Primer
tgggatggccaagaaattc

SEQ ID NO: 48 - DNA, 22 bp, Artificial Sequence
  Primer
ctaccatgtcttccgttgcttg

SEQ ID NO: 49 - DNA, 7075 bp, Artificial Sequence
  pAdv84
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaagaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagactaatctagacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcgcggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaattaa

SEQ ID NO: 50 - protein, 9 aa, Artificial Sequence
  peptide corresponding to mapped HLA-A2 restricted epitopes located at the extracellular domain of the Her2/neu molecule
HLYQGCQVV

SEQ ID NO: 51 - protein, 9 aa, Artificial Sequence
  peptide corresponding to mapped HLA-A2 restricted epitopes located at the extracellular domain of the Her2/neu molecule
KIFGSLAFL

SEQ ID NO: 52 - protein, 9 aa, Artificial Sequence
  peptide corresponding to mapped HLA-A2 restricted epitopes located at the intracellular domain of the Her2/neu molecule
RLLQETELV

SEQ ID NO: 53 - protein, 24 aa, Artificial Sequence
  KLK3 protein
MWVPVVFLTLSVTWIGAAPLILSR